(19) United States
(12) Patent Application Publication
(10) Pub. No.: US 2023/0270857 A1
(43) Pub. Date: Aug. 31, 2023

(54) COMPOSITIONS INCLUDING EX VIVO ARMED T CELLS WITH MULTI-SPECIFIC ANTIBODIES AND USES THEREOF

(71) Applicant: Memorial Sloan Kettering Cancer Center, New York, NY (US)

(72) Inventors: Jeong A. Park, New York, NY (US); Nai-Kong V. Cheung, New York, NY (US); Brian Santich, New York, NY (US)

(21) Appl. No.: 18/007,292

(22) PCT Filed: Jul. 27, 2021
(86) PCT No.: PCT/US2021/043270

§ 371 Date: Jan. 27, 2023

Related U.S. Application Data
(60) Provisional application No. 63/057,871, filed on Jul. 28, 2020.

(51) Int. Cl. A61K 39/00; C07K 16/28; A61K 51/10; A61K 39/395; A61P 35/00; C12N 5/0783; C12N 5/00

(52) U.S. Cl. CPC A61K 39/4611; C07K 16/2809; A61K 51/1093; A61K 39/4633; A61K 39/3955; A61K 39/464406; A61K 39/464471; A61P 35/00; C12N 5/0636; C12N 5/0006; C07K 2317/31; C07K 2317/622; A61K 2239/29; C12N 2502/30

(57) ABSTRACT

The present disclosure provides ex vivo armed T cell (EAT) compositions that comprise multi-specific (e.g., bispecific) antibodies that bind to CDS and at least one additional target antigen (e.g., antigen that is expressed by tumor cells and/or a DOTA label). The EAT compositions of the present technology are useful for adoptive immunotherapy in a subject in need thereof.

### CROSS-REFERENCE TO RELATED APPLICATIONS

This application is a U.S. National Stage Application under 35 U.S.C. § 371 of International Patent Application No. PCT/US2021/043270, filed Jul. 27, 2021, which claims the benefit of and priority to U.S. Provisional Appl. No. 63/057,871, filed Jul. 28, 2020, the disclosure of which is incorporated by reference herein in its entirety.

### STATEMENT OF GOVERNMENT SUPPORT

This invention was made with government support under CA008748, awarded by the National Cancer Institute. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 3, 2021, is named 115872-2263_SL.txt and is 439,087 bytes in size.

### TECHNICAL FIELD

The present technology relates generally to the preparation of compositions including T cells that are armed ex vivo with multi-specific (e.g., bispecific) antibodies, and their use in adoptive immunotherapy.

### BACKGROUND

The following description of the background of the present technology is provided simply as an aid in understanding the present technology and is not admitted to describe or constitute prior art to the present technology.

T cell-based immunotherapy using chimeric antigen receptor (CAR) or T cell engaging bispecific antibody (BsAb) has shown great promise in treating human cancers. However, considerable hurdles still exist. T cells and NK cells in cancer patients are dysfunctional or weak and unable to traffick into tumors to exploit their full capacity. See C. Menetrier-Caux et al., Journal for Immunotherapy Cancer 7, 85 (2019). Many tumor targets are heterogeneous and prone to downregulation or loss, whereby initial responses are not durable. Conversely, broad T cell activation, particularly for CAR T cell therapy, is often difficult to control, causing ‘on-target’ and ‘off-tumor’ toxicities which could be life-threatening, including cytokine release syndrome (CRS) and neurotoxicities (Klebanoff et al., Nat Med 22(1): 26-36 (2016)). One of the major toxicities of T cell based immunotherapy is cytokine release syndrome (CRS) (D. W. Lee et al., Blood 124, 188 (2014)), typically associated with IFN-γ, IL-6, and TNF-α release, although elevations of IL-2, GM-CSF, IL-10, IL-8, and IL-5 have also been reported (S. A. Grupp et al., N Engl J Med 368, 1509 (2013); J. N. Kochenderfer et al., Blood 119, 2709 (2012)). But most of all, structural designs for the optimal BsAb or CAR in solid tumors are still exploratory. In cytotherapy, these hurdles can be further complicated by the poor persistence/survival of cellular products, and the complexity/cost of their manufacture and distribution.

### SUMMARY OF THE PRESENT TECHNOLOGY

In one aspect, the present disclosure provides an ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, wherein the at least one type of anti-CD3 multi-specific antibody is an immunoglobulin comprising two heavy chains and two light chains, wherein each of the light chains is fused to a single chain variable fragment (scFv), and wherein the ex vivo armed T cell is or has been cryopreserved. The ex vivo armed T cell may be a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell. In some embodiments, the ex vivo armed T cell has been cryopreserved for a period of about 2 hours to about 6 months. Additionally or alternatively, in some embodiments, the at least one type of anti-CD3 multi-specific antibody is a bispecific antibody, a trispecific antibody, or a tetraspecific antibody.

In one aspect, the present disclosure provides an ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, wherein the at least one type of anti-CD3 multi-specific antibody is an immunoglobulin comprising two heavy chains and two light chains, wherein each of the light chains is fused to a single chain variable fragment (scFv), and wherein the ex vivo armed T cell is a γδ T cell. In some embodiments, the ex vivo armed T cell is generated by contacting peripheral blood mononuclear cells with zoledronate and IL-15. Additionally or alternatively, in some embodiments, the IL-15 is administered as an IL15Rα-IL15 complex. Additionally or alternatively, in some embodiments, the at least one type of anti-CD3 multi-specific antibody is a bispecific antibody, a trispecific antibody, or a tetraspecific antibody.

In any of the preceding embodiments of the ex vivo armed T cell disclosed herein, at least one scFv of the at least one type of anti-CD3 multi-specific antibody comprises the CD3 binding domain. Additionally or alternatively, in some embodiments, at least one scFv of the at least one type of anti-CD3 multi-specific antibody comprises a DOTA binding domain. In certain embodiments, the DOTA binding domain comprises the amino acid sequence of any one of SEQ ID NOs: 77-80.

In one aspect, the present disclosure provides an ex vivo armed T cell that is coated or complexed with an effective arming dose of at least two types of anti-CD3 multi-specific antibodies, wherein each of the at least two types of anti-CD3 multi-specific antibodies includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and wherein each of the at least two types of anti-CD3 multi-specific antibodies is an immunoglobulin comprising two heavy chains and two light chains, wherein each of the light chains is fused to a single chain variable fragment (scFv). The ex vivo armed T cell may comprise 2, 3, 4, or 5 types of anti-CD3 multi-specific antibodies. In some embodiments, at least one scFv of each of the at least two types of anti-CD3 multi-specific antibodies comprises the CD3 binding domain. In certain embodiments, one or more of the at least two types of anti-CD3 multi-specific antibodies comprises a DOTA binding domain. In a further embodiment, one or more of the at least two types of anti-CD3 multi-specific antibodies comprise a scFv that includes the DOTA binding domain. In certain embodiments, the DOTA binding domain comprises the amino acid sequence of any one of SEQ ID NOs: 77-80. The ex vivo armed T cell may be a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell. Additionally or alternatively, in some embodiments, the at least two types of anti-CD3 multi-specific antibody is a bispecific antibody, a trispecific antibody, or a tetraspecific antibody.

In one aspect, the present disclosure provides an ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, wherein the at least one type of anti-CD3 multi-specific antibody is an immunoglobulin comprising two heavy chains and two light chains, wherein each of the light chains is fused to a single chain variable fragment (scFv), wherein at least one scFv of the at least one type of anti-CD3 multi-specific antibody comprises the CD3 binding domain, and wherein at least one scFv of the at least one type of anti-CD3 multi-specific antibody comprises a DOTA binding domain. In certain embodiments, the DOTA binding domain comprises the amino acid sequence of any one of SEQ ID NOs: 77-80. The ex vivo armed T cell may be a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell. Additionally or alternatively, in some embodiments, the at least one type of anti-CD3 multi-specific antibody is a bispecific antibody, a trispecific antibody, or a tetraspecific antibody.

In any and all embodiments of the ex vivo armed T cell described herein, the at least one type of anti-CD3 multi-specific antibody or the at least two types of anti-CD3 multi-specific antibodies bind two or more additional target antigens. Examples of additional target antigens include, but are not limited to, CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Ley) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, peptide MHC complexes (with peptides derived from TP53, KRAS, MYC, EBNA1-6, PRAME, MART, tyronsinase, MAGEA1-A6, pmel17, LMP2, or WT1), and a DOTA-based hapten.

In any and all embodiments of the ex vivo armed T cell described herein, the V_{H} of the CD3 binding domain comprises the amino acid sequence of any one of SEQ ID NOs: 7-32, and/or wherein the V_{L} of the CD3 binding domain comprises the amino acid sequence of any one of SEQ ID NOs: 33-70.

In any and all embodiments of the ex vivo armed T cell described herein, the at least one type of anti-CD3 multi-specific antibody, or one or more of the at least two types of anti-CD3 multi-specific antibodies comprise a heavy chain (HC) amino acid sequence comprising SEQ ID NO: 82, SEQ ID NO: 84, SEQ ID NO: 86, SEQ ID NO: 88, SEQ ID NO: 90, SEQ ID NO: 94, SEQ ID NO: 96, SEQ ID NO: 98, SEQ ID NO: 100, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 163, SEQ ID NO: 165, SEQ ID NO: 167, SEQ ID NO: 169, or a variant thereof having one or more conservative amino acid substitutions, and/or a light chain (LC) amino acid sequence comprising SEQ ID NO: 81, SEQ ID NO: 83, SEQ ID NO: 85, SEQ ID NO: 87, SEQ ID NO: 89, SEQ ID NO: 93, SEQ ID NO: 95, SEQ ID NO: 97, SEQ ID NO: 99, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 162, SEQ ID NO: 164, SEQ ID NO: 166, SEQ ID NO: 168, or a variant thereof having one or more conservative amino acid substitutions.

In any and all embodiments of the ex vivo armed T cell described herein, the at least one type of anti-CD3 multi-specific antibody, or one or more of the at least two types of anti-CD3 multi-specific antibodies comprise a HC amino acid sequence and a LC amino acid sequence selected from the group consisting of: SEQ ID NO: 82 and SEQ ID NO: 81, SEQ ID NO: 84 and SEQ ID NO: 83, SEQ ID NO: 86 and SEQ ID NO: 85, SEQ ID NO: 88 and SEQ ID NO: 87, SEQ ID NO: 90 and SEQ ID NO: 89, SEQ ID NO: 94 and SEQ ID NO: 93, SEQ ID NO: 96 and SEQ ID NO: 95, SEQ ID NO: 98 and SEQ ID NO: 97, SEQ ID NO: 100 and SEQ ID NO: 99, SEQ ID NO: 115 and SEQ ID NO: 114, SEQ ID NO: 117 and SEQ ID NO: 116, SEQ ID NO: 119 and SEQ ID NO: 118, SEQ ID NO: 121 and SEQ ID NO: 120, SEQ ID NO: 123 and SEQ ID NO: 122, SEQ ID NO: 125 and SEQ ID NO: 124, SEQ ID NO: 127 and SEQ ID NO: 126, SEQ ID NO: 129 and SEQ ID NO: 128, SEQ ID NO: 131 and SEQ ID NO: 130, SEQ ID NO: 133 and SEQ ID NO: 132, SEQ ID NO: 135 and SEQ ID NO: 134, SEQ ID NO: 137 and SEQ ID NO: 136, SEQ ID NO: 139 and SEQ ID NO: 138, SEQ ID NO: 141 and SEQ ID NO: 140, SEQ ID NO: 143 and SEQ ID NO: 142, SEQ ID NO: 145 and SEQ ID NO: 144, SEQ ID NO: 147 and SEQ ID NO: 146, SEQ ID NO: 149 and SEQ ID NO: 148, SEQ ID NO: 151 and SEQ ID NO: 150, SEQ ID NO: 153 and SEQ ID NO: 152, SEQ ID NO: 155 and SEQ ID NO: 154, SEQ ID NO: 157 and SEQ ID NO: 156, SEQ ID NO: 163 and SEQ ID NO: 162, SEQ ID NO: 165 and SEQ ID NO: 164, SEQ ID NO: 167 and SEQ ID NO: 166, and SEQ ID NO: 169 and SEQ ID NO: 168, respectively.

In any and all embodiments of the ex vivo armed T cell described herein, the at least one type of anti-CD3 multi-specific antibody, or one or more of the at least two types of anti-CD3 multi-specific antibodies comprise a first LC amino acid sequence, a first HC amino acid sequence, a second LC amino acid sequence, and a second HC amino acid sequence selected from the group consisting of SEQ ID NO: 114, SEQ ID NO: 115, SEQ ID NO: 116, and SEQ ID NO: 117; SEQ ID NO: 118, SEQ ID NO: 119, SEQ ID NO: 120, and SEQ ID NO: 121; SEQ ID NO: 122, SEQ ID NO: 123, SEQ ID NO: 124, and SEQ ID NO: 125; SEQ ID NO: 126, SEQ ID NO: 127, SEQ ID NO: 128, and SEQ ID NO: 129; SEQ ID NO: 130, SEQ ID NO: 131, SEQ ID NO: 132, and SEQ ID NO: 133; SEQ ID NO: 134, SEQ ID NO: 135, SEQ ID NO: 136, and SEQ ID NO: 137; SEQ ID NO: 138, SEQ ID NO: 139, SEQ ID NO: 140, and SEQ ID NO: 141; SEQ ID NO: 142, SEQ ID NO: 143, SEQ ID NO: 144, and SEQ ID NO: 145; SEQ ID NO: 146, SEQ ID NO: 147, SEQ ID NO: 148, and SEQ ID NO: 149; SEQ ID NO: 150, SEQ ID NO: 151, SEQ ID NO: 152, and SEQ ID NO: 153; SEQ ID NO: 154, SEQ ID NO: 155, SEQ ID NO: 156, and SEQ ID NO: 157; SEQ ID NO: 162, SEQ ID NO: 163, SEQ ID NO: 164, and SEQ ID NO: 165; and SEQ ID NO: 166, SEQ ID NO: 167, SEQ ID NO: 168, and SEQ ID NO: 169; respectively.

In any and all embodiments of the ex vivo armed T cell described herein, the at least one type of anti-CD3 multi-specific antibody exhibits surface densities between about 500 to about 20,000 molecules per T cell and/or the at least two types of anti-CD3 multi-specific antibodies exhibit surface densities between about 1,500 to 10,000 molecules per T cell.

In any and all embodiments of the ex vivo armed T cell described herein, the effective arming dose of the at least one type of anti-CD3 multi-specific antibody or the at least two types of anti-CD3 multi-specific antibodies is between about 0.05 μg/10⁶ T cells to about 5 μg/10⁶ T cells.

In one aspect, the present disclosure provides a method for determining the antibody binding capacity of any embodiment of the ex vivo armed T cell described herein in vitro comprising (a) contacting the ex vivo armed T cell with an agent that binds to any embodiment of the anti-CD3 multi-specific antibody disclosed herein that is present on the ex vivo armed T cell, wherein the agent is directly or indirectly linked to a detectable label, and (b) determining the antibody binding capacity of the ex vivo armed T cell by detecting the level or intensity of signal emitted by the detectable label. The detectable label may be spectroscopic, photochemical, biochemical, immunochemical, electromagnetic, radioactive, fluorescent, chemifluorescent, or chemiluminescent label. In some embodiments, the antibody binding capacity is quantified using flow cytometry.

In one aspect, the present disclosure provides a method for tracking ex vivo armed T cells in a subject in vivo comprising (a) administering to the subject an effective amount of any embodiment of the ex vivo armed T cell described herein, wherein the ex vivo armed T cell is configured to localize to a tissue expressing one or more target antigens recognized by any embodiment of the anti-CD3 multi-specific antibody disclosed herein that is present on the ex vivo armed T cell; (b) administering to the subject an effective amount of a DOTA-based hapten, wherein the DOTA-based hapten is configured to bind to the anti-CD3 multi-specific antibody that is present on the ex vivo armed T cell, and comprises or is directly or indirectly linked to a detectable label; and (c) determining the biodistribution of the ex vivo armed T cell in the subject by detecting signal emitted by the detectable label of the DOTA-based hapten that is localized to the ex vivo armed T cells and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for tracking ex vivo armed T cells in a subject in vivo comprising (a) administering to the subject an effective amount of a complex comprising any embodiment of the ex vivo armed T cell described herein and a DOTA-based hapten, wherein the complex is configured to localize to a tissue expressing one or more target antigens recognized by any embodiment of the anti-CD3 multi-specific antibody disclosed herein that is present on the ex vivo armed T cell and wherein the DOTA-based hapten is configured to bind to the anti-CD3 multi-specific antibody that is present on the ex vivo armed T cell, and comprises or is directly or indirectly linked to a detectable label; and (b) determining the biodistribution of the ex vivo armed T cell in the subject by detecting signal emitted by the complex that is localized to the ex vivo armed T cells and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for detecting tumors in a subject in need thereof comprising (a) administering to the subject an effective amount of any embodiment of the ex vivo armed T cell described herein, wherein the ex vivo armed T cell is configured to localize to a tissue expressing one or more target antigens recognized by any embodiment of the anti-CD3 multi-specific antibody disclosed herein that is present on the ex vivo armed T cell; (b) administering to the subject an effective amount of a DOTA-based hapten, wherein the DOTA-based hapten is configured to bind to the anti-CD3 multi-specific antibody that is present on the ex vivo armed T cell, and comprises or is directly or indirectly linked to a detectable label; and (c) detecting the presence of tumors in the subject by detecting signal emitted by the detectable label of the DOTA-based hapten that is localized to the tumor and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for detecting tumors in a subject in need thereof comprising (a) administering to the subject an effective amount of a complex comprising any embodiment of the ex vivo armed T cell described herein and a DOTA-based hapten, wherein the complex is configured to localize to a tissue expressing one or more target antigens recognized by any embodiment of the anti-CD3 multi-specific antibody disclosed herein that is present on the ex vivo armed T cell and wherein the DOTA-based hapten is configured to bind to the anti-CD3 multi-specific antibody that is present on the ex vivo armed T cell, and comprises or is directly or indirectly linked to a detectable label; and (b) detecting the presence of tumors in the subject by detecting signal emitted by the complex that is localized to the tumor and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for assessing the in vivo durability or persistence of ex vivo armed T cells in a subject comprising (a) administering to the subject an effective amount of any embodiment of the ex vivo armed T cell described herein, wherein the ex vivo armed T cell is configured to localize to a tissue expressing one or more target antigens recognized by any embodiment of the anti-CD3 multi-specific antibody disclosed herein that is present on the ex vivo armed T cell; (b) administering to the subject a first effective amount of a DOTA-based hapten, wherein the DOTA-based hapten is configured to bind to the anti-CD3 multi-specific antibody that is present on the ex vivo armed T cell, and comprises or is directly or indirectly linked to a detectable label; (c) detecting signal emitted by the detectable label of the DOTA-based hapten that is localized to the ex vivo armed T cells and is higher than a reference value at a first time point; (d) detecting signal emitted by the detectable label of the DOTA-based hapten that is localized to the ex vivo armed T cells and is higher than a reference value at a second time point; and (e) determining that the ex vivo armed T cells show in vivo durability or persistence when the signal emitted by the detectable label of the DOTA-based hapten at the second time point is comparable to that observed at the first time point. In certain embodiments, the method further comprising administering to the subject a second effective amount of the DOTA-based hapten after step (c). The detectable label may be spectroscopic, photochemical, biochemical, immunochemical, electromagnetic, radioactive, fluorescent, chemifluorescent, or chemiluminescent label.

In one aspect, the present disclosure provides a method for assessing the in vivo durability or persistence of ex vivo armed T cells in a subject comprising (a) administering to the subject an effective amount of a complex comprising any embodiment of the ex vivo armed T cell described herein and a DOTA-based hapten, wherein the complex is configured to localize to a tissue expressing one or more target antigens recognized by any embodiment of the anti-CD3 multi-specific antibody disclosed herein that is present on the ex vivo armed T cell and wherein the DOTA-based hapten is configured to bind to the anti-CD3 multi-specific antibody that is present on the ex vivo armed T cell, and comprises or is directly or indirectly linked to a detectable label; (b) detecting signal emitted by the complex that is localized to the ex vivo armed T cells and is higher than a reference value at a first time point; (c) detecting signal emitted by the complex that is localized to the ex vivo armed T cells and is higher than a reference value at a second time point; and (d) determining that the ex vivo armed T cells show in vivo durability or persistence when the signal emitted by the complex at the second time point is comparable to that observed at the first time point. The detectable label may be spectroscopic, photochemical, biochemical, immunochemical, electromagnetic, radioactive, fluorescent, chemifluorescent, or chemiluminescent label.

In one aspect, the present disclosure provides a method for detecting the presence of a DOTA-based hapten in a subject that has been administered any embodiment of the ex vivo armed T cell described herein comprising (a) administering to the subject an effective amount of a DOTA-based hapten, wherein the DOTA-based hapten comprises a radionuclide, and is configured to localize to the ex vivo armed T cell; and (b) detecting the presence of the DOTA-based hapten in the subject by detecting radioactive levels emitted by the DOTA-based hapten that are higher than a reference value, wherein the ex vivo armed T cell is configured to localize to a tissue expressing one or more target antigens recognized by any embodiment of the anti-CD3 multi-specific antibody disclosed herein that is present on the ex vivo armed T cell. In another aspect, the present disclosure provides a method for detecting the presence of a DOTA-based hapten in a subject that has been administered a complex comprising any embodiment of the ex vivo armed T cell described herein and a DOTA-based hapten including a radionuclide, comprising detecting the presence of the DOTA-based hapten in the subject by detecting radioactive levels emitted by the complex that are higher than a reference value, wherein the ex vivo armed T cell is configured to localize to a tissue expressing one or more target antigens recognized by any embodiment of the anti-CD3 multi-specific antibody disclosed herein that is present on the ex vivo armed T cell.

Additionally or alternatively, in some embodiments, the method further comprises quantifying radioactive levels emitted by the DOTA-based hapten or complex that is localized to the tumor and/or radioactive levels emitted by the DOTA-based hapten or the complex that is localized in one or more normal tissues or organs of the subject. In certain embodiments, the one or more normal tissues or organs are selected from the group consisting of heart, muscle, gallbladder, esophagus, stomach, small intestine, large intestine, liver, pancreas, lungs, bone, bone marrow, kidneys, urinary bladder, brain, skin, spleen, thyroid, and soft tissue. In any of the preceding embodiments, the method further comprises determining biodistribution scores by computing a ratio of the radioactive levels emitted by the DOTA-based hapten or complex that is localized to the tumor relative to the radioactive levels emitted by the DOTA-based hapten or complex that is localized in the one or more normal tissues or organs of the subject. Additionally or alternatively, the method further comprises calculating estimated absorbed radiation doses for the tumor and the one or more normal tissues or organs of the subject based on the biodistribution scores. In some embodiments, the method further comprises computing a therapeutic index for the DOTA-based hapten or complex based on the estimated absorbed radiation doses for the tumor and the one or more normal tissues or organs of the subject.

In some embodiments of the preceding methods disclosed herein, the radioactive levels emitted by the complex or the detectably labeled DOTA-based hapten are detected using positron emission tomography or single photon emission computed tomography. Additionally or alternatively, in some embodiments of the methods disclosed herein, the radioactive levels emitted by the complex or the radiolabeled DOTA-based hapten are detected between 2 to 120 hours after the complex or the radiolabeled DOTA-based hapten is administered. In certain embodiments of the methods disclosed herein, the radioactive levels emitted by the complex or the radiolabeled DOTA-based hapten are expressed as the percentage injected dose per gram tissue (% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45(9):1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

Additionally or alternatively, in some embodiments of the methods disclosed herein, the ex vivo armed T cell, the complex or the detectably labeled DOTA-based hapten is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the ex vivo armed T cell, the complex or the detectably labeled DOTA-based hapten is administered into the cerebral spinal fluid or blood of the subject.

Examples of DOTA-based haptens useful in the methods disclosed herein include, but are not limited to, benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), and DOTATyrLysDOTA.

In one aspect, the present disclosure provides a method for treating cancer or inhibiting tumor growth or metastasis in a subject in need thereof comprising administering to the subject an effective amount of any embodiment of the ex vivo armed T cell described herein. In another aspect, the present disclosure provides a method for treating cancer or inhibiting tumor growth or metastasis in a subject in need thereof comprising (a) administering to the subject a first effective amount of any and all embodiments of the ex vivo armed T cell described herein, (b) administering to the subject a second effective amount of the ex vivo armed T cell about 72 hours after administration of the first effective amount of the ex vivo armed T cell, (c) administering to the subject a third effective amount of the ex vivo armed T cell about 96 hours after administration of the second effective amount of the ex vivo armed T cell, and (d) repeating steps (a)-(c) for at least three additional cycles. In certain embodiments, the subject exhibits sustained cancer remission after completion of step (d). In certain embodiments, the subject is human.

Additionally or alternatively, in some embodiments of the methods disclosed herein, the ex vivo armed T cell is autologous, non-autologous, or derived in vitro from lymphoid progenitor cells.

Additionally or alternatively, in some embodiments of the methods disclosed herein, the ex vivo armed T cell is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the ex vivo armed T cell is administered into the cerebral spinal fluid or blood of the subject. In some embodiments, the subject is diagnosed with, or is suspected of having cancer. Exemplary cancers or tumors include, but are not limited to, carcinoma, sarcoma, melanoma, hematopoietic cancer, osteosarcoma, Ewing's sarcoma, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, seminomas, skin cancers, stomach cancers, teratomas, testicular cancers, thyroid cancers, uterine cancers, vaginal cancers, vascular tumors, and metastases thereof.

Additionally or alternatively, in some embodiments, the method further comprises separately, simultaneously, or sequentially administering an additional cancer therapy. In some embodiments, the additional cancer therapy is selected from among chemotherapy, radiation therapy, immunotherapy, monoclonal antibodies, anti-cancer nucleic acids or proteins, anti-cancer viruses or microorganisms, and any combinations thereof. In certain embodiments, the additional cancer therapy is an immune checkpoint inhibitor selected from among pembrolizumab, nivolumab, cemiplimab, atezolizumab, avelumab, durvalumab, and ipilimumab.

Additionally or alternatively, in certain embodiments, the method further comprises administering a cytokine to the subject. In some embodiments, the cytokine is administered prior to, during, or subsequent to administration of the ex vivo armed T cell. Examples of suitable cytokines include, but are not limited to, interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

In any and all embodiments of the methods disclosed herein, in vivo or in vitro cytokine levels released by the ex vivo armed T cells are reduced compared to unarmed T cells mixed with an anti-CD3 multi-specific antibody.

Also disclosed herein are kits containing components suitable for treating cancer in a patient. In certain embodiments, the kit comprises any and all embodiments of the anti-CD3 multi-specific antibody disclosed herein in unit dosage form and instructions for arming T cells with the same. Additionally or alternatively, in some embodiments, the kits may further comprise instructions for isolating T cells from an autologous or non-autologous donor, and agents for culturing, differentiating and/or expanding isolated T cells in vitro such as cell culture media, CD3/CD28 beads, zoledronate, cytokines such as IL-2, IL-15 (e.g., IL15Rα-IL15 complex), buffers, diluents, excipients, and the like. Additionally or alternatively, in some embodiments, the kits comprise any and all embodiments of the EATs described herein and instructions for using the same to treat cancer in a subject in need thereof.

### BRIEF DESCRIPTION OF THE DRAWINGS

FIGS. 1A-1G demonstrate that ex vivo arming of T cells with IgG-[L]-scFv bispecific antibody (BsAb) significantly reduced cytokine release, while retaining anti-tumor activity. FIG. 1A shows the surface density of GD2-BsAb and HER2-BsAb on Ex Vivo Armed T cells (EATs) measured as antibody binding capacity (ABC) by fluorescence referenced to quantum beads. FIG. 1B shows antibody dependent T cell-mediated cytotoxicity (ADTC) assay of GD2-EATs and HER2-EATs at increasing effector to target ratios (E:T ratios) and at increasing BsAb arming doses. FIGS. 1C-1D show a comparison of cytotoxicity between EATs versus unarmed T cells in the continuous presence of BsAb, for both anti-GD2 and anti-HER2 systems.

FIG. 1E shows a comparison of cytokine release between pre-wash supernatant and post-wash supernatants at optimal arming doses of GD2-BsAb (0.05 μg/10⁶ cells to 5 μg/10⁶ cells), (ns=not significant, P≥0.05; *, P<0.05; **, P<0.01; ***, P<0.001; ****, P<0.0001). FIG. 1F shows TH1 cytokine release after co-culture with GD2(+) M14 melanoma cell line. FIG. 1G shows a comparison of serum TH1 cytokine levels after iv injection of GD2-EATs (10 μg of GD2-BsAb/2×10⁷ cells) or GD2-BsAb (10 μg) plus unarmed T cells (2×10⁷ cells) into osteosarcoma PDX bearing mice.

FIGS. 2A-2G demonstrate that the bispecific antibody platform has profound effects on the anti-tumor activity of EATs. FIG. 2A shows different BsAb structural platforms (M. Yankelevich et al., Pediatr Blood Cancer 59, 1198 (2012); B. H. Santich et al., Sci Transl Med 12, (2020); R. C. Grabert et al., Clin Cancer Res 12, 569 (2006)). FIG. 2B shows the surface BsAb density (measured by antibody binding capacity [ABC]) of EATs armed with different structural formats of GD2-BsAbs and HER2-BsAb. Previously published data on BiTE-Fc, IgG heterodimer, IgG-[H]-scFv and IgG-[L]-scFv (B. H. Santich et al., Sci Transl Med 12, (2020)) were compared to other BsAb formats. FIG. 2C shows an ADTC assay by GD2-EATs and HER2-EATs armed with different structural formats of BsAbs. FIG. 2D shows an in vivo anti-tumor effect of GD2-EATs armed with different structural formats of GD2-BsAb, or control BsAb; no treatment/placebo group was included for comparison (ns=not significant, P≥0.05; *, P<0.05; **, P<0.01; ***, P<0.001; ****, P<0.0001). FIG. 2E shows the in vivo anti-tumor effect of HER2-EATs armed with IgG chemical conjugate or IgG-[L]-scFv formats of HER2-BsAb; no treatment group was included for comparison. FIG. 2F shows immunohistochemical staining of CD3(+) T cell infiltration into neuroblastoma PDX tumors treated with GD2-EATs armed with different structural formats of GD2-BsAb (on day 10 after the initiation of treatment). FIG. 2G shows the in vivo anti-tumor effect of a patient's autologous T cells that are armed with GD2-BsAb (anti-GD2 IgG-[L]-scFv) and iv administered to mice bearing the corresponding patient's neuroblastoma PDXs.

FIGS. 3A-3F demonstrate that EATs showed faster tumor homing kinetics than unarmed T cells, bypassing lung sequestration. FIG. 3A shows a schematic overview of treatment schedule. FIG. 3B shows representative bioluminescence images of GD2-EATs trafficking after iv administration over days. FIG. 3C shows quantitation of T cell infiltration into tumors over time as measured by bioluminescence (n=5 mice/group) expressed as total flux or radiance (photons/sec) per pixel integrated over the entire tumor contour (ROI). FIG. 3D shows tumor growth curves and bioluminescence image of T cells over days. One mouse was dead after anesthesia on day 17. FIG. 3E shows Luc(+) GD2-EATs treatment and tumor growth curves post treatment (ns=not significant, P≥0.05; *, P<0.05; **, P<0.01; ***, P<0.001; ****, P<0.0001). FIG. 3F shows quantitation and bioluminescence images of GD2-EATs infiltration into tumors over time (n=4 mice).

FIGS. 4A-4F demonstrate the in vivo efficacy of EATs was dependent on cell dose and treatment schedule. FIGS. 4A-4B show that the in vivo anti-tumor effect was dependent on cell numbers of EATs infused. Anti-neuroblastoma effect and human CD45(+) T cell infiltration into tumors were enhanced by increasing numbers of GD2-EATs (ns=not significant, P≥0.05; *, P<0.05; **, P<0.01; ***, P<0.001; ****, P<0.0001). FIG. 4C shows the effect of GD2-EAT treatment schedule on in vivo anti-tumor potency. FIGS. 4D-4F show the effects of supplementing GD2-EATs or GD2-BsAb treatment with respect to enhancing anti-tumor effects in vivo.

FIGS. 5A-5D demonstrate ex vivo arming of T cells with multiple IgG-[L]-scFv bispecific antibodies. FIG. 5A shows surface BsAb densities, quantified as antibody binding capacity (ABC), for multi-EATs that were analyzed by fluorescence and referenced to quantum beads. FIG. 5B shows antibody dependent T cell-mediated cytotoxicity (ADTC) assay of multi-EATs and CD33-EATs at increasing effector to target ratios (E:T ratios) and at increasing arming doses of each BsAb. FIGS. 5C-5D show a comparison of in vitro cytotoxicity by multi-EATs with monospecific EATs against each target antigen (+) tumor cell lines at an E:T ratio of 10:1.

FIGS. 6A-6D demonstrate that ex vivo arming enables multi-EATs to achieve multi-specificity and to maintain anti-tumor properties against a panel of human tumor targets. FIG. 6A-6B show a comparison of in vivo anti-tumor responses by multi-EATs with monospecific EATs in a variety of tumor cell line xenograft mouse models (ns=not significant, P≥0.05; *, P<0.05; **, P<0.01; ***, P<0.001; ****, P<0.0001). FIGS. 6C-6D show the anti-tumor efficacy of multi-EATs against mixed cancer cell lines. GD2(+) IMR32Luc and GD2^{weak}HER2(+) HCC1954 cell lines were mixed, and anti-tumor activities of dual-targeting EATs were tested against this mixed cancer cell lines in vitro and in vivo.

FIGS. 7A-7F demonstrate that EAT is a versatile platform to arm γδ T cells. FIG. 7A shows flow cytometry analyses of γδTs and CD3/CD28 bead expanded T cells before arming. FIG. 7B shows surface BsAb density after arming of γδTs and unselected T cells with GD2-BsAb or HER2-BsAb. FIG. 7C shows ADTC assays of GD2-γδTs and HER2-γδTs compared to GD2-αβTs and HER2-αβTs. Non-specific tumor cell killing by unarmed γδ T cells and unarmed αβ T cells (background) were subtracted. FIG. 7D shows flow cytometry analyses of peripheral blood T cells after treatment with GD2-γδTs plus zoledronate and supplementary IL-2 or IL-15.

FIGS. 7E-7F: GD2-γδTs and HER2-γδTs were administered with supplementary IL-2 or IL-15 to treat osteosarcoma PDXs, and anti-tumor effects of γδ-EATs were compared among groups (ns=not significant, P≥0.05; *, P<0.05; **, P<0.01; ***, P<0.001; ****, P<0.0001).

FIGS. 8A-8C demonstrate the anti-tumor effects of GD2-EATs and anti-GD2 BsAb platform. The structural format of GD2-BsAbs have profound effects on in vivo anti-tumor effect of GD2-EATs against GD2(+) neuroblastoma PDX tumors, (ns=not significant, P≥0.05; *, P<0.05; ** P<0.01; *** P<0.001; **** P<0.0001).

FIGS. 9A-9B demonstrate that ex vivo arming of T cell reduced cytokine release. FIG. 9A shows TH1 cell cytokines (IL-2, IL-6, IL-10, IFN-γ, and TNF-α) released by T cells that were measured in the supernatants after 20 minutes of incubation (Prewash) and after 2nd washing step (Post wash). FIG. 9B shows a comparison of TH1 cytokine release after co-culture with GD2(+) M14 melanoma cell line between GD2-EATs and T cells in the presence of GD2-BsAb. Cytokine release was compared after co-culture with target cells.

FIGS. 10A-10E demonstrate that EAT treatment was effective across a broad spectrum of tumor targets and tumor types with minimal toxicity. FIG. 10A shows a schematic overview of EAT treatment. FIG. 10B shows the in vivo anti-tumor effect of GD2-EATs against a panel of human tumors including neuroblastoma PDXs, IMR32Luc neuroblastoma cell line xenografts, and M14Luc melanoma cell line xenografts (ns=not significant, P≥0.05; *, P<0.05; **, P<0.01; ***, P<0.001; ****, P<0.0001). FIG. 10C shows the in vivo anti-tumor effect of HER2-EATs against a panel of human tumors including telangiectatic osteosarcoma PDXs (TEOSC1), breast cancer PDXs (M37), and 143B osteosarcoma cell line xenografts.

FIG. 10D shows the in vivo anti-tumor effect of other antigen-specific EATs. Anti-STEAP-1(six transmembrane epithelial antigen prostate-1)-EATs. FIG. 10E shows changes in mouse weight over time (body weight relative to that before treatment started). EAT therapy did not cause toxicities or weight loss.

FIGS. 11A-11F demonstrate that cryopreserved EATs retained target-antigen specific cytotoxicity and exerted a comparable anti-tumor activity. FIG. 11A shows mean fluorescence intensities (MFIs) of BsAb density on GD2-EATs (0.5 μg/10⁶ cells) and HER2-EATs (0.5 μg/10⁶ cells) before and after cryopreservation. FIG. 11B shows an ADTC assay of GD2-EATs and HER2-EATs against GD2(+) and/or HER2(+) cell lines before and after cryopreservation. FIG. 11C shows anti-tumor response against osteosarcoma PDXs by cryopreserved (thawed) GD2-EATs and freshly armed (fresh) GD2-EATs (ns=not significant, P≥0.05; *, P<0.05; **, P<0.01; ***, P<0.001; ****, P<0.0001). FIG. 11D shows relative body weights over times. FIG. 11E shows flow cytometry analyses of peripheral blood T cells in the mice treated with thawed or fresh GD2-EATs. FIG. 11F shows in vivo anti-tumor effects of thawed GD2-EATs and HER2-EATs against telangiectatic osteosarcoma PDXs; both thawed GD2-EATs and HER2-EATs significantly suppressed tumor growth without weight loss, improving survival (P<0.0001).

FIGS. 12A-12E depict combinatorial EAT strategies. FIGS. 12A-12B show ADTC assays to test in vitro tumor cell killing by combinatorial EATs (dual-EATs, GD2/HER2-EATs; pooled-EATs, GD2-EATs+HER2-EATs). T cells were armed with a dose of 0.5 μg of each BsAb per 10⁶ of cells. FIG. 12C shows an in vivo anti-tumor response by pooled-EATs (GD2-EATs+HER2-EATs), administered i.v. at 2×10⁷ cells per injection (ns=not significant, P≥0.05; *, P<0.05; **, P<0.01; ***, P<0.001; ****, P<0.0001). FIGS. 12D-12E show a comparison of in vivo anti-tumor response by dual-EATs (GD2/HER2-EATs) to monospecific EATs and sequential combination of EATs (HER2-EATs followed by GD2-EATs).

FIGS. 13A-13D depict cytokine release by multi-EATs. FIG. 13A shows TH1 cell cytokines (IL-2, IL-6, IL-10, IFN-γ, and TNF-α) that were measured in the supernatants after 20 minutes of incubation with multiple BsAbs (prewash) and after 2nd washing step (post wash), (ns=not significant, P≥0.05; *, P<0.05; **, P<0.01; ***, P<0.001; ****, P<0.0001). FIG. 13B shows a comparison of TH1 cytokine release between multiple BsAbs plus unarmed T cells and multi-EATs after co-culture with target cells [GD2(+) IMR32Luc]. FIG. 13C: In vivo TH1 cytokine levels were analyzed 4 hours after injection of T cells and compared among groups (GD2-BsAb plus unarmed T cells, GD2-EATs, multi-EATs, and unarmed T cells) in GD2(+)HER2(+) osteosarcoma PDX model. FIG. 13D: In vivo TH1 cytokine release was analyzed 4 hours after second injection of EATs and compared among groups in GD2(+) IMR32Luc and HER2(+) HCC1954 mixed cancer cell line xenograft model.

FIG. 14 shows multiple antigens targeting strategies using EATs in vivo. FIG. 14 depicts a schematic overview of treatment of GD2(+) IMR32Luc and HER2(+) HCC1954 mixed cancer cell line xenografts using EAT strategies.

FIG. 15A shows a schematic overview of treatment of osteosarcoma PDX mice using ex vivo armed γδ T cells with supplementary IL-2. FIG. 15B shows a comparison of in vivo anti-tumor activities of ex vivo BsAb armed γδ T cells with ex vivo BsAb armed αβ T cells in osteosarcoma PDX mouse models (ns=not significant, P≥0.05; *, P<0.05; **, P<0.01; ***, P<0.001; ****, P<0.0001). Circulating T cells and tumor infiltrating lymphocytes (TILs) were also compared among groups.

FIG. 16A shows detection of human PD-L1 in OS xenografted tumors using IHC staining. FIG. 16B shows flow cytometry analyses of human PD-L1 expression in OS tumor. FIGS. 16C-16D show quantification of PD-L1 expression level using geometric MFI. The MFI of human PD-L1 expression increased with BsAb treatment (FIG. 16D). Data are shown as mean values±SEM. Two-sided unpaired t-test or one-way ANOVA test: ns, P≥0.05; *, P<0.05; **, P<0.01; ***, P<0.001; ****, P<0.0001.

FIG. 17A shows flow cytometry analyses of TILs in treatment-resistant OS tumors. FIG. 17B shows the frequency of human PD-1(+)CD4(+) TILs and PD-1(+)CD8(+) TILs (most of CD8(+) TILs expressed PD-1). FIG. 17C shows the frequencies of mouse PD-1(+) or mouse PD-L1(+) populations among mouse CD45(+) tumor infiltrating myeloid cells (TIMs). FIG. 17D shows human PD-1 expression in treatment-resistant OS tumor (143B xenograft), as determined by IHC staining. FIG. 17E shows flow cytometry analyses of human PD-1 expression in CD3(+) T cells in peripheral blood after GD2-BsAb or HER2-BsAb treatment. Data are shown as mean values±SEM. Two-sided unpaired t-test or one-way ANOVA test: ns, P>0.05; *, P<0.05; **, P<0.01; ***, P<0.001; ****, P<0.0001.

FIG. 18A shows a schematic overview of a combination therapy treatment schedule. FIG. 18B shows the anti-tumor response of anti-PD-1 antibody combined with GD2-EAT or HER2-EAT (P>0.05) and anti-PD-L1 antibody combined with GD2-EAT or HER2-EAT compared to GD2-EAT alone or HER2-EAT alone (P=0.0123 and P=0.0004). Data are shown as mean values±SEM. Two-sided unpaired t-test or one-way ANOVA test: ns, P>0.05; *, P<0.05; **, P<0.01; ***, P<0.001; ****, P<0.0001.

FIGS. 19A-19B show mouse IgG3-3F8 staining of fresh frozen tumor sections of each group depicted in FIG. 18B. GD2 expression was scored by staining intensity. FIG. 19C shows flow cytometric analyses of peripheral blood T cells and TILs at different time points. Data are shown as mean values±SEM. Two-sided unpaired t-test or one-way ANOVA test: ns, P>0.05; *, P<0.05; ** P<0.01; *** P<0.001; **** P<0.0001.

FIG. 20A shows a schematic overview of combination treatment of PD-1/PD-L1 blockade and GD2-EATs: a comparison of 3 different schedules of PD-1/PD-L1 [concurrent therapy (CT) vs. sequential therapy (ST) vs. sequential and continuous therapy (SCT)] are depicted. FIG. 20B shows the effects of anti-PD-1 antibody and GD2-EAT combination treatment: CT of anti-PD-1 produced an inferior response compared to GD2-EAT, and neither ST nor SCT had benefit over GD2-EAT alone. FIG. 20C shows the effects of combination treatment of anti-PD-L1 antibody and GD2-EAT: ST or SCT of anti-PD-L1 showed a significant improvement of tumor control compared to GD2-EAT alone (P=0.018 and P<0.0001). FIG. 20D shows the analyses of overall survival according to three different schedules of PD-1/PD-L1 blockades: SCT of anti-PD-L1 significantly improved the survival for GD2-EAT (P=0.0057).

FIG. 21A shows flow cytometry analyses of PB on day 21 and 34 post tumor transplantation. FIG. 21B shows flow cytometry analyses of tumor infiltrating lymphocytes (TILs) and tumor infiltrating CD8(+) T-cells (while CT of anti-PD-1 had significantly fewer circulating T-cells and TILs, ST or SCT of anti-PD-L1 significantly increased the frequencies of circulating T cells and hCD45(+) or CD8 (+) TILs compared to GD2-EAT alone). FIG. 21C shows the analyses of human PD-1 expression in peripheral blood T-cells on day 21 and human PD-1 expression in TILs when the tumors reached 2000 mm³ or the last day of experiment (CT of anti-PD-1 or anti-PD-L1 had significantly greater frequencies of PD-1 expression on CD8(+) TILs compared to GD2-EAT alone—data are shown as mean values±SEM).

FIG. 22 shows formalin-fixed paraffin-embedded (FFPE) tumor sections of each group that were stained with anti-human CD3 antibody. (G1) control BsAb-EATs, (G2) anti-PD-1 and ATCs, (G3) anti-PD-L1 and ATCs, (G4) GD2-EATs, (G5) GD2-EATs and CT of anti-PD-1, (G6) GD2-EATs and ST of anti-PD-1, (G7) GD2-EATs and SCT of anti-PD-1, (G8) GD2-EATs and CT of anti-PD-L1, (G9) GD2-EATs and ST of anti-PD-L1, and (G10) GD2-EATs and SCT of anti-PD-L1. Tumors were collected when they reached 2000 mm³ or on the last day of the experiment. (200× magnifications of CD3 IHC staining).

FIG. 23A shows geometric mean fluorescence intensity (MFI) of GD2 and HER2 antigen expression in each osteosarcoma cell line (143B, U-2 OS, MG-63, HOS, and Saos-2) and osteoblast cell line, hFOB 1.19 (cells were stained with GD2 or HER2 monoclonal antibodies and secondary PE-conjugated anti-human IgG antibody, and mouse IgG1 monoclonal antibody or rituximab (anti-CD20) were used as negative control). FIG. 23B shows antibody-dependent T-cell mediated cytotoxicity (ADTC) by ⁵¹Cr release assay using activated T-cells (Effector to target cell ratio was 10 to 1) at decreasing concentrations of BsAb.

FIGS. 24A-24B show a schematic overview of the treatment schedule and mean tumor growth curves, and AUC analyses of the tumor growth. FIG. 24C shows immunohistochemical staining of tumor infiltrating lymphocytes (TILs), where tumors were harvested on day 30 post treatment and stained with anti-human CD3 antibody. FIG. 24D shows immunohistochemical staining of xenograft tumors for CD4 and CD8. FIG. 24E shows in vivo anti-tumor effect of decreasing doses of GD2-BsAb or HER2-BsAb. Data are shown as mean values±SEM. Two-sided unpaired t-test and one-way ANOVA test: ns, P≥0.05; *, P<0.05; **, P<0.01; ***, P<0.001; ****, P<0.0001 vs. control.

FIG. 25A shows a schematic overview of treatment schedule for EAT therapy. FIG. 25B shows in vivo testing of GD2-EATs over a range of BsAb arming dosages. FIG. 25C shows in vivo testing of HER2-EAT over a range of BsAb arming dosages. FIG. 25D shows that an intermediate dose (0.5 μg/1×10⁶ T cells) of armed GD2-EAT and HER2-EAT had a potent anti-tumor effect against OS PDX tumor and significantly improved survival. FIG. 25E shows binding, in vitro cytotoxicity and in vivo anti-tumor activity of cryopreserved GD2-EAT and HER2-EAT. Data are shown as mean values±SEM. Two-sided unpaired t-test and one-way ANOVA test: ns, P>0.05; *, P<0.05; **, P<0.01; ***, P<0.001; ****, P<0.0001

FIG. 26A shows in vitro cytotoxicity analyses using GD2-EAT, HER2-EAT and combinatorial GD2+HER2-EAT over range of BsAb dose and E:T ratio. FIG. 26B shows treatment of OS PDXs with 10×10⁶ cells of GD2-EAT, HER2-EAT or combination of both EATs (GD2-EAT+HER2-EAT). EATs were injected three times. FIG. 26C: four doses (20×10⁶ cells/dose) of each EATs (GD2-EATs, HER2-EATs and GD2-EATs+HER2-EATs) were compared their anti-tumor effect against OS PDXs. FIG. 26D shows a schematic overview of treatment schedule of combinatorial treatment (Six doses (2×10⁷ cells/dose) of each EATs (GD2-EATs, HER2-EATs or dual specificity GD2/HER2-EATs) or 3 doses of HER2-EATs followed by 3 doses of GD2-EATs). FIG. 26E shows in vivo anti-tumor response by treatment groups depicted in FIG. 26D.

FIG. 27A shows a schematic overview of combination treatment of PD-1/PD-L1 blockade and GD2-EAT: a comparison of 3 different schedules of PD-1/PD-L1 [concurrent therapy (CT) vs. sequential therapy (ST) vs. sequential and continuous therapy (SCT)]. FIG. 27B shows anti-PD-1 antibody and GD2-EAT combination treatment and analysis of treatment response: a comparison of the three schedules of anti-PD-1 antibody depicted in FIG. 27A. FIG. 27C shows combination treatment of anti-PD-L1 antibody and GD2-EAT and analysis of treatment response: a comparison of the three schedules of anti-PD-L1 antibody depicted in FIG. 27A. FIG. 27D shows the effect of different schedule of immune check point inhibitors (ICIs) on peripheral blood T cells on day 11 and 24 post treatment. FIG. 27E shows flow cytometry analyses of tumor infiltrating lymphocytes (TILs) and tumor infiltrating CD8(+) T-cells. FIG. 27F shows the analyses of PD-1 expression on peripheral blood T-cells (on day 11) and TILs (when the tumors reached 2000 mm³ or the last day of experiment). FIG. 27G shows anti-human CD3 antibody staining of formalin-fixed paraffin-embedded (FFPE) tumor sections of each treatment group (200× magnification). CD3(+) T cell numbers were compared. Data are shown as mean values±SEM. Two-sided unpaired t-test or one-way ANOVA test: ns, P≥0.05; *, P<0.05; **, P<0.01; ***, P<0.001; ****, P<0.0001.

FIGS. 28A-28D show the effects of high-dose BsAb on T cell activation and exhaustion. CD3(+) T cells were incubated with increasing concentrations of GD2-BsAb or HER2-BsAb and analyzed by flow cytometry using 7-AAD, PE-labeled anti-human FasL, APC-labeled anti-human CD25, PE-labeled anti-human CD69, APC-labeled anti-human PD-1, APC-labeled anti-human TIM-3, and PE-labeled anti-human LAG-3. The frequency of each marker-positive subpopulation was calculated after subtracting away that for the no-BsAb control. EC₅₀ for CD25, CD69, PD-1, TIM-3 and LAG-3 marker upregulation ranged from 0.05 μg/1×10⁶ T-cells for GD2-BsAb and 0.5 to 5 μg/1×10⁶ T-cells for HER2-BsAb. EC₅₀ for 7AAD, FasL and Annexin V was higher than 50 μg/1×10⁶ T-cells and registering 15-30% without plateau yet at 50 μg/1×10⁶ T-cells.

FIG. 29A shows the mean fluorescence intensity (MFI) of bound BsAb using anti-idiotype or anti-human IgG antibodies. FIG. 29B shows antibody dependent T-cell mediated cytotoxicity assay (ADTC) using GD2-EATs and HER2-EATs. FIG. 29C shows in vitro cytotoxicity by different doses of BsAb arming. FIG. 29D shows antibody binding capacity (ABC), i.e., T-cell bound BsAb density (molecules per cell), which was estimated using quantum beads by FACS analyses.

FIG. 30A shows a schematic overview of treatment schedule. FIGS. 30B-30C show in vivo anti-tumor effect of ICI combination with GD2-EATs or HER2-EATs.

FIG. 31 shows tumor associated antigen expression (MFI, Mean Fluorescence Intensity) in osteosarcoma. Abbreviations, GD2, disialoganglioside GD2; GD3, disialohematoside; HER2, human epidermal growth factor receptor 2; HMW, high-molecular weight melanoma antigen; CSPG4, Chondroitin-sulfate proteoglycan 4; GPA, glycoprotein A33; L1CAM, L1 cell adhesion molecule; GPC-3, glypican-3; PSA, polysialic acid; PD-L1, programmed death-ligand 1; PSMA, prostate-specific membrane antigen; IGF2R; Insulin-like growth factor 2 receptor.

FIG. 32 shows in vitro sensitivities (EC50, pM) to target antigen specific bispecific antibodies in osteosarcoma cell lines.

FIG. 33 shows exemplary amino acid sequences of anti-CD3 multi-specific antibodies that are useful for arming the EATs of the present technology.

FIGS. 34A-34C show multi-antigens targeting strategies using Ex vivo Armed T cells (EATs) complexed with IgG-[L]-scFv platform BsAb. FIG. 34A shows representative models of mono-EATs (GD2-EATs or HER2-EATs), pooled-EATs, dual- or multi-EATs, and TriAb-EATs, respectively. FIG. 34B: In vitro cytotoxicity against GD2(+) and/or HER2(+) cancer cell lines was tested and compared among mono-EATs, pooled EATs, and dual-EATs at increasing E:T ratios (effector to target ratio). EATs were armed with 0.5 μg of each BsAb per 1×10⁶ of T cells. GD2(+) IMR32Luc neuroblastoma cell line, HER2(+) HCC1954 breast cancer cell line, HER2(+) NCI-N87 gastric cancer cell line, and both GD2 and HER2 weakly positive (GD2^{lo} HER2^{lo}) 143B osteosarcoma cell lines were used respectively. FIG. 34C: In vivo anti-tumor response of mono-EATs [GD2-EATs (10 μg of GD2-BsAb/2×10⁷ cells) or HER2-EATs (10 μg of HER2-BsAb/2×10⁷ cells)], pooled-EATs (5 μg/1×10⁷ of GD2-EATs plus 5 μg/1×10⁷ of HER2-EATs), and dual-EATs (5 μg of GD2-BsAb+5 μg of HER2-BsAb/2×10⁷ cells) was tested against GD2(+) and HER2(+) osteosarcoma PDX (OS1B). Tumor growth curves and overall survival were compared among groups.

FIGS. 35A-35C show anti-tumor activity of GD2×HER2×CD3 trispecific antibody (TriAb) armed T cells (TriAb-EATs). FIG. 35A shows bispecific antibody structure of GD2×HER2×CD3 TriAb. FIG. 35B shows antibody-dependent T cell-mediated cytotoxicity (ADTC) of TriAb-EAT was compared with mono-EAT (GD2-EAT or HER2-EAT) and dual-EAT against GD2(+) and/or HER2(+) cancer cell lines at increasing E:T ratios. FIG. 35C shows in vivo anti-tumor effect of TriAb-EATs against GD2(+) and HER2(+) osteosarcoma PDX (TEOSC1). Three doses of unarmed T cells (2×10⁷ cells) or EATs (10 μg of each BsAb/2×10⁷ cells) were administered.

FIGS. 36A-36C show ex vivo armed T cells with multiple BsAbs (multi-EATs). FIG. 36A shows surface BsAb density on multi-EAT was analyzed using anti-human IgG Fc-specific antibody and anti-rat quantum beads. Geometric mean fluorescence intensities (MFIs) of EATs were measured with increasing arming doses of each BsAb, and BsAb density (MFI) of EAT was referenced to antibody-binding capacity (ABC). FIG. 36B shows in vitro cytotoxicity of multi-EATs and CD33-EATs against CD33(+) MOLM13 cell line at increasing E:T ratios and increasing BsAb arming doses. The optimal BsAb densities on T cells were extrapolated from the ADTC assays. FIG. 36C shows in vitro cytotoxicity of multi-EATs was tested against a panel of tumor cell lines (E:T ratio was 10:1) and compared with mono-EATs.

FIGS. 37A-37C show cytokine release by multi-EATs. FIG. 37A: TH1 cell cytokines (IL-2, IL-6, IL-10, IFN-γ, and TNF-α) were measured in the supernatants after 4 hours of incubation of 5 BsAbs plus T cells or 5 BsAb armed T cells (5BsAbs-EATs) with target cells at increasing doses of each BsAb (0.0003 μg/1×10⁶ cells to 25 μg/1×10⁶ cells). Mixture of multiple cancer cell lines consisting GD2(+) M14, HER2(+) HCC1954, CD33(+) HL60, PSMA(+) LNCaP-AR, and STEAP1(+) TC32 were used as target cells. ET ratio (effector to target cell ratio) was 20:1. FIG. 37B: In vitro cytokine release of multi-EATs was compared following an increase in the number of BsAb. ET ratio was 20:1, and mixture of multiple cancer cell lines such as GD2(+) M14, HER2(+) HCC1954, CD33(+) HL60, PSMA(+) LNCaP-AR, and STEAP1(+) TC32 were used as target cells. FIG. 37C: In vivo TH1 cytokine levels were analyzed 4 hours after second dose of EAT treatment in GD2(+) and HER2(+) 143B osteosarcoma cell line xenograft (CDX) mouse model. G1, GD2-BsAb and unarmed T cells; G2, multi-EATs (GD2/HER2/CD33/PSMA/STEAP1-EATs); G3, GD2-EATs; G4, HER2-EATs; G5, unarmed T cells. BsAb dose and T cell number were fixed at 10 μg for each BsAb and 2×10⁷ for T cell per injection.

FIGS. 38A-38B show in vivo anti-tumor activities of multi-EATs. FIG. 38A: In vivo anti-tumor effect of multi-EATs was tested against a variety of cancer xenografts including M37 breast cancer patient-derived xenografts (PDXs), LNCaP-AR prostate cancer CDXs, and IMR32Luc neuroblastoma CDXs. Six does of EATs or unarmed T cells were administered.

FIG. 38B: In vivo anti-tumor effect of multi-EATs was compared with single antigen targeted STEAP1-EATs against Ewing sarcoma family of tumor (EFT) PDXs. Two doses of EATs or unarmed T cells were administered. BsAb dose and T cell number were fixed at 2 μg for each BsAb and 2×10⁷ for T cell per injection.

FIGS. 39A-39E show anti-tumor efficacy of multi-EATs against mixed lineage targets. FIG. 39A: In vitro cytotoxicity was tested against IMR32Luc and HCC1954 mixed lineage. FIG. 39B shows a schematic overview of treatment for MR32Luc and HCC1954 mixed lineage xenografts using multi-antigens targeting EAT strategies. BsAb dose and T cell number were fixed at 10 μg for each BsAb and 2×10⁷ for T cell per injection. FIG. 39C shows mouse body weight during follow-up period. FIG. 39D shows overall survival by treatment. FIG. 39E shows tumor response by treatment groups.

FIGS. 40A-40E show analysis of tumor response by immunohistochemical (IHC) staining. FIG. 40A shows gross phenotypes of tumors in each treatment group: a, unarmed T cells; b, GD2-EATs; c, HER2-EATs; d, TriAb-EATs; e, alternate EATs; f, dual-EATs; g, multi-EATs. FIG. 40B shows H&E staining of tumors in each treatment group: a, unarmed T cells; b, GD2-EATs; c, HER2-EATs; d, TriAb-EATs; e, alternate EATs; f, dual-EATs; g, multi-EATs. FIG. 40C shows fresh frozen tumor staining with anti-human GD2 antibody (hu3F8): a, unarmed T cells; b, GD2-EATs; c, HER2-EATs; d, TriAb-EATs; e, alternate EATs; f, dual-EATs, g, multi-EATs. FIG. 40D shows IHC staining of formalin-fixed paraffin-embedded (FFPE) tumor sections with anti-human HER2 antibody (trastuzumab): a, unarmed T cells; b, GD2-EATs; c, HER2-EATs; d, TriAb-EATs; e, alternate EATs; f, dual-EATs; g, multi-EATs. FIG. 40E shows IHC staining of FFPE tumor sections with anti-human CD3 antibody: a, unarmed T cells, b, GD2-EATs; c, HER2-EATs; d, TriAb-EATs; e, alternate EATs, f, dual-EATs; g, multi-EAT.

FIGS. 41A-41B show in vivo anti-tumor efficacy of dual- or alternate EATs. FIG. 41A shows a schematic overview of treatment. Six doses of unarmed T cells or EATs were administered intravenously into GD2(+) and HER2(+) osteosarcoma 143B cell line xenograft (CDX). BsAb dose and T cell number were fixed at 10 μg for each BsAb and 2×10⁷ for T cell per injection. Alternate EATs were given by administering GD2-EATs and HER2-EATs alternately. FIG. 41B: In vivo anti-tumor response was compared among groups. Tumor growth, body weight of mice during follow-up period, and overall survival were plotted and compared among groups.

FIGS. 42A-42B show in vivo anti-tumor effect of TriAb-EATs. FIG. 42A shows a schematic overview of treatment. Three doses of unarmed T cells or EATs were given intravenously into osteosarcoma patient-derived xenografts (HGSOC1). 2×10⁷ of unarmed T cells or EATs (10 μg of each BsAb/2×10⁷ of T cell) were administered iv twice per week. FIG. 42B: In vivo anti-tumor response was compared among groups.

FIGS. 43A-43B show in vitro and in vivo anti-tumor activity of multi-EATs against mixed lineages. FIG. 43A: In vitro cytotoxicity of multi-EATs was tested against GD2(+)IMR32Luc and HER2(+) HCC1954 mixed lineage cells and compared with TriAb-EATs and mono-EATs. FIG. 43B shows a schematic overview of treatment for IMR32Luc and HCC1954 mixed lineage xenograft using multiple EAT strategies, and in vivo anti-tumor activity of multi-EATs was compared among groups including TriAb-EATs. BsAb dose and T cell number were fixed at 10 μg for each BsAb and 2×10⁷ for T cell per injection.

FIGS. 44A-44D show histologic features of IMR32Luc-, HCC1954-, and IMR32Luc and HCC1954 mixed lineage-xenografts. FIG. 44A shows gross phenotypes of tumors: a, IMR32Luc cell line xenograft (CDX); b, HCC1954 CDX; c, IMR32Luc and HCC1954 mixed lineage CDX. FIG. 44B shows H&E staining of tumors: a, IMR32Luc CDX; b, HCC1954 CDX; c, IMR32Luc and HCC1954 mixed lineage CDX. FIG. 44C shows fresh frozen tumor staining with anti-human GD2 antibody (hu3F8): a, IMR32Luc CDX; b, HCC1954 CDX; c, IMR32Luc and HCC1954 mixed lineage CDX. FIG. 44D shows IHC staining of formalin-fixed paraffin-embedded (FFPE) tumor sections with anti-human HER2 antibody (trastuzumab): IMR32Luc CDX; b, HCC1954 CDX; c, IMR32Luc and HCC1954 mixed lineage CDX.

FIGS. 45A-45B show in vivo anti-tumor response of multi-EATs against relapsed tumors. FIG. 45A shows a schematic overview of treatment. For multi-EAT therapy, 2×10⁷ of T cells were armed with 5 BsAbs (2 μg of GD2-BsAb, 2 μg of HER2-BsAb, 2 μg of CD33-BsAb, 2 μg of PSMA-BsAb, and 2 μg of STEAP1-BsAb) and administered intravenously on day 0 and day 3 post-treatment. When tumors relapsed, identical doses of multi-EATs were given on day 54, day 90, and day 217, respectively. FIG. 45B shows that in vivo anti-tumor response was monitored.

FIG. 46 shows purity, affinity and endotoxin content of the bispecific antibody preparations of the present technology. ND=Not done because the binding epitope was lost from soluble STEAP1 protein.

FIG. 47 shows binding of the bispecific antibodies disclosed herein to tumor cell lines by flow cytometry (MFI).

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrates that ex vivo expanded T cells armed with multi-specific antibodies that target at least one tumor antigen can be transformed into living drugs that produce robust anti-tumor immune responses. These methods entail harvesting autologous T cells from a patient before intensive chemotherapy or using third party donor's T cells with reduced alloreactivity, and rejuvenating/expanding said T cells ex vivo using appropriate cytokines.

IL-6 and TNF-α have been implicated as the central mediators of CRS (D. W. Lee et al., Blood 124, 188 (2014)). When BsAbs engage polyclonal T cells to undergo synchronous activation, TNF-α acts as the initial signal for monocyte activation, resulting in release of IL-6 and IL-1, preventable by anti-TNF-α antibodies without compromising anti-tumor activity (J. Li et al., Sci Transl Med 11, (Sep. 4, 2019)). The present disclosure demonstrates that multi-specific antibodies carried on T cells produce significantly less cytokines than direct antibody injections, while still able to drive T cells rapidly into tumors to achieve significant anti-tumor effects. The Examples described herein demonstrate that while T cells produced TNF-α during the initial 20 minutes of arming; post-wash EATs released significantly less cytokines in vitro and in vivo without affecting their trafficking ability or tumoricidal activity. These anti-tumor effects are equally effective in the setting of autologous paired T cell-tumor systems, eliminating the confounding allogeneic effect common in humanized mouse models. Moreover, cryopreserved EATs showed >85% viability, unexpectedly retained anti-tumor properties, and showed no clinical signs of graft versus host disease. Compare with Chong, E. A. et al, Blood 132 (Suppl 1), 197 (2018); Roddie, C. et al., Cytotherapy 21, 327-340 (2019); Elavia, N. et al., Blood 130, 4475 (2017). Likewise, γδ EATs in combination with IL15Rα-IL15 cytokines also possess tumoricidal activity with minimized ‘graft versus host’ side effects.

Another challenge to immunotherapy is antigen loss or down-regulation, well known following CAR T cells, BsAbs, or monoclonal antibodies (N. N. Shah, T. J. Fry, Nat Rev Clin Oncol 16, 372 (2019); S. L. Maude et al., N Engl J Med 371, 1507 (2014); T. J. Fry et al., Nat Med 24, 20 (2018)). Even with highly efficient CART cells, a minimum threshold of antigen expression is required (K. Watanabe et al., J Immunol 194, 911 (2015)), and any decrease in antigen expression significantly affects immunotherapy efficacy (H. G. Caruso et al., Cancer Res 75, 3505 (2015)). The present disclosure demonstrates that multi-EATs retain functionality against each individual target (thus overcoming the hurdles of tumor heterogeneity and suboptimal antigen density) both in vitro and in vivo, showed reduced cytokine-related toxicities compared to treatment with multiple BsAbs, and improved overall survival. Multi-EATs targeting GD2, HER2, CD33, PSMA, and STEAP1 demonstrated identical and in some cases robust anti-tumor efficacy to mono-EATs against designated tumor targets. More importantly, dual- or multi-EATs drove more T cells into tumors and overcome tumor heterogeneity of mixed lineage tumor targets, avoiding treatment resistance and preventing clonal escape. Given the minimal requirement of anti-CD3 multi-specific antibodies per T cell for anti-tumor activity, multiple anti-CD3 multi-specific antibodies built on the same IgG-[L]-scFv platform can be installed on each T cell before the maximum capacity is reached. Since T cell loading is mediated through the same anti-CD3 scFv domain in IgG-[L]-scFv constructs, multi-specific antibody surface density would be predictable and consistent, thus permitting fine-tuning of the relative density of each multi-specific antibody on each T cell by adjusting the arming doses.

As disclosed herein, dual- or multi-EATs showed a synergistic anti-tumor effect when simultaneously encountering multiple antigens. The formation of bi- or multi-valent immune synapses when dual- or multi-EATs exposed to heterogeneous tumors co-expressing multiple TAAs would be crucial to exert synergistic anti-tumor effect and prevent antigen escape. In contrast to the tumors treated with mono-specific GD2-EATs or HER2-EATs forcing target antigen loss, the tumors that escaped after treatment with alternate-, dual- or multi-EATs retained their target antigen expression, and escaped EFT PDX after multi-EAT therapy responded to rechallenges, implicating a major advantage over conventional single antigen targeted immunotherapy.

One of the concerns of multi-antigen targeted T cell immunotherapies is on-target off-tumor toxicities. On-target off-tumor toxicities following the infusion of CAR T cells or BsAbs can cause serious or life-threatening adverse effects, and the extent and severity of toxicity could be amplified by increasing numbers of targeting antigens. It has been reported that high target antigen affinity increased the severity of on-target off-tumor toxicities (Chmielewski et al., J Immunol 173, 7647-7653 (2004)). While multi-specific CAR T cells are life-long and such toxicities could be prolonged and life-threatening, the EATs of the present technology have limited functional life expectancy; as the BsAbs get metabolized, T cells should revert to their nonspecific states, thereby alleviating the risk of life-threatening long-term toxicities.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e., heterologous T-cell receptor), modified lymphocytes, and CAR (i.e., chimeric antigen receptor) modified lymphocytes. In another embodiment, the adoptive cell therapeutic composition comprises a cell type selected from a group consisting of T-cells, CD8+ cells, CD4+ cells, NK-cells, delta-gamma T-cells, regulatory T-cells and peripheral blood mononuclear cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” (includes intact immunoglobulins) and “antigen binding fragments” specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

More particularly, antibody refers to a polypeptide ligand comprising at least a light chain immunoglobulin variable region or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Typically, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds CD3 protein will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). “Immunoglobulin-related compositions” as used herein, refers to antibodies (including monoclonal antibodies, polyclonal antibodies, humanized antibodies, chimeric antibodies, recombinant antibodies, multi-specific antibodies, bispecific antibodies, etc.) as well as antibody fragments. An antibody or antigen binding fragment thereof specifically binds to an antigen.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the technology are any combinations of variable region(s) and hinge region, CH₁, CH₂, and CH₃ domains. Antibody-related molecules useful in the present methods, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” or “antigen binding fragments” can comprise a portion of a full length antibody, generally the antigen binding or variable region thereof. Examples of antibody fragments or antigen binding fragments include Fab, Fab′, F(ab′)₂, and Fv fragments; diabodies; linear antibodies; single-chain antibody molecules; and multi-specific antibodies formed from antibody fragments.

“Bispecific antibody” or “BsAb”, as used herein, refers to an antibody that can bind simultaneously to two targets that have a distinct structure, e.g., two different target antigens, two different epitopes on the same target antigen, or a hapten and a target antigen or epitope on a target antigen. A variety of different bispecific antibody structures are known in the art. In some embodiments, each antigen binding moiety in a bispecific antibody includes V_{H} and/or V_{L} regions; in some such embodiments, the V_{H} and/or V_{L} regions are those found in a particular monoclonal antibody. In some embodiments, the bispecific antibody contains two antigen binding moieties, each including V_{H} and/or V_{L} regions from different monoclonal antibodies. In some embodiments, the bispecific antibody contains two antigen binding moieties, wherein one of the two antigen binding moieties includes an immunoglobulin molecule having V_{H} and/or V_{L} regions that contain CDRs from a first monoclonal antibody, and the other antigen binding moiety includes an antibody fragment (e.g., Fab, F(ab′), F(ab′)₂, Fd, Fv, dAB, scFv, etc.) having V_{H} and/or V_{L} regions that contain CDRs from a second monoclonal antibody.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and Hollinger et al., Proc. Natl. Acad. Sci. USA, 90: 6444-6448 (1993).

As used herein, the terms “single-chain antibodies” or “single-chain Fv (scFv)” refer to an antibody fusion molecule of the two domains of the Fv fragment, V_{L} and V_{H}. Single-chain antibody molecules may comprise a polymer with a number of individual molecules, for example, dimer, trimer or other polymers. Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single-chain Fᵥ (scFᵥ)). Bird et al. (1988) Science 242:423-426 and Huston et al. (1988) Proc. Natl. Acad Sci. USA 85:5879-5883. Such single-chain antibodies can be prepared by recombinant techniques or enzymatic or chemical cleavage of intact antibodies.

Any of the above-noted antibody fragments are obtained using conventional techniques known to those of skill in the art, and the fragments are screened for binding specificity and neutralization activity in the same manner as are intact antibodies.

As used herein, an “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the target antigen may be a polypeptide (e.g., a CD3 polypeptide). An antigen may also be administered to an animal to generate an immune response in the animal.

The term “antigen binding fragment” refers to a fragment of the whole immunoglobulin structure which possesses a part of a polypeptide responsible for binding to antigen. Examples of the antigen binding fragment useful in the present technology include scFv, (scFv)₂, scFvFc, Fab, Fab′ and F(ab′)₂, but are not limited thereto.

As used herein, an “armed T cell” refers to any white blood cell expressing CD3 on its cell surface that has been coated with one or more multi-specific antibodies (e.g., BsAbs) having antineoplastic and/or immunomodulating activities. By way of example only, but not by way of limitation, T cells may be expanded and/or activated ex vivo and then armed with an anti-CD3 multi-specific antibody (e.g., a BsAb). Upon administration, the multi-specific antibody-armed activated T cells are configured to localize to a tumor cell expressing a target antigen (e.g., tumor antigen) recognized by the anti-CD3 multi-specific antibody, and selectively cross-link with the tumor cells; this may result in the recruitment and activation of cytotoxic T lymphocytes (CTLs), CTL perforin-mediated tumor cell cytolysis, and/or the secretion of antitumor cytokines and chemokines.

By “binding affinity” is meant the strength of the total noncovalent interactions between a single binding site of a molecule (e.g., an antibody) and its binding partner (e.g., an antigen or antigenic peptide). The affinity of a molecule X for its partner Y can generally be represented by the dissociation constant (K_{D}). Affinity can be measured by standard methods known in the art, including those described herein. A low-affinity complex contains an antibody that generally tends to dissociate readily from the antigen, whereas a high-affinity complex contains an antibody that generally tends to remain bound to the antigen for a longer duration.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 0125,023; Better et al., Science 240: 1041-1043, 1988; Liu et al., Proc. Natl. Acad. Sci. USA 84: 3439-3443, 1987; Liu et al., J. Immunol 139: 3521-3526, 1987; Sun et al., Proc. Natl. Acad. Sci. USA 84: 214-218, 1987; Nishimura et al., Cancer Res 47: 999-1005, 1987; Wood et al., Nature 314: 446-449, 1885; and Shaw et al., J. Natl. Cancer Inst. 80: 1553-1559, 1988.

As used herein, the term “consensus FR” means a framework (FR) antibody region in a consensus immunoglobulin sequence. The FR regions of an antibody do not contact the antigen.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

“Dosage form” and “unit dosage form”, as used herein, the term “dosage form” refers to physically discrete unit of a therapeutic agent for a subject (e.g., a human patient) to be treated. Each unit contains a predetermined quantity of active material calculated or demonstrated to produce a desired therapeutic effect when administered to a relevant population according to an appropriate dosing regimen. For example, in some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). It will be understood, however, that the total dosage administered to any particular patient will be selected by a medical professional (e.g., a medical doctor) within the scope of sound medical judgment.

“Dosing regimen” (or “therapeutic regimen”), as used herein is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in certain embodiments, a dosing regimen comprises a plurality of doses and at least two different time periods separating individual doses. In some embodiments, the therapeutic agent is administered continuously (e.g., by infusion) over a predetermined period. In other embodiments, a therapeutic agent is administered once a day (QD) or twice a day (BID). In some embodiments, a dosing regimen comprises a plurality of doses each of which are separated from one another by a time period of the same length; in other embodiments, a dosing regimen comprises a plurality of doses and at least two different time periods separating individual doses. In some embodiments, all doses within a dosing regimen are of the same unit dose amount. In certain embodiments, different doses within a dosing regimen are of different amounts. In some embodiments, a dosing regimen comprises a first dose in a first dose amount, followed by one or more additional doses in a second dose amount different from the first dose amount. In other embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some embodiments, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains (e.g., Fab, Fab′, F(ab′)₂, or Fv), in which all or substantially all of the hypervariable loops correspond to those of a non-human immunoglobulin and all or substantially all of the FR regions are those of a human immunoglobulin consensus FR sequence although the FR regions may include one or more amino acid substitutions that improve binding affinity. The number of these amino acid substitutions in the FR are typically no more than 6 in the H chain, and in the L chain, no more than 3. The humanized antibody optionally may also comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992). See e.g., Ahmed & Cheung, FEBS Letters 588(2):288-297 (2014).

As used herein, the term “hypervariable region” refers to the amino acid residues of an antibody which are responsible for antigen-binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity”, when used in the context of two or more nucleic acids or polypeptide sequences, refer to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein)), when compared and aligned for maximum correspondence over a comparison window or designated region as measured using a BLAST or BLAST 2.0 sequence comparison algorithms with default parameters described below, or by manual alignment and visual inspection (e.g., NCBI web site). Such sequences are then said to be “substantially identical.” This term also refers to, or can be applied to, the complement of a test sequence. The term also includes sequences that have deletions and/or additions, as well as those that have substitutions. In some embodiments, identity exists over a region that is at least about 25 amino acids or nucleotides in length, or 50-100 amino acids or nucleotides in length.

As used herein, the term “intact antibody” or “intact immunoglobulin” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the terms “individual”, “patient”, or “subject” can be an individual organism, a vertebrate, a mammal, or a human. In some embodiments, the individual, patient or subject is a human.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 amino acid residues. In certain embodiments, the linker comprises amino acids having the sequence GGGGSGGGGSGGGGS (i.e., [G₄S]₃) (SEQ ID NO: 158), GGGGSGGGGSGGGGSGGGGS (i.e., [G₄S]₄) (SEQ ID NO: 159), GGGGSGGGGSGGGGSGGGGSGGGGS (i.e., [G₄S]₅) (SEQ ID NO: 160), or GGGGSGGGGSGGGGSGGGGSGGGGSGGGGS (i.e., [G₄S]₆) (SEQ ID NO: 161).

The term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present methods may be made by the hybridoma method first described by Kohler et al., Nature 256:495 (1975), or may be made by recombinant DNA methods (See, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, “specifically binds” refers to a molecule (e.g., an antibody or antigen binding fragment thereof) which recognizes and binds another molecule (e.g., an antigen), but that does not substantially recognize and bind other molecules. The terms “specific binding,” “specifically binds to,” or is “specific for” a particular molecule (e.g., a polypeptide, or an epitope on a polypeptide), as used herein, can be exhibited, for example, by a molecule having a K_{D} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. The term “specifically binds” may also refer to binding where a molecule (e.g., an antibody or antigen binding fragment thereof) binds to a particular polypeptide (e.g., a CD3 polypeptide), or an epitope on a particular polypeptide, without substantially binding to any other polypeptide, or polypeptide epitope.

As used herein, the term “simultaneous” therapeutic use refers to the administration of at least two active ingredients by the same route and at the same time or at substantially the same time.

As used herein, the term “T-cell” includes naïve T cells, CD4+ T cells, CD8+ T cells, memory T cells, activated T cells, anergic T cells, tolerant T cells, chimeric B cells, and antigen-specific T cells.

As used herein, the term “therapeutic agent” is intended to mean a compound that, when present in an effective amount, produces a desired therapeutic effect on a subject in need thereof.

As used herein “tumor-infiltrating lymphocytes” or “TILs” refer to white blood cells that have left the bloodstream and migrated into a tumor.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Amino acid sequence modification(s) of the anti-CD3 antibodies described herein are contemplated. For example, it may be desirable to improve the binding affinity and/or other biological properties of the antibody. Amino acid sequence variants of an anti-CD3 antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to obtain the antibody of interest, as long as the obtained antibody possesses the desired properties. The modification also includes the change of the pattern of glycosylation of the protein. The sites of greatest interest for substitutional mutagenesis include the hypervariable regions, but FR alterations are also contemplated. “Conservative substitutions” are shown in the Table below.

TABLE 1
Amino Acid Substitutions

| Original | | Conservative |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

### EATs of the Present Technology

The present disclosure provides ex vivo armed T cells (EATs) that are coated or complexed with an effective arming dose of multi-specific (e.g., bispecific) antibodies that bind to CD3 and at least one additional target antigen (e.g., antigen that is expressed by tumor cells and/or a DOTA-based hapten). The EATs of the present disclosure may be armed with an effective arming dose of at least one type of anti-CD3 multi-specific antibody described herein. In certain embodiments, the EATs of the present disclosure may be armed with an effective arming dose of at least 1, 2, 3, 4, 5, 6, 7, 8, 9, 10 or more types of anti-CD3 multi-specific antibodies described herein.

T cells are lymphocytes that mature in the thymus and are chiefly responsible for cell-mediated immunity. T cells are involved in the adaptive immune system. The T cells included in the EATs of the presently disclosed subject matter can be any type of T cells, including, but not limited to, T helper cells, cytotoxic T cells, memory T cells (including central memory T cells), stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T (MAIT) cells, EBV-specific cytotoxic T cells (EBV-CTLs), αβ T cells and γδ T cells. Cytotoxic T cells (CTL or killer T cells) are a subset of T lymphocytes capable of inducing the death of infected somatic or tumor cells.

In any and all embodiments of the EATs disclosed herein, the at least one type of anti-CD3 multi-specific antibody exhibits surface densities between about 500 to about 20,000 molecules per T cell or between about 1,500 to 10,000 molecules per T cell. In certain embodiments, the at least one type of anti-CD3 multi-specific antibody exhibits surface densities of about 500, about 550, about 600, about 650, about 700, about 750, about 800, about 850, about 900, about 950, about 1000, about 1250, about 1500, about 1750, about 2000, about 2250, about 2500, about 2750, about 3000, about 3250, about 3500, about 3750, about 4000, about 4250, about 4500, about 4750, about 5000, about 5500, about 6000, about 6500, about 7000, about 7500, about 8000, about 8500, about 9000, about 9500, about 10,000, about 11,000, about 12,000, about 13,000, about 14,000, about 15,000, about 16,000, about 17,000, about 18,000, about 19,000, about 20,000, about 25,000, about 30,000, or about 35,000 molecules per T cell. Values and ranges intermediate to the recited values are also contemplated.

In any and all embodiments of the EATs disclosed herein, T cells are armed ex vivo with the at least one type of anti-CD3 multi-specific antibody at doses (e.g., effective arming dose) ranging between about 0.05 μg/10⁶ T cells to about 5 μg/10⁶ T cells. In certain embodiments, T cells are armed ex vivo with the at least one type of anti-CD3 multi-specific antibody at a dose (e.g., effective arming dose) of about 0.05 μg/10⁶ T cells, about 0.06 μg/10⁶ T cells, about 0.07 μg/10⁶ T cells, about 0.08 μg/10⁶ T cells, about 0.09 μg/10⁶ T cells, about 0.1 μg/10⁶ T cells, about 0.2 μg/10⁶ T cells, about 0.3 μg/10⁶ T cells, about 0.4 μg/10⁶ T cells, about 0.5 μg/10⁶ T cells, about 0.6 μg/10⁶ T cells, about 0.7 μg/10⁶ T cells, about 0.8 μg/10⁶ T cells, about 0.9 μg/10⁶ T cells, about 1.0 μg/10⁶ T cells, about 1.5 μg/10⁶ T cells, about 2.0 μg/10⁶ T cells, about 2.5 μg/10⁶ T cells, about 3.0 μg/10⁶ T cells, about 3.5 μg/10⁶ T cells, about 4.0 μg/10⁶ T cells, about 4.5 μg/10⁶ T cells, or about 5.0 μg/10⁶ T cells. Values and ranges intermediate to the recited values are also contemplated. Additionally or alternatively, in some embodiments, T cells are armed ex vivo by contacting T cells with an effective arming dose of the at least one type of anti-CD3 multi-specific antibody for about 5-60 minutes at room temperature. In certain embodiments, T cells are armed ex vivo by contacting T cells with an effective arming dose of the at least one type of anti-CD3 multi-specific antibody for about 5 mins, about 10 mins, about 15 mins, about 20 mins, about 25 mins, about 30 mins, about 35 mins, about 40 mins, about 45 mins, about 50 mins, about 55 mins, or about 60 mins at room temperature. Values and ranges intermediate to the recited values are also contemplated.

Additionally or alternatively, in some embodiments, the EATs are freshly prepared or have been cryopreserved. In certain embodiments, the EATs are cryopreserved for a period of about 2 hours to about 1 or more years. In some embodiments, the EATs are cryopreserved for a period of at least 2 hours, at least 3 hours, at least 4 hours, at least 5 hours, at least 6 hours, at least 7 hours, at least 8 hours, at least 9 hours, at least 10 hours, at least 12 hours, at least 24 hours, at least 48 hours, at least 3 days, at least 4 days, at least 5 days, at least 6 days, at least 1 week, at least 2 weeks, at least 3 weeks, at least 5 weeks, at least 1 month, at least 2 months, at least 3 months, at least 4 months, at least 5 months, at least 6 months, at least 7 months, at least 8 months, at least 9 months, at least 10 months, at least 11 months, or at least 12 months. Values and ranges intermediate to the recited values are also contemplated.

The EATs can be generated using peripheral donor lymphocytes, e.g., those disclosed in Panelli et al., J Immunol 164:495-504 (2000); Panelli et al., J Immunol 164:4382-4392 (2000) (disclosing lymphocyte cultures derived from tumor infiltrating lymphocytes (TILs) in tumor biopsies). The EATs can be autologous, non-autologous (e.g., allogeneic), or derived in vitro from lymphoid progenitor or stem cells.

The unpurified source of T cells may be any source known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cells initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Suitably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Usually, the cells are collected in a medium comprising 2% fetal calf serum (FCS) or 0.2% bovine serum albumin (BSA) or any other suitable (e.g., sterile), isotonic medium.

Administration. EATs of the presently disclosed subject matter can be provided systemically or directly to a subject for treating or preventing a neoplasia. In certain embodiments, EATs are directly injected into an organ of interest (e.g., an organ affected by a neoplasia). Alternatively or additionally, the EATs are provided indirectly to the organ of interest, for example, by administration into the circulatory system (e.g., the tumor vasculature) or into the solid tumor. Expansion and differentiation agents can be provided prior to, during or after administration of cells and compositions to promote maintenance/survival of T cells in vitro or in vivo.

EATs of the presently disclosed subject matter can be administered in any physiologically acceptable vehicle, systemically or regionally, normally intravascularly, intraperitoneally, intrathecally, or intrapleurally, although they may also be introduced into bone or other convenient site. In certain embodiments, at least 1×10⁵ cells, at least 1×10⁶ cells or 1×10¹⁰ or more cells can be administered. A cell population comprising EATs can comprise a purified population of cells. Those skilled in the art can readily determine the percentage of EATs in a cell population using various well-known methods, such as fluorescence activated cell sorting (FACS). The ranges of purity in cell populations comprising EATs can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The EATs can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising EATs coated or complexed with an effective arming dose of at least one type of anti-CD3 multi-specific antibody described herein and a pharmaceutically acceptable carrier. Administration can be autologous or non-autologous. For example, EATs coated or complexed with an effective arming dose of at least one type of anti-CD3 multi-specific antibody described herein and compositions comprising thereof can be obtained from one subject, and administered to the same subject or a different, compatible subject. Peripheral blood derived EATs of the presently disclosed subject matter can be administered via localized injection, including catheter administration, systemic injection, localized injection, intravenous injection, or parenteral administration. When administering a pharmaceutical composition of the presently disclosed subject matter (e.g., a pharmaceutical composition comprising EATs coated or complexed with an effective arming dose of at least one type of anti-CD3 multi-specific antibody described herein), it can be formulated in a unit dosage injectable form (solution, suspension, emulsion).

Formulations. EATs coated or complexed with an effective arming dose of at least one type of anti-CD3 multi-specific antibody described herein and compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising EATs, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the EATs of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the EATs as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the EATs of the presently disclosed subject matter is the quantity of cells necessary to achieve an optimal effect. The quantity of cells to be administered will vary for the subject being treated. In certain embodiments, from about 10² to about 10¹², from about 10³ to about 10¹¹, from about 10⁴ to about 10¹⁰, from about 10⁵ to about 10⁹, or from about 10⁶ to about 10⁸ EATs of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more EATs of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, EATs are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %.

Toxicity. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation. Optimally, an effective amount (e.g., dose) of an EAT described herein will provide therapeutic benefit without causing substantial toxicity to the subject. Toxicity of the EAT described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the EAT described herein lies within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

### Anti-CD3 Multi-Specific Antibodies Useful in Arming the EATs of the Present Technology

Anti-CD3 multi-specific antibodies that arm the EATs of the present technology include, e.g., but are not limited to, monoclonal, chimeric, humanized, bispecific antibodies, trispecific antibodies, or tetraspecific antibodies that specifically bind a CD3 target polypeptide, a homolog, derivative or a fragment thereof. In any and all embodiments of the EATs disclosed herein, the anti-CD3 multi-specific antibody that arms the EATs of the present technology is an immunoglobulin comprising two heavy chains and two light chains, wherein each of the light chains is fused to a single chain variable fragment (scFv). Such an anti-CD3 multi-specific antibody includes a CD3 binding domain comprising a heavy chain immunoglobulin variable domain (V_{H}) and a light chain immunoglobulin variable domain (V_{L}). Additionally or alternatively, in some embodiments, at least one scFv of the anti-CD3 multi-specific antibody disclosed herein comprises the CD3 binding domain. The CDR sequences of the V_{H} and V_{L} of the CD3 binding domain based on the IMGT annotation system are summarized below:

| Region | Definition | Sequence Fragment | Residues | Length |
|---|---|---|---|---|
| CDR-H1 | IMGT | GYTFTRYT | 26-33 | 8 |
| | | (SEQ ID NO: 1) | | |
| CDR-H2 | IMGT | INPSRGYT | 51-58 | 8 |
| | | (SEQ ID NO: 2) | | |
| CDR-H3 | IMGT | ARYYDDHYSLDY | 97-108 | 2 |
| | | (SEQ ID NO: 3) | | |
| CDR-L1 | IMGT | SSVSY | 27-31 | 5 |
| | | (SEQ ID NO: 4) | | |
| CDR-L2 | IMGT | DT (SEQ ID NO: 5) | 49-50 | 2 |
| CDR-L3 | IMGT | QQWSSNPFT | 88-96 | 9 |
| | | (SEQ ID NO: 6) | | |

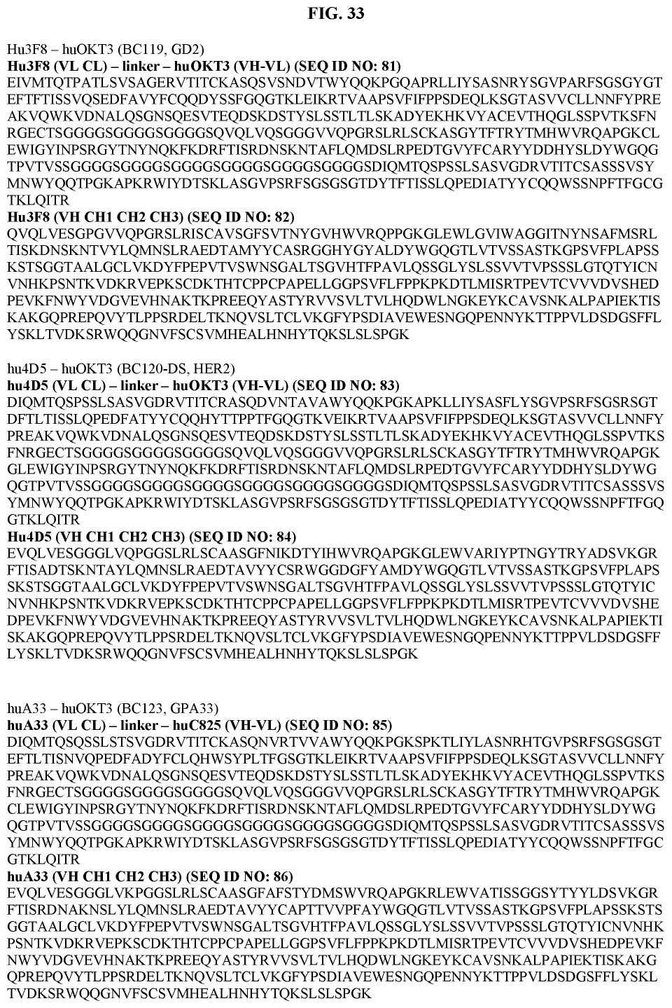

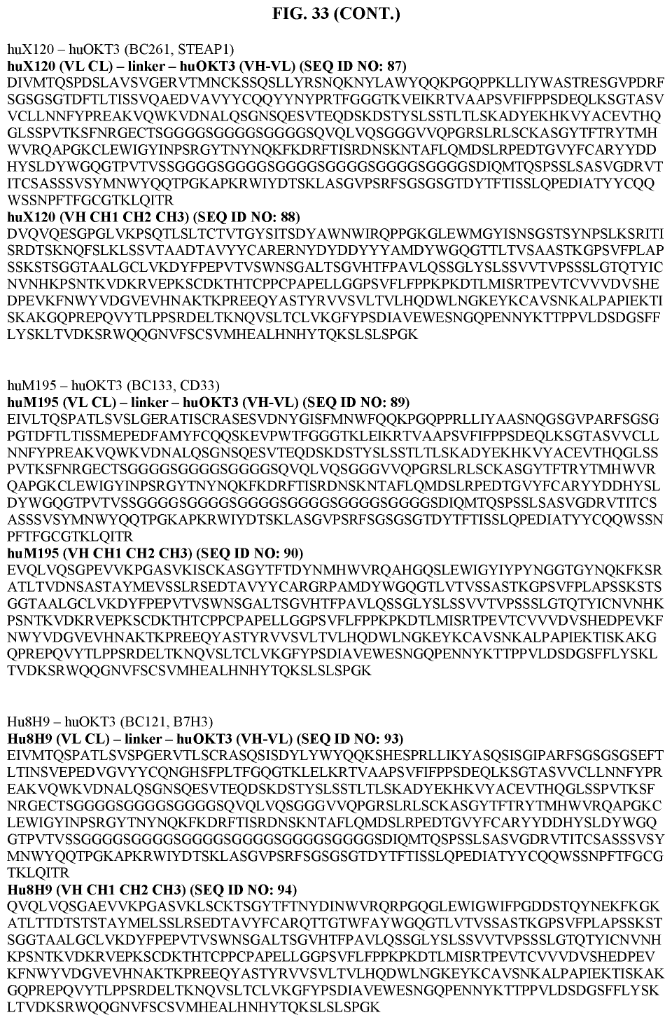

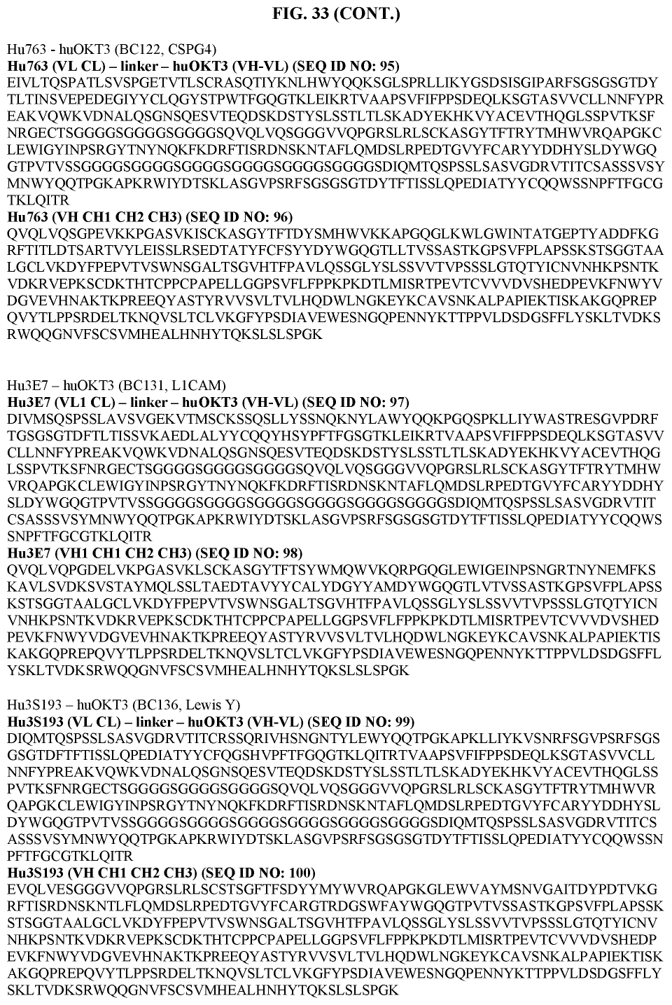

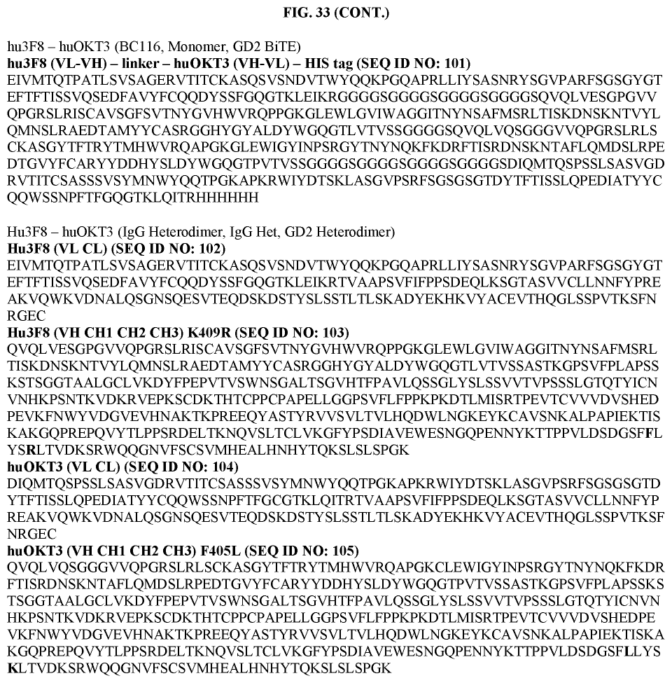

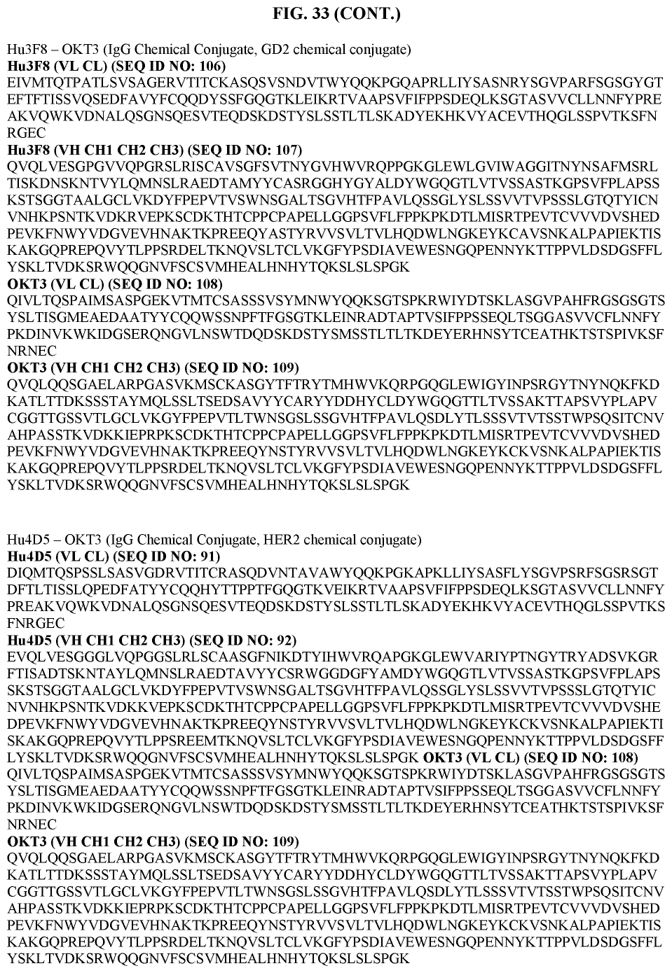

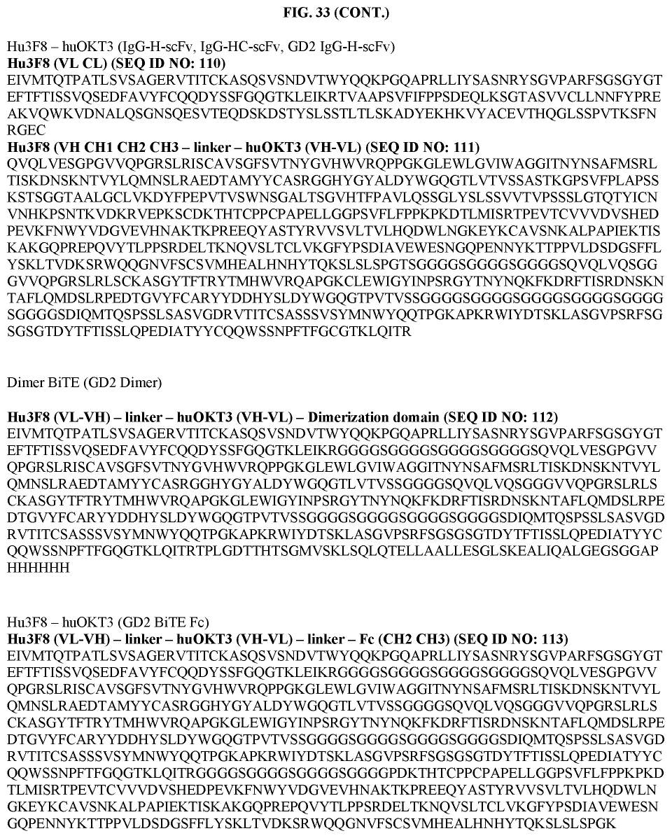

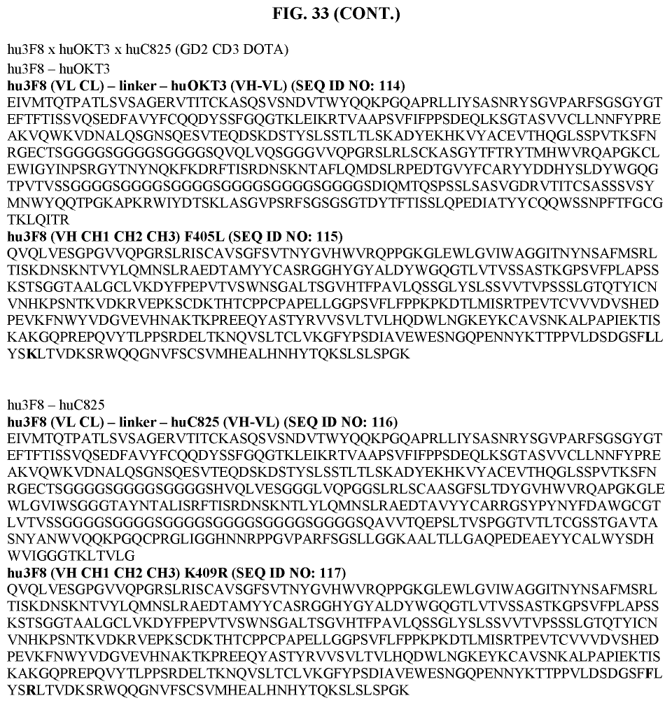

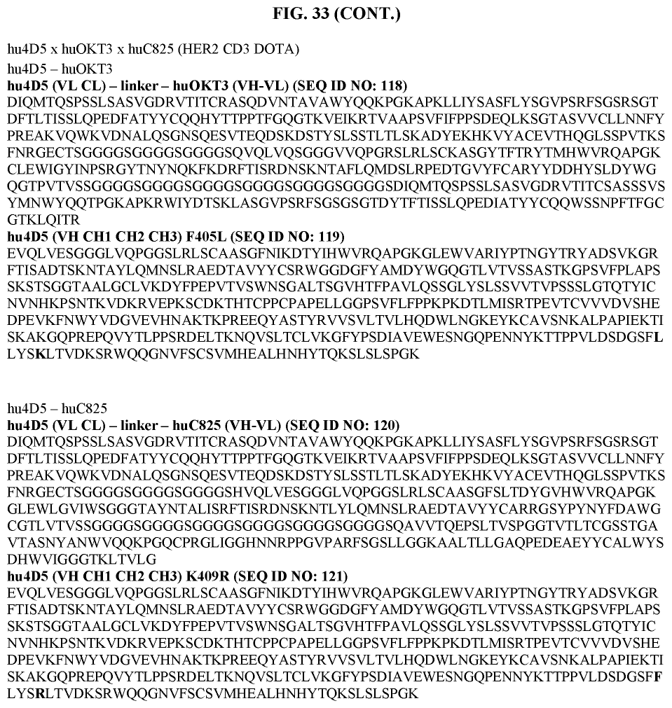

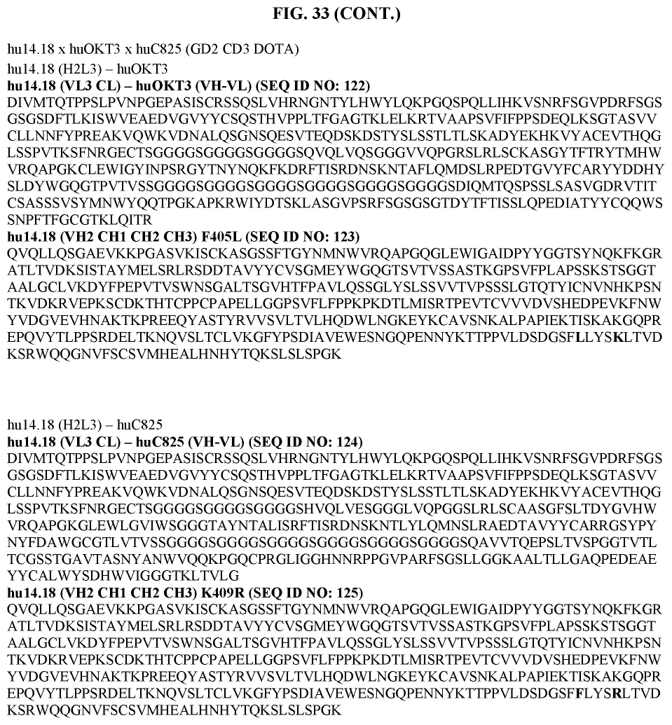

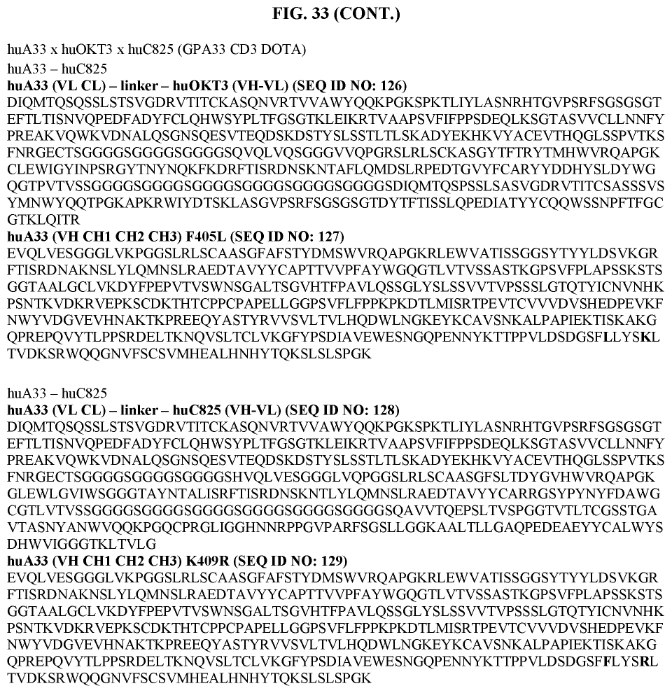

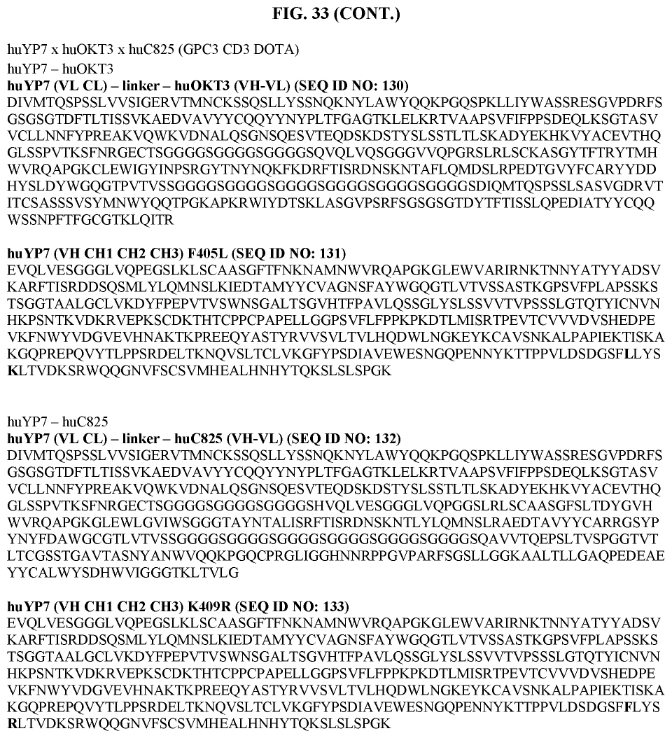

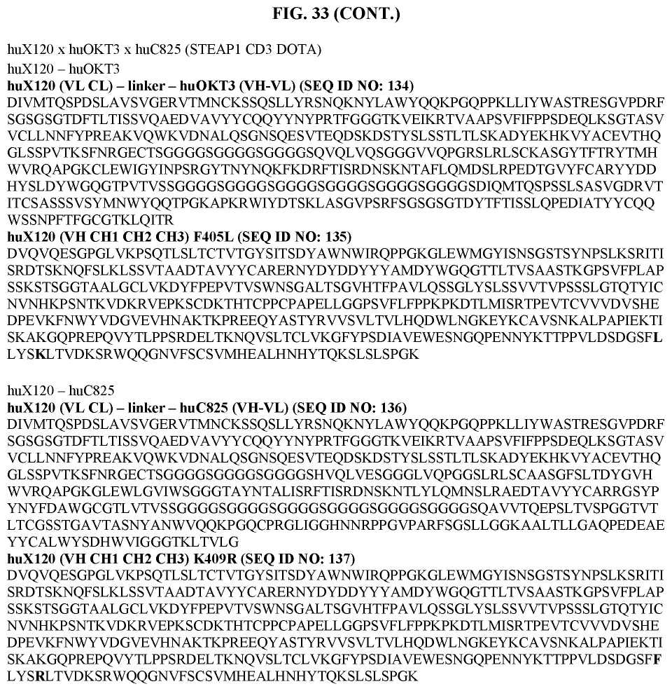

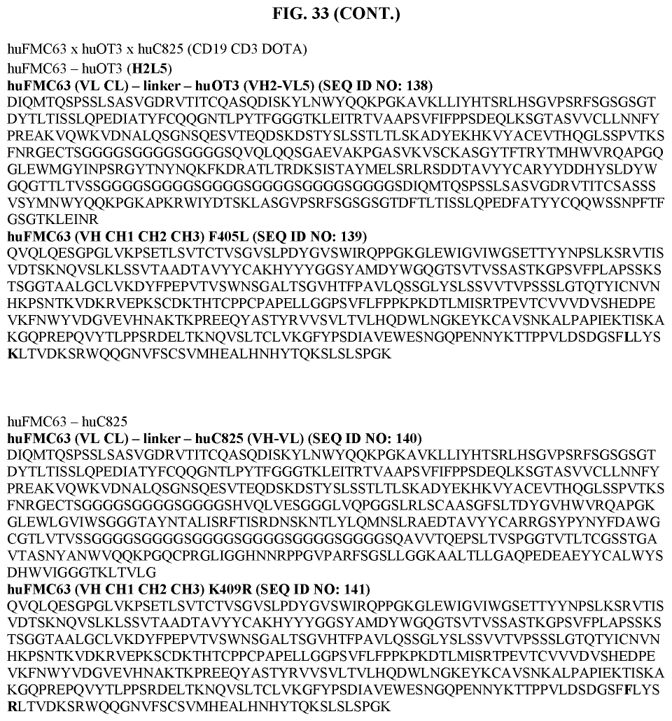

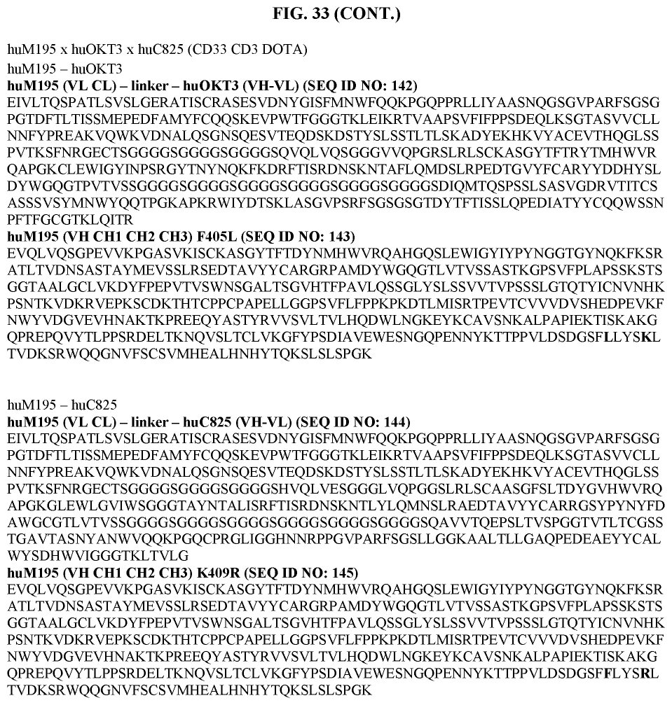

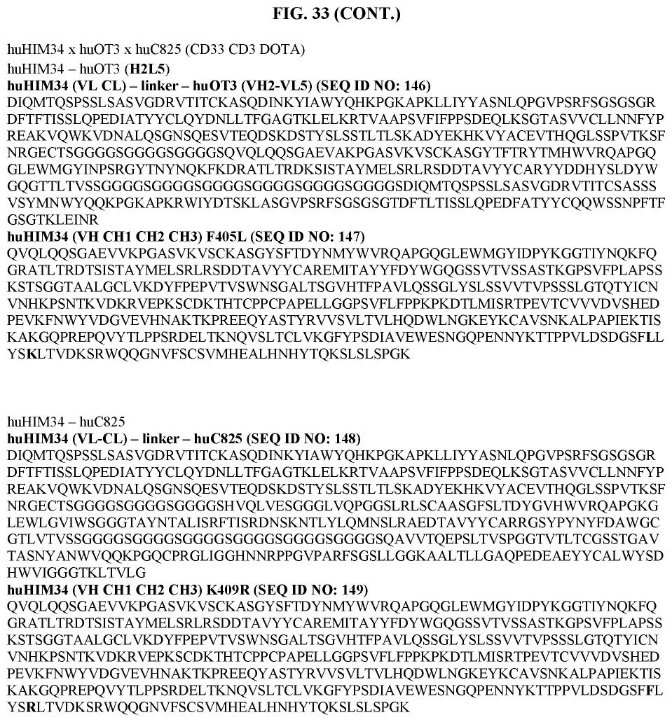

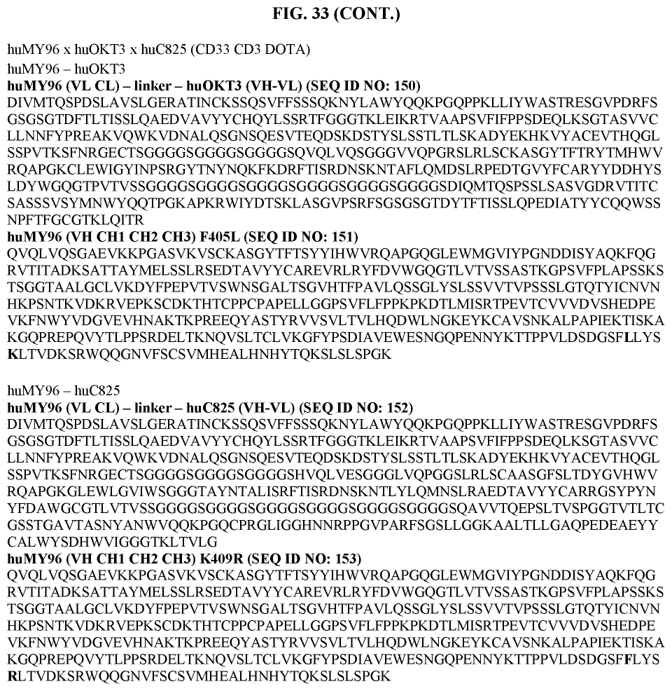

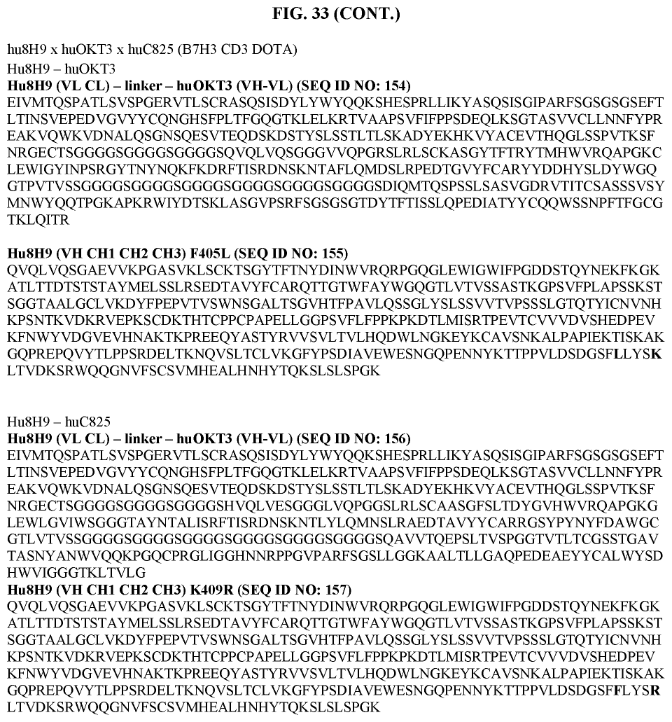

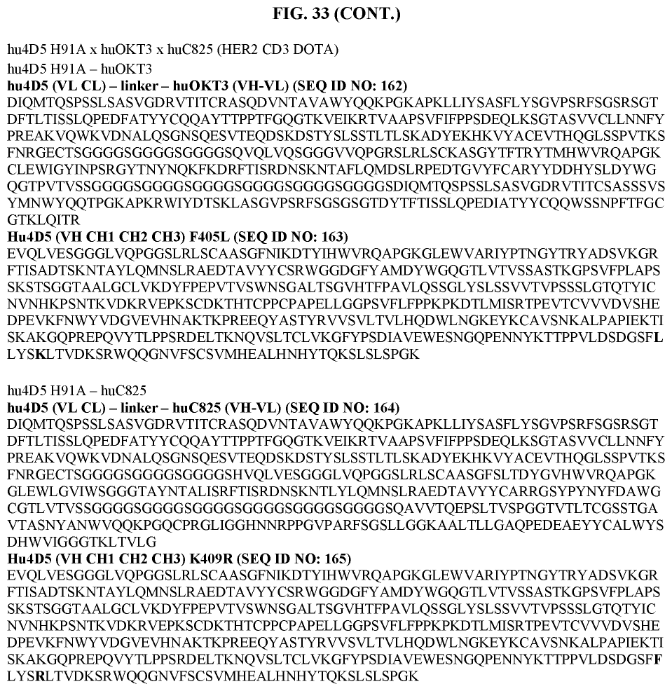

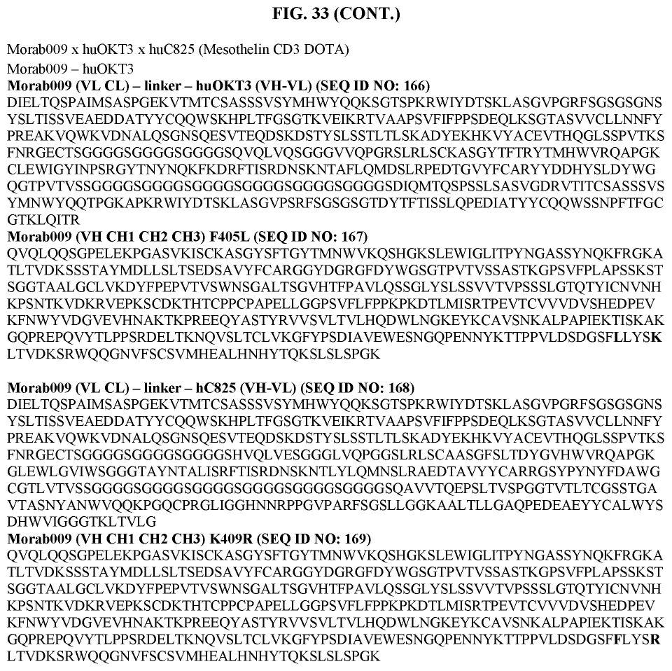

FIG. 33 shows exemplary amino acid sequences of anti-CD3 multi-specific antibodies that are useful for arming the EATs of the present technology.

In some embodiments, the anti-CD3 multi-specific antibodies that arm the EATs of the present technology include a CD3 binding domain comprising a heavy chain immunoglobulin variable domain (V_{H}) and a light chain immunoglobulin variable domain (V_{L}), wherein (a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and/or (b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6.

Exemplary heavy chain immunoglobulin variable domain amino acid sequences of the anti-CD3 antibodies of the present technology include:

huOKT3
(SEQ ID NO: 7)
QVQLVQSGGGVVQPGRSLRLSCKASGYTFTRYTMHWVRQAPGKCLEWIGYINPSRGYT
NYNQKFKDRFTISRDNSKNTAFLQMDSLRPEDTGVYFCARYYDDHYSLDYWGQGTPVT
VSS
huOKT3-DS
(SEQ ID NO: 8)
QVQLVQSGGGVVQPGRSLRLSCKASGYTFTRYTMHWVRQAPGKGLEWIGYINPSRGYT
NYNQKFKDRFTISRDNSKNTAFLQMDSLRPEDTGVYFCARYYDDHYSLDYWGQGTPVT
VSS
VH-1 (humanness 85.7%)
(SEQ ID NO: 9)
QVQLQQSGAEVAKPGASVKMSCKASGYTFTRYTMHWVRQAPGQGLEWIGYINPSRGY
TNYAQKFQGRATLTTDKSISTAYMELSRLRSDDTAVYYCARYYDDHYSLDYWGQGTT
LTVSS
VH-2 (humanness 85.7%)
(SEQ ID NO: 10)
QVQLQQSGAEVAKPGASVKVSCKASGYTFTRYTMHWVRQAPGQGLEWMGYINPSRG
YTNYNQKFKDRATLTRDKSISTAYMELSRLRSDDTAVYYCARYYDDHYSLDYWGQGT
TLTVSS
VH-3 (humanness 85.7%)
(SEQ ID NO: 11)
QVQLVQSGAEVKKPGASVKMSCKASGYTFTRYTMHWVRQAPGQGLEWIGYINPSRGY
TNYNQKFKDRATMTTDKSISTAYMELSRLRSDDTAVYYCARYYDDHYSLDYWGQGTT
LTVSS
VH-4 (humanness 85.7%)
(SEQ ID NO: 12)
QVQLQQSGAEVKKPGASVKMSCKASGYTFTRYTMHWVRQAPGQGLEWIGYINPSRGY
TNYNQKFKDRVTLTTDTSISTAYMELSRLRSDDTAVYYCARYYDDHYSLDYWGQGTTL
TVSS
VH-1 H105 (humanness 85.7%)
(SEQ ID NO: 13)
QVQLQQSGAEVAKPGASVKMSCKASGYTFTRYTMHWVRQAPGQGLEWIGYINPSRGY
TNYAQKFQGRATLTTDKSISTAYMELSRLRSDDTAVYYCARYYDDHYSLDYWGCGTTL
TVSS
VH-2 H105 (humanness 85.7%)
(SEQ ID NO: 14)
QVQLQQSGAEVAKPGASVKVSCKASGYTFTRYTMHWVRQAPGQGLEWMGYINPSRG
YTNYNQKFKDRATLTRDKSISTAYMELSRLRSDDTAVYYCARYYDDHYSLDYWGCGT
TLTVSS
VH-3 H105 (humanness 85.7%)
(SEQ ID NO: 15)
QVQLVQSGAEVKKPGASVKMSCKASGYTFTRYTMHWVRQAPGQGLEWIGYINPSRGY
TNYNQKFKDRATMTTDKSISTAYMELSRLRSDDTAVYYCARYYDDHYSLDYWGCGTT
LTVSS
VH-4 H105 (humanness 85.7%)
(SEQ ID NO: 16)
QVQLQQSGAEVKKPGASVKMSCKASGYTFTRYTMHWVRQAPGQGLEWIGYINPSRGY
TNYNQKFKDRVTLTTDTSISTAYMELSRLRSDDTAVYYCARYYDDHYSLDYWGCGTTL
TVSS
VH-1 H44 (humanness 85.7%)
(SEQ ID NO: 17)
QVQLQQSGAEVAKPGASVKMSCKASGYTFTRYTMHWVRQAPGQCLEWIGYINPSRGY
TNYAQKFQGRATLTTDKSISTAYMELSRLRSDDTAVYYCARYYDDHYSLDYWGQGTT
LTVSS
VH-2 H44 (humanness 85.7%)
(SEQ ID NO: 18)
QVQLQQSGAEVAKPGASVKVSCKASGYTFTRYTMHWVRQAPGQCLEWMGYINPSRG
YTNYNQKFKDRATLTRDKSISTAYMELSRLRSDDTAVYYCARYYDDHYSLDYWGQGT
TLTVSS
VH-3 H44 (humanness 85.7%)
(SEQ ID NO: 19)
QVQLVQSGAEVKKPGASVKMSCKASGYTFTRYTMHWVRQAPGQCLEWIGYINPSRGY
TNYNQKFKDRATMTTDKSISTAYMELSRLRSDDTAVYYCARYYDDHYSLDYWGQGTT
LTVSS
VH-4 H44 (humanness 85.7%)
(SEQ ID NO: 20)
QVQLQQSGAEVKKPGASVKMSCKASGYTFTRYTMHWVRQAPGQCLEWIGYINPSRGY
TNYNQKFKDRVTLTTDTSISTAYMELSRLRSDDTAVYYCARYYDDHYSLDYWGQGTTL
TVSS
VH-1 H100B (humanness 85.7%)
(SEQ ID NO: 21)
QVQLQQSGAEVAKPGASVKMSCKASGYTFTRYTMHWVRQAPGQGLEWIGYINPSRGY
TNYAQKFQGRATLTTDKSISTAYMELSRLRSDDTAVYYCARYYDDHYSCDYWGQGTT
LTVSS
VH-2 H100B (humanness 85.7%)
(SEQ ID NO: 22)
QVQLQQSGAEVAKPGASVKVSCKASGYTFTRYTMHWVRQAPGQGLEWMGYINPSRG
YTNYNQKFKDRATLTRDKSISTAYMELSRLRSDDTAVYYCARYYDDHYSCDYWGQGT
TLTVSS
VH-3 H100B (humanness 85.7%)
(SEQ ID NO: 23)
QVQLVQSGAEVKKPGASVKMSCKASGYTFTRYTMHWVRQAPGQGLEWIGYINPSRGY
TNYNQKFKDRATMTTDKSISTAYMELSRLRSDDTAVYYCARYYDDHYSCDYWGQGTT
LTVSS
VH-4 H100B (humanness 85.7%)
(SEQ ID NO: 24)
QVQLQQSGAEVKKPGASVKMSCKASGYTFTRYTMHWVRQAPGQGLEWIGYINPSRGY
TNYNQKFKDRVTLTTDTSISTAYMELSRLRSDDTAVYYCARYYDDHYSCDYWGQGTTL
TVSS
VH-1 H100 (humanness 85.7%)
(SEQ ID NO: 25)
QUQLQQSGAEVAKPGASVKMSCKASGYTFTRYTMHWVRQAPGQGLEWIGYINPSRGY
TNYAQKFQGRATLTTDKSISTAYMELSRLRSDDTAVYYCARYYDDHCSLDYWGQGTTL
TVSS
VH-2 H100 (humanness 85.7%)
(SEQ ID NO: 26)
QVQLQQSGAEVAKPGASVKVSCKASGYTFTRYTMHWVRQAPGQGLEWMGYINPSRG
YTNYNQKFKDRATLTRDKSISTAYMELSRLRSDDTAVYYCARYYDDHCSLDYWGQGT
TLTVSS
VH-3 H100 (humanness 85.7%)
(SEQ ID NO: 27)
QVQLVQSGAEVKKPGASVKMSCKASGYTFTRYTMHWVRQAPGQGLEWIGYINPSRGY
TNYNQKFKDRATMTTDKSISTAYMELSRLRSDDTAVYYCARYYDDHCSLDYWGQGTT
LTVSS
VH-4 H100 (humanness 85.7%)
(SEQ ID NO: 28)
QVQLQQSGAEVKKPGASVKMSCKASGYTFTRYTMHWVRQAPGQGLEWIGYINPSRGY
TNYNQKFKDRVTLTTDTSISTAYMELSRLRSDDTAVYYCARYYDDHCSLDYWGQGTTL
TVSS
VH-1 H101 (humanness 85.7%)
(SEQ ID NO: 29)
QVQLQQSGAEVAKPGASVKMSCKASGYTFTRYTMHWVRQAPGQGLEWIGYINPSRGY
TNYAQKFQGRATLTTDKSISTAYMELSRLRSDDTAVYYCARYYDDHYSLCYWGQGTTL
TVSS
VH-2 H101 (humanness 85.7%)
(SEQ ID NO: 30)
QVQLQQSGAEVAKPGASVKVSCKASGYTFTRYTMHWVRQAPGQGLEWMGYINPSRG
YTNYNQKFKDRATLTRDKSISTAYMELSRLRSDDTAVYYCARYYDDHYSLCYWGQGT
TLTVSS
VH-3 H101 (humanness 85.7%)
(SEQ ID NO: 31)
QVQLVQSGAEVKKPGASVKMSCKASGYTFTRYTMHWVRQAPGQGLEWIGYINPSRGY
TNYNQKFKDRATMTTDKSISTAYMELSRLRSDDTAVYYCARYYDDHYSLCYWGQGTT
LTVSS
VH-4 H101 (humanness 85.7%)
(SEQ ID NO: 32)
QVQLQQSGAEVKKPGASVKMSCKASGYTFTRYTMHWVRQAPGQGLEWIGYINPSRGY
TNYNQKFKDRVTLTTDTSISTAYMELSRLRSDDTAVYYCARYYDDHYSLCYWGQGTTL
TVSS
Exemplary light chain immunoglobulin variable domain amino acid
sequences of the anti-CD3 antibodies of the present technology include:
huOKT3
(SEQ ID NO: 33)
DIQMTQSPSSLSASVGDRVTITCSASSSVSYMNWYQQTPGKAPKRWIYDTSKLASGVPS
RFSGSGSGTDYTFTISSLQPEDIATYYCQQWSSNPFTFGCGTKLQITR
huOKT3-DS
(SEQ ID NO: 34)
DIQMTQSPSSLSASVGDRVTITCSASSSVSYMNWYQQTPGKAPKRWIYDTSKLASGVPS
RFSGSGSGTDYTFTISSLQPEDIATYYCQQWSSNPFTFGQGTKLQITR
VL-1 (humanness 85.2%)
(SEQ ID NO: 35)
DIQMTQSPSSLSASVGDRVTMTCSASSSVSYMNWYQQKPGKAPKRLIYDTSKLASGVPS
RFSGSGSGTDFTLTISSLQPEDFATYYCQQWSSNPFTFGSGTKLEINR
VL-2 (humanness 85.2%)
(SEQ ID NO: 36)
DIQMTQSPSSLSASVGDRVTMTCSASSSVSYMNWYQQKPGKAPKLLIYDTSKLASGVPS
RFSGSGSGTDFTLTISSMQPEDFATYYCQQWSSNPFTFGSGTKLEINR
VL-3 (humanness 85.2%)
(SEQ ID NO: 37)
DIQLTQSPSSLSASVGDRVTITCSASSSVSYMNWYQQKPGKAPKLLIYDTSKLASGVPSR
FSGSGSGTDYTLTISSLQPEDFATYYCQQWSSNPFTFGSGTKLEINR
VL-4 (humanness 85.2%)
(SEQ ID NO: 38)
DIQLTQSPSSLSASVGDRVTITCSASSSVSYMNWYQQKPGKAPKLLIYDTSKLASGVPSR
FSGSGSGTDFTLTISSMQPEDFATYYCQQWSSNPFTFGSGTKLEINR
VL-5 (humanness 85.2%)
(SEQ ID NO: 39)
DIQMTQSPSSLSASVGDRVTITCSASSSVSYMNWYQQKPGKAPKRWIYDTSKLASGVPS
RFSGSGSGTDFTLTISSLQPEDFATYYCQQWSSNPFTFGSGTKLEINR
VL-6 (humanness 85.2%)
(SEQ ID NO: 40)
DIQMTQSPSSLSASVGDRVTITCSASSSVSYMNWYQQKPGKAPKLWIYDTSKLASGVPS
RFSGSGSGTDYTLTISSLQPEDFATYYCQQWSSNPFTFGSGTKLEINR
VL-1 L100 (humanness 85.2%)
(SEQ ID NO: 41)
DIQMTQSPSSLSASVGDRVTMTCSASSSVSYMNWYQQKPGKAPKRLIYDTSKLASGVPS
RFSGSGSGTDFTLTISSLQPEDFATYYCQQWSSNPFTFGCGTKLEINR
VL-2 L100 (humanness 85.2%)
(SEQ ID NO: 42)
DIQMTQSPSSLSASVGDRVTMTCSASSSVSYMNWYQQKPGKAPKLLIYDTSKLASGVPS
RFSGSGSGTDFTLTISSMQPEDFATYYCQQWSSNPFTFGCGTKLEINR
VL-3 L100 (humanness 85.2%)
(SEQ ID NO: 43)
DIQLTQSPSSLSASVGDRVTITCSASSSVSYMNWYQQKPGKAPKLLIYDTSKLASGVPSR
FSGSGSGTDYTLTISSLQPEDFATYYCQQWSSNPFTFGCGTKLEINR
VL-4 L100 (humanness 85.2%)
(SEQ ID NO: 44)
DIQLTQSPSSLSASVGDRVTITCSASSSVSYMNWYQQKPGKAPKLLIYDTSKLASGVPSR
FSGSGSGTDFTLTISSMQPEDFATYYCQQWSSNPFTFGCGTKLEINR
VL-5 L100 (humanness 85.2%)
(SEQ ID NO: 45)
DIQMTQSPSSLSASVGDRVTITCSASSSVSYMNWYQQKPGKAPKRWIYDTSKLASGVPS
RFSGSGSGTDFTLTISSLQPEDFATYYCQQWSSNPFTFGCGTKLEINR
VL-6 L100 (humanness 85.2%)
(SEQ ID NO: 46)
DIQMTQSPSSLSASVGDRVTITCSASSSVSYMNWYQQKPGKAPKLWIYDTSKLASGVPS
RFSGSGSGTDYTLTISSLQPEDFATYYCQQWSSNPFTFGCGTKLEINR
VL-1 L43 (humanness 85.2%)
(SEQ ID NO: 47)
DIQMTQSPSSLSASVGDRVTMTCSASSSVSYMNWYQQKPGKCPKRLIYDTSKLASGVPS
RFSGSGSGTDFTLTISSLQPEDFATYYCQQWSSNPFTFGSGTKLEINR
VL-2 L43 (humanness 85.2%)
(SEQ ID NO: 48)
DIQMTQSPSSLSASVGDRVTMTCSASSSVSYMNWYQQKPGKCPKLLIYDTSKLASGVPS
RFSGSGSGTDFTLTISSMQPEDFATYYCQQWSSNPFTFGSGTKLEINR
VL-3 L43 (humanness 85.2%)
(SEQ ID NO: 49)
DIQLTQSPSSLSASVGDRVTITCSASSSVSYMNWYQQKPGKCPKLLIYDTSKLASGVPSR
FSGSGSGTDYTLTISSLQPEDFATYYCQQWSSNPFTFGSGTKLEINR
VL-4 L43 (humanness 85.2%)
(SEQ ID NO: 50)
DIQLTQSPSSLSASVGDRVTITCSASSSVSYMNWYQQKPGKCPKLLIYDTSKLASGVPSR
FSGSGSGTDFTLTISSMQPEDFATYYCQQWSSNPFTFGSGTKLEINR
VL-5 L43 (humanness 85.2%)
(SEQ ID NO: 51)
DIQMTQSPSSLSASVGDRVTITCSASSSVSYMNWYQQKPGKCPKRWIYDTSKLASGVPS
RFSGSGSGTDFTLTISSLQPEDFATYYCQQWSSNPFTFGSGTKLEINR
VL-6 L43 (humanness 85.2%)
(SEQ ID NO: 52)
DIQMTQSPSSLSASVGDRVTITCSASSSVSYMNWYQQKPGKCPKLWIYDTSKLASGVPS
RFSGSGSGTDYTLTISSLQPEDFATYYCQQWSSNPFTFGSGTKLEINR
VL-1 L49 (humanness 85.2%)
(SEQ ID NO: 53)
DIQMTQSPSSLSASVGDRVTMTCSASSSVSYMNWYQQKPGKAPKRLICDTSKLASGVPS
RFSGSGSGTDFTLTISSLQPEDFATYYCQQWSSNPFTFGSGTKLEINR
VL-2 L49 (humanness 85.2%)
(SEQ ID NO: 54)
DIQMTQSPSSLSASVGDRVTMTCSASSSVSYMNWYQQKPGKAPKLLICDTSKLASGVPS
RFSGSGSGTDFTLTISSMQPEDFATYYCQQWSSNPFTFGSGTKLEINR
VL-3 L49 (humanness 85.2%)
(SEQ ID NO: 55)
DIQLTQSPSSLSASVGDRVTITCSASSSVSYMNWYQQKPGKAPKLLICDTSKLASGVPSR
FSGSGSGTDYTLTISSLQPEDFATYYCQQWSSNPFTFGSGTKLEINR
VL-4 L49 (humanness 85.2%)
(SEQ ID NO: 56)
DIQLTQSPSSLSASVGDRVTITCSASSSVSYMNWYQQKPGKAPKLLICDTSKLASGVPSR
FSGSGSGTDFTLTISSMQPEDFATYYCQQWSSNPFTFGSGTKLEINR
VL-5 L49 (humanness 85.2%)
(SEQ ID NO: 57)
DIQMTQSPSSLSASVGDRVTITCSASSSVSYMNWYQQKPGKAPKRWICDTSKLASGVPS
RFSGSGSGTDFTLTISSLQPEDFATYYCQQWSSNPFTFGSGTKLEINR
VL-6 L49 (humanness 85.2%)
(SEQ ID NO: 58)
DIQMTQSPSSLSASVGDRVTITCSASSSVSYMNWYQQKPGKAPKLWICDTSKLASGVPS
RFSGSGSGTDYTLTISSLQPEDFATYYCQQWSSNPFTFGSGTKLEINR
VL-1 L50 (humanness 85.2%)
(SEQ ID NO: 59)
DIQMTQSPSSLSASVGDRVTMTCSASSSVSYMNWYQQKPGKAPKRLIYCTSKLASGVPS
RFSGSGSGTDFTLTISSLQPEDFATYYCQQWSSNPFTFGSGTKLEINR
VL-2 L50 (humanness 85.2%)
(SEQ ID NO: 60)
DIQMTQSPSSLSASVGDRVTMTCSASSSVSYMNWYQQKPGKAPKLLIYCTSKLASGVPS
RFSGSGSGTDFTLTISSMQPEDFATYYCQQWSSNPFTFGSGTKLEINR
VL-3 L50 (humanness 85.2%)
(SEQ ID NO: 61)
DIQLTQSPSSLSASVGDRVTITCSASSSVSYMNWYQQKPGKAPKLLIYCTSKLASGVPSR
FSGSGSGTDYTLTISSLQPEDFATYYCQQWSSNPFTFGSGTKLEINR
VL-4 L50 (humanness 85.2%)
(SEQ ID NO: 62)
DIQLTQSPSSLSASVGDRVTITCSASSSVSYMNWYQQKPGKAPKLLIYCTSKLASGVPSR
FSGSGSGTDFTLTISSMQPEDFATYYCQQWSSNPFTFGSGTKLEINR
VL-5 L50 (humanness 85.2%)
(SEQ ID NO: 63)
DIQMTQSPSSLSASVGDRVTITCSASSSVSYMNWYQQKPGKAPKRWIYCTSKLASGVPS
RFSGSGSGTDFTLTISSLQPEDFATYYCQQWSSNPFTFGSGTKLEINR
VL-6 L50 (humanness 85.2%)
(SEQ ID NO: 64)
DIQMTQSPSSLSASVGDRVTITCSASSSVSYMNWYQQKPGKAPKLWIYCTSKLASGVPS
RFSGSGSGTDYTLTISSLQPEDFATYYCQQWSSNPFTFGSGTKLEINR
VL-1 L46 (humanness 85.2%)
(SEQ ID NO: 65)
DIQMTQSPSSLSASVGDRVTMTCSASSSVSYMNWYQQKPGKAPKCLIYDTSKLASGVPS
RFSGSGSGTDFTLTISSLQPEDFATYYCQQWSSNPFTFGSGTKLEINR
VL-2 L46 (humanness 85.2%)
(SEQ ID NO: 66)
DIQMTQSPSSLSASVGDRVTMTCSASSSVSYMNWYQQKPGKAPKCLIYDTSKLASGVPS
RFSGSGSGTDFTLTISSMQPEDFATYYCQQWSSNPFTFGSGTKLEINR
VL-3 L46 (humanness 85.2%)
(SEQ ID NO: 67)
DIQLTQSPSSLSASVGDRVTITCSASSSVSYMNWYQQKPGKAPKCLIYDTSKLASGVPSR
FSGSGSGTDYTLTISSLQPEDFATYYCQQWSSNPFTFGSGTKLEINR
VL-4 L46 (humanness 85.2%)
(SEQ ID NO: 68)
DIQLTQSPSSLSASVGDRVTITCSASSSVSYMNWYQQKPGKAPKCLIYDTSKLASGVPSR
FSGSGSGTDFTLTISSMQPEDFATYYCQQWSSNPFTFGSGTKLEINR
VL-5 L46 (humanness 85.2%)
(SEQ ID NO: 69)
DIQMTQSPSSLSASVGDRVTITCSASSSVSYMNWYQQKPGKAPKCWIYDTSKLASGVPS
RFSGSGSGTDFTLTISSLQPEDFATYYCQQWSSNPFTFGSGTKLEINR
VL-6 L46 (humanness 85.2%)
(SEQ ID NO: 70)
DIQMTQSPSSLSASVGDRVTITCSASSSVSYMNWYQQKPGKAPKCWIYDTSKLASGVPS
RFSGSGSGTDYTLTISSLQPEDFATYYCQQWSSNPFTFGSGTKLEINR

Additionally or alternatively, in some embodiments, the anti-CD3 multi-specific antibodies that arm the EATs of the present technology include a CD3 binding domain comprising a heavy chain immunoglobulin variable domain (V_{H}) and a light chain immunoglobulin variable domain (V_{L}), wherein: (a) the V_{H} comprises an amino acid sequence selected from any one of SEQ ID NOs: 7-32; and/or (b) the V_{L} comprises an amino acid sequence selected from any one of SEQ ID NOs: 33-70.

In certain embodiments, the anti-CD3 multi-specific antibodies that arm the EATs of the present technology includes one or more of the following characteristics: (a) a light chain immunoglobulin variable domain sequence that is at least 80%, at least 85%, at least 90%, at least 95%, or at least 99% identical to the light chain immunoglobulin variable domain sequence of any one of SEQ ID NOs: 33-70; and/or (b) a heavy chain immunoglobulin variable domain sequence that is at least 80%, at least 85%, at least 90%, at least 95%, or at least 99% identical to the heavy chain immunoglobulin variable domain sequence of any one of SEQ ID NOs: 7-32. In another aspect, one or more amino acid residues in the immunoglobulin-related compositions provided herein are substituted with another amino acid. The substitution may be a “conservative substitution” as defined herein.

In some embodiments, the anti-CD3 multi-specific antibodies that arm the EATs of the present technology bind to the extracellular domain of a CD3 polypeptide. In certain embodiments, the epitope is a conformational epitope or non-conformational epitope. In some embodiments, the CD3 polypeptide has the amino acid sequence of SEQ ID NO: 71.

NCBI Ref: NP_000724.1 Homo sapiens T-cell surface glycoprotein CD3 epsilon chain precursor (SEQ ID NO: 71)

MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI

Additionally or alternatively, in some embodiments, the anti-CD3 multi-specific antibodies bind to the extracellular domain of a CD3 polypeptide. In certain embodiments, the extracellular domain comprises a CD3ε subunit including a linear stretch of sequence on the F-G loop. In some embodiments, the CD3ε subunit may comprise three discontinuous regions: residues 79ε-85c (the F-G loop), residue 34ε (the first residue of the BC strand), and residues 46ε and 48ε (the C′-D loop).

In any of the above embodiments, the anti-CD3 multi-specific antibodies further comprises a Fc domain of any isotype, e.g., but are not limited to, IgG (including IgG1, IgG2, IgG3, and IgG4).

Non-limiting examples of constant region sequences include:

Human IgG1 constant region, Uniprot: P01857
(SEQ ID NO: 72)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK
Human IgG2 constant region, Uniprot: P01859
(SEQ ID NO: 73)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKTKPREEQFNST
FRVVSVLTVVHQDWLNGKEYKCKVSNKGLPAPIEKTISKTKGQPREPQVYTLPPSREEM
TKNQVSLTCLVKGFYPSDISVEWESNGQPENNYKTTPPMLDSDGSFFLYSKLTVDKSRW
QQGNVFSCSVMHEALHNHYTQKSLSLSPGK
Human IgG3 constant region, Uniprot: P01860
(SEQ ID NO: 74)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDTLMI
SRTPEVTCVVVDVSHEDPEVQFKWYVDGVEVHNAKTKPREEQYNSTFRVVSVLTVLHQ
DWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMH
EALHNRFTQKSLSLSPGK
Human IgG4 constant region, Uniprot: P01861
(SEQ ID NO: 75)
ASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPSCPAPE
FLGGPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
Human Ig kappa constant region, Uniprot: P01834
(SEQ ID NO: 76)
TVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC

In some embodiments, the anti-CD3 multi-specific antibodies that arm the EATs of the present technology comprise a heavy chain constant region that is at least 80%, at least 85%, at least 90%, at least 95%, at least 99%, or is 100% identical to SEQ ID NOs: 72-75. Additionally or alternatively, in some embodiments, the anti-CD3 multi-specific antibodies that arm the EATs of the present technology comprise a light chain constant region that is at least 80%, at least 85%, at least 90%, at least 95%, at least 99%, or is 100% identical to SEQ ID NO: 76. In certain embodiments, the anti-CD3 multi-specific antibodies that arm the EATs of the present technology contain an IgG1 constant region comprising one or more amino acid substitutions selected from the group consisting of N297A and K322A. Additionally or alternatively, in some embodiments, the immunoglobulin-related compositions contain an IgG4 constant region comprising a S228P mutation.

Additionally or alternatively, in some embodiments, the anti-CD3 multi-specific antibodies comprises a DOTA binding domain. The DOTA binding domain may include a V_{H} having the amino acid sequence of SEQ ID NO: 77 and/or a V_{L} having the amino acid sequence of SEQ ID NO: 78.

(SEQ ID NO: 77)
HVQLVESGGGLVQPGGSLRLSCAASGFSLTDYGVHWVRQAPGKGLEWLG
VIWSGGGTAYNTALISRFTISRDNSKNTLYLQMNSLRAEDTAVYYCARR
GSYPYNYFDAWGCGTLVTVSS
(SEQ ID NO: 78)
QAVVTQEPSLTVSPGGTVTLTCGSSTGAVTASNYANWVQQKPGQCPRGL
IGGHNNRPPGVPARFSGSLLGGKAALTLLGAQPEDEAEYYCALWYSDHW
VIGGGTKLTVLG

In certain embodiments, the DOTA binding domain is a scFv and/or may comprise an amino acid sequence selected from the group consisting of:

(SEQ ID NO: 79)
HVQLVESGGGLVQPGGSLRLSCAASGFSLTDYGVHWVRQAPGKGLEWLGVIWSGGGT
AYNTALISRFTISRDNSKNTLYLQMNSLRAEDTAVYYCARRGSYPYNYFDAWGCGTLV
TVSSGGGGSGGGGSGGGGSQAVVTQEPSLTVSPGGTVTLTCGSSTGAVTASNYANWV
QQKPGQCPRGLIGGHNNRPPGVPARFSGSLLGGKAALTLLGAQPEDEAEYYCALWYSD
HWVIGGGTKLTVLG;
and
(SEQ ID NO: 80)
HVQLVESGGGLVQPGGSLRLSCAASGFSLTDYGVHWVRQAPGKGLEWLGVIWSGGGT
AYNTALISRFTISRDNSKNTLYLQMNSLRAEDTAVYYCARRGSYPYNYFDAWGCGTLV
TVSSGGGGSGGGGSGGGGSGGGGSGGGGSGGGGSQAVVTQEPSLTVSPGGTVTLTC
GSSTGAVTASNYANWVQQKPGQCPRGLIGGHNNRPPGVPARFSGSLLGGKAALTLLGA
QPEDEAEYYCALWYSDHWVIGGGTKLTVLG .
*(G4S)3 linker sequence (SEQ ID NO: 158) is shown in boldface

Additionally or alternatively, in some embodiments, the anti-CD3 multi-specific antibody comprises a heavy chain (HC) amino acid sequence comprising SEQ ID NO: 82, SEQ ID NO: 84, SEQ ID NO: 86, SEQ ID NO: 88, SEQ ID NO: 90, SEQ ID NO: 94, SEQ ID NO: 96, SEQ ID NO: 98, SEQ ID NO: 100, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 163, SEQ ID NO: 165, SEQ ID NO: 167, SEQ ID NO: 169, or a variant thereof having one or more conservative amino acid substitutions. Additionally or alternatively, in some embodiments, the anti-CD3 multi-specific antibody comprises a light chain (LC) amino acid sequence comprising SEQ ID NO: 81, SEQ ID NO: 83, SEQ ID NO: 85, SEQ ID NO: 87, SEQ ID NO: 89, SEQ ID NO: 93, SEQ ID NO: 95, SEQ ID NO: 97, SEQ ID NO: 99, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 162, SEQ ID NO: 164, SEQ ID NO: 166, SEQ ID NO: 168, or a variant thereof having one or more conservative amino acid substitutions.

In other embodiments, the anti-CD3 multi-specific antibody comprises (a) a LC sequence that is at least 80%, at least 85%, at least 90%, at least 95%, or at least 99% identical to the LC sequence present in SEQ ID NO: 81, SEQ ID NO: 83, SEQ ID NO: 85, SEQ ID NO: 87, SEQ ID NO: 89, SEQ ID NO: 93, SEQ ID NO: 95, SEQ ID NO: 97, SEQ ID NO: 99, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 162, SEQ ID NO: 164, SEQ ID NO: 166, or SEQ ID NO: 168; and/or (b) a HC sequence that is at least 80%, at least 85%, at least 90%, at least 95%, or at least 99% identical to the HC sequence present in SEQ ID NO: 82, SEQ ID NO: 84, SEQ ID NO: 86, SEQ ID NO: 88, SEQ ID NO: 90, SEQ ID NO: 94, SEQ ID NO: 96, SEQ ID NO: 98, SEQ ID NO: 100, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 163, SEQ ID NO: 165, SEQ ID NO: 167, or SEQ ID NO: 169.

Additionally or alternatively, in some embodiments, the anti-CD3 multi-specific antibody comprises a HC amino acid sequence and a LC amino acid sequence selected from the group consisting of: SEQ ID NO: 82 and SEQ ID NO: 81, SEQ ID NO: 84 and SEQ ID NO: 83, SEQ ID NO: 86 and SEQ ID NO: 85, SEQ ID NO: 88 and SEQ ID NO: 87, SEQ ID NO: 90 and SEQ ID NO: 89, SEQ ID NO: 94 and SEQ ID NO: 93, SEQ ID NO: 96 and SEQ ID NO: 95, SEQ ID NO: 98 and SEQ ID NO: 97, SEQ ID NO: 100 and SEQ ID NO: 99, SEQ ID NO: 115 and SEQ ID NO: 114, SEQ ID NO: 117 and SEQ ID NO: 116, SEQ ID NO: 119 and SEQ ID NO: 118, SEQ ID NO: 121 and SEQ ID NO: 120, SEQ ID NO: 123 and SEQ ID NO: 122, SEQ ID NO: 125 and SEQ ID NO: 124, SEQ ID NO: 127 and SEQ ID NO: 126, SEQ ID NO: 129 and SEQ ID NO: 128, SEQ ID NO: 131 and SEQ ID NO: 130, SEQ ID NO: 133 and SEQ ID NO: 132, SEQ ID NO: 135 and SEQ ID NO: 134, SEQ ID NO: 137 and SEQ ID NO: 136, SEQ ID NO: 139 and SEQ ID NO: 138, SEQ ID NO: 141 and SEQ ID NO: 140, SEQ ID NO: 143 and SEQ ID NO: 142, SEQ ID NO: 145 and SEQ ID NO: 144, SEQ ID NO: 147 and SEQ ID NO: 146, SEQ ID NO: 149 and SEQ ID NO: 148, SEQ ID NO: 151 and SEQ ID NO: 150, SEQ ID NO: 153 and SEQ ID NO: 152, SEQ ID NO: 155 and SEQ ID NO: 154, SEQ ID NO: 157 and SEQ ID NO: 156, SEQ ID NO: 163 and SEQ ID NO: 162, SEQ ID NO: 165 and SEQ ID NO: 164, SEQ ID NO: 167 and SEQ ID NO: 166, and SEQ ID NO: 169 and SEQ ID NO: 168, respectively.

Additionally or alternatively, in some embodiments, the anti-CD3 multi-specific antibody comprise a first LC amino acid sequence, a first HC amino acid sequence, a second LC amino acid sequence, and a second HC amino acid sequence selected from the group consisting of SEQ ID NO: 114, SEQ ID NO: 115, SEQ ID NO: 116, and SEQ ID NO: 117; SEQ ID NO: 118, SEQ ID NO: 119, SEQ ID NO: 120, and SEQ ID NO: 121; SEQ ID NO: 122, SEQ ID NO: 123, SEQ ID NO: 124, and SEQ ID NO: 125; SEQ ID NO: 126, SEQ ID NO: 127, SEQ ID NO: 128, and SEQ ID NO: 129; SEQ ID NO: 130, SEQ ID NO: 131, SEQ ID NO: 132, and SEQ ID NO: 133; SEQ ID NO: 134, SEQ ID NO: 135, SEQ ID NO: 136, and SEQ ID NO: 137; SEQ ID NO: 138, SEQ ID NO: 139, SEQ ID NO: 140, and SEQ ID NO: 141; SEQ ID NO: 142, SEQ ID NO: 143, SEQ ID NO: 144, and SEQ ID NO: 145; SEQ ID NO: 146, SEQ ID NO: 147, SEQ ID NO: 148, and SEQ ID NO: 149; SEQ ID NO: 150, SEQ ID NO: 151, SEQ ID NO: 152, and SEQ ID NO: 153; SEQ ID NO: 154, SEQ ID NO: 155, SEQ ID NO: 156, and SEQ ID NO: 157; SEQ ID NO: 162, SEQ ID NO: 163, SEQ ID NO: 164, and SEQ ID NO: 165; and SEQ ID NO: 166, SEQ ID NO: 167, SEQ ID NO: 168, and SEQ ID NO: 169; respectively.

Additionally or alternatively, in some embodiments, the anti-CD3 multi-specific antibodies that arm the EATs of the present disclosure bind one or more additional target antigens selected from the group consisting of CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Leg) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, peptide MHC complexes (with peptides derived from TP53, KRAS, MYC, EBNA1-6, PRAME, MART, tyronsinase, MAGEA1-A6, pmel17, LMP2, or WT1), or a small molecule DOTA-based hapten.

In some aspects, the anti-CD3 multi-specific antibodies that arm the EATs described herein contain structural modifications to facilitate rapid binding and cell uptake and/or slow release. In some aspects, the anti-CD3 multi-specific antibodies that arm the EATs of the present technology (e.g., an antibody) may contain a deletion in the CH2 constant heavy chain region to facilitate rapid binding and cell uptake and/or slow release. Additionally or alternatively, in some embodiments, the anti-CD3 multi-specific antibodies that arm the EATs described herein are bispecific antibodies, trispecific antibodies, or tetraspecific antibodies.

In any of the above embodiments of the anti-CD3 multi-specific antibodies that arm the EATs of the present technology, the anti-CD3 multi-specific antibodies may be optionally conjugated to an agent selected from the group consisting of isotopes, dyes, chromagens, contrast agents, drugs, toxins, cytokines, enzymes, enzyme inhibitors, hormones, hormone antagonists, growth factors, radionuclides, metals, liposomes, nanoparticles, RNA, DNA or any combination thereof.

In some embodiments, the anti-CD3 multi-specific antibodies that arm the EATs of the present technology bind specifically to at least one CD3 polypeptide. In some embodiments, the anti-CD3 multi-specific antibodies that arm the EATs of the present technology bind at least one CD3 polypeptide with a dissociation constant (K_{D}) of about 10⁻³ M, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some embodiments, the antibodies comprise a human antibody framework region.

### Uses of the EATs of the Present Technology

In one aspect, the present disclosure provides a method for determining the antibody binding capacity of any embodiment of the ex vivo armed T cell described herein in vitro comprising (a) contacting the ex vivo armed T cell with an agent that binds to any embodiment of the anti-CD3 multi-specific antibody disclosed herein that is present on the ex vivo armed T cell, wherein the agent is directly or indirectly linked to a detectable label, and (b) determining the antibody binding capacity of the ex vivo armed T cell by detecting the level or intensity of signal emitted by the detectable label. The detectable label may be spectroscopic, photochemical, biochemical, immunochemical, electromagnetic, radioactive, fluorescent, chemifluorescent, or chemiluminescent label. In some embodiments, the antibody binding capacity is quantified using flow cytometry or mean fluorescence intensity (MFI)-flow cytometry.

In one aspect, the present disclosure provides a method for tracking ex vivo armed T cells in a subject in vivo comprising (a) administering to the subject an effective amount of any embodiment of the ex vivo armed T cell described herein, wherein the ex vivo armed T cell is configured to localize to a tissue expressing one or more target antigens recognized by any embodiment of the anti-CD3 multi-specific antibody disclosed herein that is present on the ex vivo armed T cell; (b) administering to the subject an effective amount of a DOTA-based hapten, wherein the DOTA-based hapten is configured to bind to the anti-CD3 multi-specific antibody that is present on the ex vivo armed T cell, and comprises or is directly or indirectly linked to a detectable label; and (c) determining the biodistribution of the ex vivo armed T cell in the subject by detecting signal emitted by the detectable label of the DOTA-based hapten that is localized to the ex vivo armed T cells and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for tracking ex vivo armed T cells in a subject in vivo comprising (a) administering to the subject an effective amount of a complex comprising any embodiment of the ex vivo armed T cell described herein and a DOTA-based hapten, wherein the complex is configured to localize to a tissue expressing one or more target antigens recognized by any embodiment of the anti-CD3 multi-specific antibody disclosed herein that is present on the ex vivo armed T cell and wherein the DOTA-based hapten is configured to bind to the anti-CD3 multi-specific antibody that is present on the ex vivo armed T cell, and comprises or is directly or indirectly linked to a detectable label; and (b) determining the biodistribution of the ex vivo armed T cell in the subject by detecting signal emitted by the complex that is localized to the ex vivo armed T cells and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for detecting tumors in a subject in need thereof comprising (a) administering to the subject an effective amount of any embodiment of the ex vivo armed T cell described herein, wherein the ex vivo armed T cell is configured to localize to a tissue expressing one or more target antigens recognized by any embodiment of the anti-CD3 multi-specific antibody disclosed herein that is present on the ex vivo armed T cell; (b) administering to the subject an effective amount of a DOTA-based hapten, wherein the DOTA-based hapten is configured to bind to the anti-CD3 multi-specific antibody that is present on the ex vivo armed T cell, and comprises or is directly or indirectly linked to a detectable label; and (c) detecting the presence of tumors in the subject by detecting signal emitted by the detectable label of the DOTA-based hapten that is localized to the tumor and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for detecting tumors in a subject in need thereof comprising (a) administering to the subject an effective amount of a complex comprising any embodiment of the ex vivo armed T cell described herein and a DOTA-based hapten, wherein the complex is configured to localize to a tissue expressing one or more target antigens recognized by any embodiment of the anti-CD3 multi-specific antibody disclosed herein that is present on the ex vivo armed T cell and wherein the DOTA-based hapten is configured to bind to the anti-CD3 multi-specific antibody that is present on the ex vivo armed T cell, and comprises or is directly or indirectly linked to a detectable label; and (b) detecting the presence of tumors in the subject by detecting signal emitted by the complex that is localized to the tumor and/or is higher than a reference value. The detectable label may be spectroscopic, photochemical, biochemical, immunochemical, electromagnetic, radioactive, fluorescent, chemifluorescent, or chemiluminescent label.

In one aspect, the present disclosure provides a method for assessing the in vivo durability or persistence of ex vivo armed T cells in a subject comprising (a) administering to the subject an effective amount of any embodiment of the ex vivo armed T cell described herein, wherein the ex vivo armed T cell is configured to localize to a tissue expressing one or more target antigens recognized by any embodiment of the anti-CD3 multi-specific antibody disclosed herein that is present on the ex vivo armed T cell; (b) administering to the subject a first effective amount of a DOTA-based hapten, wherein the DOTA-based hapten is configured to bind to the anti-CD3 multi-specific antibody that is present on the ex vivo armed T cell, and comprises or is directly or indirectly linked to a detectable label; (c) detecting signal emitted by the detectable label of the DOTA-based hapten that is localized to the ex vivo armed T cells and is higher than a reference value at a first time point; (d) detecting signal emitted by the detectable label of the DOTA-based hapten that is localized to the ex vivo armed T cells and is higher than a reference value at a second time point; and (e) determining that the ex vivo armed T cells show in vivo durability or persistence when the signal emitted by the detectable label of the DOTA-based hapten at the second time point is comparable to that observed at the first time point. In certain embodiments, the method further comprising administering to the subject a second effective amount of the DOTA-based hapten after step (c). The detectable label may be spectroscopic, photochemical, biochemical, immunochemical, electromagnetic, radioactive, fluorescent, chemifluorescent, or chemiluminescent label.

In one aspect, the present disclosure provides a method for assessing the in vivo durability or persistence of ex vivo armed T cells in a subject comprising (a) administering to the subject an effective amount of a complex comprising any embodiment of the ex vivo armed T cell described herein and a DOTA-based hapten, wherein the complex is configured to localize to a tissue expressing one or more target antigens recognized by any embodiment of the anti-CD3 multi-specific antibody disclosed herein that is present on the ex vivo armed T cell and wherein the DOTA-based hapten is configured to bind to the anti-CD3 multi-specific antibody that is present on the ex vivo armed T cell, and comprises or is directly or indirectly linked to a detectable label; (b) detecting signal emitted by the complex that is localized to the ex vivo armed T cells and is higher than a reference value at a first time point; (c) detecting signal emitted by the complex that is localized to the ex vivo armed T cells and is higher than a reference value at a second time point; and (d) determining that the ex vivo armed T cells show in vivo durability or persistence when the signal emitted by the complex at the second time point is comparable to that observed at the first time point. The detectable label may be spectroscopic, photochemical, biochemical, immunochemical, electromagnetic, radioactive, fluorescent, chemifluorescent, or chemiluminescent label.

In one aspect, the present disclosure provides a method for detecting the presence of a DOTA-based hapten in a subject that has been administered any embodiment of the ex vivo armed T cell described herein comprising (a) administering to the subject an effective amount of a DOTA-based hapten, wherein the DOTA-based hapten comprises a radionuclide, and is configured to localize to the ex vivo armed T cell; and (b) detecting the presence of the DOTA-based hapten in the subject by detecting radioactive levels emitted by the DOTA-based hapten that are higher than a reference value, wherein the ex vivo armed T cell is configured to localize to a tissue expressing one or more target antigens recognized by any embodiment of the anti-CD3 multi-specific antibody disclosed herein that is present on the ex vivo armed T cell. In another aspect, the present disclosure provides a method for detecting the presence of a DOTA-based hapten in a subject that has been administered a complex comprising any embodiment of the ex vivo armed T cell described herein and a DOTA-based hapten including a radionuclide, comprising detecting the presence of the DOTA-based hapten in the subject by detecting radioactive levels emitted by the complex that are higher than a reference value, wherein the ex vivo armed T cell is configured to localize to a tissue expressing one or more target antigens recognized by any embodiment of the anti-CD3 multi-specific antibody disclosed herein that is present on the ex vivo armed T cell.

Additionally or alternatively, in some embodiments, the method further comprises quantifying radioactive levels emitted by the DOTA-based hapten or complex that is localized to the tumor and/or radioactive levels emitted by the DOTA-based hapten or the complex that is localized in one or more normal tissues or organs of the subject. In certain embodiments, the one or more normal tissues or organs are selected from the group consisting of heart, muscle, gallbladder, esophagus, stomach, small intestine, large intestine, liver, pancreas, lungs, bone, bone marrow, kidneys, urinary bladder, brain, skin, spleen, thyroid, and soft tissue. In any of the preceding embodiments, the method further comprises determining biodistribution scores by computing a ratio of the radioactive levels emitted by the DOTA-based hapten or complex that is localized to the tumor relative to the radioactive levels emitted by the DOTA-based hapten or complex that is localized in the one or more normal tissues or organs of the subject. Additionally or alternatively, the method further comprises calculating estimated absorbed radiation doses for the tumor and the one or more normal tissues or organs of the subject based on the biodistribution scores. In some embodiments, the method further comprises computing a therapeutic index for the DOTA-based hapten or complex based on the estimated absorbed radiation doses for the tumor and the one or more normal tissues or organs of the subject.

In some embodiments of the preceding methods disclosed herein, the radioactive levels emitted by the complex or the detectably labeled DOTA-based hapten are detected using positron emission tomography or single photon emission computed tomography. Additionally or alternatively, in some embodiments of the methods disclosed herein, the radioactive levels emitted by the complex or the radiolabeled DOTA-based hapten are detected between 2 to 120 hours after the complex or the radiolabeled DOTA-based hapten is administered. In certain embodiments of the methods disclosed herein, the radioactive levels emitted by the complex or the radiolabeled DOTA-based hapten are expressed as the percentage injected dose per gram tissue (% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45(9):1431-4 (2004). In some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

Additionally or alternatively, in some embodiments of the methods disclosed herein, the ex vivo armed T cell, the complex or the detectably labeled DOTA-based hapten is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the ex vivo armed T cell, the complex or the detectably labeled DOTA-based hapten is administered into the cerebral spinal fluid or blood of the subject.

Examples of DOTA-based haptens useful in the methods disclosed herein include, but are not limited to, benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), and DOTATyrLysDOTA.

In any and all embodiments of the methods disclosed herein, the subject is human.

Adoptive Cell Therapy with the EATs of the Present Technology

For treatment, the amount of the EATs provided herein administered is an amount effective in producing the desired effect, for example, treatment of a cancer or one or more symptoms of a cancer. An effective amount can be provided in one or a series of administrations of the EATs provided herein. An effective amount can be provided in a bolus or by continuous perfusion. For adoptive immunotherapy using EATs, cell doses in the range of about 10⁴ to about 10¹⁰ are typically infused.

The EATs of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the EATs and the compositions comprising thereof are intravenously administered to the subject in need. Methods for administering cells for adoptive cell therapies, including, for example, donor lymphocyte infusion and cellular immunotherapies, and regimens for administration are known in the art and can be employed for administration of the EATs provided herein.

The presently disclosed subject matter provides various methods of using the EATs (e.g., T cells) provided herein, which are coated or complexed with an effective arming dose of at least one type of anti-CD3 multi-specific antibody described herein. For example, the presently disclosed subject matter provides methods of reducing tumor burden in a subject. In one non-limiting example, the method of reducing tumor burden comprises administering an effective amount of the presently disclosed EATs to the subject and optionally administering a suitable antibody targeted to the tumor, thereby inducing tumor cell death in the subject. In some embodiments, the EATs and the antibody are administered at different times. For example, in some embodiments, the EATs are administered and then the antibody is administered. In some embodiments, the antibody is administered 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 26 hours, 48 hours, 72 hours, 96 hours, or longer after the administration of the EATs.

The presently disclosed EATs either alone or in combination with a suitable therapeutic antibody targeted to the tumor can reduce the number of tumor cells, reduce tumor size, and/or eradicate the tumor in the subject. In certain embodiments, the method of reducing tumor burden comprises administering an effective amount of EATs to the subject, thereby inducing tumor cell death in the subject. Non-limiting examples of suitable tumors include adrenal cancers, bladder cancers, blood cancers, bone cancers, osteosarcomas, brain cancers, breast cancers including triple negative breast cancer, carcinoma, cervical cancers, colon cancers, colorectal cancers, corpus uterine cancers, ear, nose and throat (ENT) cancers, endometrial cancers, esophageal cancers, Ewing's sarcoma, gastrointestinal cancers including gastric cancer, head and neck cancers, Hodgkin's disease, intestinal cancers, kidney cancers, larynx cancers, acute and chronic leukemias including acute myeloid leukemia, liver cancers, lymph node cancers, lymphomas, lung cancers including non-small cell lung cancer, melanomas, mesothelioma, myelomas including multiple myeloma, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In some embodiments, the cancer is a relapsed or refractory cancer. In some embodiments, the cancer is resistant to one or more cancer therapies, e.g., one or more chemotherapeutic drugs.

The presently disclosed subject matter also provides methods of increasing or lengthening survival of a subject having a neoplasia (e.g., a tumor). In one non-limiting example, the method of increasing or lengthening survival of a subject having neoplasia (e.g., a tumor) comprises administering an effective amount of the presently disclosed EATs to the subject, thereby increasing or lengthening survival of the subject. The presently disclosed subject matter further provides methods for treating or preventing a neoplasia (e.g., a tumor) in a subject, comprising administering the presently disclosed EATs to the subject.

Cancers whose growth may be inhibited using the EATs of the presently disclosed subject matter comprise cancers typically responsive to immunotherapy. Non-limiting examples of cancers for treatment include multiple myeloma, neuroblastoma, glioma, melanoma, sarcomas, acute myeloid leukemia, breast cancer, colon cancer, esophageal cancer, gastric cancer, non-small cell lung cancer, ovarian cancer, pancreatic cancer, prostate cancer, thyroid cancer, small cell lung cancer, and NK cell lymphoma. In certain embodiments, the cancer is triple negative breast cancer or ovarian cancer. In some embodiments, the cancer is prostate cancer. In some embodiments, the cancer is acute myeloid leukemia. In some embodiments, the cancer is ovarian cancer, sarcoma, non-small cell lung cancer, esophageal cancer, gastric cancer, colorectal cancer, or triple negative breast cancer.

Additionally or alternatively, in some embodiments of the methods disclosed herein, the immune-activating cytokine levels released by the EATs of the present technology are lower compared to unarmed T cells mixed with an anti-CD3 multi-specific antibody, thus reducing the likelihood of CRS. Examples of immune-activating cytokines include granulocyte macrophage colony stimulating factor (GM-CSF), IFNα, IFN-γ, TNF-α, IL-2, IL-3, IL-6, IL-10, IL-11, IL-7, IL-12, IL-15, IL-21, interferon regulatory factor 7 (IRF7), and combinations thereof.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition embodied in the presently disclosed subject matter is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes. Another group has a genetic predisposition to neoplasia but has not yet evidenced clinical signs of neoplasia. For instance, women testing positive for a genetic mutation associated with breast cancer, but still of childbearing age, can wish to receive one or more of the EATs described herein in treatment prophylactically to prevent the occurrence of neoplasia until it is suitable to perform preventive surgery.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

In one aspect, the present disclosure provides a method for treating cancer or inhibiting tumor growth or metastasis in a subject in need thereof comprising administering to the subject an effective amount of any embodiment of the ex vivo armed T cell described herein. In another aspect, the present disclosure provides a method for treating cancer or inhibiting tumor growth or metastasis in a subject in need thereof comprising (a) administering to the subject a first effective amount of any and all embodiments of the ex vivo armed T cell described herein, (b) administering to the subject a second effective amount of the ex vivo armed T cell about 72 hours after administration of the first effective amount of the ex vivo armed T cell, (c) administering to the subject a third effective amount of the ex vivo armed T cell about 96 hours after administration of the second effective amount of the ex vivo armed T cell, and (d) repeating steps (a)-(c) for at least three additional cycles. In certain embodiments, the subject exhibits sustained cancer remission after completion of step (d). In certain embodiments, the subject is human.

Additionally or alternatively, in some embodiments of the methods disclosed herein, the ex vivo armed T cell is autologous, non-autologous, or derived in vitro from lymphoid progenitor cells.

Additionally or alternatively, in some embodiments of the methods disclosed herein, the ex vivo armed T cell is administered intravenously, intramuscularly, intraarterially, intrathecally, intracapsularly, intraorbitally, intradermally, intraperitoneally, transtracheally, subcutaneously, intracerebroventricularly, orally, intratumorally, or intranasally. In certain embodiments, the ex vivo armed T cell is administered into the cerebral spinal fluid or blood of the subject. In some embodiments, the subject is diagnosed with, or is suspected of having cancer. Exemplary cancers or tumors include, but are not limited to, carcinoma, sarcoma, melanoma, hematopoietic cancer, osteosarcoma, Ewing's sarcoma, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, seminomas, skin cancers, stomach cancers, teratomas, testicular cancers, thyroid cancers, uterine cancers, vaginal cancers, vascular tumors, and metastases thereof.

Additionally or alternatively, in some embodiments, the method further comprises separately, simultaneously, or sequentially administering an additional cancer therapy. In some embodiments, the additional cancer therapy is selected from among chemotherapy, radiation therapy, immunotherapy, monoclonal antibodies, anti-cancer nucleic acids or proteins, anti-cancer viruses or microorganisms, and any combinations thereof. In certain embodiments, the additional cancer therapy is an immune checkpoint inhibitor selected from among pembrolizumab, nivolumab, cemiplimab, atezolizumab, avelumab, durvalumab, and ipilimumab.

Additionally or alternatively, in certain embodiments, the method further comprises administering a cytokine to the subject. In some embodiments, the cytokine is administered prior to, during, or subsequent to administration of the ex vivo armed T cell. Examples of suitable cytokines include, but are not limited to, interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2.

In any and all embodiments of the methods disclosed herein, in vivo or in vitro cytokine levels released by the ex vivo armed T cells are reduced compared to unarmed T cells mixed with an anti-CD3 multi-specific antibody.

### Kits

The presently disclosed subject matter provides kits for the treatment of cancer. In certain embodiments, the kit comprises any and all embodiments of the anti-CD3 multi-specific antibody disclosed herein in unit dosage form and instructions for arming T cells with the same. Additionally or alternatively, in some embodiments, the kits may further comprise instructions for isolating T cells from an autologous or non-autologous donor, and agents for culturing, differentiating and/or expanding isolated T cells in vitro such as cell culture media, CD3/CD28 beads, zoledronate, cytokines such as IL-2, IL-15 (e.g., IL15Rα-IL15 complex), buffers, diluents, excipients, and the like. Additionally or alternatively, in some embodiments, the kits comprise any and all embodiments of the EATs described herein and instructions for using the same to treat cancer in a subject in need thereof. The instructions will generally include information about the use of the composition for the treatment or prevention of a neoplasia (e.g., solid tumor).

In any of the preceding embodiments of the kits disclosed herein, the kit comprises a sterile container which contains a therapeutic agent disclosed herein (e.g., any and all embodiments of the anti-CD3 multi-specific antibody and/or EATs described herein); such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. Additionally or alternatively, in some embodiments, the instructions include at least one of the following: description of the therapeutic agent (e.g., any and all embodiments of the anti-CD3 multi-specific antibody and/or EATs described herein); dosage schedule and administration for treatment or prevention of a neoplasia (e.g., solid tumor) or symptoms thereof precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The present technology is further illustrated by the following Examples, which should not be construed as limiting in any way.

### Example 1: Materials and Methods

T cells expansion ex vivo. Peripheral blood mononuclear cells (PBMCs) were separated from buffy coats (New York Blood Center) by Ficoll. These naïve T cells were purified from human PBMC using Pan T cell isolation kit (Miltenyi Biotec) and expanded by CD3/CD28 Dynabeads (Invitrogen, Carlsbad, Calif.) for 7 to 14 days in the presence of 30 IU/mL of IL-2 according to manufacturer's instructions. Unless stated otherwise, these cultured T cells were used for all T cell experiments.

Gamma delta (γδ) T cells activation. Gamma delta T cells were expanded through 2 different ways. 1) Fresh PBMCs separated from buffy coats were cultured with 2 μM of zoledronic acid and 800 IU/mL of IL-2 for 12 to 14 days according to protocols. 2) Fresh PBMCs were cultured with 2 μM of zoledronic acid and 30 ng/mL of IL15Rα-IL15 complex for 12 to 14 days. Cultured PBMCs were harvested and their surface antigen expression examined using antibodies against human CD3, CD4, CD8, γδ T cell receptor (TCR), and αβ TCR.

Autologous T cell activation. Naïve T cells were separated from unused cryopreserved peripheral blood stem cell collections with IRB approval. These cells were purified using Dynabeads untouched human T cell kit (Invitrogen, Carlsbad, Calif.) and expanded with CD3/CD28 Dynabeads (Invitrogen, Carlsbad, Calif.) and 30 IU/mL of IL-2 for 10 to 14 days.

Tumor cell lines. Representative neuroblastoma cell line, IMR-32 (ATCC-CCL-127), osteosarcoma cell line, 143B (ATCC-CRL-8303) and U-2 OS (ATCC-HTB-96), primitive neuroectodermal tumor cell line TC-71 (ATCC CRL-1598) and TC-32 (RRID:CVCL-7151), breast cancer cell line HCC1954 (ATCC-CRL-2338), acute monocytic leukemia (AML-M5a) cell line MOLM13, prostate cancer cell line LNCaP-AR(ATCC-CRL-1740), and melanoma cell line M14 (UCLA-SO-M14) were used. All cells were authenticated by short tandem repeats profiling using PowerPlex 1.2 System (Promega, Madison, Wis.), and periodically tested for mycoplasma infection using a commercial kit (Lonza, Basel, Switzerland). The luciferase-labeled osteosarcoma cell line 143BLuc, melanoma cell line M14Luc, and neuroblastoma cell line IMR32Luc were generated by retroviral infection with an SFG-GF Luc vector.

GD2-BsAb or HER2-BsAb were used for arming T cells. Hu3F8-BsAb specific for GD2 was built using the IgG-[L]-scFv format, in which the anti-CD3 huOKT3 single-chain variable fragment (scFv) was linked to the carboxyl end of the anti-GD2 hu3F8 IgG1 light chain, where the N297A mutation was introduced to remove glycosylation and the K322A to remove complement activation—a combination to reduce spontaneous cytokine release (H. Xu et al., Cancer immunology research 3, 266 (March, 2015)). HER2-BsAb built with the IgG-[L]-scFv format, carried a V_{H} identical to that of transtuzumab IgG1, again with both N297A and K322A mutations to silence Fc functions (A. Lopez-Albaitero et al., Oncoimmunology 6, e1267891 (2017)). Hu3F8×OKT3 and HerceptinxOKT3 chemical conjugates were made as previously described by Sen et al (M. Yankelevich et al., Pediatr Blood Cancer 59, 1198 (2012); M. Sen et al., J Hematother Stem Cell Res 10, 247 (2001)). In these chemical conjugates, the mouse OKT3 antibody instead of huOKT3 was used. The other BsAbs were synthesized as previously described. (H. Xu et al., Cancer immunology research 3, 266 (2015), S. S. Hoseini, H. Guo, Z. Wu, M. N. Hatano, N. V. Cheung, Blood advances 2, 1250 (2018), Z. Wu, H. F. Guo, H. Xu, N. V. Cheung, Mol Cancer Ther 17, 2164 (2018); A. Lopez-Albaitero et al., OncoImmunology, 6(3):e1267891 (2017)).

Antibody Dependent T cell mediated Cytotoxicity (ADTC). EAT-mediated cytotoxicity was performed using ⁵¹Cr release as described previously (H. Xu et al., Cancer immunology research 3, 266 (March, 2015)), and EC₅₀ was calculated using SigmaPlot software. Target cell lines were cultured in RPMI-1640 (Cellgro) supplemented with 10% fetal bovine serum (FBS, Life Technologies, Carlsbad, Calif.) and harvested with EDTA/Trypsin. These target cells were labeled with sodium ⁵¹Cr chromate (Amersham, Arlington Height, Ill.) at 100 μCi/10⁶ cells at 37° C. for 1 hour. After washing twice, these radiolabeled target cells were plated in 96-well plates. EATs were added to target cells at decreasing effector:target cell (E:T) ratios, at 2-fold dilutions from 50:1. After incubation at 37° C. for 4 hours, the released ⁵¹Cr was measured by a gamma counter (Packed Instrument, Downers Grove, Ill.). Percentage of specific lysis was calculated using the formula where cpm represented counts per minute of ⁵¹Cr released.

$$\frac{100 \% \times ( experimental cpm - background cpm )}{( total cpm - background cpm )}$$

Total release of ⁵¹Cr was assessed by lysis with 10% SDS (Sigma, St Louis, Mo.) and background release was measured in the absence of effector cells and antibodies.

Cytokine release assays. EAT-induced human cytokine release was analyzed in vitro and in vivo. Human Th1 cell released cytokines were analyzed by LEGENDplex™ Human Th1 Panel (Biolegend, San Diego, Calif.). Five human T cell cytokines including IL-2, IL-6, IL-10, IFN-γ and TNF-α were analyzed after arming or after exposure to target antigen(+) tumor cells (in vitro). Mouse serum cytokines were analyzed 4 hours after EAT injection.

T cell arming. Ex vivo expanded polyclonal T cells were harvested between day 7 and day 14 and armed with each BsAb for 20 minutes at room temperature. After incubation, the T cells were washed with PBS twice. After washing, EATs were tested for cell surface density of BsAb (MFI) using anti-idiotype antibody or anti-human IgG Fc antibody. For quantification of surface bound BsAb, antibody binding capacity (ABC) by flow cytometry referenced to Quantum™ Simply Cellular® (QSC) microspheres. EATs were tested in vitro for cytotoxicity against the appropriate targets in ADTC assays.

Cryopreservation and thawing of Ex vivo BsAb armed T cells (EAT). After arming with BsAbs, EATs were centrifuged at 1800 rpm for 5 minutes at 4° C. and the supernatant discarded. The cell pellet was resuspended in T cell freezing medium (90% of FCS and 10% DMSO) to achieve a cell concentration of 50×10⁶ cells/1 mL, chilled to 4° C. and aliquoted into 2 mL cryovials. Vials were immediately transferred to freeze at −80° C. for 24 hours before transferring to liquid nitrogen. After storage cryovials were thawed in a 37° C. water bath with gentle swirling for 1 minute. The thawed cells were transferred to F10 media and centrifuged at 1800 rpm for 5 minutes. Thawed cells were analyzed for viability, phenotype, antibody binding, and ADTC assays to determine the impact of cryopreservation on cellular performance.

T cell transduction with tdTomato and click beetle red luciferase. T cells isolated from PBMCs were stimulated with CD3/CD28 Dynabeads (Invitrogen, Carlsbad, Calif.) for 24 hours. T cells were transduced with retroviral constructs containing tdTomato and click beetle red luciferase in RetroNectin-coated 6-well plates in the presence of IL-2 (100 IU/mL) and protamine sulfate (4 μg/mL). Transduced T cells were cultured for 8 days before use in animal experiments.

In vivo anti-tumor effects of EATs. All animal experiments were performed according to Institutional Animal Care and Use Committee (IACUC) guidelines. Tumors were suspended in Matrigel (Corning Corp, Tewksbury Mass.) and implanted in the flank of 6-10 week-old BALB-Rag2^{-/-}IL-2R-γc-KO (BRG) mice (Taconic Biosciences, Germantown, N.Y.) (D. Andrade et al., Arthritis Rheum 63, 2764 (September, 2011)). The following tumor lines and cell doses were used: 1×10⁶ of 143BLuc, 5×10⁶ IMR32-Luc, 5×10⁶ M14Luc, 5×10⁶ HCC1954, 5×10⁶ TC-32 and 5×10⁶ TC-71. Three different osteosarcoma and 2 different neuroblastoma patient-derived tumor xenografts (PDXs) established from fresh surgical specimens with IRB approval were also utilized. T cells were purified and expanded in vitro as described above. Prior to injection into mice, these T cells were analyzed by FACS for the frequencies of CD3+, CD8+, CD4+ populations. For arming, cultured T cells harvested after 7 to 14 days of ex vivo expansion were used. Treatment was initiated after tumors were established (average tumor volume of 100 mm³ when measured using TM900 scanner) (Piera, Brussels, BE). When tumor growth reached 2 cm³ or greater, mice were euthanized. CBC analyses, body weight, general activity, physical appearance and GVHD scoring were monitored. All animal experiments were repeated twice more with different donor's T cells to ensure that our results were reliable.

Bioluminescence imaging. Luc(+) T cell engraftment and trafficking were quantified after intravenous injection of 3 mg D-luciferin (Gold Biotechnology) on different days post T cell injection. Bioluminescence images were acquired using IVIS Spectrum CT In vivo Imaging System (Caliper Life Sciences, Waltham, Mass.) and overlaid onto visible light images, to allow Living image 2.60 (Xenogen, Alameda, Calif.) to quantify bioluminescence in the tumor regions of interest (ROI). The total counts (photon/s) over time were quantified, and the bioluminescence signals before T cell injection were used as baselines.

Flow cytometry of blood, spleen and tumor. Peripheral blood, spleen and tumors were collected and analyzed by flow cytometry. Antibodies against human CD3, CD4, CD8, and CD45 (BD Bioscience) were used to quantify T cell engraftment and subpopulations. Fluorescence of stained cells was acquired using either a BD FACS Calibur Tnr or a BD LSRFORTESSA (BD Biosciences, Heidelberg, Germany) and analyzed using FlowJo software (FlowJo, LLC, Ashland, Oreg.).

Immunohistochemistry (IHC) for T cell infiltration. Harvested xenografts were tested for T cell infiltration using immunohistochemistry (IHC). Human CD3, CD4 and CD8 staining were performed using Discovery XT processor (Ventana Medical Systems, Oro Valley, Ariz.). Paraffin-embedded tumor sections were deparaffinized with EZPrep buffer (Ventana Medical Systems, Oro Valley, Ariz.), antigen retrieval was performed with CC1 buffer (Ventana Medical Systems, Oro Valley, Ariz.) and sections were blocked for 30 minutes with background buffer solution (Innovex). Anti-CD3 (DAKO, cat #A0452, 1.2 μg/mL) antibody was applied, and sections were incubated for 5 hours, followed by 60 min incubation with biotinylated goat anti-rabbit IgG (Vector laboratories, cat #PK6101) at 1:200 dilution. Control antibody staining was done with biotinylated goat anti-rat IgG (Vector Labs, Burlingame, Calif., cat #MKB-22258). All images were captured from tumor sections using Nikon ECLIPSE Ni-U microscope and NIS-Elements 4.0 imaging software.

Statistics. Differences between groups indicated in the figures were tested for significance by one-way ANOVA or student's t-test, and survival outcomes were analyzed using GraphPad Prism 7.0. P-value<0.05 was considered statistically significant.

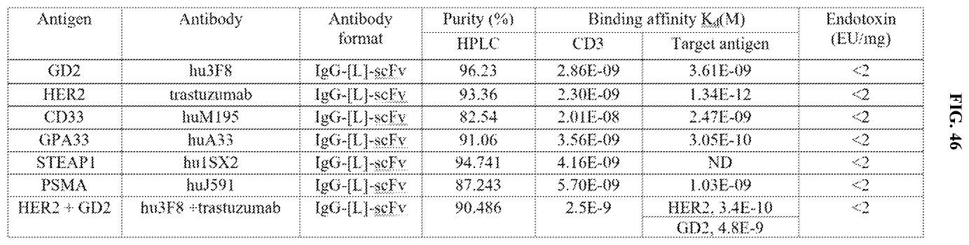

Bispecific antibodies. All BsAbs were synthesized as previously described (U.S. Patent Application No. 62/896,415) (Hoseini et al., Blood advances 2, 1250-1258 (2018), Wu et al., Mol Cancer Ther 17, 2164-2175 (2018), Xu et al., Cancer Immunol Res 3, 266-277 (2015), and Lopez-Albaitero et al., OncoImmunology 6, e1267891). For each BsAb, scFv of huOKT3 was fused to the C-terminus of the light chain of human IgG1 via a C-terminal (G4S)₃ linker (SEQ ID NO: 158). N297A and K322A on Fc were generated with site-directed mutagenesis via primer extension in polymerase chain reactions. The nucleotide sequence encoding each BsAb was synthesized by GenScript and subcloned into a mammalian expression vector. Each BsAb was produced using Expi293™ expression system (Thermo Fischer Scientific, Waltham, Mass.) separately. Antibodies were purified with protein A affinity column chromatography. The purity of BsAbs was evaluated by size-exclusion high performance liquid chromatography (SE-HPLC) and showed high levels of purity (>90%). The BsAbs remained stable after multiple freeze-thaw cycles. Biochemistry data of the BsAbs used in this study were summarized in FIG. 46.

Tumor cell lines. Neuroblastoma cell line, IMR-32 (ATCC Cat #CCL-127, RRID:CVCL 0346), osteosarcoma cell line, 143B (ATCC Cat #CRL-8303, RRID:CVCL 2270) and U-2 OS (ATCC Cat #HTB-96, RRID:CVCL 0042), primitive neuroectodermal tumor cell line TC-32 (RRID:CVCL-7151), breast cancer cell line HCC1954 (ATCC Cat #CRL-2338, RRID:CVCL 1259), gastric cancer cell line NCI-N87 (ATCC Cat #CRL-2338, RRID:CVCL 1259), acute monocytic leukemia (AML-M5a) cell line MOLM13 (DSMZ Cat #ACC-554, RRID:CVCL 2119), prostate cancer cell line LNCaP-AR (ATCC Cat #CRL-1740, RRID:CVCL 1379), and melanoma cell line M14 (NCI-DTP Cat #M14, RRID:CVCL 1395) were used for experiments. All cancer cells were authenticated by short tandem repeats profiling using PowerPlex 1.2 System (Promega, Madison, Wis., Cat #DC8942), and periodically tested for mycoplasma infection using a commercial kit (Lonza, Basel, Switzerland, Cat #LT07-318). The luciferase-labeled melanoma cell line M14Luc and neuroblastoma cell line IMR32Luc were generated by retroviral infection with an SFG-GF Luc vector.

In vivo experiments. All animal experiments were performed in compliance with Memorial Sloan Kettering Cancer Center's institutional Animal Care and Use Committee (IACUC) guidelines. In vivo anti-tumor response was evaluated using cancer cell line- or patient-derived xenografts (CDXs or PDXs). Cancer cells suspended in Matrigel (Corning Corp, Tewksbury Mass.) or PDXs were implanted in the right flank of 6-10-week-old BALB-Rag2^{-/-}IL-2R-γc-KO (BRG) mice (Taconic Biosciences, Germantown, N.Y.) (Andrade et al., Arthritis Rheum 63, 2764-2773 (2011)). The following cancer cell lines and cell doses were used: 1×10⁶ of 143BLuc, 5×10⁶ of IMR32Luc, 5×10⁶ of HCC1954, 5×10⁶ of LNCaP-AR, and 5×10⁶ of TC-32. For mixed lineage CDX, 2.5×10⁶ of IMR32Luc and 2.5×10⁶ of HCC1954 were mixed and implanted into each mouse. Three osteosarcoma, one Ewing sarcoma family of tumors (EFT), and one breast cancer PDXs were established from fresh surgical specimens with MSKCC IRB approval. To avoid biological variables, only female mice were used for in vivo experiments except LNCaP-AR CDXs using male mice. Treatment was initiated after tumors were established, average tumor volume of 100 mm³ when measured using TM900 scanner (Piera, Brussels, BE). Before treatment, mice with small tumors (<50 mm³) or infection signs were excluded from the experiments, and the included mice were randomly assigned to each group. Tumor growth curves and overall survival was analyzed, and the overall survival was defined as the time from start of treatment to when tumor volume reached 2000 mm³. To define the well-being of mice, CBC analyses, body weight, general activity, physical appearance, and GVHD scoring were monitored. All animal experiments were repeated twice more with different donor's T cells to ensure that our results were reliable.

Immunohistochemistry (IHC) for T cell infiltration and HER2 expression. Harvested xenografts were Formalin-Fixed Paraffin-Embedded (FFPE) and tested for T cell infiltration using immunohistochemistry (IHC). IHC staining was performed by Molecular Cytology Core Facility of MSKCC using Discovery XT processor (Ventana Medical Systems, Oro Valley, Ariz.). FFPE tumor sections were deparaffinized with EZPrep buffer (Ventana Medical Systems, Oro Valley, Ariz.), antigen retrieval was performed with CC1 buffer (Ventana Medical Systems, Oro Valley, Ariz.), and sections were blocked for 30 minutes with background buffer solution (Innovex). Anti-CD3 antibody (Agilent, Cat #A0452, RRID: AB 2335677, 1.2 μg/mL) and anti-HER2 (Enzo Life Sciences Cat #ALX-810-227-L001, RRID: AB 11180914, 5 μg/mL) were applied, and sections were incubated for 5 hours, followed by 60 min incubation with biotinylated goat anti-rabbit IgG (Vector laboratories, cat #PK6101) at 1:200 dilution. Control antibody staining was done with biotinylated goat anti-rat IgG (Vector Labs, Burlingame, Calif., cat #MKB-22258). The detection was performed with DAB detection kit (Ventana Medical Systems, Oro Valley, Ariz.) according to manufacturer's instruction. All images were captured from tumor sections using Nikon ECLIPSE Ni-U microscope and NIS-Elements 4.0 imaging software.

### Example 2: Ex Vivo BsAb Armed T Cells Acquired Target Antigen-Specific Cytotoxicity

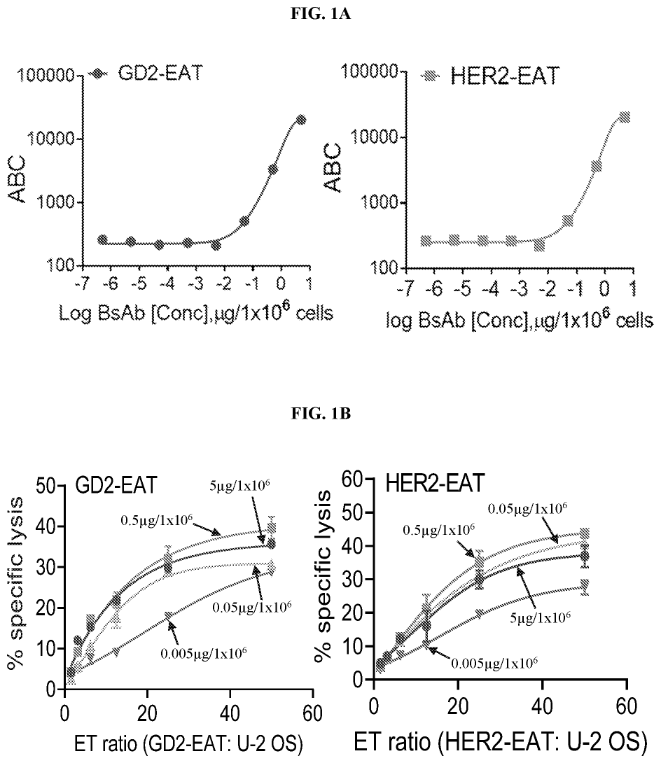

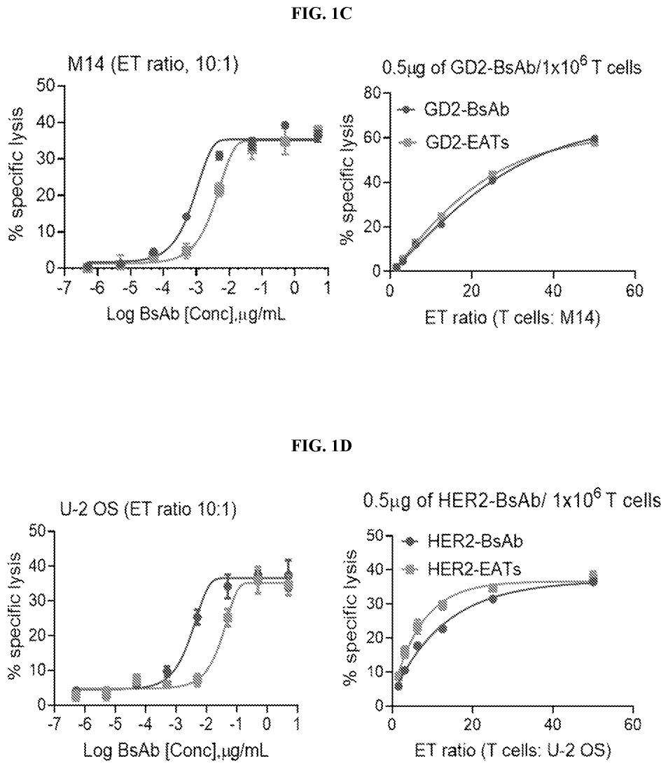

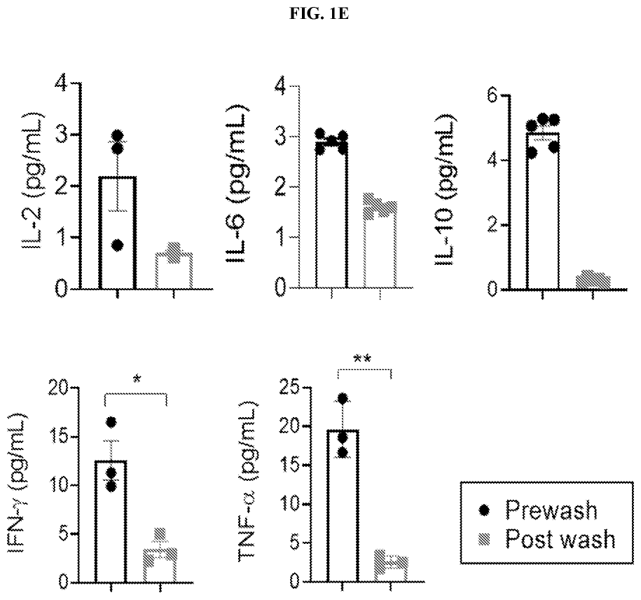

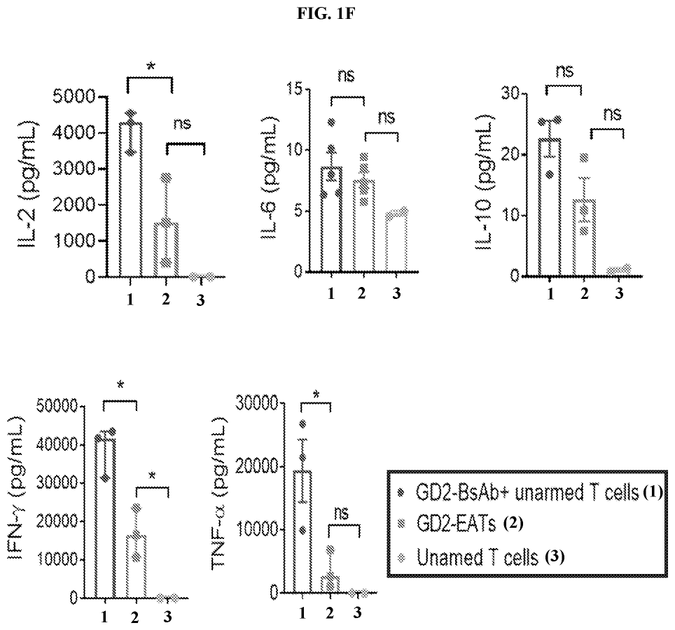

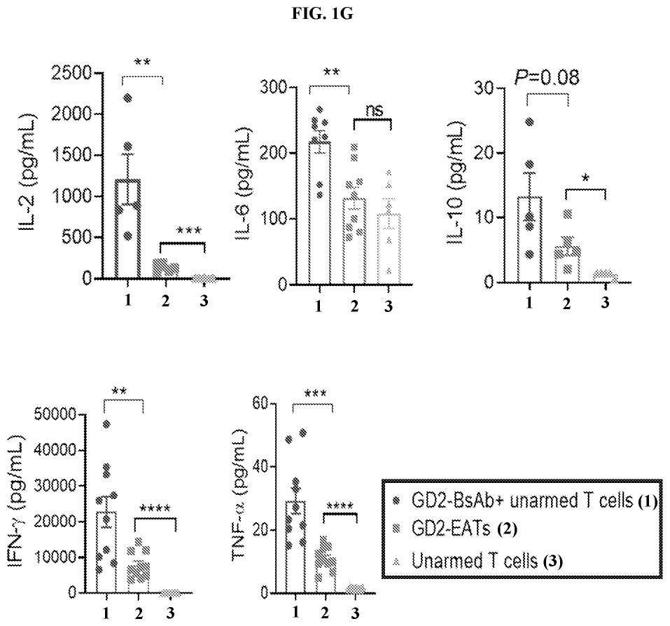

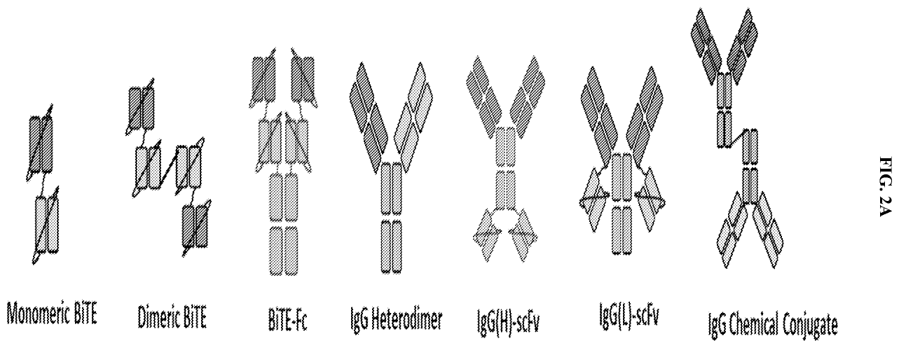

Given the finite T cell receptor density on human T cells (J. D. Stone et al., J Immunol 187, 6281 (Dec. 15, 2011)), the range and the optimal T cell surface density of BsAb as a function of arming dose was evaluated. Surface BsAb on EAT was analyzed using anti-idiotype or anti-human IgG Fc-specific antibodies for anti-GD2 BsAb armed T cells (GD2-EATs) or for anti-HER2 BsAb armed T cells (HER2-EATs), respectively. GD2-EATs and HER2-EATs showed increasing MFIs with increasing arming dose of either GD2-BsAb or HER2-BsAb, and more precise quantitation of BsAb density was measured as antibody-binding capacity (ABC) by flow cytometry referenced to anti-mouse quantum beads (FIG. 1A). Antibody-dependent T cell-mediated cytotoxicity (ADTC) was studied over a range of effector to target ratios (E:T ratios from 50:1 to 1.5:1) and BsAb arming doses (FIG. 1B). GD2-EATs and HER2-EATs both showed strong cytotoxicity against GD2(+) HER2(+) osteosarcoma cell lines (U-2 OS), with maximal cytotoxicity by GD2-EATs or by HER2-EATs at arming BsAb doses between 0.05 μg/10⁶ T cells to 5 μg/10⁶ T cells, at BsAb surface densities between 500 to 20,000 molecules per T cell. When compared to unarmed T cells in the continual presence of BsAb, the potencies of GD2-EATs and HER2-EATs were −10 fold lower (EC50); however, their maximal killing efficacy was comparable (FIGS. 1C-1D).

### Example 3: Bispecific Antibody Format has Profound Effects on Anti-Tumor Activity of EATs

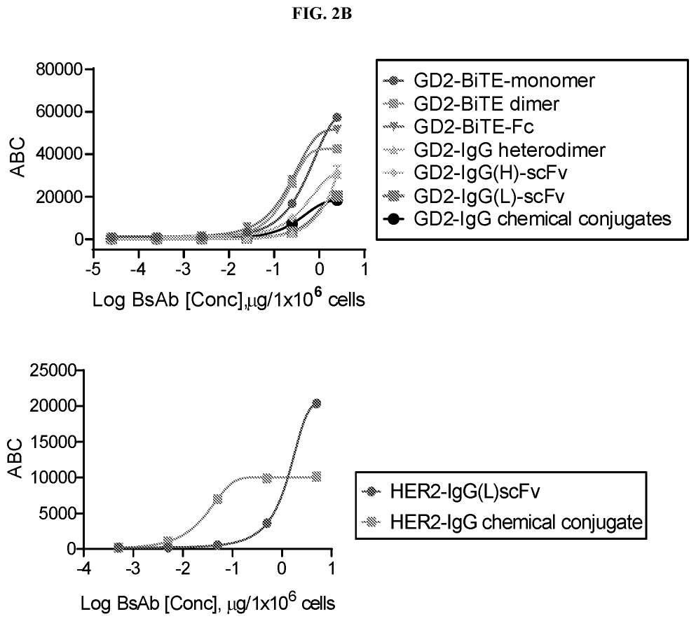

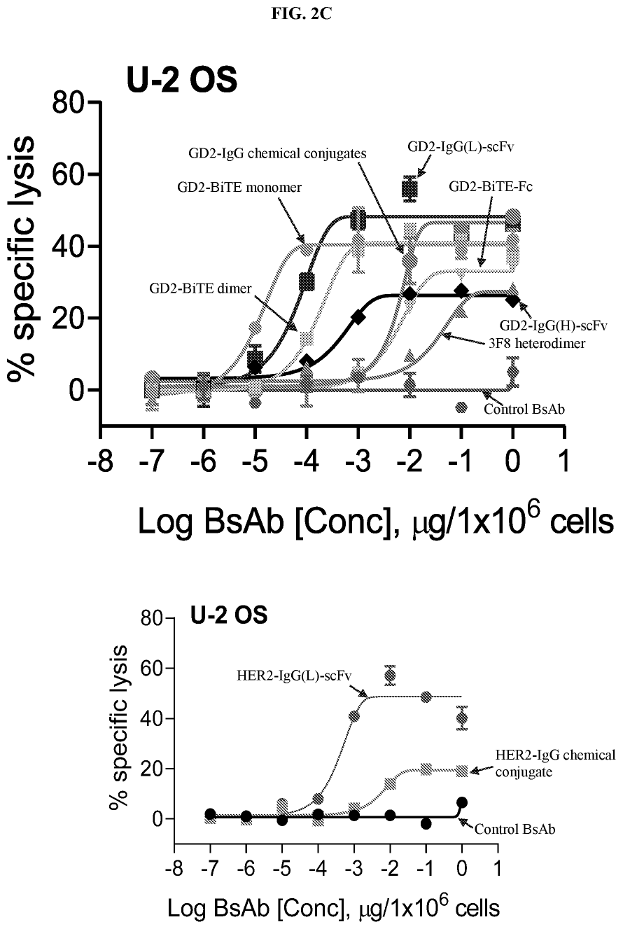

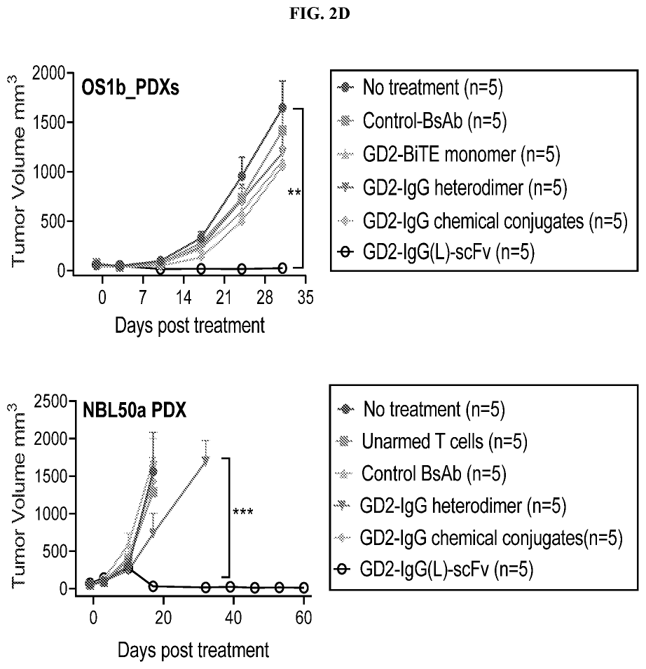

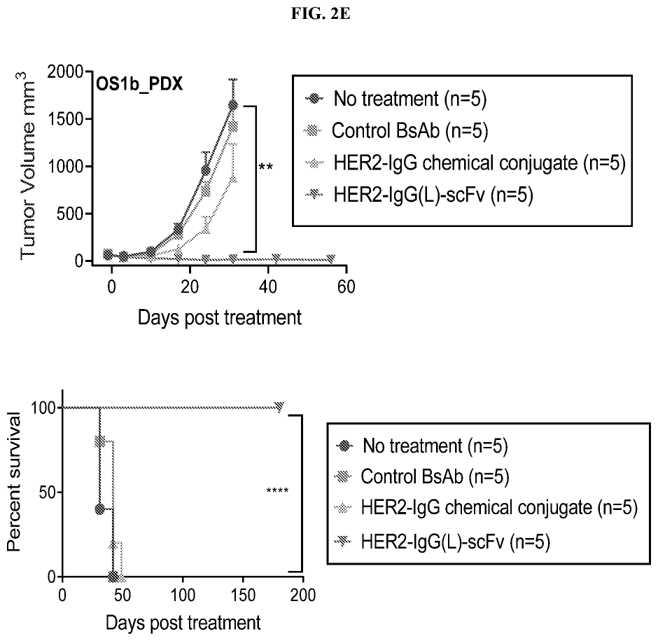

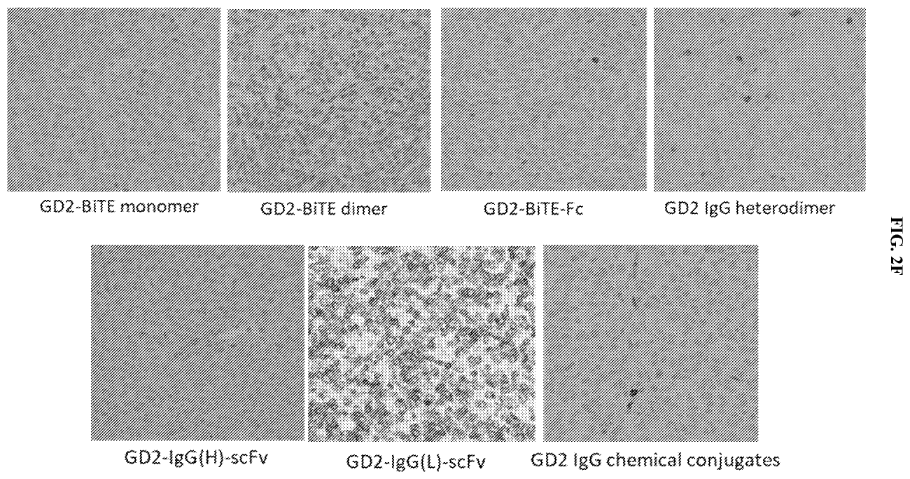

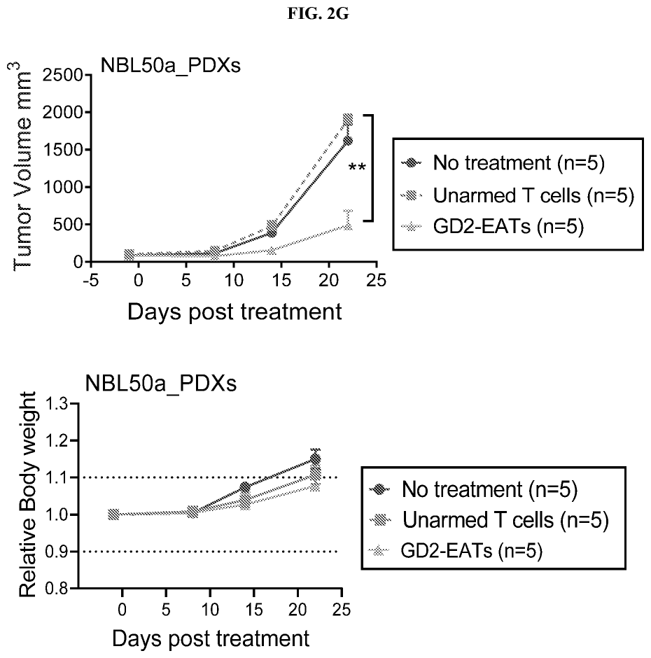

Anti-tumor potency of EATs armed with different anti-GD2 BsAb structural formats, all derived from the hu3F8 (anti-GD2) and huOKT (anti-CD3) sequences, were compared, including BiTE-monomer, BiTE-dimer, BiTE-Fc, IgG heterodimer, IgG chemical conjugate (hu3F8×OKT3), IgG-[H]-scFv, and IgG-[L]-scFv (FIG. 2A).

Additionally, HER2-EATs armed with HER2 IgG chemical conjugates (Herceptin×OKT3) and compared to EATs armed with HER2 IgG-[L]-scFv formats. Anti-GD2 EATs armed with IgG-[L]-scFv and IgG chemical conjugate showed similar surface BsAb densities (ABC) as a function of arming dose; for HER2-EATs, IgG-[L]-scFv had higher ABC than IgG chemical conjugates (FIG. 2B). Both GD2-EATs and HER-EATs armed with IgG-[L]-scFv format demonstrated superior potency and efficacy over EATs armed with each respective IgG chemical conjugate (FIG. 2C). In vivo anti-tumor activities were then compared in PDX models (FIG. 2D and FIGS. 8A-8C). T cells armed with different structural formats of GD2-BsAbs or HER2-BsAbs were injected iv twice a week for 2 to 3 weeks. Each EATs were armed with a fixed dose at 2 μg of each BsAb/2×10⁷ T cells for equivalent ABCs among groups (1,000 to 10,000 molecules/T cell). EAT therapy was well tolerated irrespective of BsAbs formats. GD2-EATs armed with IgG-[L]-scFv was superior over all other formats of GD2-BsAbs for tumor response and survival against both osteosarcoma PDX and against neuroblastoma PDXs. For HER2-EATs, IgG-[L]-scFv format was also more effective than IgG chemical conjugate, significant for tumor response (P<0.01) and for survival (P=0.0020) (FIG. 2E). This difference in efficacy among GD2-EATs armed with different structural formats strongly correlated with the density of tumor infiltrating CD3(+) T cells (TILs) by IHC staining of neuroblastoma PDXs harvested on day 10 after the beginning of treatment (FIG. 2F). GD2-EATs armed with IgG-[L]-scFv format showed significantly more abundant TILs compared to EATs armed with other formats of GD2-BsAb.

### Example 4: Autologous EATs Armed with Anti-GD2 IgG-[L]-scFv were Equally Effective In Vivo

With the IgG-[L]-scFv formatted GD2-BsAb, autologous EATs were generated using patient-derived T cells purified from cryopreserved PBMCs and expanded in vitro. Autologous GD2-EATs (0.1 μg of GD2-BsAb/10⁶ cells) were administered iv into mice xenografted with the corresponding patient's neuroblastoma PDXs (FIG. 2G). Autologous GD2-EATs suppressed tumor growth as well as EATs derived from unrelated donor, confirming that the anti-tumor property of EATs was independent of allogeneic ‘graft-versus-cancer’ effect. Since autologous T cell-PDX pairs are in short supply, the rest of the EAT experiments disclosed herein were performed using random donor T cells.

### Example 5: Ex Vivo T Cell Arming Reduces TNF-α Release by T Cells Exposed to BsAbs

Cytokine release was evaluated throughout each step of T cell arming: during the 20-minute incubation of T cells with BsAb (arming), after the wash with PBS, after co-culture with antigen-positive tumor cell lines (E:T ratio of 50:1), and finally after in vivo administration. TH1 cell cytokines (IL-2, IL-6, IL-10, IFN-γ, and TNF-α) released by T cells were measured in the supernatants after arming (Prewash) and after 2^{nd} washing step (Post wash) (FIG. 1E). Although the released cytokine levels during arming were generally low, IFN-γ and TNF-α did increase, especially at high arming doses of GD2-BsAb, which were removed after two washing steps (FIG. 9A).

After 4 hours of co-culture with target cells at 37° C., T cell cytokines were measured again (FIG. 1F). The cytokines surged after exposure to antigen-positive tumor cells. Unarmed T cells co-incubated with GD2-BsAb released more cytokines than GD2-EATs over a broad dose titration of GD2-BsAb (0.005 to 10 μg/10⁶ cells) (FIG. 9B). At the optimal arming doses (0.05 to 5 μg/10⁶ cells), GD2-BsAb plus unarmed T cells released median levels of 4,000 pg/mL of IL-2, 40,000 pg/mL of IFN-γ, and 20,000 pg/mL of TNF-α; in contrast, GD2-EATs released 1,500 pg/mL of IL-2, 15,000 pg/mL of IFN-γ and 2,000 pg/mL of TNF-α. While, the levels of IL-6 and IL-10 did not show significant difference among T cell groups.

The in vivo cytokine release after 4 hours of GD2-EAT treatment (10 μg of GD2-BsAb/2×10⁷ cells) was next analyzed and compared with that released by unarmed T cells (2×10⁷ cells) with iv GD2-BsAb (10 μg) in GD2(+) osteosarcoma PDX mouse model (FIG. 1G). Both GD2-EATs and iv GD2-BsAb with unarmed T cells induced measurable cytokine release. Most notably, the major cytokine levels (IL-2, IL-6, IFN-γ and TNF-α) released by GD2-EATs were significantly lower (50%) than those released by the conventional iv GD2-BsAb plus unarmed T cell injection.

### Example 6: EATs Traffic into Tumors Bypassing their Initial Pulmonary Sequestration

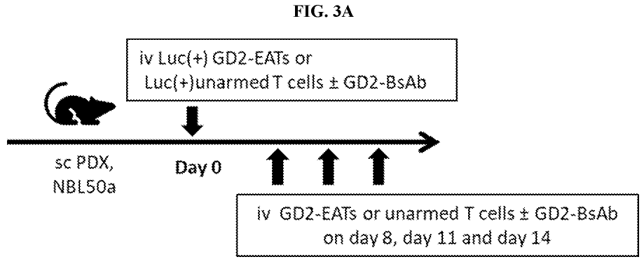

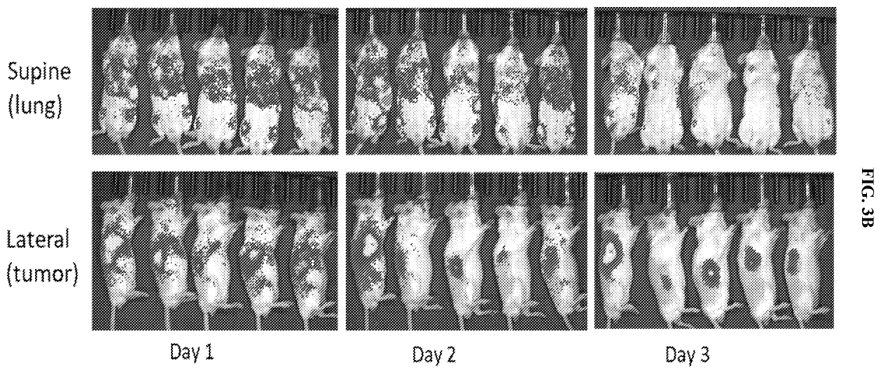

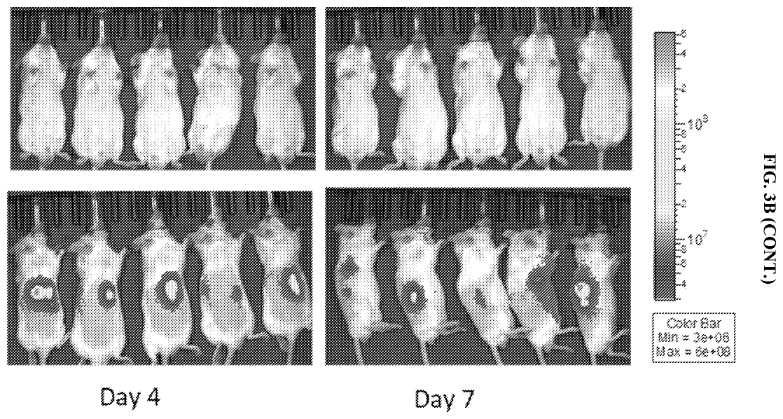

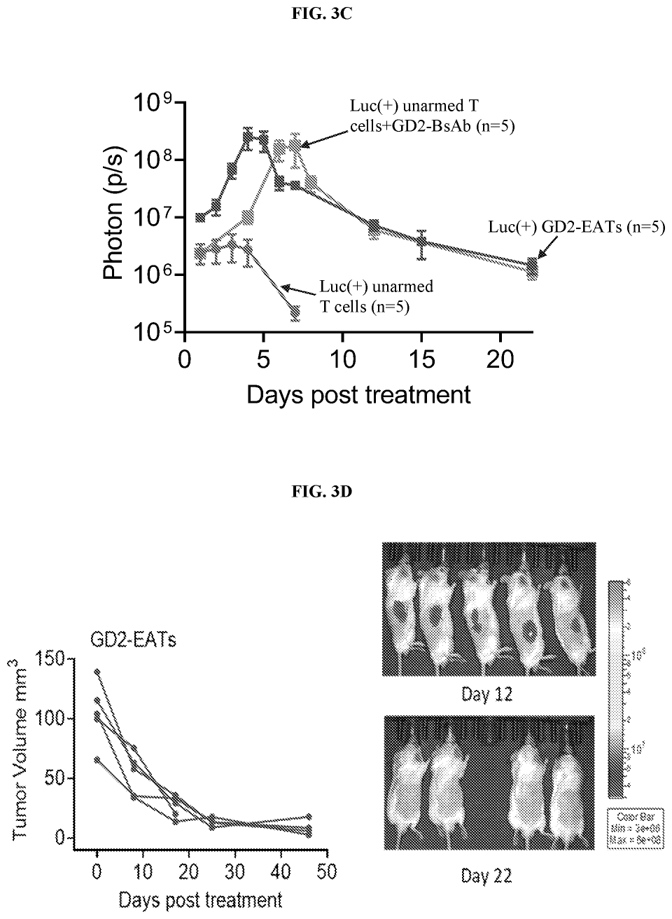

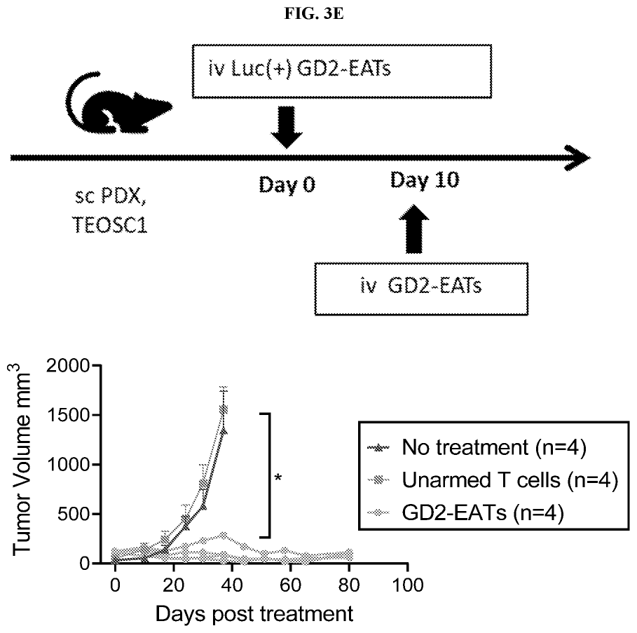

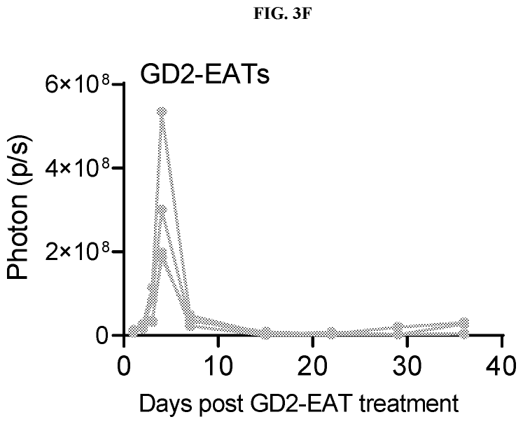

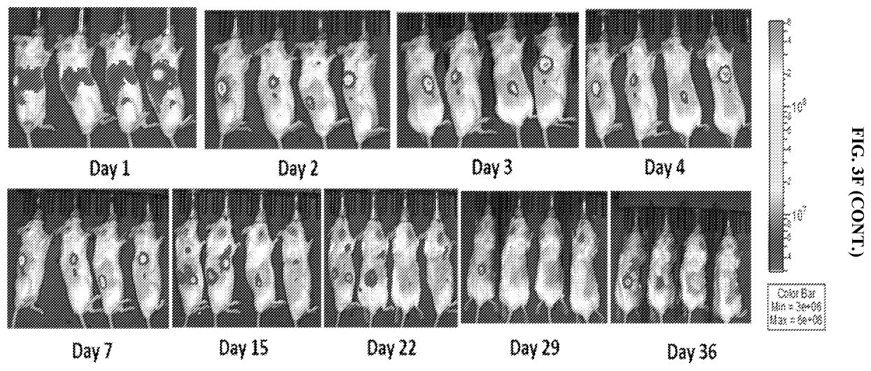

To quantitate how efficiently EATs traffic into solid tumors, luciferase transduced T cells and armed ex vivo with GD2-BsAb [Luc(+) GD2-EATs] were generated. After first iv injection of Luc(+) GD2-EATs (10 μg of GD2-BsAb/2×10⁷ cells) or Luc(+) unarmed T cells (2×10⁷ cells) into neuroblastoma PDX bearing mice, subsequent T cells used were untransduced (FIG. 3A). Without GD2-BsAb arming, Luc(+) unarmed T cells did not localize to tumors and dissipated. In contrast, Luc(+) GD2-EATs rapidly trafficked into GD2(+) tumors (FIG. 3B), following a transient rest in the lungs on day 1, as the TILs signal increased over time to peak on day 4 (FIG. 3C), while, Luc(+) unarmed T cells (2×10⁷ cells) with iv GD2-BsAb (10 μg) were visible in tumors by day 3 and peaking around day 6 and 7. As tumor regressed, the total bioluminescence of Luc(+) GD2-EATs also diminished (FIG. 3D). In a second set of T cell trafficking studies (FIG. 3E), subtherapeutic dose of GD2-EATs in an osteosarcoma PDX model was tested by administering only 2 doses of GD2-EATs every 10 days. Luciferin signal of the tumor infiltrating GD2-EATs persisted over 36 days in mice with residual tumors (FIG. 3F).

### Example 7: EATs Showed Potent Anti-Tumor Activity with Minimal Toxicities In Vivo

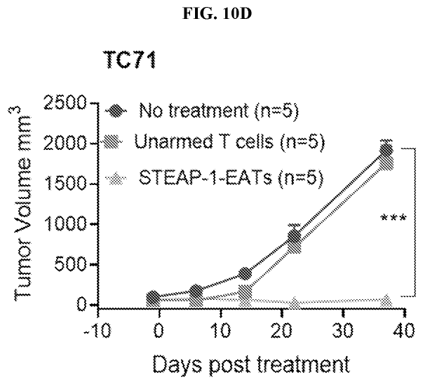

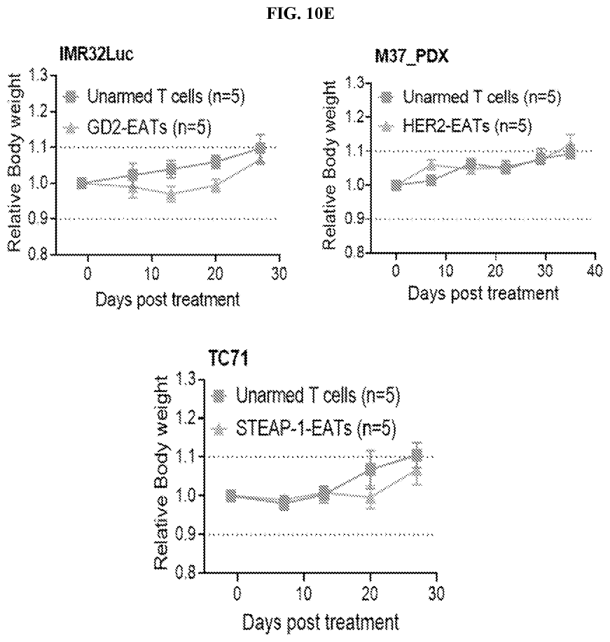

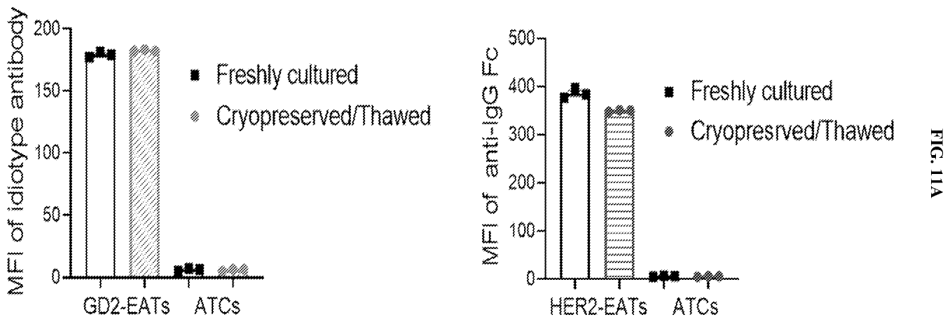

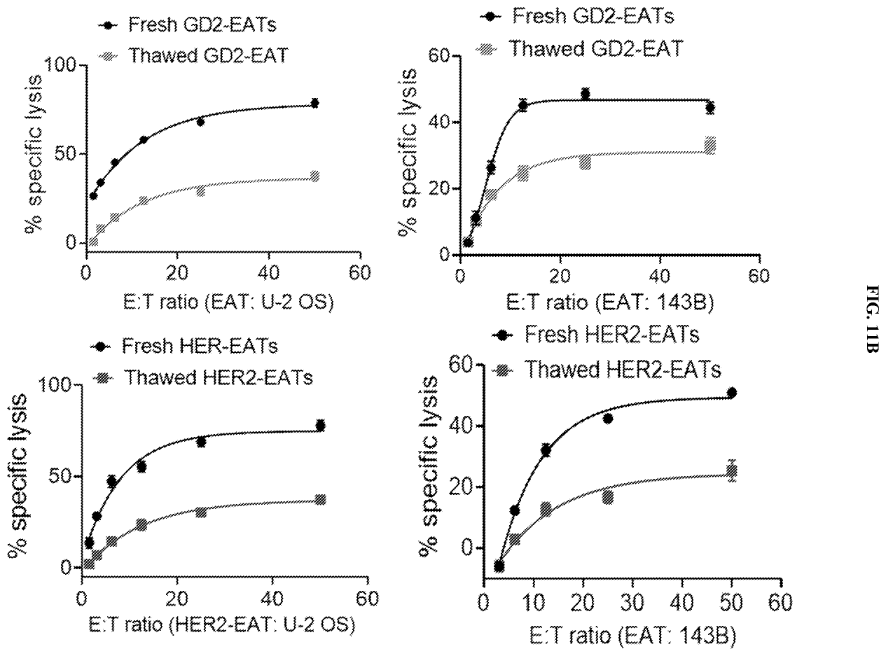

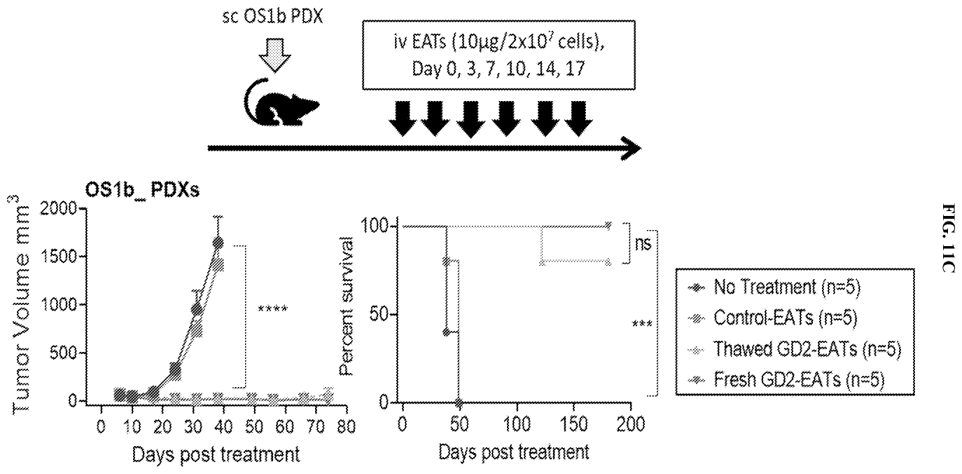

Adoptive T cell cytotherapy using EATs was tested against a panel of xenograft mouse models (FIG. 10A). GD2-EATs were tested against neuroblastoma PDXs (Piro20Lung), neuroblastoma cell line (IMR32Luc) xenografts, and melanoma cell line (M14Luc) xenografts (FIG. 10B). HER2-EATs were tested against osteosarcoma PDXs (TEOSC1), breast cancer PDXs (M37), and osteosarcoma cell line (143B) xenografts (FIG. 10C). Beyond GD2 and HER2, EATs targeted to antigens including STEAP-1 (six transmembrane epithelial antigen prostate-1) on Ewing sarcoma cell line (TC71) were tested against each target cell line xenografts; in each instance, EATs showed potent anti-tumor effects (FIG. 10D), without weight loss or adverse effects during follow-up period (FIG. 10E).

### Example 8: Critical Determinants for Effective EAT Therapy

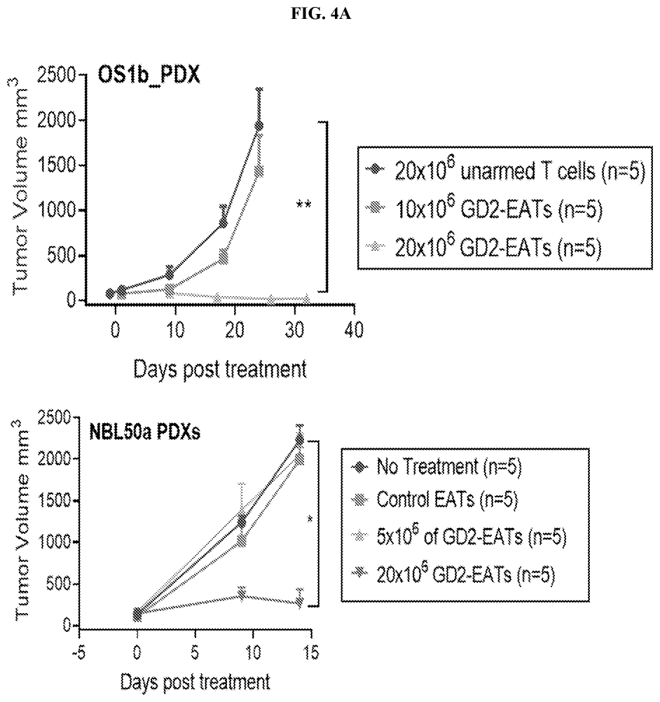

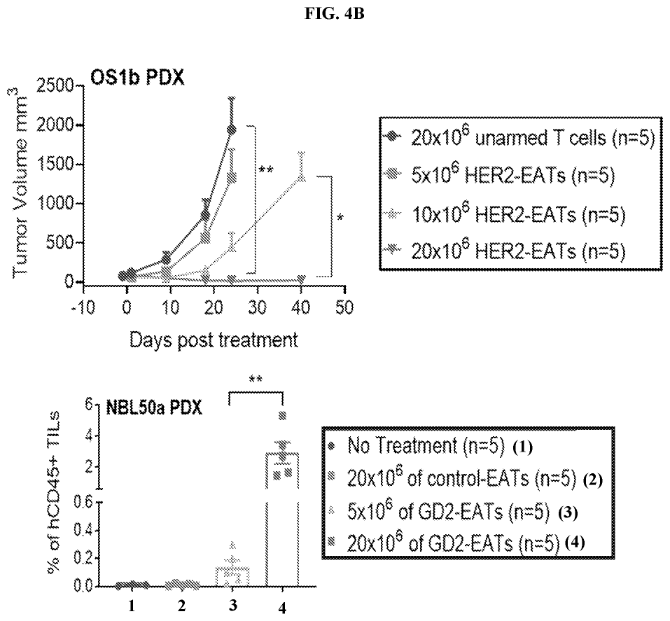

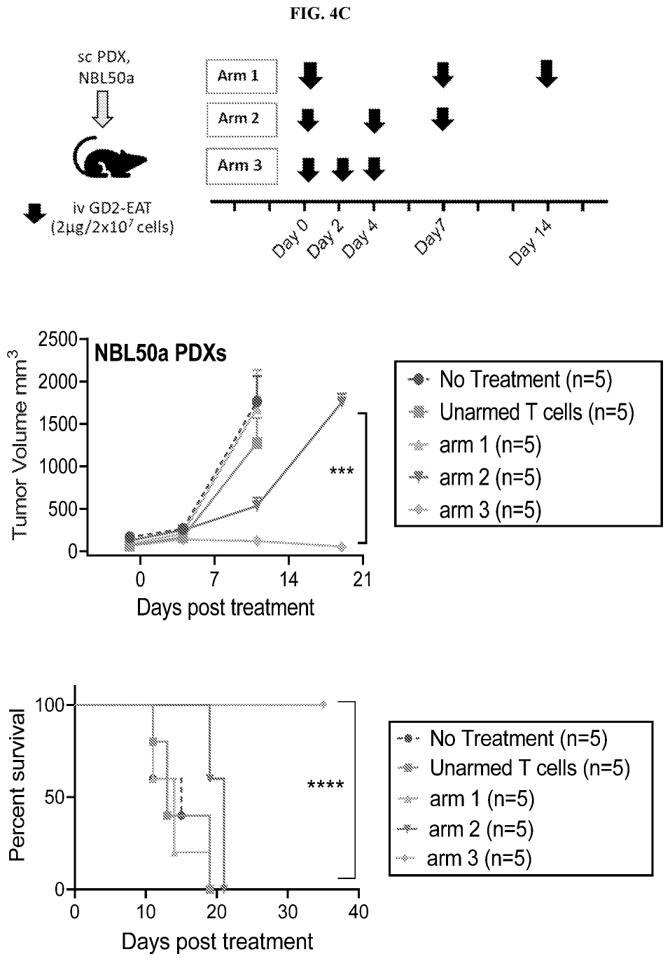

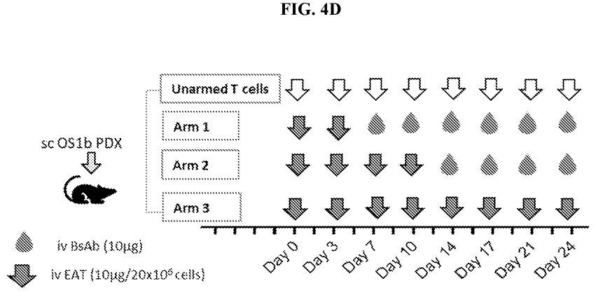

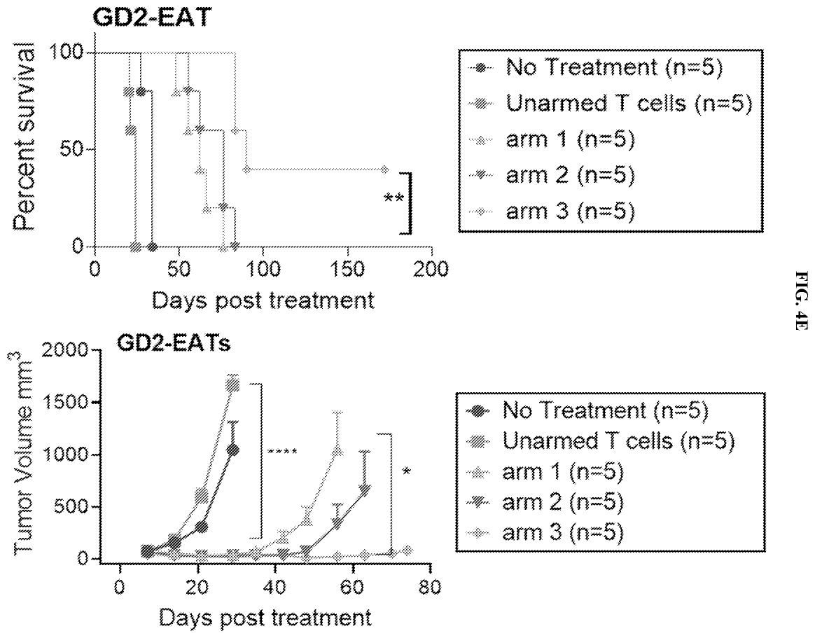

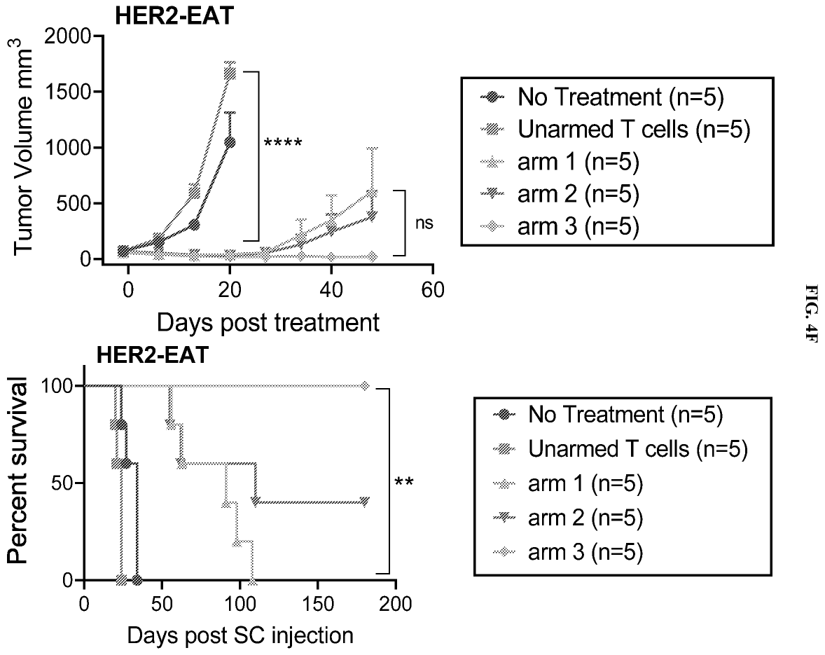

Anti-tumor activity of EAT depends on infused T cell number. To optimize preclinical treatment, different variables were assessed to study their impact on the therapeutic efficacy of EATs. First, the effect of infused EAT cell number was evaluated in osteosarcoma and neuroblastoma PDX models (FIGS. 4A-4B). At an arming dose of 0.1 μg of BsAb/10⁶ cells, increasing cell dose of GD2-EATs or HER2-EATs (5×10⁶ cells, 10×10⁶ cells and 20×10⁶ cells, respectively) were administered twice-weekly. Anti-tumor effect consistently increased with the number of EATs infused; while 20×10⁶ of EATs were effective in eliminating these tumors, 5×10⁶ and 10×10⁶ of EATs were insufficient. This anti-tumor response was correlated with the percentage of human CD45(+) TILs, which was evident with 20×10⁶ of GD2-EATs, but negligible with 5×10⁶ of GD2-EATs.

EAT efficacy in vivo is schedule dependent. To identify the optimal treatment schedule, neuroblastoma PDXs were treated with 3 different EAT schedules: arm 1, low intensity (1 dose/week); arm 2, standard (2 doses/week); or arm 3, dose-dense (3 doses/week), with GD2-EATs armed at fixed dose of 2 μg of GD2-BsAb/2×10⁷ cells (FIG. 4C). The dose-dense schedule (arm 3) demonstrated superior anti-tumor efficacy against rapidly growing PDXs compared to standard or low intensity schedules (P=0.0001), which also translated into survival benefit (P<0.0001).

Enhancing EAT efficacy by supplemental EAT vs supplemental BsAb. Next, to test how many doses of EATs are needed to sustain anti-tumor effect and if supplemental BsAb injection can replace subsequent EATs, osteosarcoma PDXs were treated with three different schedules (FIGS. 4D-4F): arm 1, two doses of EATs followed by 6 doses of iv BsAb; arm 2, 4 doses of EATs followed by 4 doses of BsAbs; arm 3, 8 doses of EATs. Arming doses were fixed at 10 μg of BsAb/2×10⁷ cells, while supplemental BsAb was fixed at 10 μg per injection. In contrast to the rapid tumor growth with no treatment or 8 doses of unarmed T cells, two doses of GD2-EATs and HER2-EATs significantly suppressed tumor growth. However, additional doses of EATs were necessary for durable responses. Contrary to the mice treated with two doses of GD2-EATs and HER2-EATs showing short-term response, among those treated with 8 doses of EATs, 2 of 5 mice in GD2-EATs and 5 of 5 mice in HER2-EATs showed sustained remission past 6 months, confirming the superior dose effect of EATs not correctable by supplemental BsAb injections.

### Example 9: Following Cryopreservation EATs Retain Anti-Tumor Properties

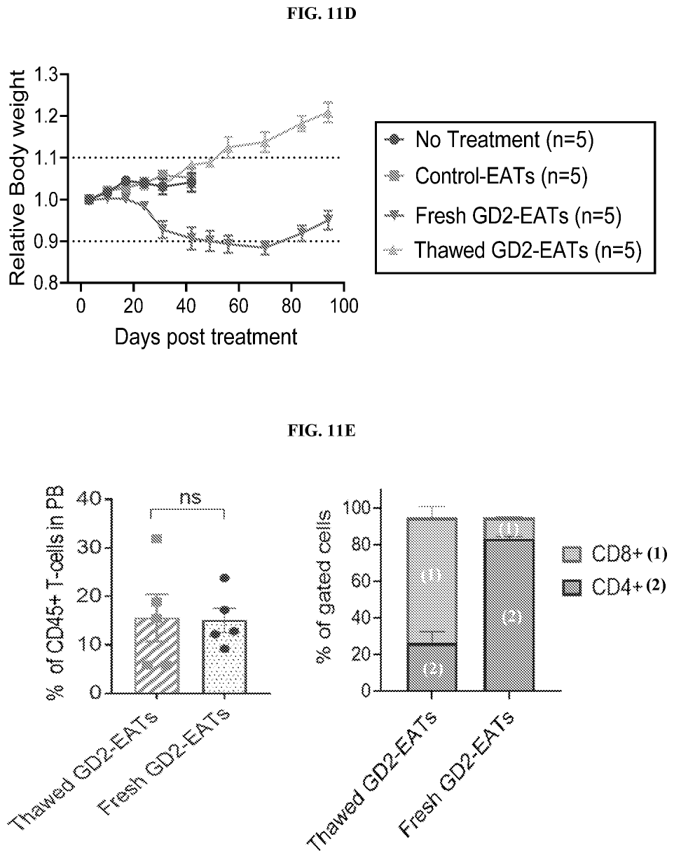

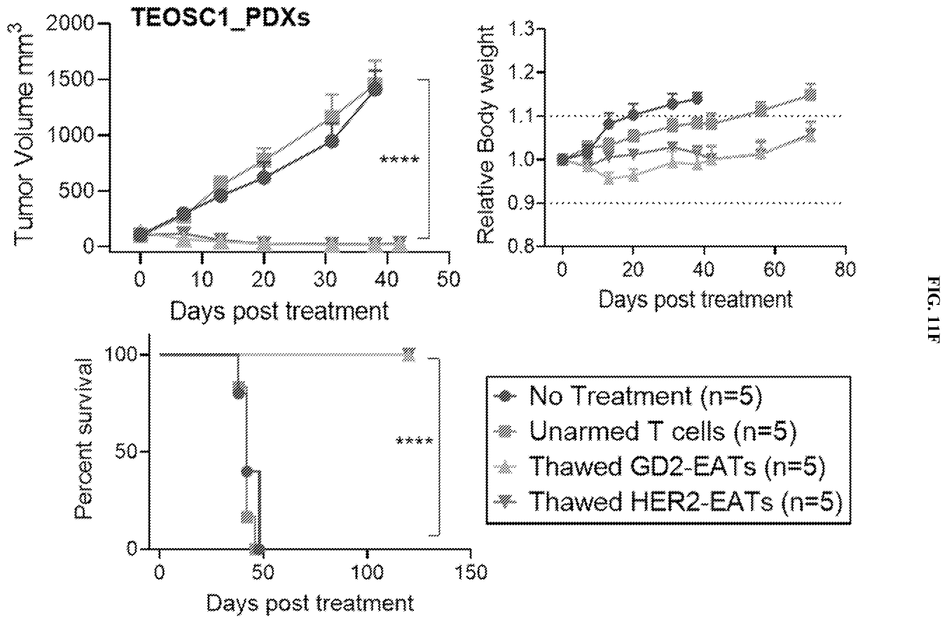

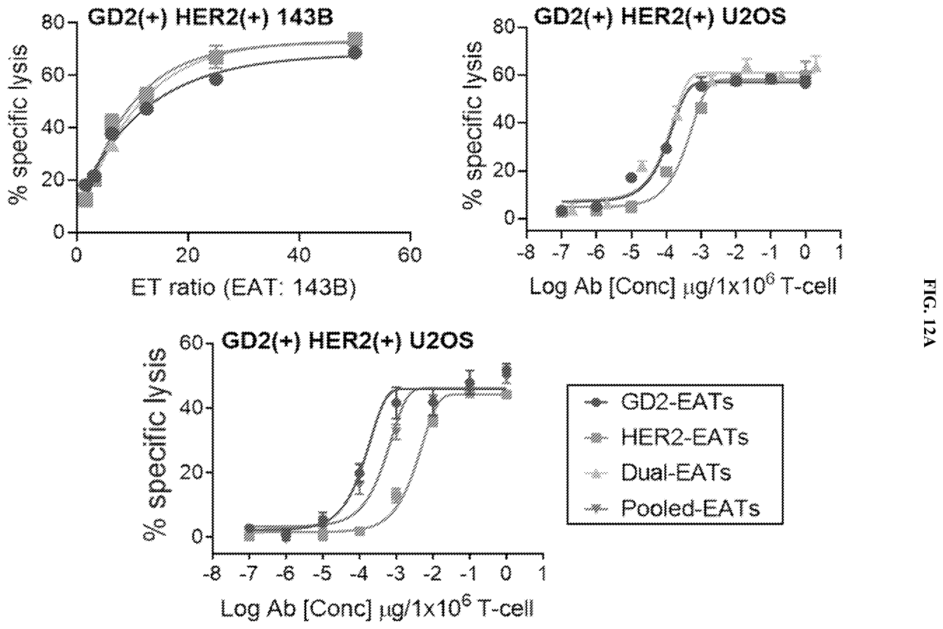

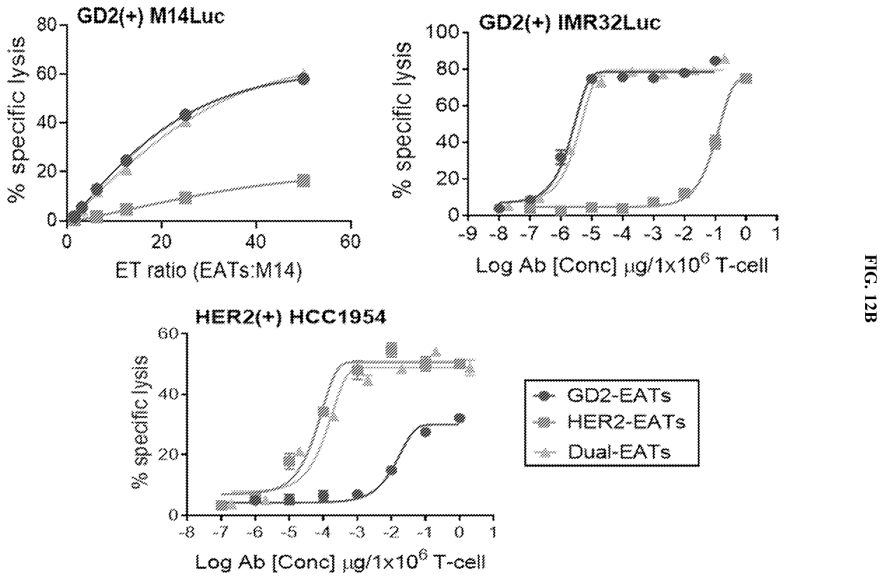

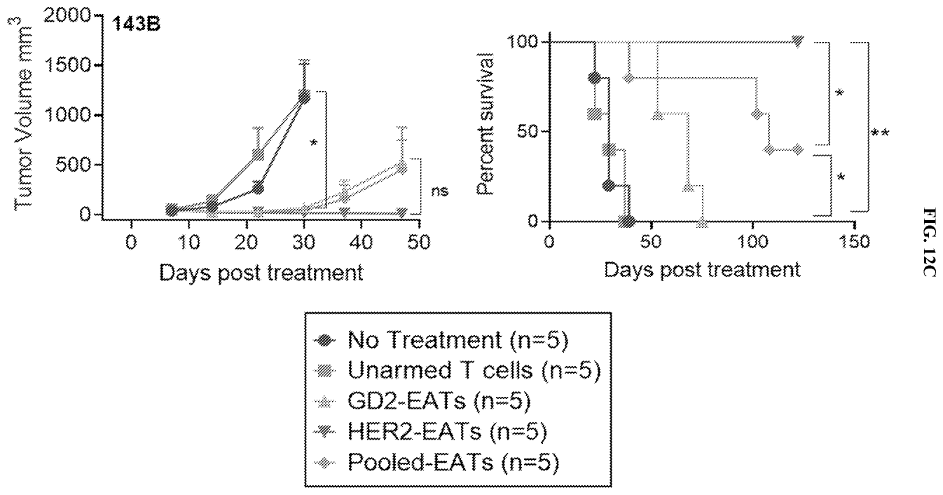

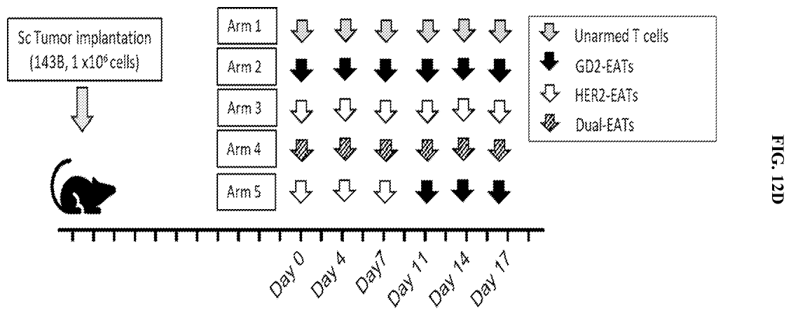

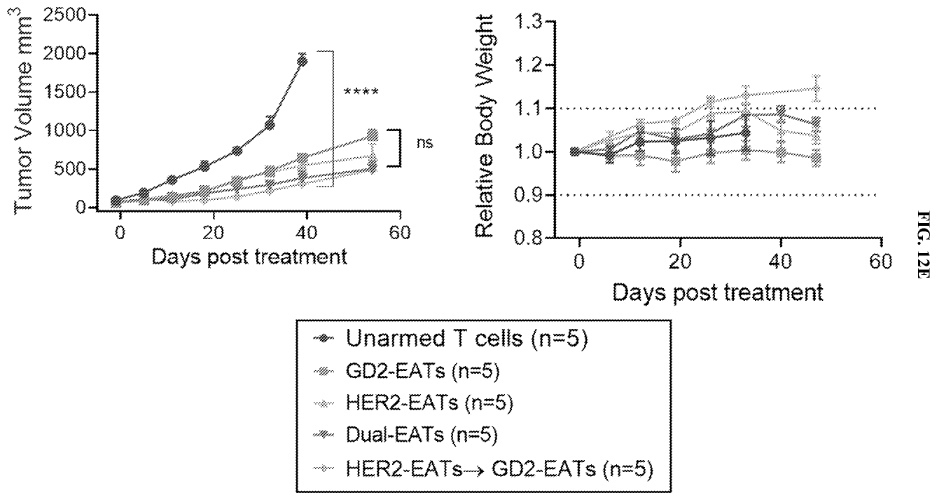

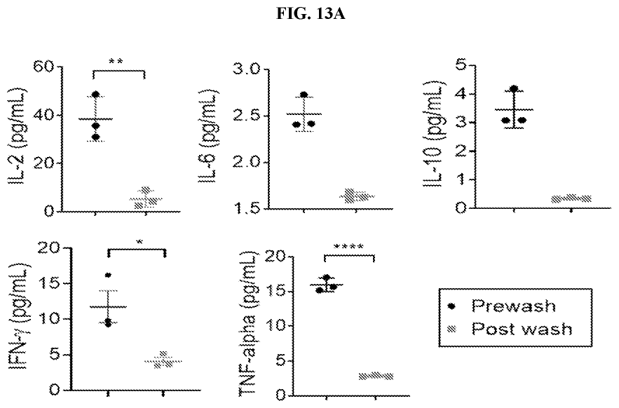

To ensure transportability and clinical utility of EATs, cryopreserved EATs were tested for their viability, BsAb surface density, and tumoricidal properties. After thawing at 37° C., EATs remained over 85% viable, irrespective of whether they were frozen for 2 hrs at −80° C. or up to 3 months in liquid nitrogen. When these EATs (thawed EATs) were stained with anti-idiotype antibody or anti-human IgG Fc antibody, BsAb surface density remained comparable to freshly armed EATs (fresh EATs) by MFIs (FIG. 11A). Although cytotoxicity of thawed EATs did diminish after cryopreservation and thawing (50% of maximal killing efficacy of fresh EATs) as a result of not enough recovery time after thawing, antigen-specificity was maintained (FIG. 11B). Suitable recovery time after thawing include 1-2 days.

In vivo anti-tumor efficacies of thawed EATs were evaluated using two different osteosarcoma PDX models. In the first PDX (OSOS1B PDX) model, both fresh and thawed GD2-EATs exerted potent anti-tumor effects and prolonged survival (FIG. 11C). Four of 5 mice treated with thawed GD2-EATs showed long-term remission past 6 months post treatment. Interestingly, while mice treated with fresh GD2-EATs developed mild to moderate GVHD 1 to 2 months post treatment, mice treated with thawed GD2-EATs displayed no clinical signs of GVHD throughout the entire follow-up period, maintaining body weight, good coat condition and general activity (FIG. 11D). When blood samples of each group were analyzed on day 45 post treatment (FIG. 11E), thawed GD2-EAT treated mice displayed a predominance of CD8(+) T cells in the blood, while the fresh GD2-EAT group showed mostly CD4(+) T cells, correlating with their clinical manifestations of GVHD. In the second tumor model (FIG. 11F), both thawed GD2-EATs and thawed HER2-EATs exerted strong anti-tumor effects against telangiectatic osteosarcoma PDXs. All tumors regressed without significant toxicities, and there were no signs of GVHD or tumor relapse past 4 months post treatment.

### Example 10: T Cells Armed with Multiple BsAbs (Multi-EATs) Achieved Multi-Specificity Against Multiple Tumor Targets

Combinatorial EAT strategies. To further improve anti-tumor effects against solid tumors, strategies for overcoming tumor heterogeneity and target antigen downregulation or loss are needed. Multiple antigen-targeting EAT (multi-EAT) strategies to address these obstacles in single antigen targeted treatment were studied. Without wishing to be bound by theory, it is believed that BsAbs built on the same IgG-[L]-scFv platform should arm T cells through the identical huOKT3-scFv domain and thus exert comparable activation. Dual specificities were tested in two ways: by arming T cells with a combination of 2 different BsAbs (dual-EATs) and by combining two EATs each separately armed with a different BsAb (pooled-EATs), administered together or sequentially. GD2-BsAb and HER2-BsAb were used for arming, and in vitro cytotoxicity was tested. Pooled-EATs (GD2-EATs+HER2-EATs) or dual-EATs (GD2/HER2-EATs) showed comparable tumor cell killing against GD2(+) and/or HER2(+) tumor cell lines (FIGS. 12A-12B).

To evaluate in vivo anti-tumor effects of these combinatorial approaches, pooled-EATs were tested first, with 4 doses of EATs (2×10⁷ cells per injection) armed at a fixed dose 0.5 μg of total BsAb/10⁶ cells (FIG. 12C). Pooled-EATs (5 μg/1×10⁷ of GD2-EATs and 5 μg/1×10⁷ of HER2-EATs) showed a comparable anti-tumor response against GD2(+) HER2(+) osteosarcoma PDXs. 5 of 5 mice in the HER2-EATs group, none of 5 in the GD2-EATs group, and 2 of 5 in the pooled-EATs group (n=5) remained progression-free. The dual-EATs approach was also tested (FIGS. 12D-12E). T cells were armed with either GD2-BsAb (10 μg/2×10⁷ T cells), HER2-BsAb (10 μg/2×10⁷ T cells), or a mixture of both BsAbs (dual-EATs, 10 μg of GD2-BsAb and 10 μg of HER2-BsAb/2×10⁷ T cells) and evaluated in vivo. Additionally, sequential combination of EATs (HER2-EATs followed by GD2-EATs) was also compared. Dual-EATs approach did not compromise anti-tumor activities of either BsAb, nor did it increase toxicities. The dual-EATs (GD2/HER2-EATs) significantly suppressed osteosarcoma tumor growth, demonstrating comparable potency to GD2-EATs, HER2-EATs, and sequential combination of EATs.

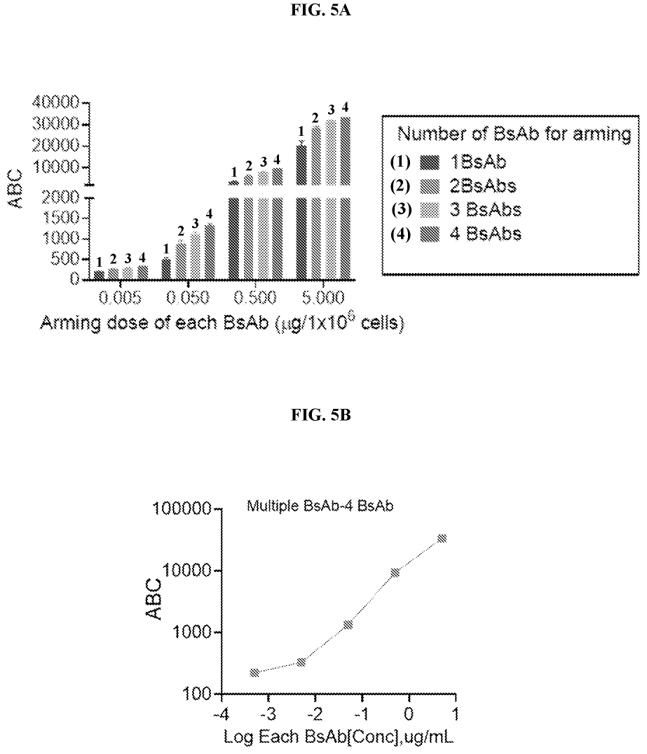

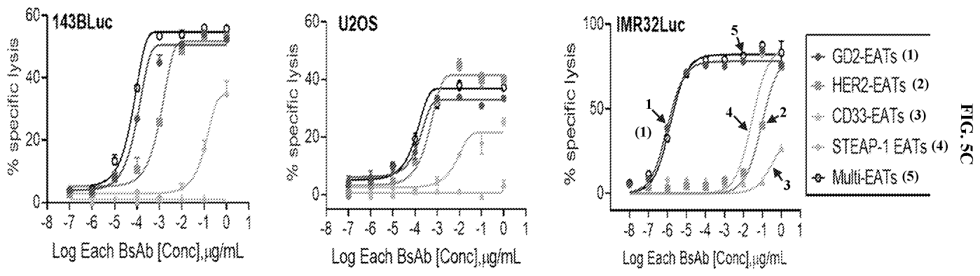

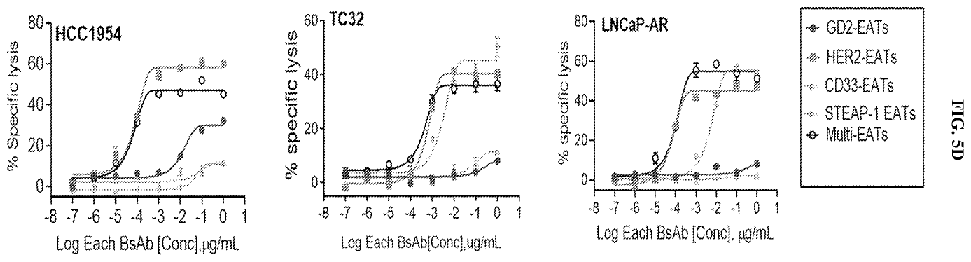

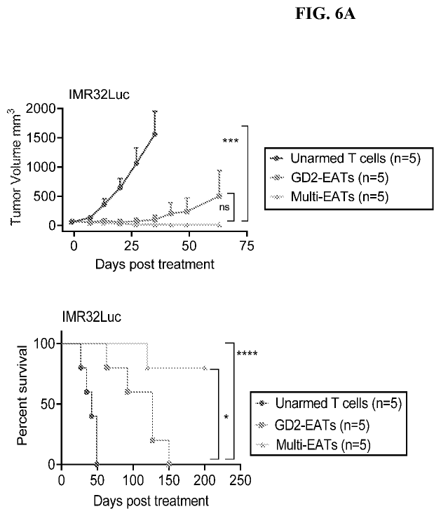

Multispecific EATs (multi-EATs) using a mixture of BsAbs. Furthermore, multi-EATs using multiple BsAbs, were constructed on the same IgG-[L]-scFv platform, targeting tumor antigens including GD2, HER2, CD33, or STEAP-1. Multi-EATs were evaluated for BsAb surface density (ABC) and in vitro cytotoxicity. As the number of BsAb for arming and arming doses of each BsAb increased, BsAb surface density has increased (FIG. 5A). With more than 3 BsAbs at high arming doses (5 μg of each BsAb/10⁶ cells), surface density plateaued at approximately 33,500 molecules per T cell.

To identify the range of optimal surface density of BsAbs for multi-EATs, ADTC was studied over a range of E:T ratios (from 50:1 to 1.5:1) and BsAb arming doses (FIG. 5B). Multi-EATs (armed with multiple BsAbs targeting tumor antigens including GD2, HER2, CD33, or STEAP-1) showed comparable cytotoxicity against CD33(+) leukemia cell line (Molm13) at arming doses of each BsAb between 0.05 μg/10⁶ T cells to 5 μg/10⁶ T cells, at ABCs between 1,500 to 30,000 molecules per T cell. At surface BsAb density between 1,500 to 10,000 molecules per T cell, multi-EATs showed the best tumoricidal activity.

The anti-tumor activities of the multi-EATs were evaluated using multiple tumor cell lines (FIGS. 5C-5D). Despite the presence of multiple BsAbs on the same EAT, killing potencies of multi-EATs against each target were comparable to those of monospecific EATs, although the maximal cytotoxicity (Emax) did vary depending on the specific target studied.

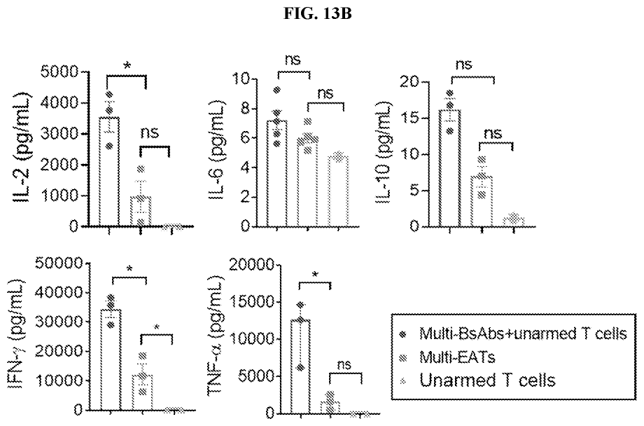

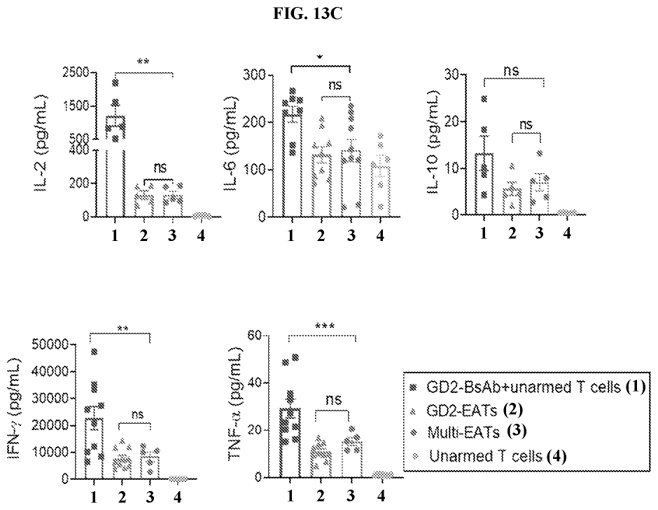

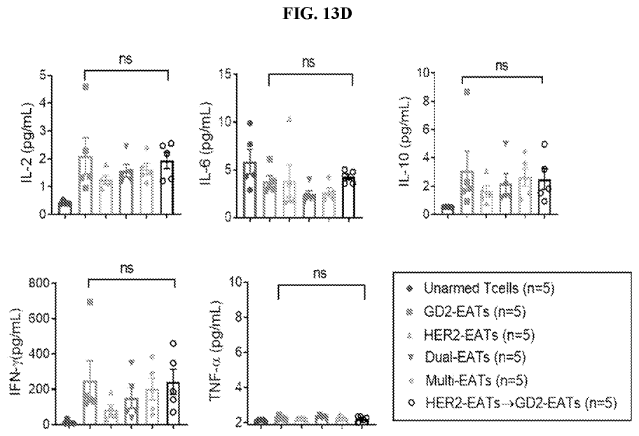

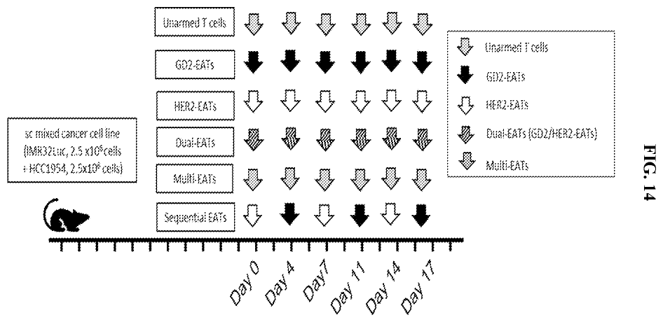

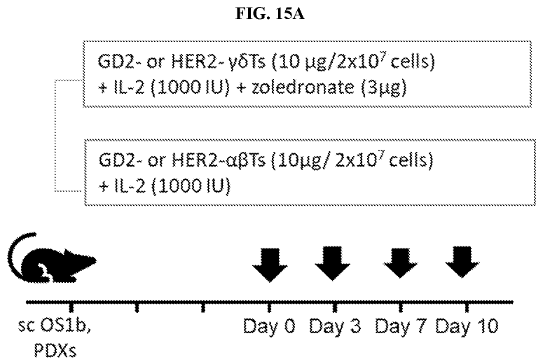

Multi-specific EATs had comparable anti-tumor activity in vivo with reduced cytokine release. When multiple BsAbs were administered together, cytokine release could increase substantially. To determine clinical feasibility of multi-EATs, cytokine release was evaluated. See, e.g., D. W. Lee et al., Blood 124, 188 (2014); S. A. Grupp et al., N Engl J Med 368, 1509 (2013); J. N. Kochenderfer et al., Blood 119, 2709 (2012) (demonstrating that high cytokine release is a critical factor that adversely impacts the clinical feasibility of an immunotherapeutic agent). Cytokine release between multiple BsAbs mixed with unarmed T cells (co-incubation with 5 BsAbs) and multi-EATs (T cells armed with 5 BsAbs and washed) were compared. T cells were incubated for 20 minutes at arming doses of 0.05 μg to 5 μg of each BsAb/10⁶ T cells, washed twice with PBS, and co-cultured with target cells (E:T ratio of 50:1) for 4 hours at 37° C. Low levels of cytokines were released during BsAb incubation and completely removed after wash (FIG. 13A). After co-culture with target cells (IMR32-Luc), cytokine levels released by multi-EATs (IL-2, IFN-γ and TNF-α) were significantly lower than those by multiple BsAbs mixed with unarmed T cells (FIG. 13B).

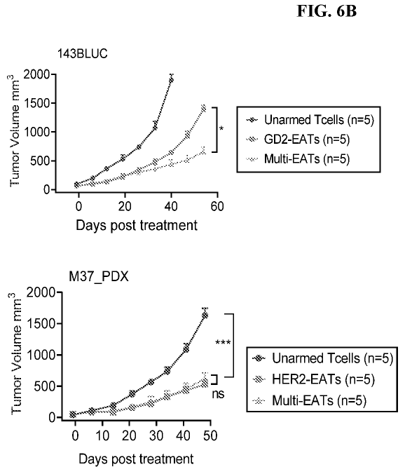

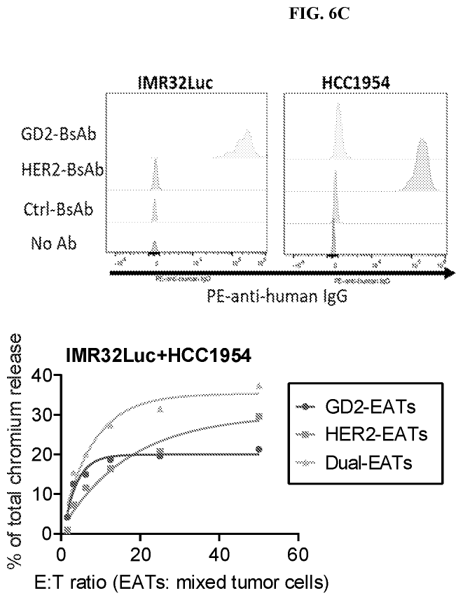

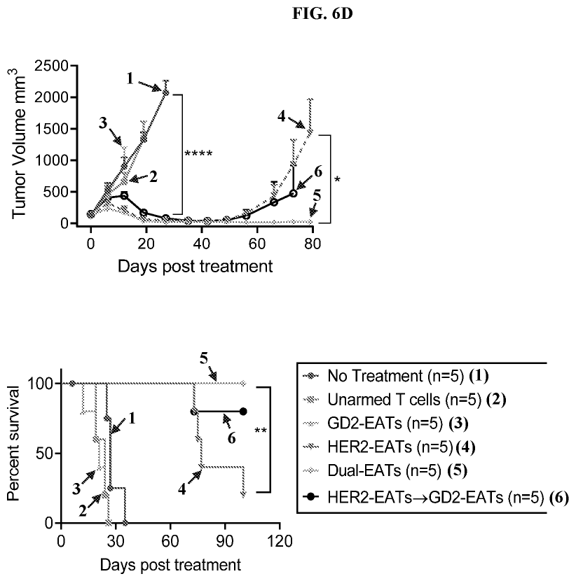

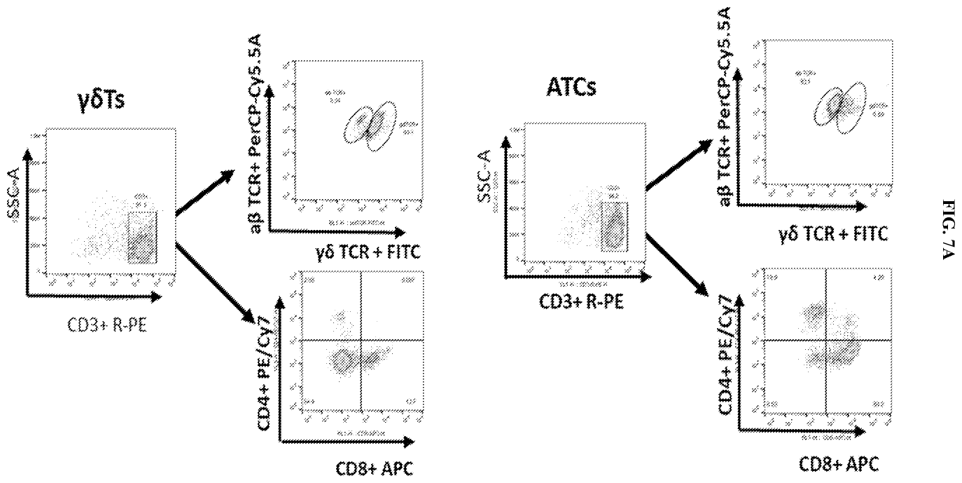

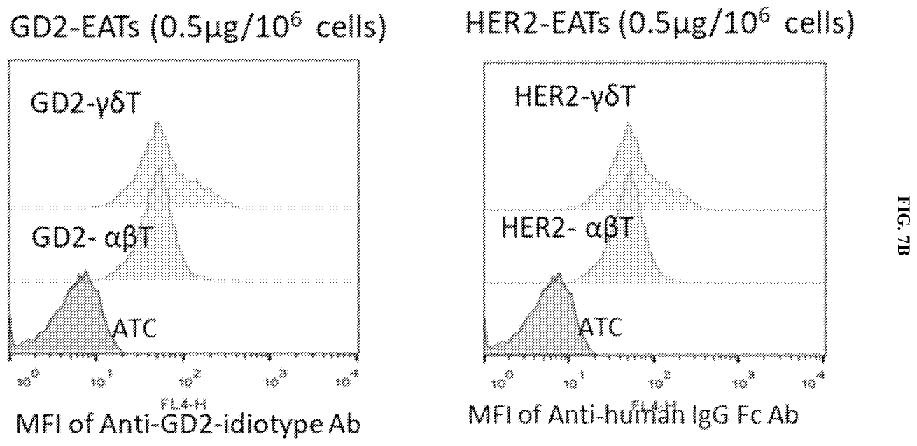

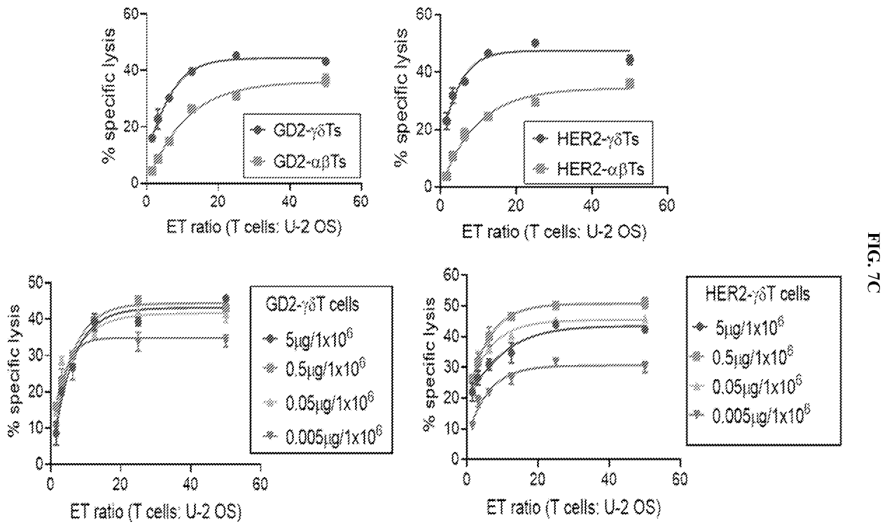

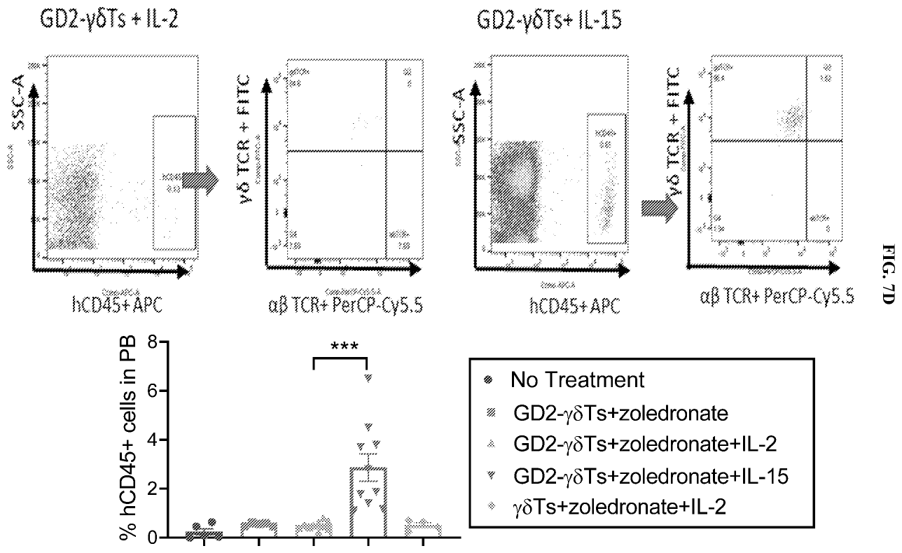

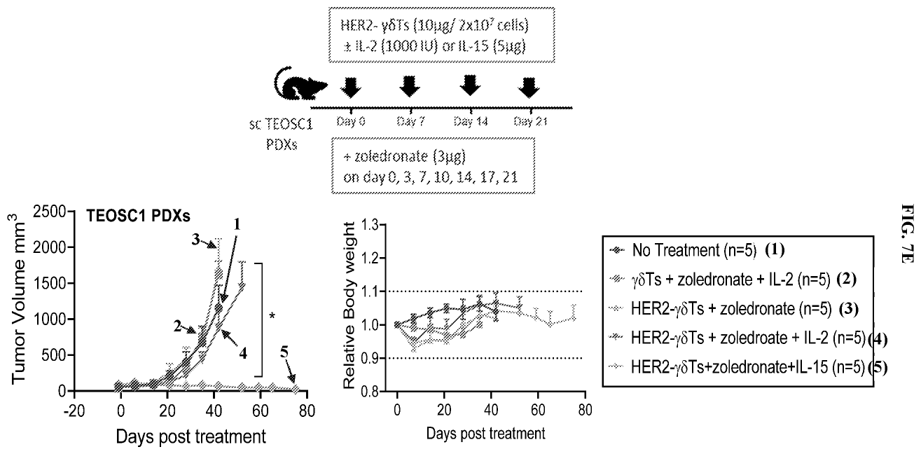

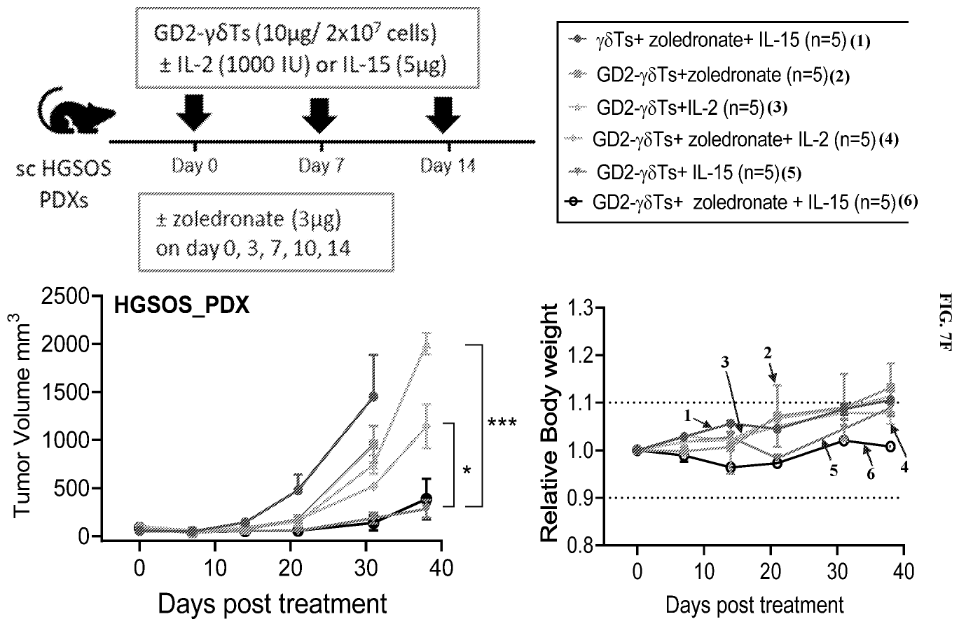

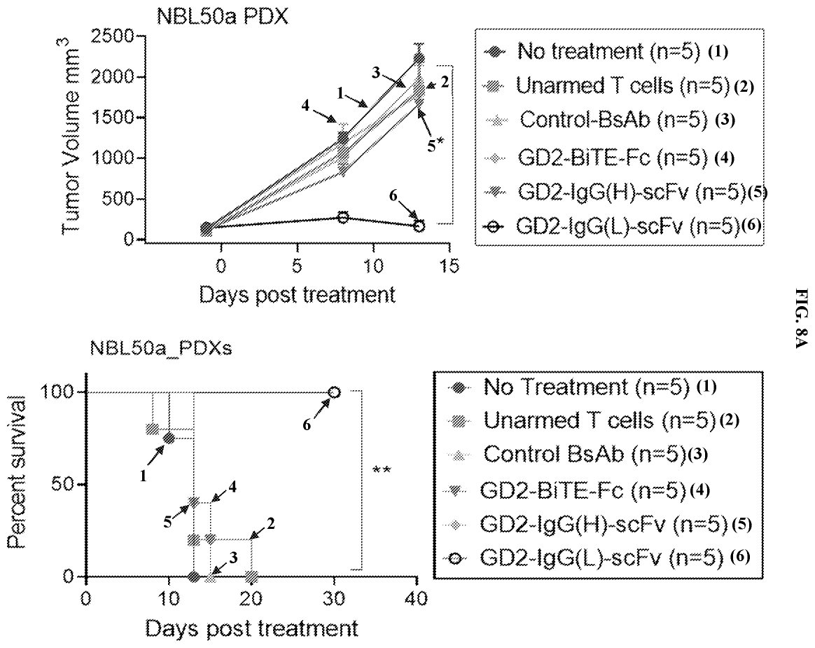

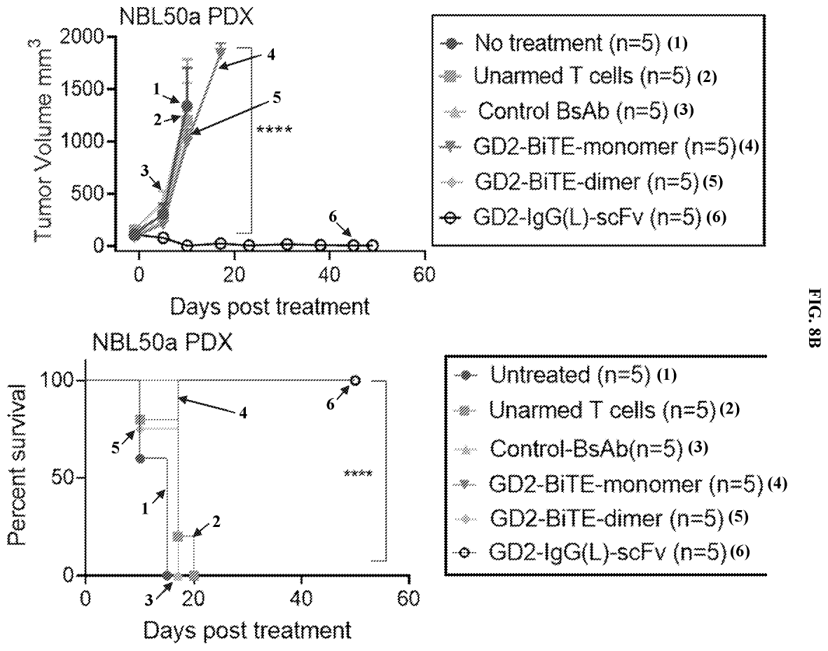

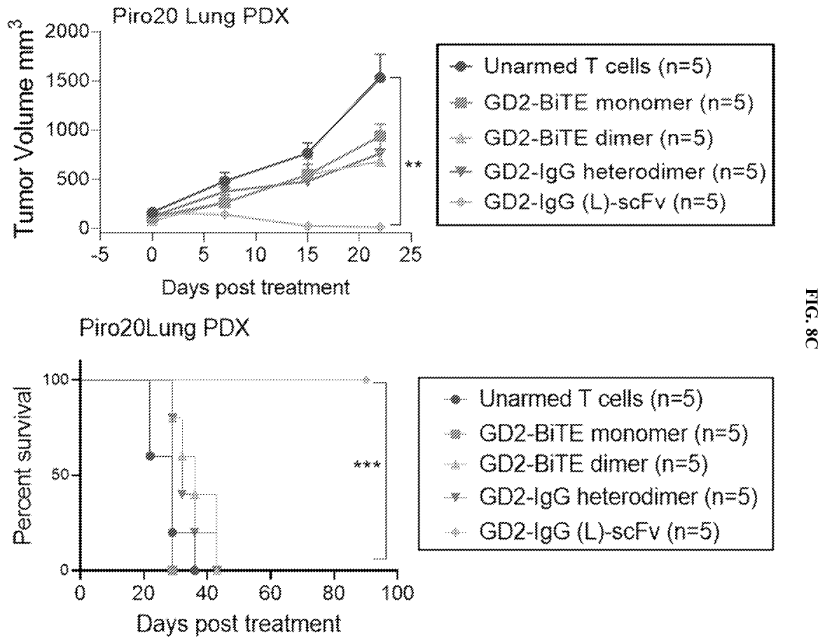

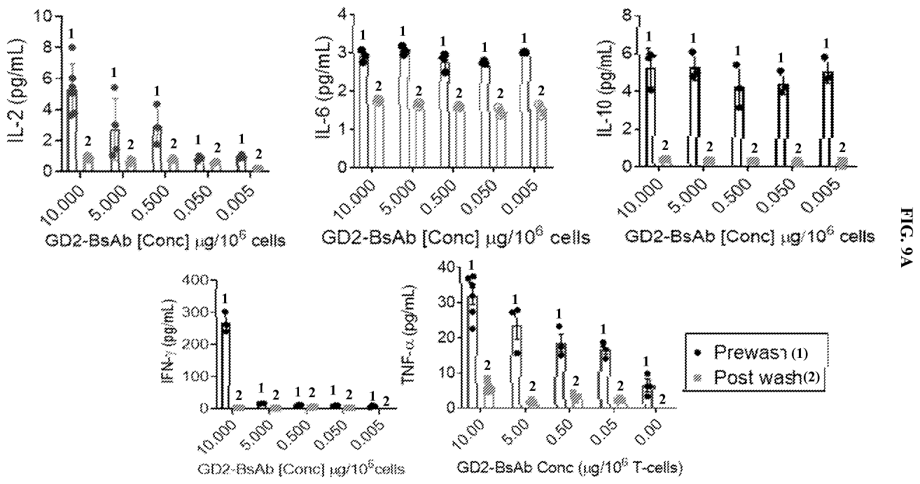

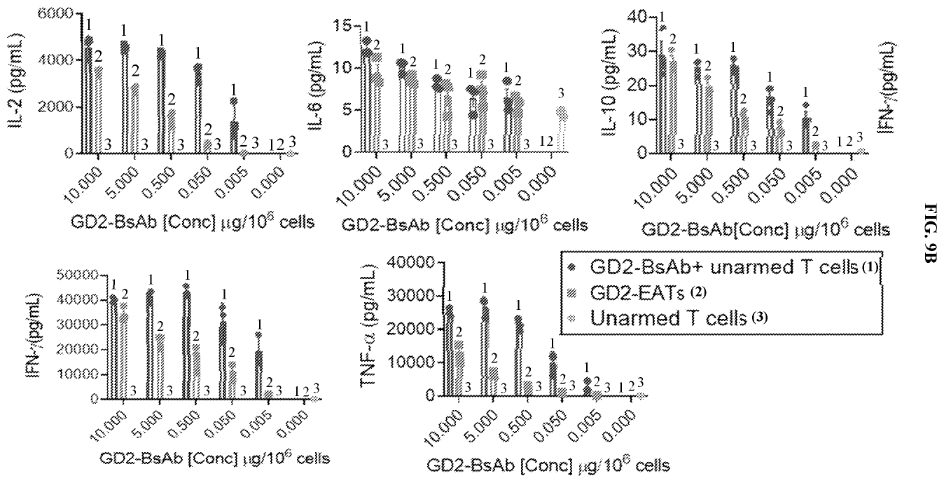

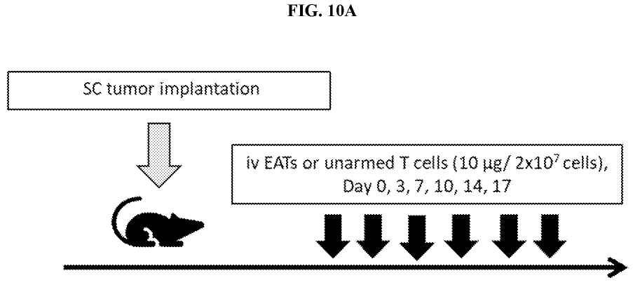

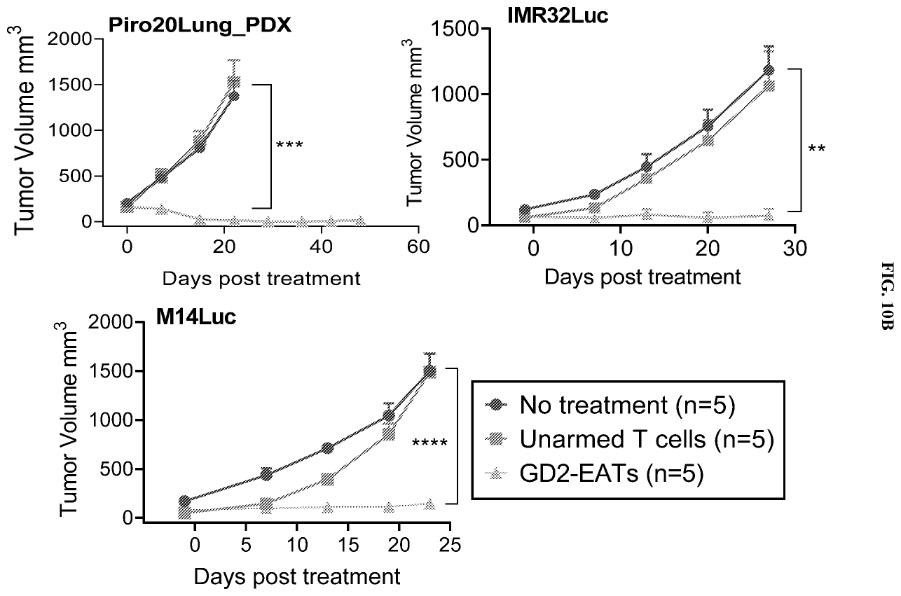

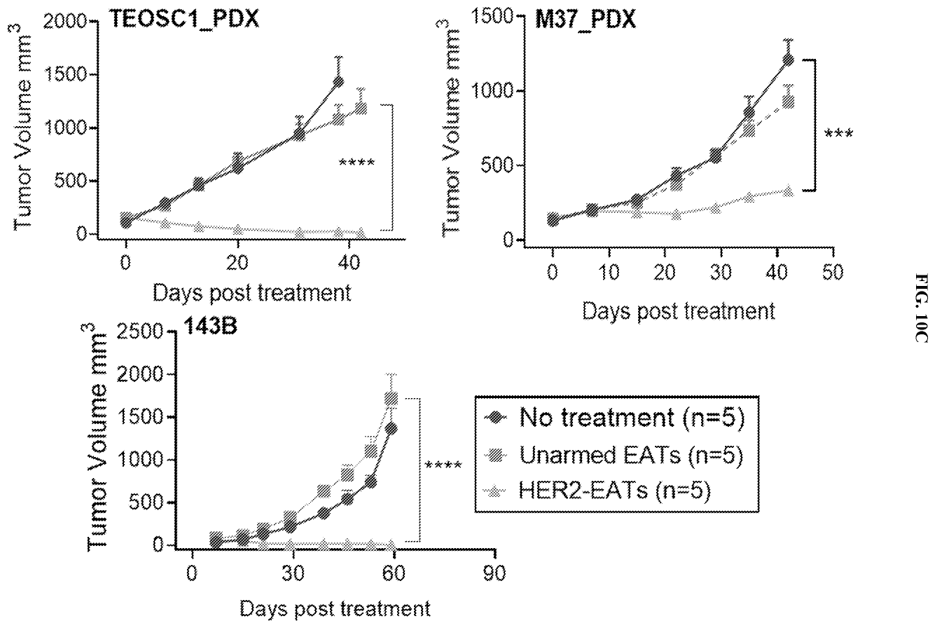

In vivo potency of multi-EATs was tested in multiple tumor cell line xenograft mouse models (FIGS. 6A-6B). At an arming dose of 2 μg of each BsAb per 2×10⁷ of T cells, multi-EATs (10 μg of total BsAb/2×10⁷ cells per injection) significantly suppressed tumor growth and exerted equivalent anti-tumor responses to monospecific EATs against the panel of target appropriate tumor xenografts. Multi-EATs improved tumor control and overall survival of mice harboring IMR32Luc or 143BLuc xenografts, suggesting that multi-EATs could potentially reduce or prevent tumor escape.

To further examine if multi-EATs can overcome tumor heterogeneity, their anti-tumor activity against mixed cancer cell lines of GD2(+)HER(−) IMR32Luc and GD2^{low}HER2(+) HCC1954 (mixture in 1:1 ratio) were tested (FIGS. 6C-6D). Compared to the low efficacy (Emax) of monospecific GD2-EATs and HER2-EATs, dual-EATs (GD2/HER2-EATs) and multi-EATs (against tumor antigens including GD2, HER2, CD33, STEAP-1) induced greater cytotoxicity in vitro. This increased cytotoxicity of EATs with multiple specificities translated into superior in vivo anti-tumor response against mixed cancer cell line xenografts.

A mixture of two cell lines (2.5×10⁶ of IMR32Luc and 2.5×10⁶ of HCC1954) was subcutaneously implanted into mice and treated with GD2-EATs, HER2-EATs, dual-EATs (GD2/HER2-EATs), multi-EATs, and sequential combination of EATs (HER2-EATs followed by GD2-EATs), respectively (FIG. 14). BsAb and T cells were fixed at 10 μg per each BsAb and 2×10⁷ T cells per injection. All EAT treatments were well tolerated irrespective of total BsAb doses for arming. While monospecific GD2-EATs failed to suppress tumor growth, dual-EATs, multi-EATs, and sequential combination of EATs successfully regressed tumors and significantly improved survival compared to monospecific EATs (vs. HER2-EATs, P=0.0033; vs. GD2-EATs, P<0.0001), demonstrating the advantage of multi-targeting EAT strategies against heterogenous solid tumors.

In vivo cytokine release by multi-EATs was also measured and compared to that of GD2-BsAb plus unarmed T cells, GD2-EATs, HER2-EATs, and unarmed T cells alone in GD2(+) HER2(+) osteosarcoma PDX model and mixed cancer cell line [(GD2(+)IMR32Luc and HER2(+) HCC1954)] xenograft model (FIGS. 13C-13D). BsAb dose and T cell number were again fixed at 10 μg for each BsAb and 2×10⁷ for cell per injection. Multi-EATs (50 μg of total BsAb/2×10⁷ cells) released significantly lower levels of IL-2, IL-6, IFN-γ, and TNF-α than GD2-BsAb (10 μg) plus T cells (2×10⁷ cells), and there was no significant difference in cytokine release among EATs.

### Example 11: Ex Vivo BsAb Armed γδ T Cells are Equally Active as αβ T Cells

Since CD3 is present on diverse subpopulations of T cell types, the functionality of gamma delta (γδ) T cells was tested. γδ T cells have reduced alloreactivities with potential as an allogeneic “off-the-shelf” T cell source (J. Fisher & J. Anderson, Frontiers in immunology 9, 1409 (2018)). After expansion from fresh PBMCs with IL-2 and zoledronate for 12 days, more than 90% of CD3(+) T cells were γδ TCR (+), and less than 10% were αβ TCR (+) (FIG. 7A). The majority of γδ T cells were CD3(+) and CD4(−) CD8(−) double negative, contrasting with T cells expanded using CD3/CD28 beads where the majority were T cells (>90%). After arming γδ T cells with GD2-BsAb (GD2-γδTs) or HER2-BsAb (HER2-γδTs), surface BsAb density was measured by flow cytometry. The MFIs were comparable to those of αβ-EATs (FIG. 7B). In the presence of GD2-BsAb or HER2-BsAb, γδ T cells mediated potent tumoricidal activity against GD2(+) and/or HER2(+) tumor cell lines in vitro, with maximal cytotoxic efficiency of GD2-γδTs and HER2-γδTs achieved at arming doses between 0.05 μg/1×10⁶ cells to 5 μg/1×10⁶ cells (FIG. 7C).

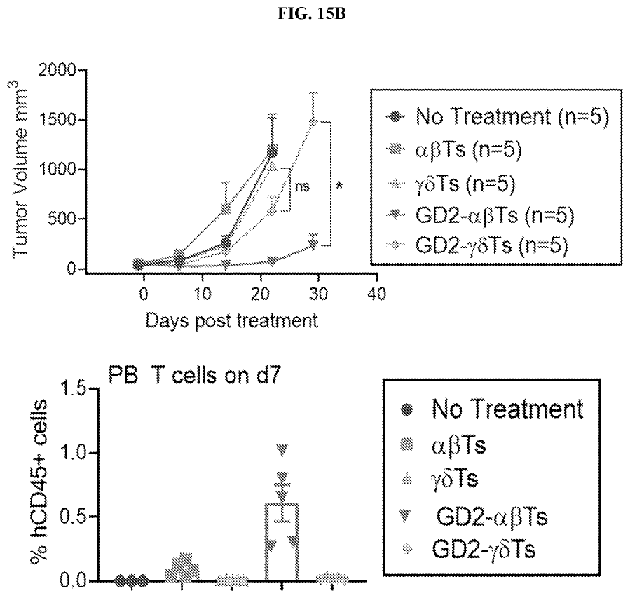

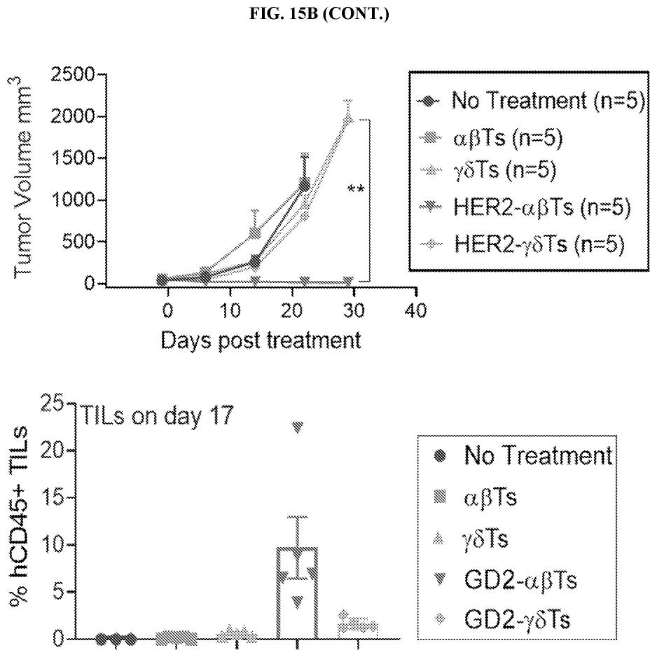

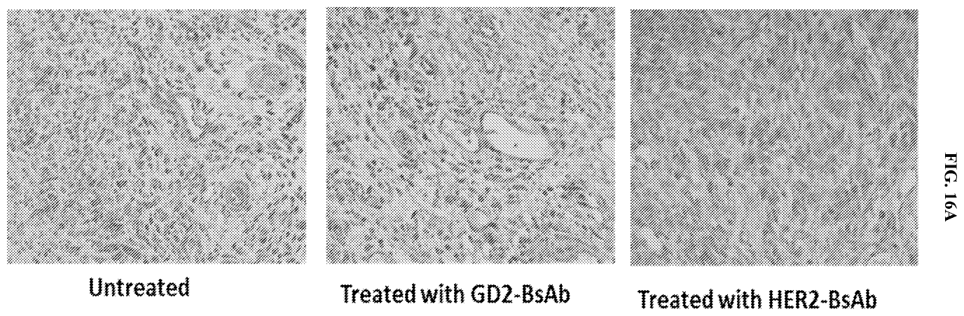

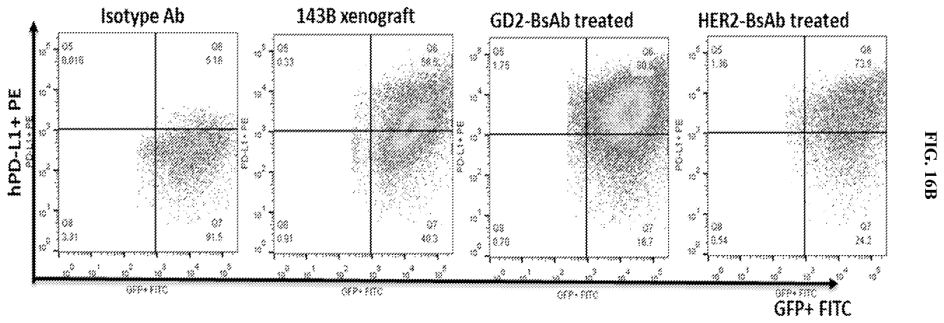

The in vivo anti-tumor activity of GD2-γδTs and HER2-γδTs in osteosarcoma PDX models, was compared to corresponding αβ-EATs (FIG. 15A-15B). Despite supplementary IL-2, GD2-γδTs and HER2-γδTs did not produce significant anti-tumor responses against antigen (+) tumors irrespective of additional zoledronate. Measurement of T cells in the blood and tumors after GD2-γδT therapy showed substantially fewer human CD45(+) T cells compared to GD2-αβTs by flow cytometry, suggesting a poor survival of γδ-EATs in vivo.

However, when exogenous IL-15 (as IL15Rα-IL15 complex) was used instead of IL-2, in vivo survival and function of γδ-EATs were significantly improved (FIGS. 7D-7F). γδ T cells expanded from fresh PBMCs using 204 of zoledronate plus 30 ng/mL of IL-15 for 12 to 14 days were armed with GD2-BsAb or HER2-BsAb and administered iv into xenografted mice, with 5 μg of subcutaneous IL-15 or 1,000 IU of IL-2. GD2-γδTs and HER2-γδTs sustained with IL-15 exerted significant anti-tumor effects against GD2(+) HER2 (+) osteosarcoma PDXs without toxicities or weight loss, in contrast to the same EATs sustained with IL-2, demonstrating the potential utility of allogenic γδ-EATs instead of autologous T cells.

### Example 12: Osteosarcoma Cell Lines Tested Positive for GD2 and/or HER2

Osteosarcoma Cell lines. Representative human osteosarcoma cell lines, 143B (ATCC—CRL-8303), U-2 OS (ATCC—HTB-96), MG-63 (ATCC—CRL-1427), HOS (ATCC—CRL-1543), and Saos-2 (ATCC—HTB-85), and osteoblast cell line, hFOB 1.19 (CRL-1137), were purchased from ATCC (Manassa Va.). All cells were authenticated by short tandem repeats profiling using PowerPlex 1.2 System (Promega, Madison, Wis.), and periodically tested for mycoplasma infection using a commercial kit (Lonza, Basel, Switzerland). The cells were cultured in RPMI1640 (Sigma) supplemented with 10% heat-inactivated fetal bovine serum (FBS, Life Technologies, Carlsbad, Calif.) at 37° C. in a 5% CO₂ humidified incubator.

Flow cytometry. For flow cytometric analysis of antigen expression by human osteosarcoma cell lines, cells were harvested, cell viability was determined. 1×10⁶ cells were stained with 1 μg of antigen specific mAbs in a total volume of 100 μL for 30 min at 4° C. Anti-CD20 chimeric mAb, rituximab, or mouse IgG1 monoclonal antibody was used as isotype control. After washing with PBS, cells were re-incubated with 0.1 μg PE-conjugated anti-human IgG Ab (Biolegend, San Diego, Calif., 409304). For each sample, 20,000 live cells were analyzed using a BD FACS Calibur™ (BD Biosciences, Heidelberg, Germany). Data were analyzed with FlowJo V10 software (Ashland, Oreg., USA) using geometric mean fluorescence intensity (MFI). The MFI for isotype control antibody was set to 5, and the MFIs for antibody binding were normalized based on isotype control.

Effector cell preparation. Effector peripheral blood mononuclear cells (PBMC) were separated by ficoll from buffy coats purchased from the New York Blood Center. T cells were purified from PBMC using Pan T cell isolation kit (Miltenyi Biotec). These T cells were activated by CD3/CD28 Dynabeads (Invitrogen, Carlsbad, Calif.) for 7 to 14 days in the presence of 30 IU/mL of IL-2 according to manufacturer's protocol. PBMCs and ATCs were analyzed by FACS for their proportion of CD3(+), CD4(+), CD8(+), and CD56(+) cells.

Cytotoxicity assays (⁵¹ chromium release assay). Antibody dependent T cell-mediated cytotoxicity (ADTC) was assessed by ⁵¹Cr release assay, and EC₅₀ was calculated using Sigma Plot software. Tumor cells were labeled with sodium ⁵¹Cr chromate (Amersham, Arlington Height, Ill.) at 100 mCi/10⁶ cells at 37° C. for 1 hour. After two washes, tumor cells were plated in a 96-well plate before mixing with activated T cells (ATCs) at decreasing concentrations of T-BsAb. Effector to target cells ratio (E:T ratio) was 10:1, and cytotoxicity was analyzed after incubation at 37° C. for 4 hours. The released ⁵¹Cr was measured by a gamma counter (Packed Instrument, Downers Grove, Ill.). Percentage of specific lysis was calculated using the formula: 100% (experimental cpm−background cpm)/(total cpm−background cpm), where cpm represented counts per minute of ⁵¹Cr released. Total release of ⁵¹Cr was assessed by lysis with 10% SDS (Sigma, St Louis, Mo.) and background release was measured in the absence of effector cells and antibodies.

Antibodies. For each BsAb, scFv of huOKT3 was fused to the C-terminus of the light chain of human IgG1 via a C-terminal (G₄S)₃ linker (SEQ ID NO: 158) (Orcutt K D et al., Protein Eng Des Sel 2010; 23(4):221-8). N297A and K322A on Fc were generated with site-directed mutagenesis via primer extension in polymerase chain reactions (Reikofski J, Tao B Y. Biotechnol Adv 1992; 10(4):535-47). The nucleotide sequence encoding each BsAb was synthesized by GenScript and was subcloned into a mammalian expression vector. Each BsAb was produced using Expi293™ expression system (Thermo Fisher Scientific) separately. Antibodies were purified with protein A affinity column chromatography. The purity of these antibodies was evaluated by size-exclusion high-performance liquid chromatography (SE-HPLC). GD2-BsAb was linked to the carboxyl end of the anti-GD2 hu3F8 IgG1 light chain (Xu H, Cheng M, Guo H, Chen Y, Huse M, Cheung N K. Cancer Immunol Res 2015; 3(3):266-77), and HER2-BsAb linked to the anti-HER2 trastuzumab IgG1 light chain (Lopez-Albaitero A, Xu H, Guo H, Wang L, Wu Z, Tran H, et al., Oncoimmunology 2017; 6(3):e1267891). Anti-GPA/anti-CD3 BsAb were used as a control BsAb for ADTC and in vivo animal experiments (Wu Z, Guo H F, Xu H, Cheung N V. Mol Cancer Ther 2018; 17(10):2164-75).

T cell arming. Ex vivo activated T cells were harvested between day 7 and day 14 and armed with each BsAb for 20 minutes at room temperature. After incubation, the T cells were washed with PBS twice. Properties of ex vivo bispecific antibody armed T cells (EATs) were tested with cell surface density of BsAb using idiotype antibodies and in vitro cytotoxicity against target antigens. For quantification of BsAb bound to T cells (antibody binding capacity, ABC), EATs were stained with anti-human IgG Fc antibody or anti-idiotypic antibody (A1G4 for hu3F8) and analyzed by flow cytometry along with Quantum™ Simply Cellular® (QSC) microspheres.

In vivo experiments. All animal experiments were performed according to Institutional Animal Care and Use Committee (IACUC) guidelines. For in vivo experiments, BALB-Rag2^{-/-}IL-2R-γc-KO (DKO) mice (Taconic Biosciences, Germantown, N.Y.) were used (Andrade D et al., Arthritis Rheum 2011; 63(9):2764-73). In vivo experiments were performed in 6-10-week-old mice. Tumor cells were suspended in Matrigel (Corning Corp, Tewksbury Mass.) and implanted in the flank of DKO mice. Besides tumor cell line xenografts, 3 different patient-derived tumor xenografts (PDXs) both positive for GD2 and HER2 were established from fresh surgical specimens with IRB approval. Tumor size was measured using TM900 scanner (Piera, Brussels, BE), and treatment was initiated when tumor size reached 100 mm³. Tumor growth curves and overall survival was analyzed, and overall survival was defined as the time from start of treatment to when tumor volume reached 2000 mm³. To define the well-being of mice, CBC analyses, changes in body weight, behavior and physical appearance were monitored.

Flow cytometry of blood and tumor. Peripheral blood and tumors were collected and analyzed by flow cytometry. Anti-human antibodies against CD3, CD4, CD8, and CD45 (Biolegend, San Diego, Calif.) were used to define T cell engraftment and subpopulation, and anti-human PD-1 and PD-L1 antibodies (Biolegend, San Diego, Calif.) were used to quantify their expression by T cells and osteosarcoma tumor cells. Stained cells were processed with BD LSRFORTESSA (BD Biosciences, Heidelberg, Germany) and analyzed with FlowJo software (FlowJo, LLC, Ashland, Oreg.).

Immunohistochemical (IHC) staining. Formalin-fixed paraffin-embedded tumor sections were made, and immunohistochemical (IHC) staining for human CD3, CD4 and CD8 T cells was done to confirm T cell infiltration inside tumors. The IHC staining was performed using Discovery XT processor (Ventana Medical Systems, Oro Valley, Ariz.). Paraffin-embedded tumor sections were deparaffinized with EZPrep buffer (Ventana Medical Systems, Oro Valley, Ariz.), antigen retrieval was performed with CC1 buffer (Ventana Medical Systems, Oro Valley, Ariz.), and sections were blocked for 30 minutes with background buffer solution (Innovex). Anti-CD3 (DAKO, cat #A0452, 1.2 μg/mL), anti-CD4 (Ventana, cat #A790-4423, 0.5 μg/mL), and anti-CD8 (Ventana, cat #790-4460, 0.07 μg/mL) were applied, and sections were incubated for 5 hours, followed by 60 min incubation with biotinylated goat anti-rabbit IgG (Vector laboratories, cat #PK6101) at 1:200 dilution. For PD-L1 staining, the sections were pre-treated with Leica Bond ER2 Buffer (Leica Biosystems) for 20 min at 100° C., stained with PD-L1 rabbit monoclonal antibody (Cell signaling, cat #29122, 2.5 mg/mL) for 1 hour on Leica Bond RX (Leica Biosystems). Control antibody staining was done with biotinylated goat anti-rat IgG (Vector Labs, Burlingame, Calif., cat #MKB-22258). All images were captured from tumor sections using Nikon ECLIPSE Ni-U microscope and NIS-Elements 4.0 imaging software. Antigen positive cells were counted with Qupath 0.1.2.

GD2 expression by IHC. Fresh frozen tumor sections were made using Tissue-Tek OCT (Miles Laboratories, Inc, Elkhart, Ind.) with liquid nitrogen and stored at −80° C. The tumor sections were stained with mouse IgG3 mAb 3F8 as previously described (Dobrenkov K, Ostrovnaya I, Gu J, Cheung I Y, Cheung N K. Pediatr Blood Cancer 2016; 63(10):1780-5). Stained slides were captured using a Nikon ECLIPSE Ni-U microscope and analyzed, and the tissue staining intensity was compared with positive and negative controls and scored from 0 to 4 according to 2 components: staining intensity and percentage of positive cells. Each sample was assessed and graded by 2 independent observers.

Statistics. Differences among groups indicated in the figures were tested for significance by one-way ANOVA or student's t-test, and survival outcomes were analyzed using GraphPad Prism 7.0. P-value<0.05 was considered statistically significant.

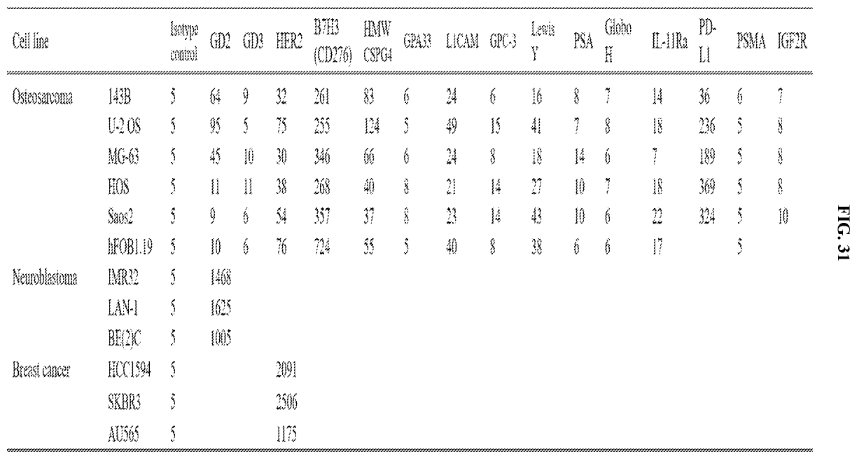

To identify potential therapeutic targets for osteosarcoma, the expression of GD2, GD3, HER2, B7H3 (CD276), high-molecular weight melanoma antigen (HMW), chondroitin-sulfate proteoglycan-4 (GSPG-4), L1 cell adhesion molecule (L1CAM), glypican-3 (GPC-3), prostate-specific antigen (PSA), prostate-specific membrane antigen (PSMA), insulin-like growth factor 2 receptor (IGF2R), interleukin 11 receptor-α (IL-11Rα), and PD-L1 by osteosarcoma tumor cell lines was assessed (FIG. 31).

Surface antigens on osteosarcoma cell lines were semi-quantitated by flow cytometric analysis and normalized with the MFI for control antibody (FIG. 23A). The majority of osteosarcoma cell lines expressed GD2 and/or HER2 antigen on their cell surface; binding intensities (MFIs) for GD2 was generally much lower than those for neuroblastoma cell lines, while MFIs for HER2 were less than HER2-positive breast cancer cell lines. Based on their MFIs, GD2, HER2, B7H3, CSPG4, L1CAM (CD171), and Lewis Y were chosen as tumor targets for further in vitro screening.

### Example 13: GD2-BsAb and HER2-BsAb Exerted Strong Cytotoxicity Against Osteosarcoma Cell Lines In Vitro

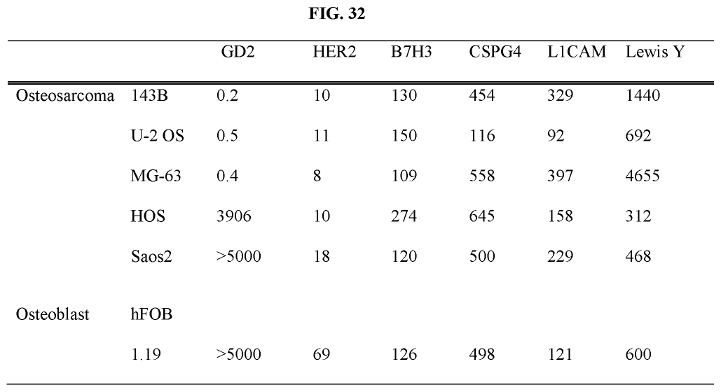

Osteosarcoma cell lines were used as targets in an ADTC assay using activated T cells (ATCs) (effector to target (E:T) ratio of 10:1) in the presence of decreasing concentrations of BsAbs (1 μg/mL (5 nM) and serial 10-fold dilutions). All tested BsAbs were made using the IgG(L)-scFv format with silenced Fc, and anti-GPA/anti-CD3 BsAb was used for control BsAb (Wu Z et al. Mol Cancer Ther 2018; 17(10):2164-75). Among them, anti-GD2 and anti-HER2-BsAb showed the most potent ADTC against the panel of osteosarcoma cell lines (FIG. 32 and FIG. 23B). For GD2-targeted BsAb (GD2-BsAb), cytotoxicity was robust (EC50 of 0.2 to 0.5 pM) for GD2(+) osteosarcoma cell lines (143B, U-2 OS, and M-63), where maximal killing was observed between 5 pM and 500 pM. In contrast, cytotoxicity for cell lines with low expression of GD2 (Saos2, HOS, and hFOB [fetal osteoblast cell line]) was much weaker. Anti-HER2-BsAb (HER2-BsAb) also mediated potent ADTC against most of the osteosarcoma cell lines which were HER2 positive (143B, U-2 OS, MG-63, HOS, and Saos2) and against hFOB, with maximal cytotoxicity at 5 pM to 500 pM. EC50 (a measure of in vitro sensitivity to ADTC) was inversely correlated with MFIs of each target antigen. Although B7H3, L1CAM, CSPG-4, and Lewis Y were also overexpressed in some osteosarcoma cell lines, their respective ADTC potency was much weaker. Based on these findings, the targets GD2 and HER2 were chosen for further in-depth T cell-based immunotherapy studies.

### Example 14: GD2-BsAb and HER2-BsAb Showed Potent Cytotoxicity Against Osteosarcoma In Vivo

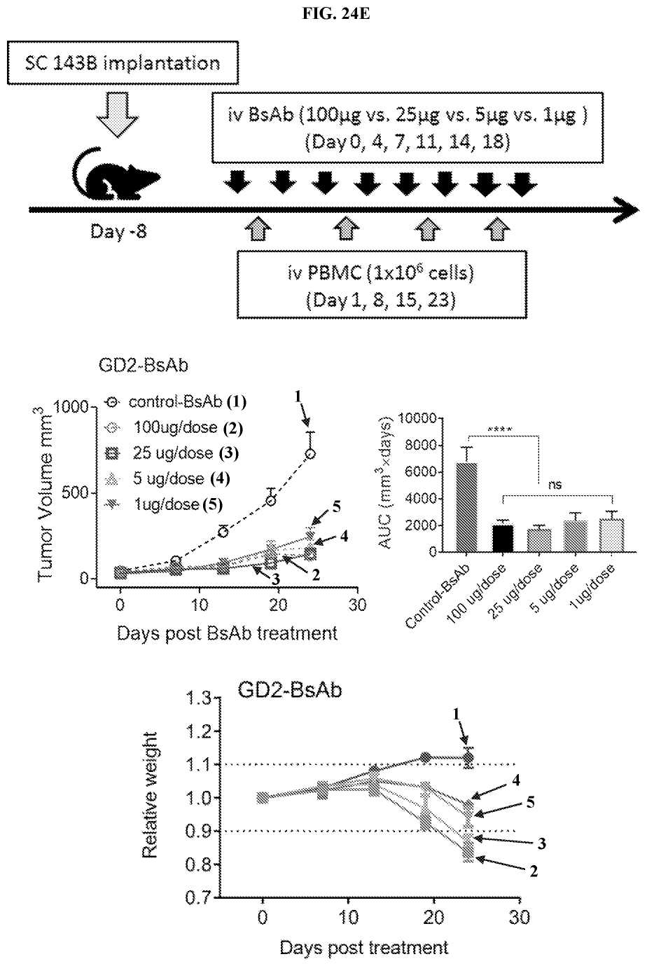

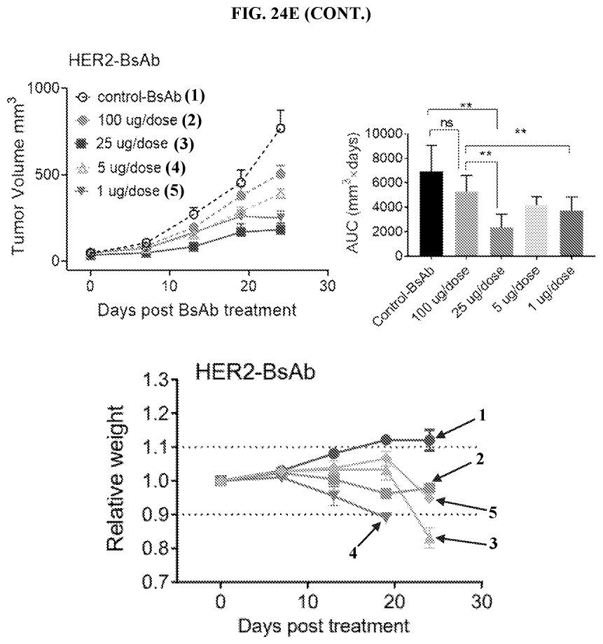

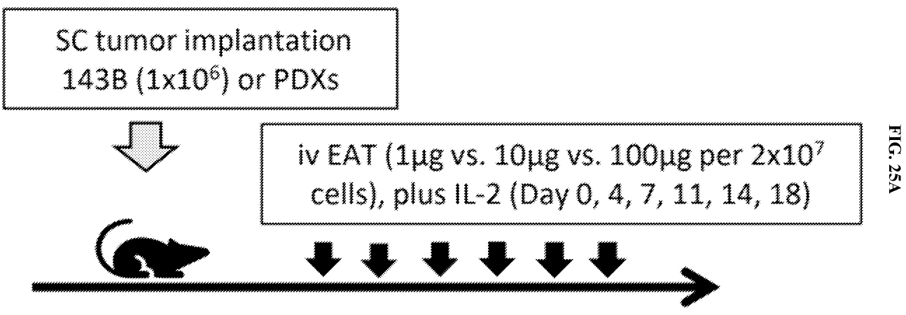

GD2-BsAb or HER2-BsAb suppressed osteosarcoma tumor growth in the presence of human T cells. Building on these in vitro ADTC assays, the in vivo anti-tumor effects of GD2-BsAb and HER2-BsAb against osteosarcoma xenografts was tested (FIG. 24A). In the first xenograft model, osteosarcoma 143B tumor cells were mixed with PBMCs and implanted subcutaneously (sc) into DKO mice. Mice were treated with intravenous (iv) GD2- or HER2-BsAb twice per week for 4 weeks. Osteosarcoma tumor growth was delayed by GD2-BsAb (P=0.0005) and HER2-BsAb (P=0.10) compared to control BsAb (anti-GPA/anti-CD3 BsAb). This finding was reproduced in a second tumor model where PBMCs were administered iv instead of s.c. (FIG. 24B). Both BsAbs significantly suppressed tumor growth compared to controls (P=0.0025 and P=0.0248, respectively).

Both GD2-BsAb and HER2-BsAb drove T cell into osteosarcoma xenografts. To test if GD2-BsAb and HER2-BsAb could drive exogenous T cells into osteosarcomas, T cell infiltration was investigated in tumors using IHC staining. CD3(+) TILs were detected in both GD2-BsAb- and HER2-BsAb-treated tumors, but not in tumors treated with control BsAb (FIG. 24C). Serial T cell infiltration was investigated by staining tumors on days 6, 9, 16, 23 and 30 post treatment. After GD2-BsAb or HER2-BsAb treatment (FIG. 24B), TILs substantially increased by day 9. While TILs showed CD4(+) T cell dominance on day 9, CD8(+) T cells became predominant at later time points (day 23 and day 30) (FIG. 24D).

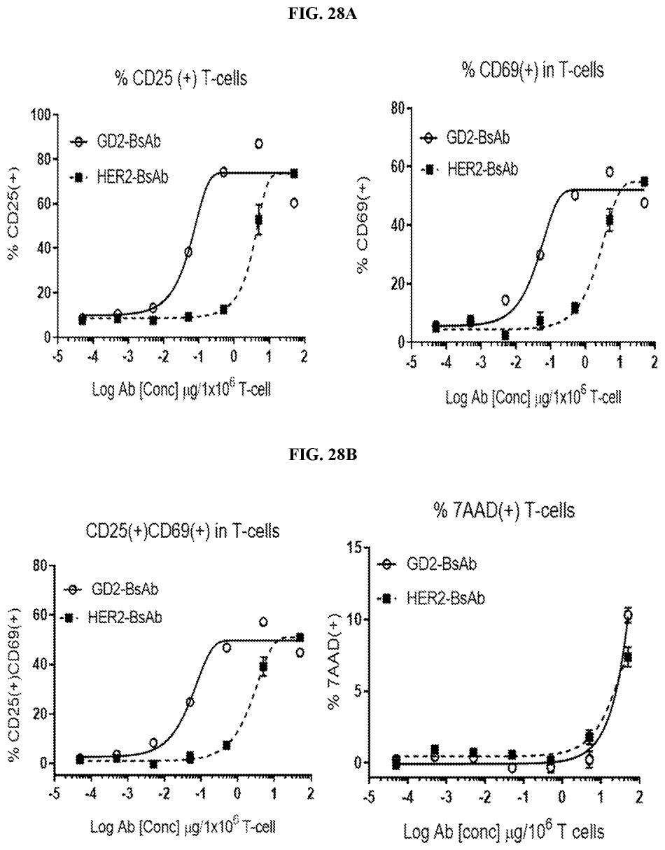

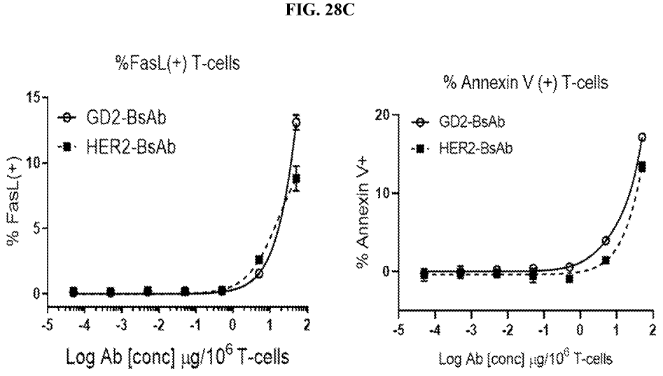

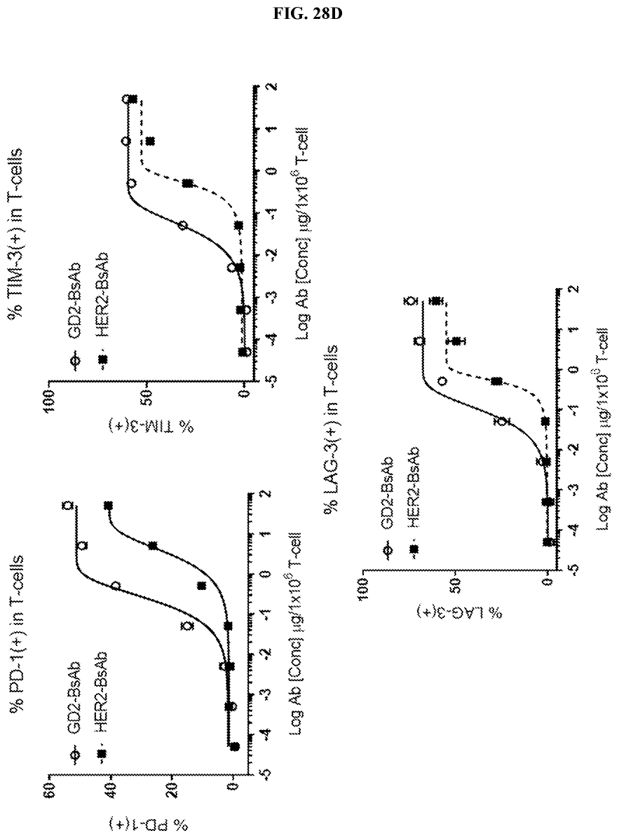

High-dose BsAbs diminished anti-tumor activity of T cells. To study the dose-response relationship on T cell activity, CD3(+) T cells were incubated at 37° C. for 24 hours in the presence of increasing concentrations of GD2-BsAb or HER2-BsAb [5×10⁻⁵ μg/1×10⁶ cells to 50 μg/1×10⁶ cells] and analyzed for cell death (annexin V and 7-aminoactinomycin D, 7-AAD), Fas-ligand (FasL), activation markers (CD25 and CD69), and exhaustion markers (PD-1, TIM-3, and LAG-3) (FIGS. 28A-28D). T cells incubated in high concentrations of GD2-BsAb or HER2-BsAb showed increased frequencies of CD25(+), CD69(+), and CD25(+) and CD69(+) double positive populations compared to control T cells incubated without T-BsAbs. CD25 and CD69 expression surged when the concentration of T-BsAb was above 0.005 μg/1×10⁶ cells for GD2-BsAb and 0.5 μg/1×10⁶ cells for HER2-BsAb. On the other hand, the frequencies of 7AAD(+) and FasL(+) populations started to increase when both T-BsAb reached 0.5 μg/1×10⁶ cells. Among exhaustion markers, PD-1 expression on CD3(+) T cells rapidly increased with high concentrations of GD2-BsAb or HER2-BsAb. TIM-3 and LAG-3 also rose with increased BsAb concentrations. T cells exposed to high concentrations of BsAb expressed more PD-1, TIM-3, and LAG-3 than those exposed to lower concentrations of BsAb. These in vitro observations were validated in osteosarcoma xenografts treated with iv PBMCs and decreasing doses of BsAbs (FIG. 24E). Anti-tumor effect of 100 μg of HER2-BsAb was inferior to those of 25 μg (P=0.0054). On the other hand, there was no significant difference over a wide dose range for GD2-BsAb (1 to 100 μg), although 25 μg seemed optimal.

### Example 15: Adoptive T Cell Therapy Using Ex Vivo Armed T Cells (EATs) Carrying GD2-BsAb or HER2-BsAb Effectively Suppressed Osteosarcoma Tumor Growth and Prolonged Survival

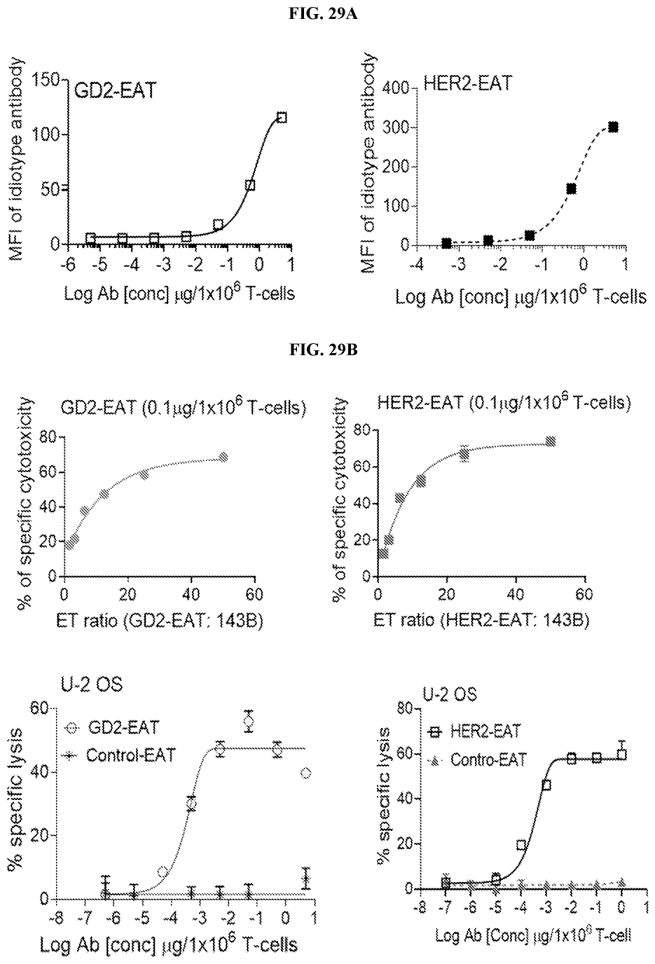

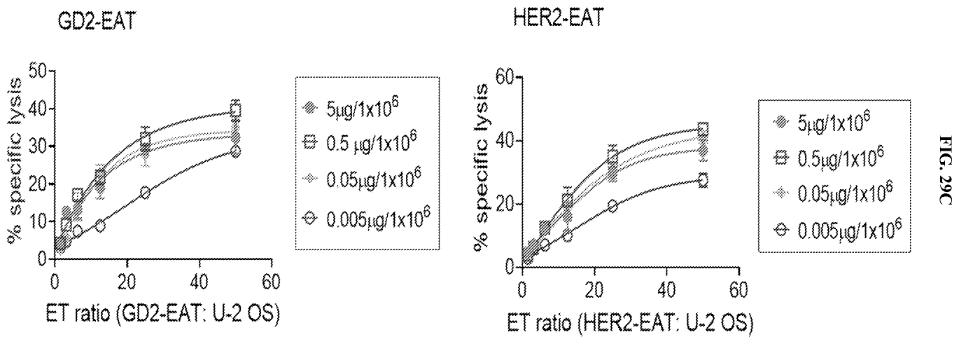

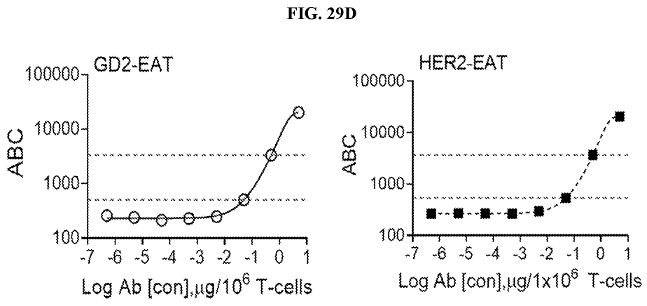

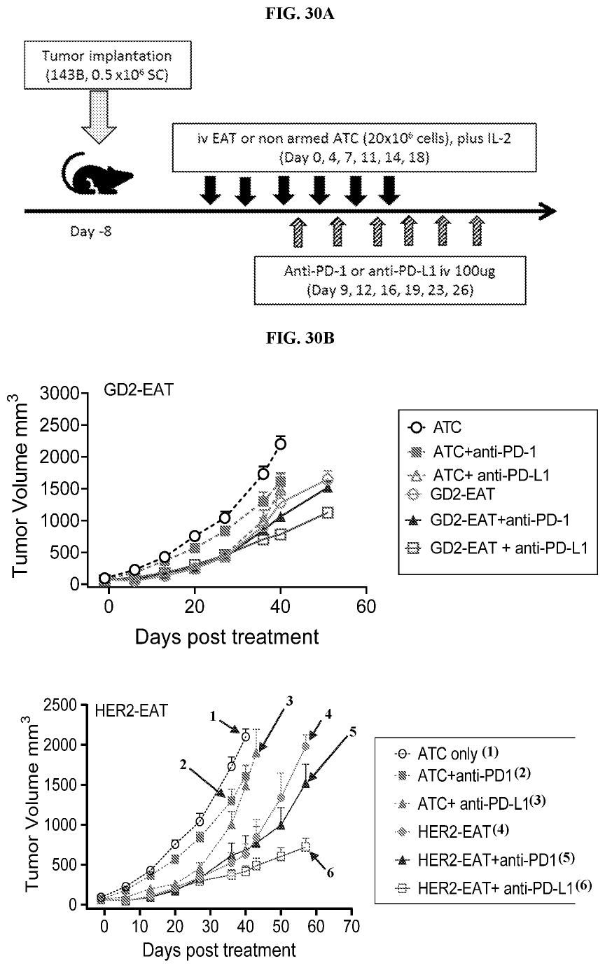

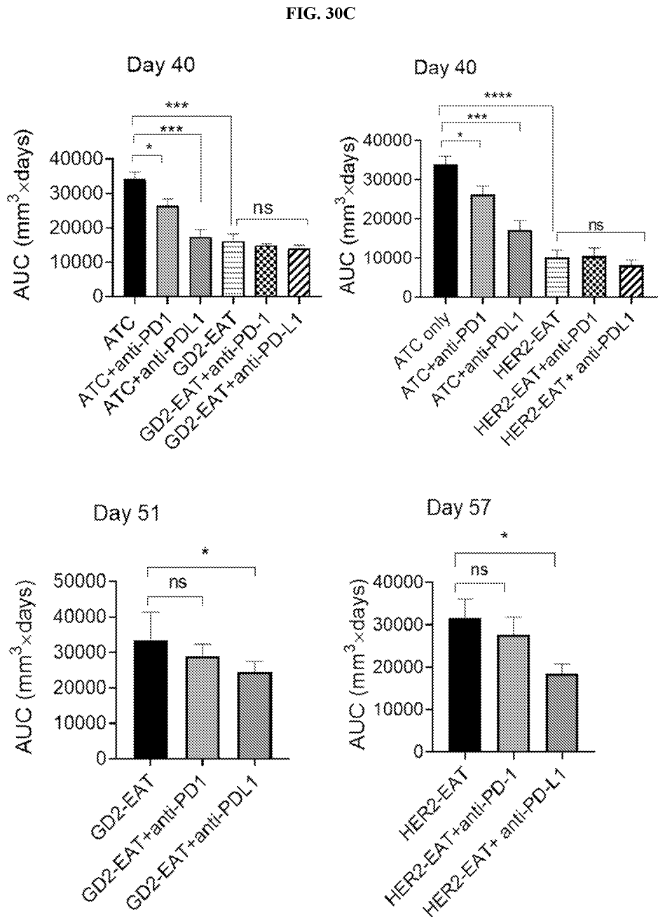

EATs showed stable BsAb arming and potent cytotoxicity. Ex vivo activated T cells were armed with GD2-BsAb or HER2-BsAb and tested for cell surface density of each BsAb using anti-idiotype or anti-human IgG Fc antibodies, and their cytotoxicity was evaluated in a 4-hour ⁵¹Cr release assay. GD2-BsAb armed T cells (GD2-EATs) and HER2-BsAb armed T cells (HER2-EATs) showed stable binding to idiotype antibody (FIG. 29A). When in vitro cytotoxicity was tested, GD2-EATs and HER2-EATs both displayed strong antigen-specific cytotoxicity against osteosarcoma cell lines over a range of E:T ratios and over a range of antibody doses (FIG. 29B). Maximum killing was observed between 0.05 μg to 5 μg/10⁶ cells of BsAb arming concentration. To quantify the density of BsAb bound to T cells after arming, ABC was measured by flow cytometry and referenced to commercial quantum beads (FIGS. 29C-29D). Optimal arming per T cell required 600 to 20,350 molecules for GD2-BsAb or HER2-BsAb, corresponding to 0.05 μg/10⁶ cells to 5 μg/10⁶ cells of BsAb; the molar amount of BsAb bound per T cell ranged from 1 to 35 zeptomoles (1×10⁻²¹) for GD2-BsAb or HER2-BsAb.

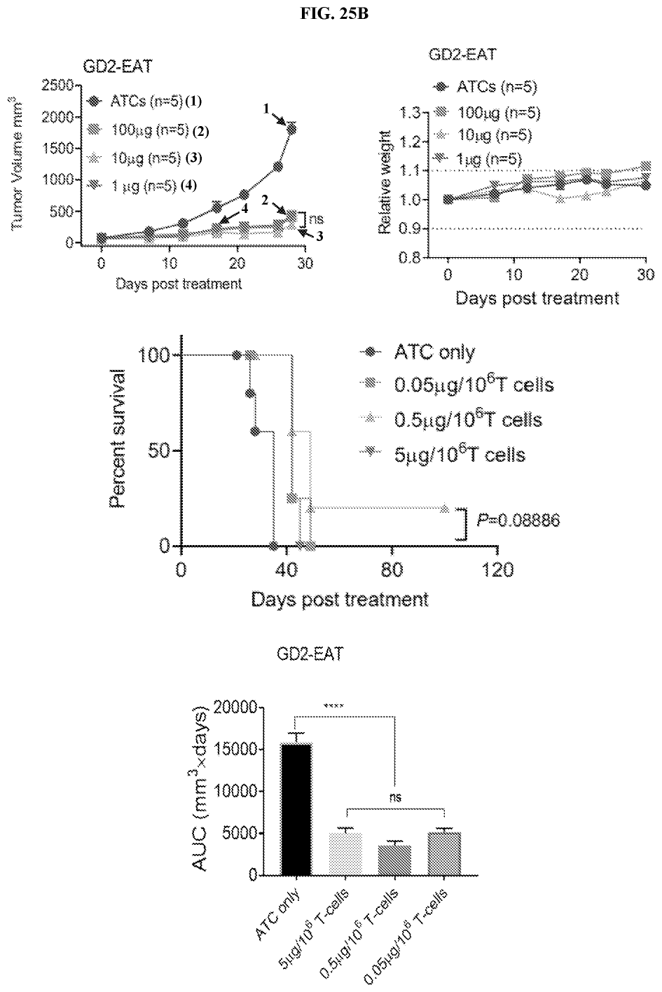

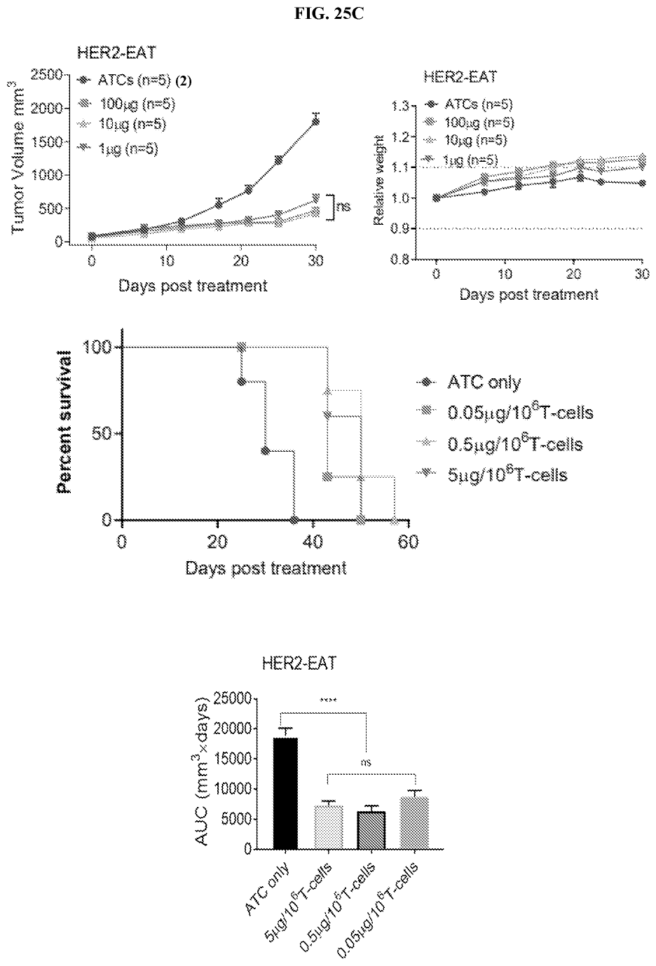

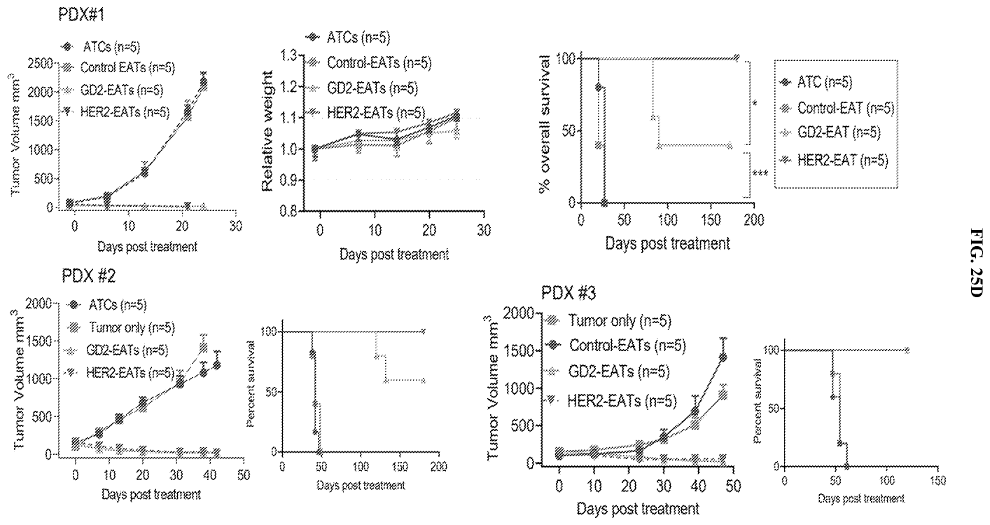

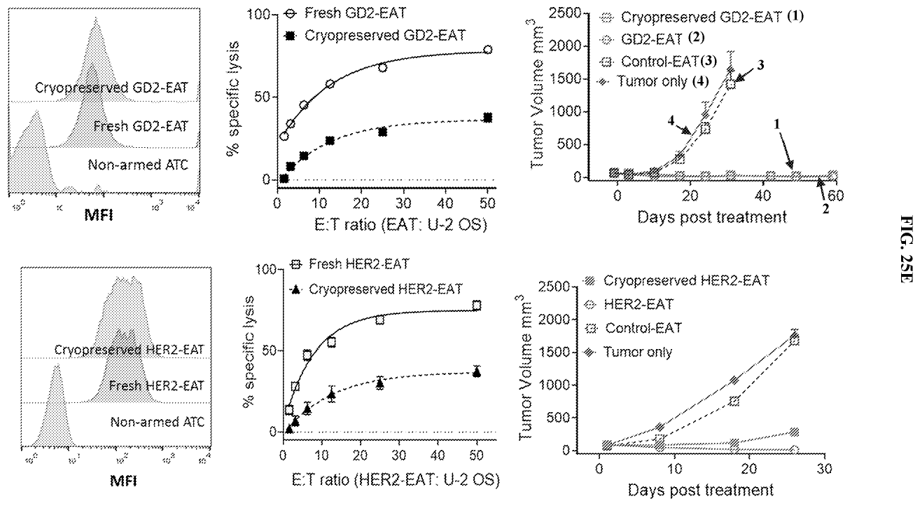

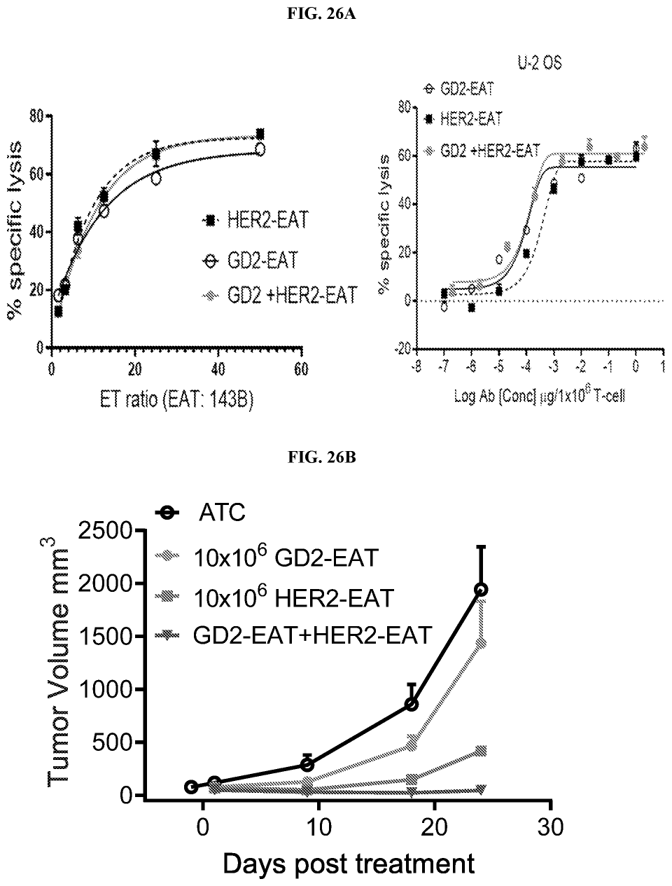

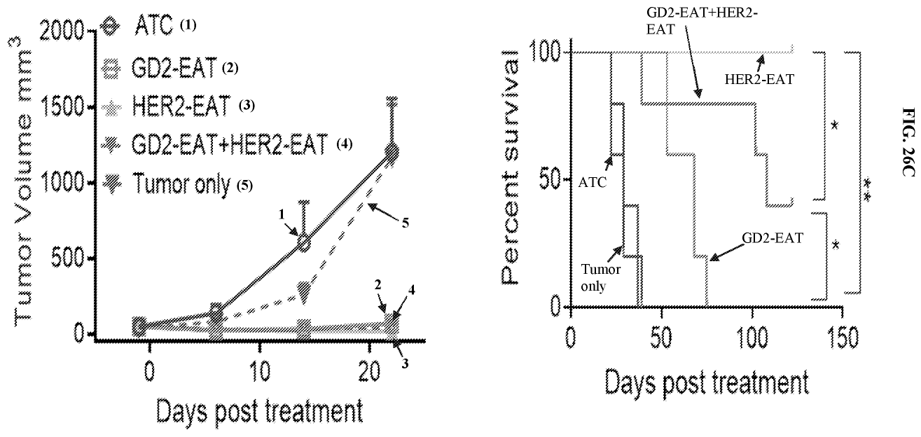

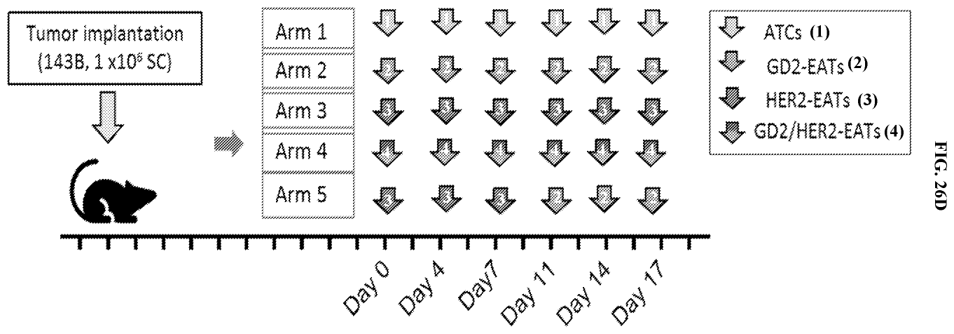

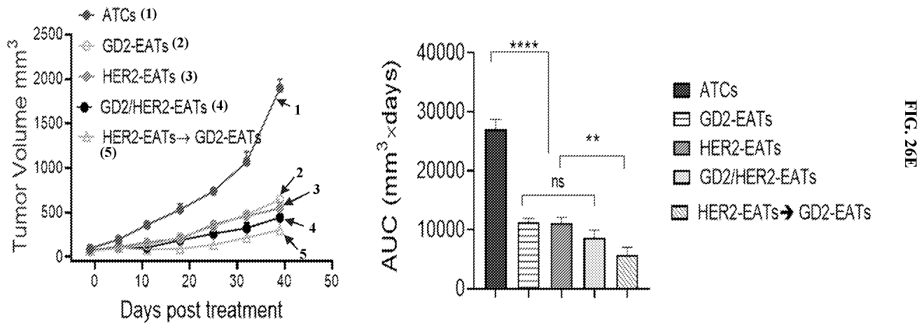

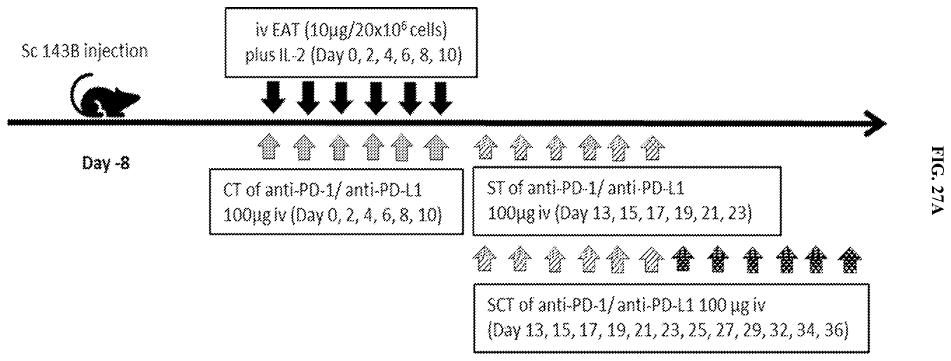

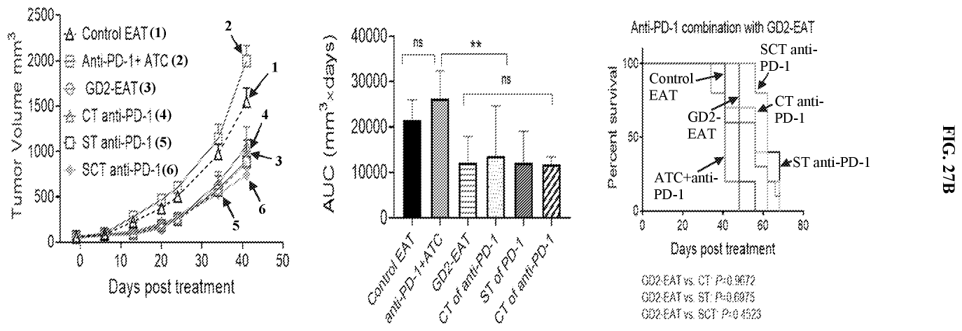

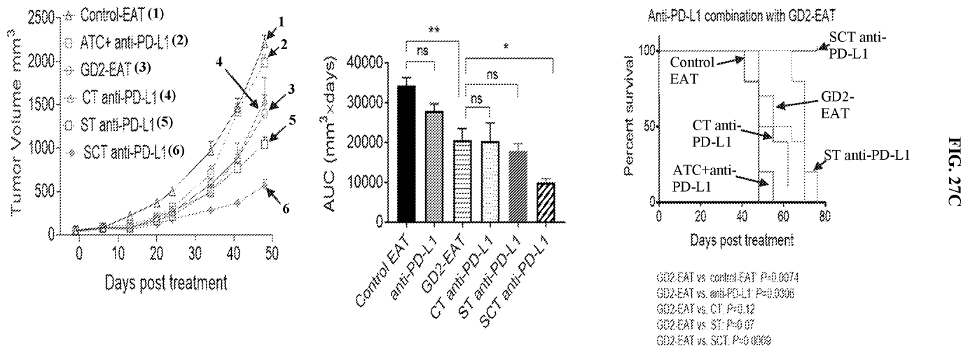

EATs exerted potent anti-tumor effects in vivo. To address the anti-tumor properties of EATs in vivo, their efficacy in multiple osteosarcoma PDX models was tested (FIG. 25A). First, 143B cell line xenografts were treated with 20×10⁶ of T cells armed with different concentrations (0.05 to 5 μg/10⁶ cells) of GD2-BsAb or HER2-BsAb (FIGS. 25B-25C). Most mice maintained their body weights throughout treatment and did not exhibit any significant clinical toxicities, contrasting to the separately administered BsAb and PBMC treatment (FIG. 24E). Tumor growth was suppressed over a range of BsAb doses (0.05 μg/10⁶ cells to 5 μg/10⁶ cells) compared to the unarmed control group (ATCs only), (P<0.0001). Of note, the immunosuppressive effect of high-dose BsAb, particularly for HER2-EAT, was effaced by arming. Both EATs exerted significant tumor suppressing effects over a range of BsAb concentrations, and there was no significant difference among three different concentrations tested.

Similar anti-tumor effects were observed when osteosarcoma PDX tumors were used (FIG. 25D). PDX tumors treated with 6 doses of GD2-EATs or HER2-EATs showed complete ablation, translating into significant improvements in survival compared to control (anti-CEA/anti-CD3) BsAb armed T cells (P<0.0001). Again, there were no clinical toxicities during treatment. While all mice in the control group had to be euthanized due to tumor burden within 30 days of posttreatment, GD2-EATs and HER2-EATs regressed tumors and displayed long-term remission (P<0.0001). 2 of 5 that received GD2-EATs and 5 of 5 that received HER2-EATs maintained remission past 180 days of observation. This strong in vivo anti-tumor activities of GD2-EATs and HER2-EATs were reproduced in another 2 different osteosarcoma PDX models.

To test anti-tumor properties of GD2-EATs and HER2-EATs after freezing and thawing, GD2-BsAb or HER2-BsAb armed T cells were cryopreserved in liquid nitrogen (−196° C.). After 4 to 6 weeks, these EATs were thawed and tested their anti-tumor activities (FIG. 25E). The cell viability after thawing was >85% and their MFI values of individual EATs were comparable to those of freshly armed EATs. When cytotoxicity was evaluated right after thawing, fresh unfrozen EATs had superior killing compared to cryopreserved EATs, being partly attributed to no recovery time for cryopreserved EATs. Yet, in vivo, thawed EATs exerted comparable anti-tumor activity to fresh unfrozen EATs against osteosarcoma PDXs.

### Example 16: Anti-PD-L1 Antibody Augmented Anti-Tumor Immune Response of GD2-EATs and HER2-EATs Against Osteosarcoma

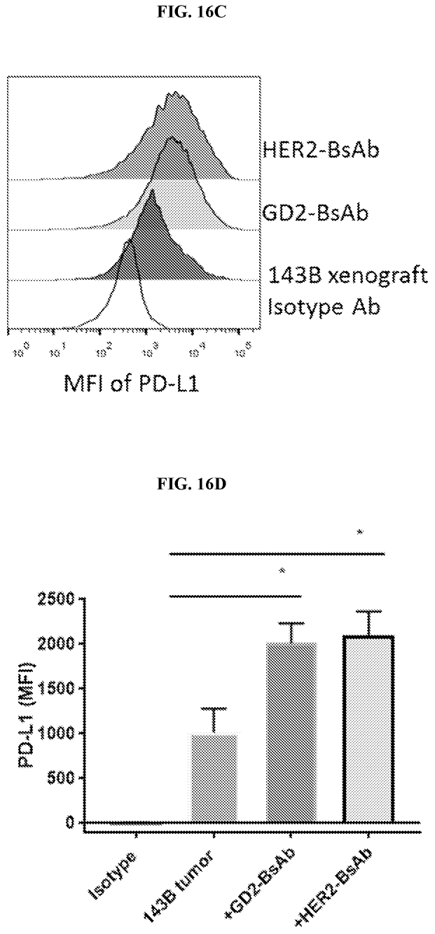

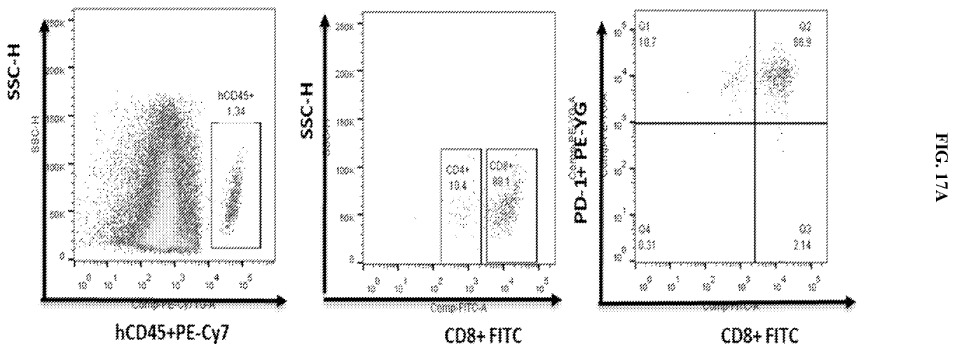

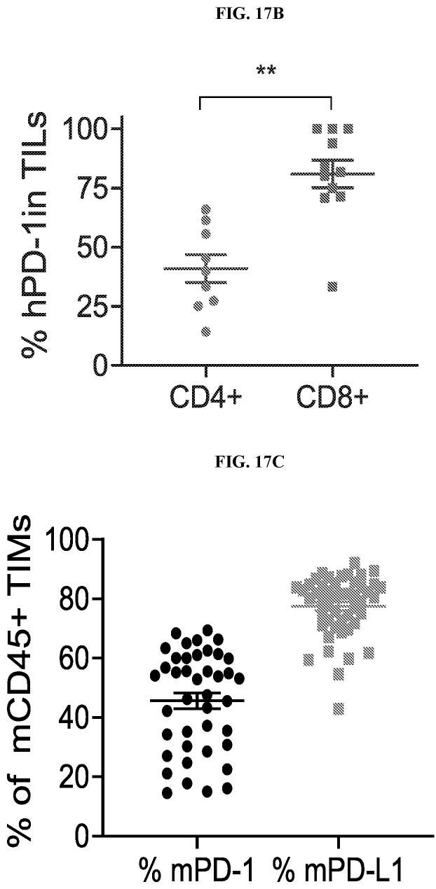

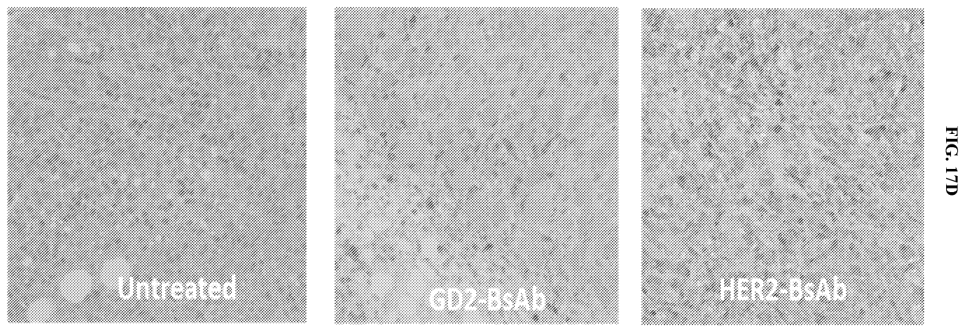

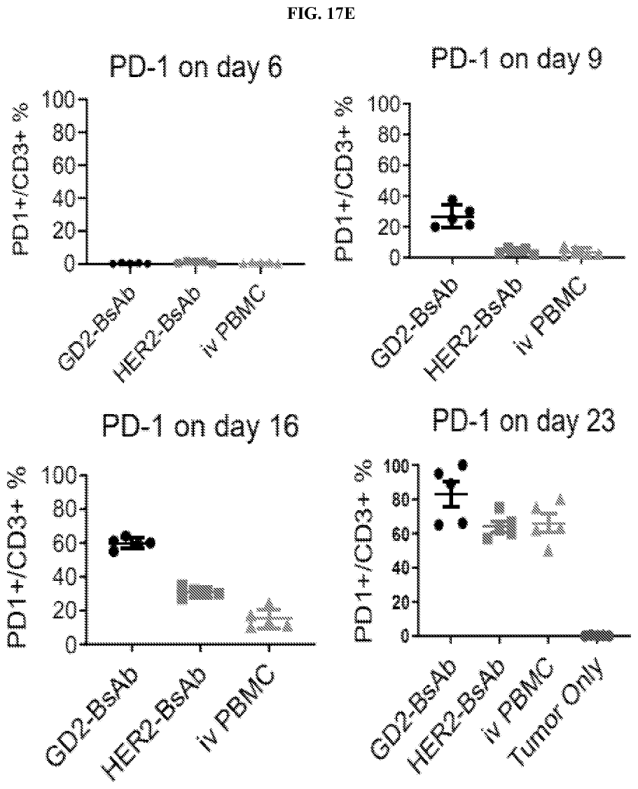

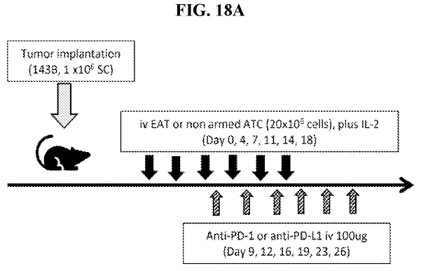

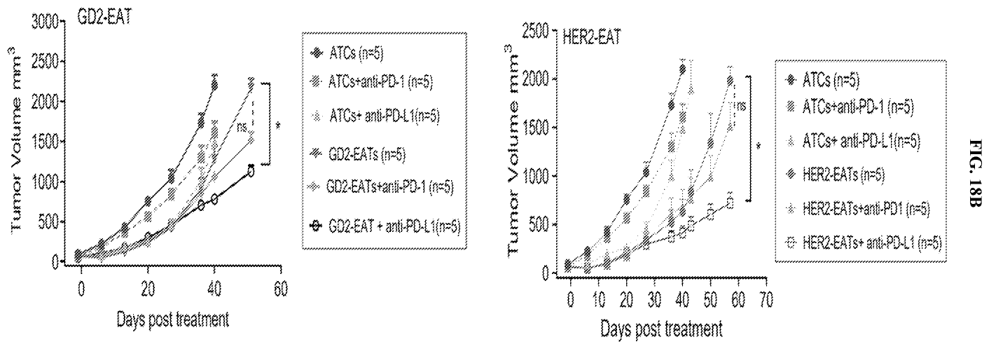

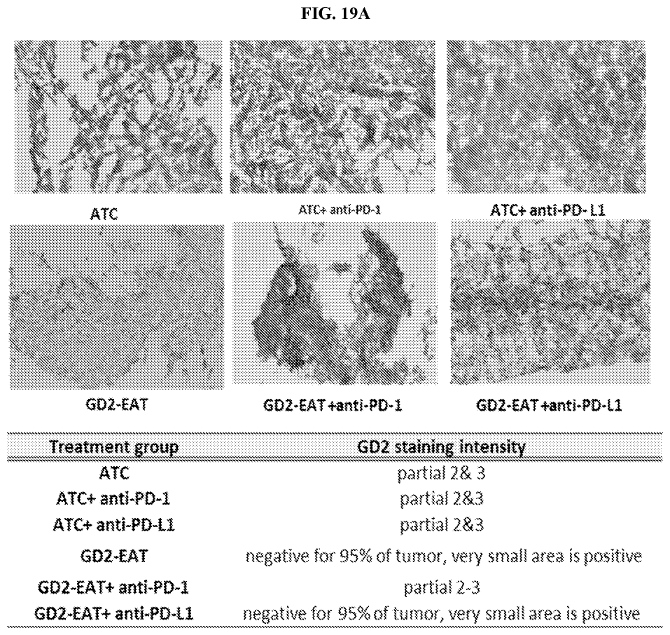

Although GD2-BsAb and HER2-BsAb recruited substantial numbers of T cells into the tumor and successfully suppressed tumor growth compared to control groups, some tumors were resistant or relapsed following the initial response. In these tumors, TILs showed predominance of CD8(+) T cells, the majority of which expressed PD-1 on their surface (FIG. 17A-17C). Circulating CD3(+) T cells in peripheral blood on day 6, 9, 16, and day 23 post treatment showed gradual increase of PD-1 expression from less than 5% to over 75% after treatment with GD2-BsAb (FIG. 17D). In addition to PD-1 expression on T cells, osteosarcoma xenografts were PD-L1 positive by IHC staining and FACS analyses and upregulated PD-L1 expression following BsAb treatment (FIGS. 16A-16D).

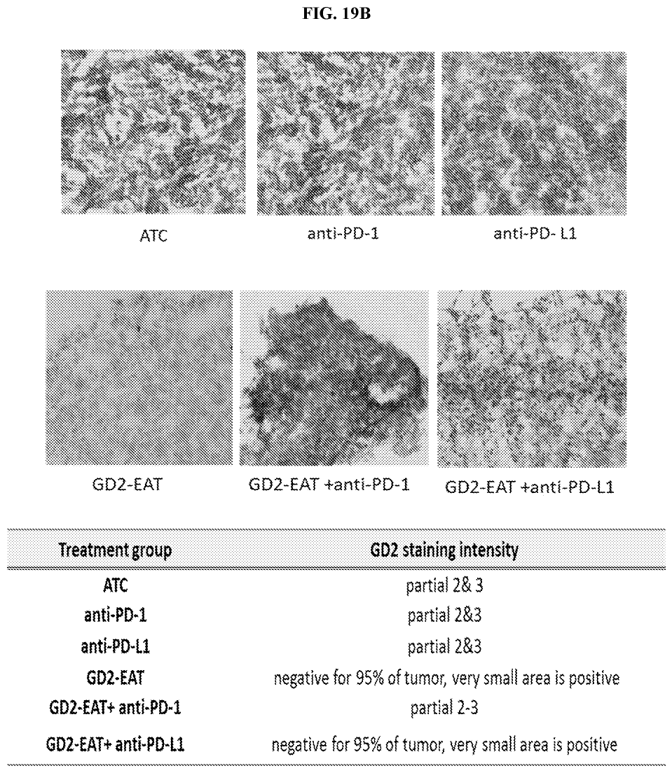

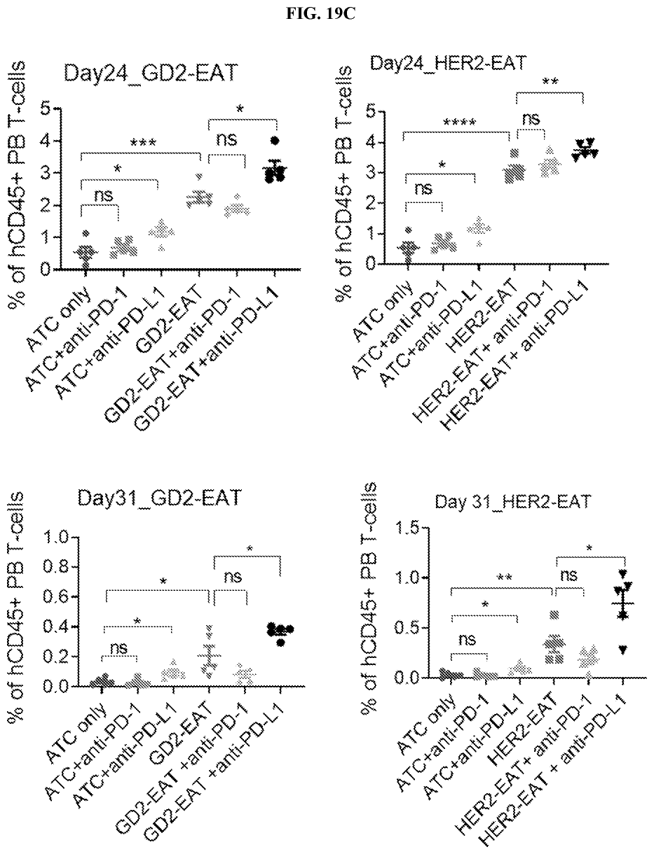

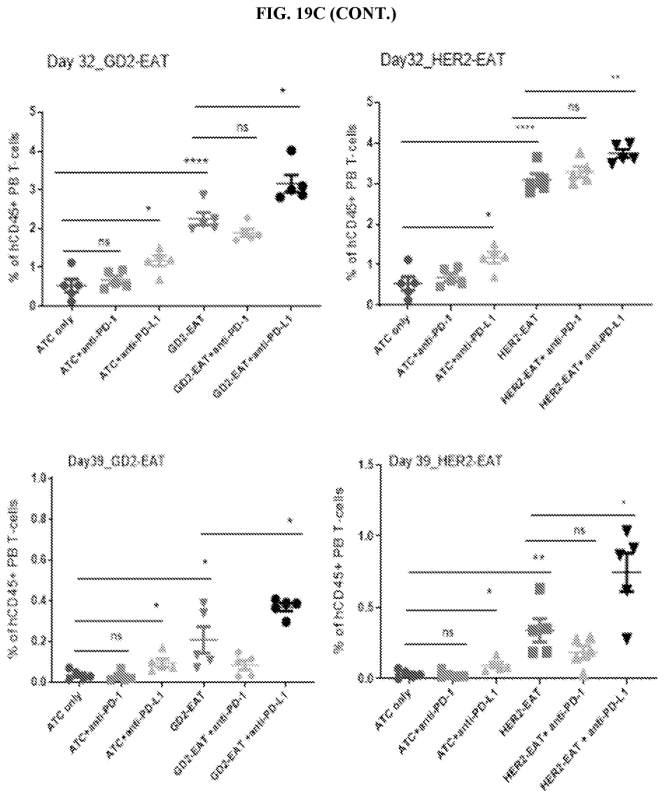

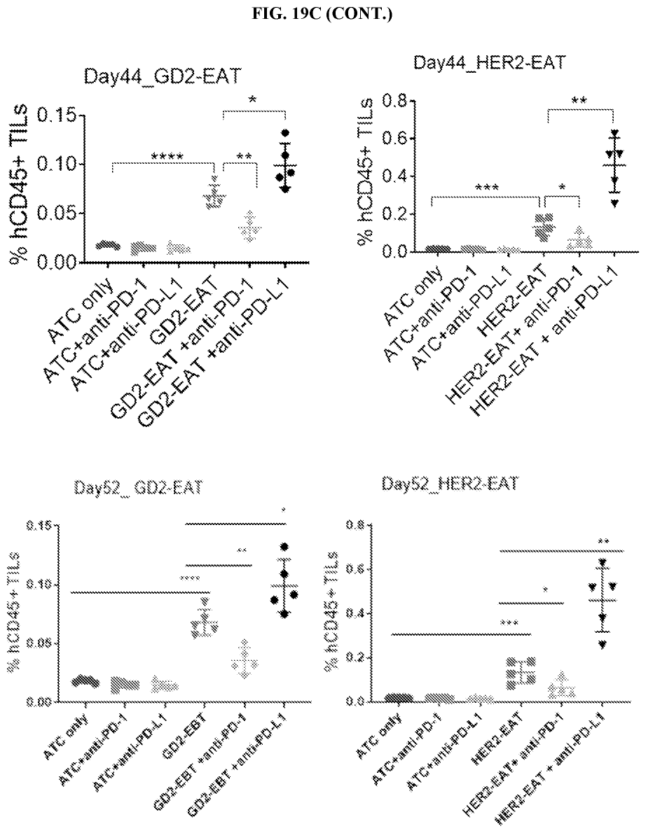

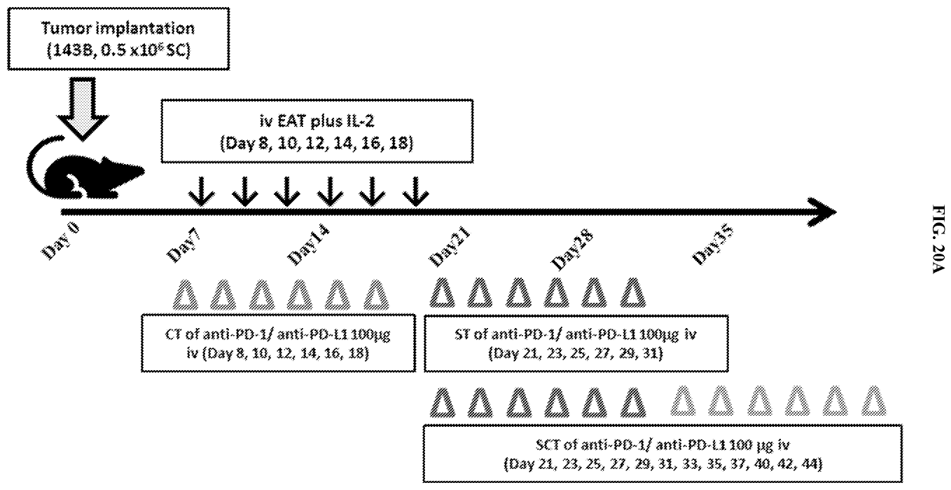

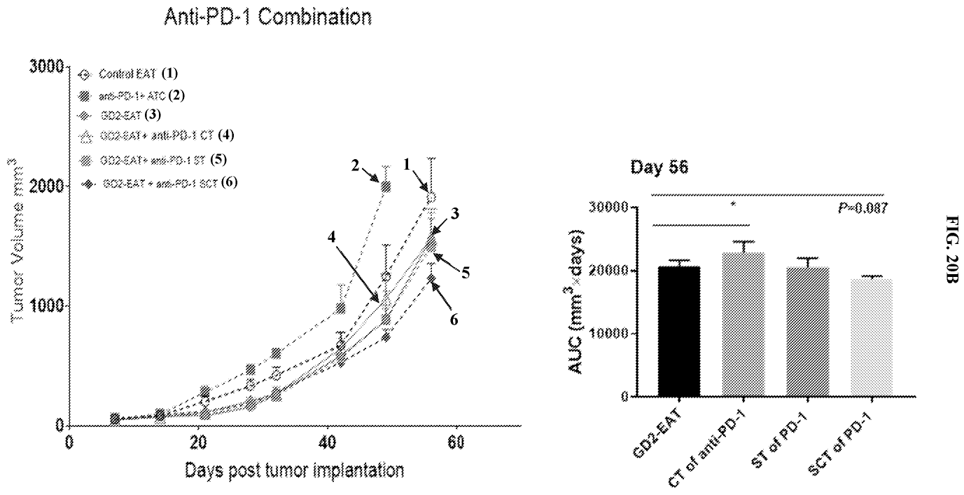

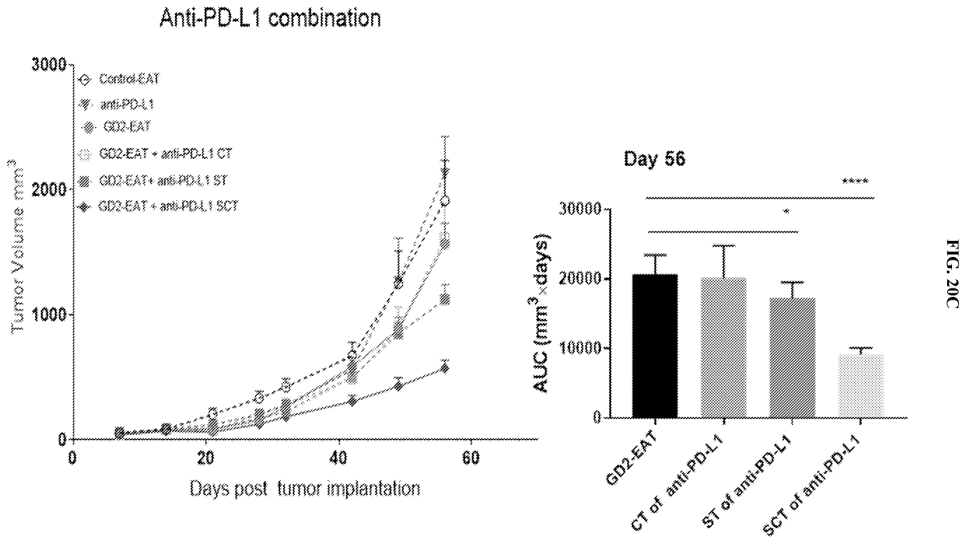

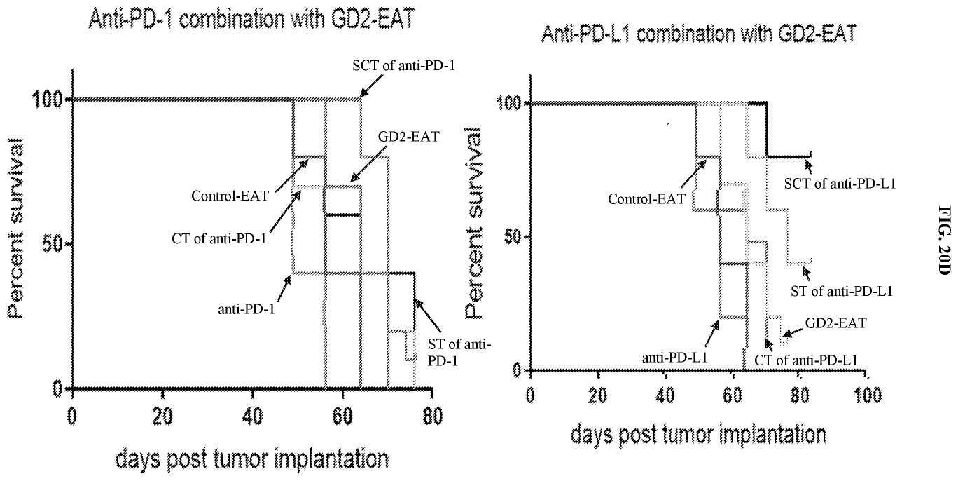

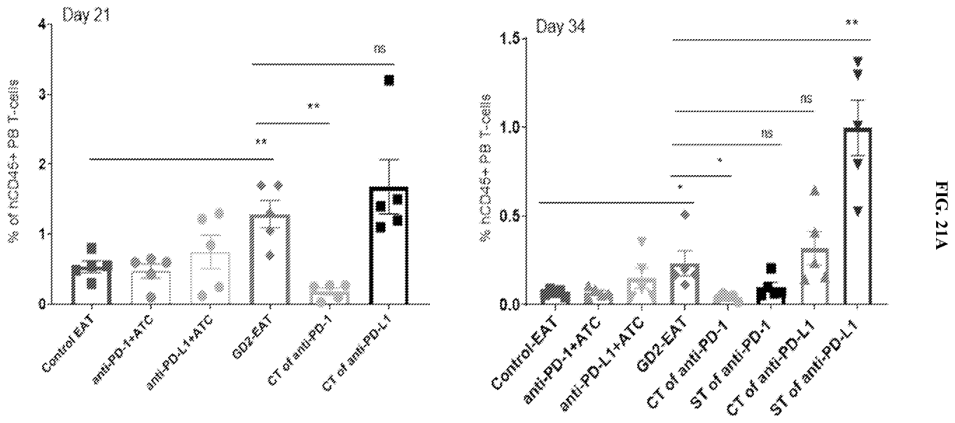

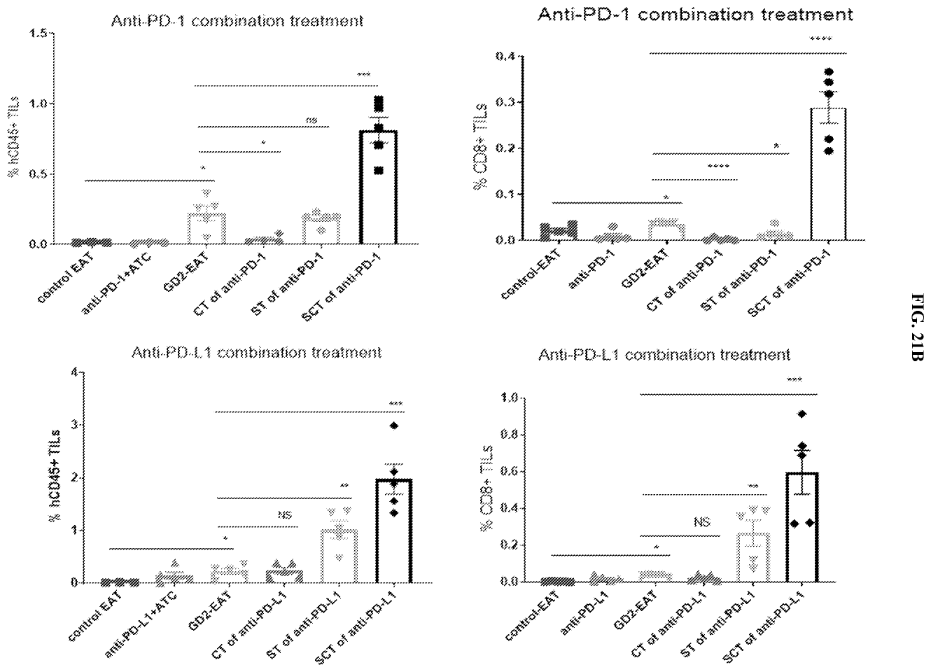

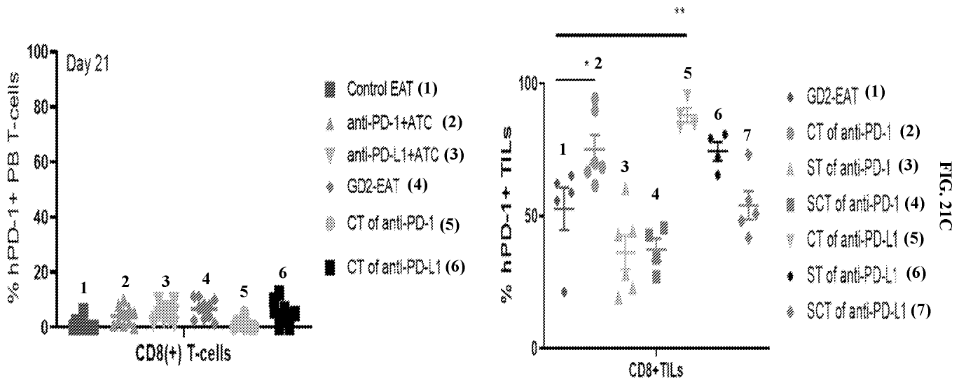

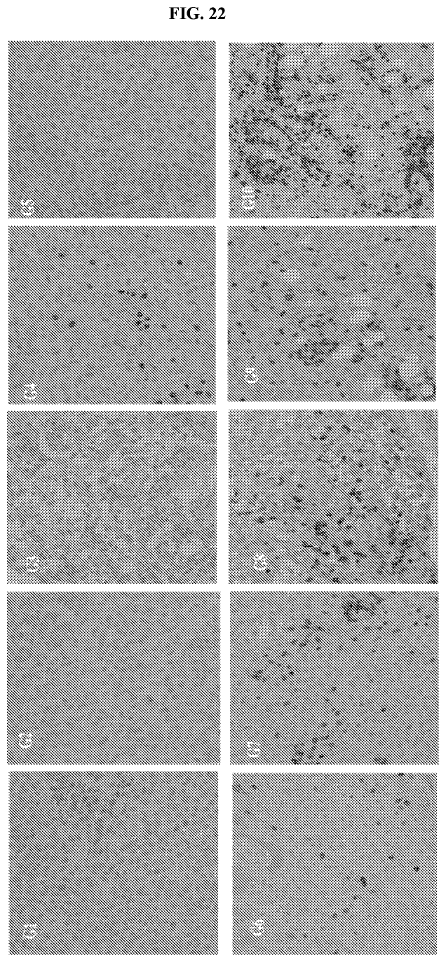

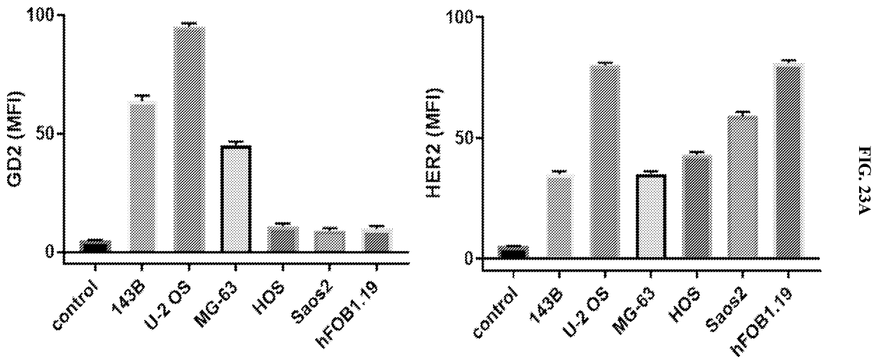

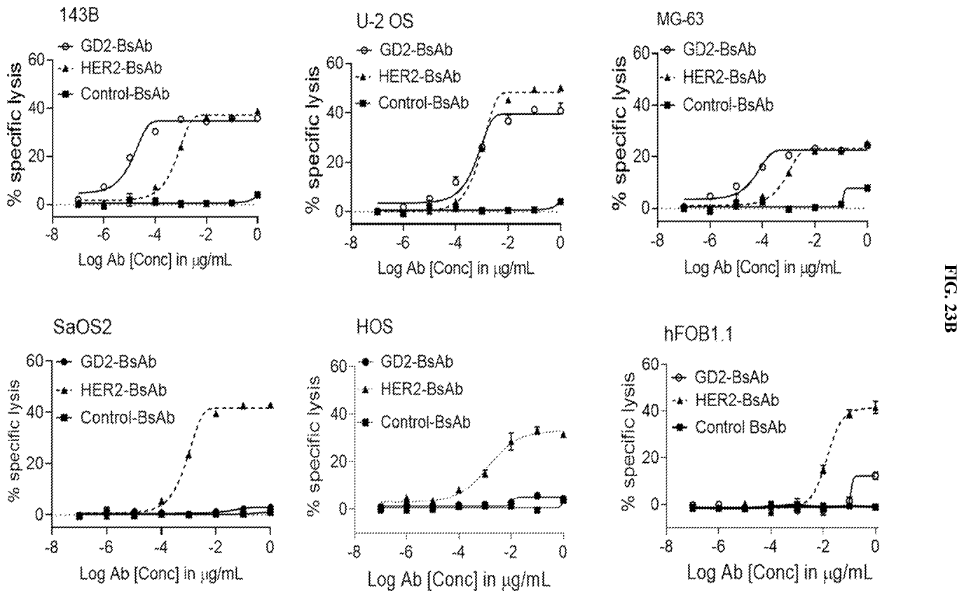

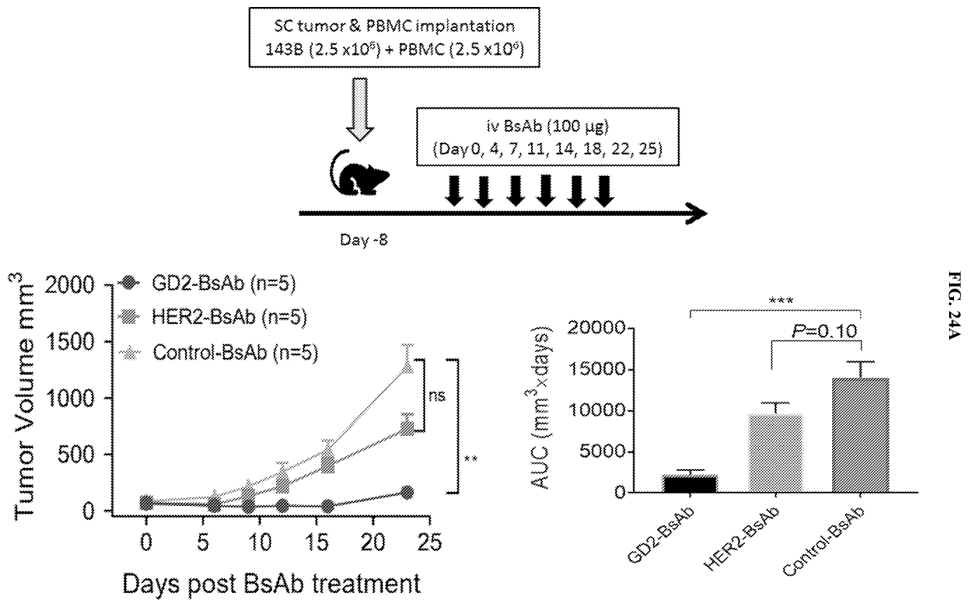

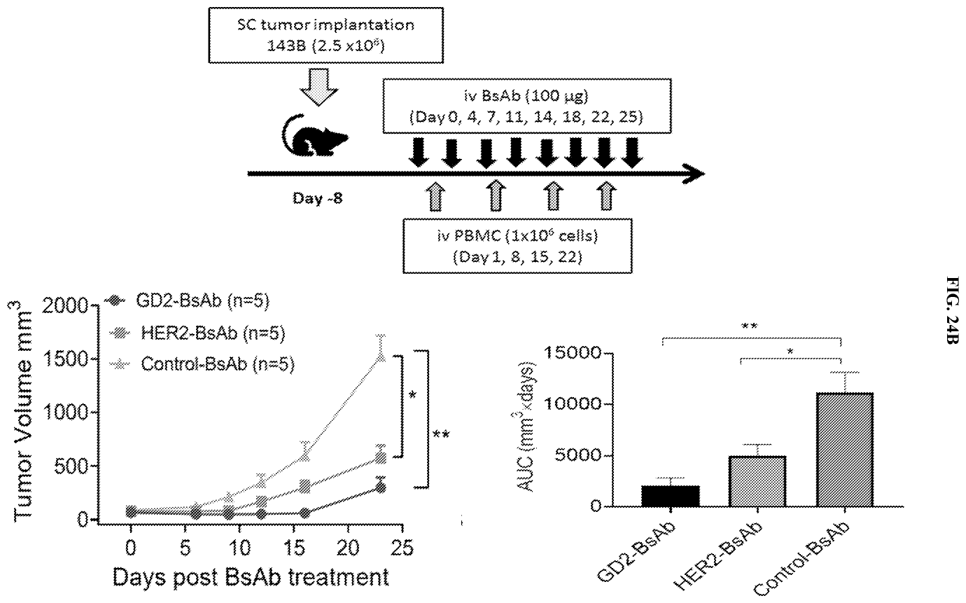

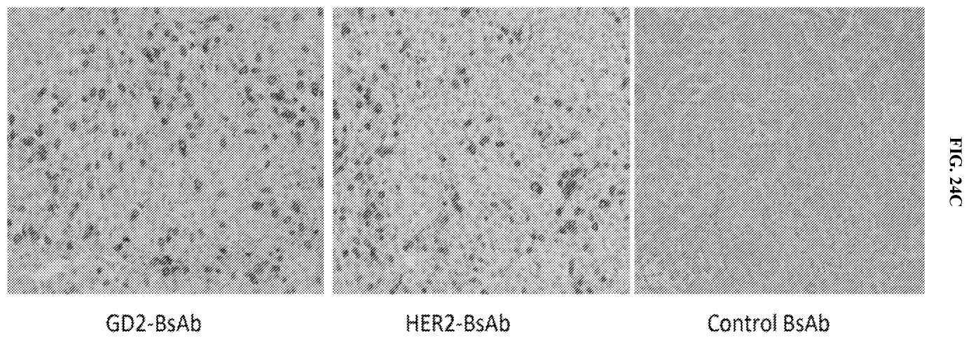

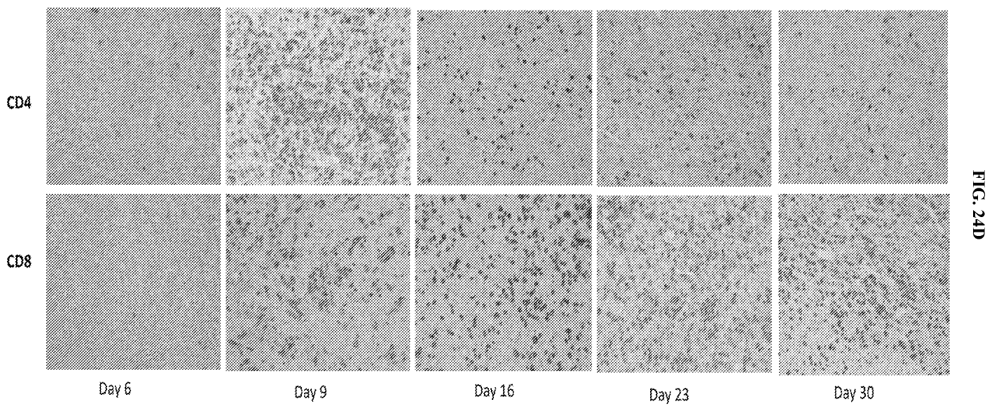

PD-L1 blockade augmented anti-tumor effect of EAT therapy. To test if ICIs can overcome T cell exhaustion related to treatment resistance, anti-PD-1 (pembrolizumab) or anti-PD-L1 (atezolizumab) monoclonal antibodies were combined with EATs to treat osteosarcoma xenografts (FIGS. 18A-18B). GD2-EATs or HER2-EATs were administered twice a week for 3 weeks, and iv anti-PD-1 or anti-PD-L1 was initiated on day 9 post EAT treatment and given twice per week for 3 weeks, based on the anticipated upregulation of PD-1 in T cells by day 9 (FIG. 17E). Anti-PD-L1 plus GD2-EATs or HER2-EATs combination showed benefit over GD2-EATs or HER2-EATs alone (P=0.0257, respectively), while combination with anti-PD-1 had no significant benefit. Anti-PD-L1 combination resulted in significantly greater frequencies of T cells in tumors compared to GD2-EAT or HER2-EAT monotherapy, whereas anti-PD-1 combination did not (FIG. 19C). Interestingly, GD2-EATs and GD2 EATs plus anti-PD-L1 combination appeared to eliminate GD2^{high} tumors while leaving GD2^{low} tumors behind (by IHC), but GD2-EATs plus anti-PD-1 combination did not show such effects (FIGS. 19A-19B).

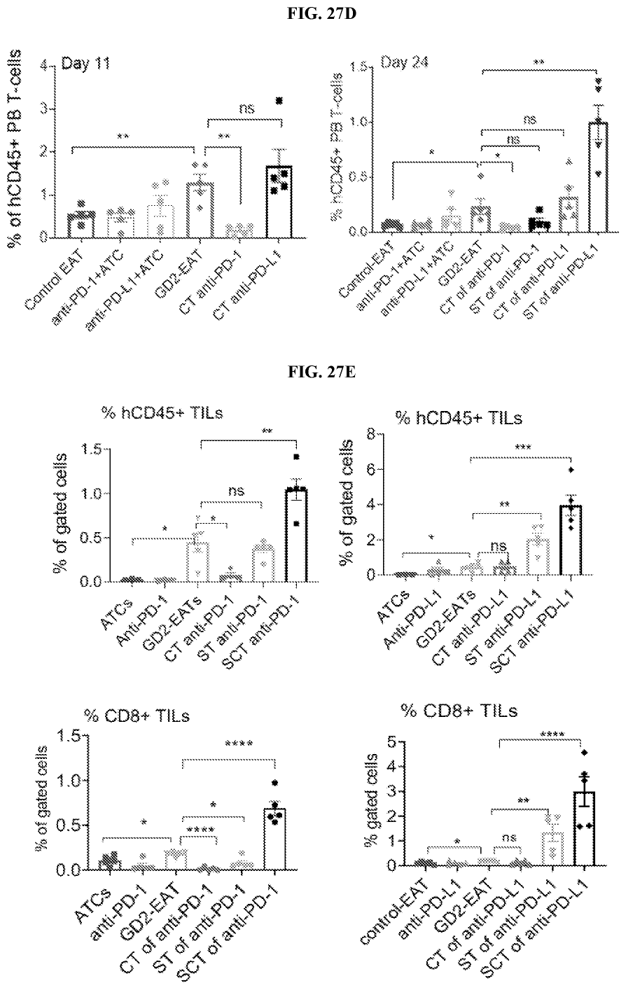

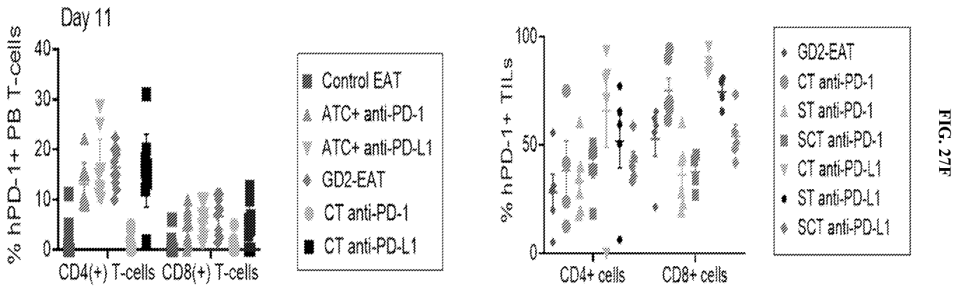

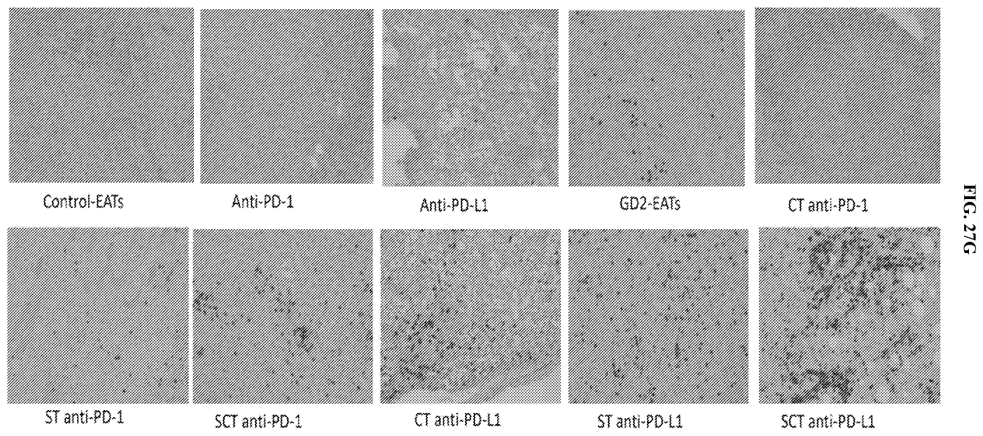

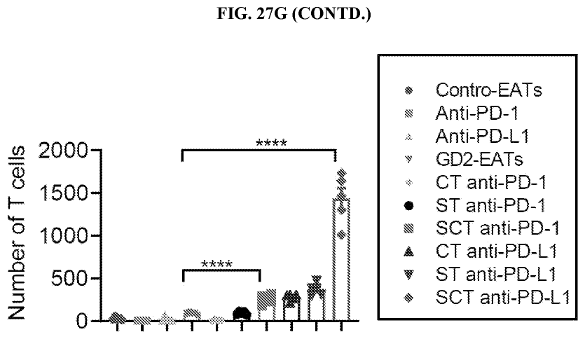

Timing of anti-PD-L1 during GD2 EATs therapy affected anti-tumor response in vivo. Given the upregulation of PD-1/PD-L1 pathway following EATs therapy, three different time schedules of PD-1 blockades were tested (FIGS. 27A-27C). GD2-EATs were given three times per week for 2 weeks. Six doses of anti-PD-1 or anti-PD-L1 were added either (1) concurrently (concurrent therapy, CT) or (2) sequentially after 6 doses of EATs (sequential therapy, ST), or (3) additional 6 doses of PD-1 blockades were administered post ST (sequential continuous therapy, SCT). Combination with anti-PD-1 had no effect, either using CT, ST or SCT regimens when compared to GD2-EATs alone. CT of anti-PD-L1 also failed to enhance efficacy of GD2-EATs. However, anti-PD-L1 given as ST slowed the tumor growth, and SCT significantly suppressed tumor growths compared to GD2-EATs alone (P=0.0149), which translated into improved survival. While none of the anti-PD-1 regimens improved survival over GD2-EATs, SCT of anti-PD-L1 significantly improved the survival for GD2-EATs (P=0.0009).

To address the effect of ICIs on T cell infiltration into tumors, tumors were harvested when they reached 2000 mm³ or on the last day of the experiment. TILs were analyzed by flow cytometry (FIG. 27E). The frequencies of TILs differed by treatment: GD2-EATs recruited more T cells into the tumors compared to control-EATs (P=0.0295) or anti-PD-1 plus ATCs group (P=0.0236). CT of anti-PD-1 resulted in significantly fewer TILs than GD2-EATs (P=0.0194). With ST regimen, anti-PD-1 showed comparable TIL frequency with GD2-EATs (P=0.54); with SCT regimen, anti-PD-1 increased TIL frequency over GD2-EATs alone (P=0.0056). On the other hand, CT of anti-PD-L1 did not affect TIL frequencies over GD2-EATs, but ST of anti-PD-L1 increased TIL frequencies over GD2-EATs alone (P=0.0018), and SCT regimen resulted in the highest TIL frequency among groups (P=0.0005). Among the TIL subsets, tumors treated with SCT regimen (irrespective of anti-PD-1 or anti-PD-L1) had significantly greater frequencies of CD8(+) T cells when compared to GD2-EATs alone (P<0.0001). The difference in TIL frequencies by treatment was confirmed by IHC staining using anti-CD3 antibody (FIG. 27G). Anti-PD-L1 combinations consistently had greater frequencies of TILs providing a rationale for combining EATs with anti-PD-L1 for synergy with BsAb-based T cell immunotherapy.

### Example 17: Dual Antigens Targeting Strategies Using EAT

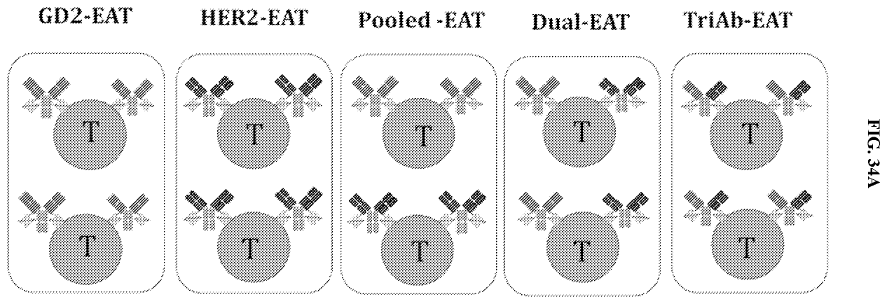

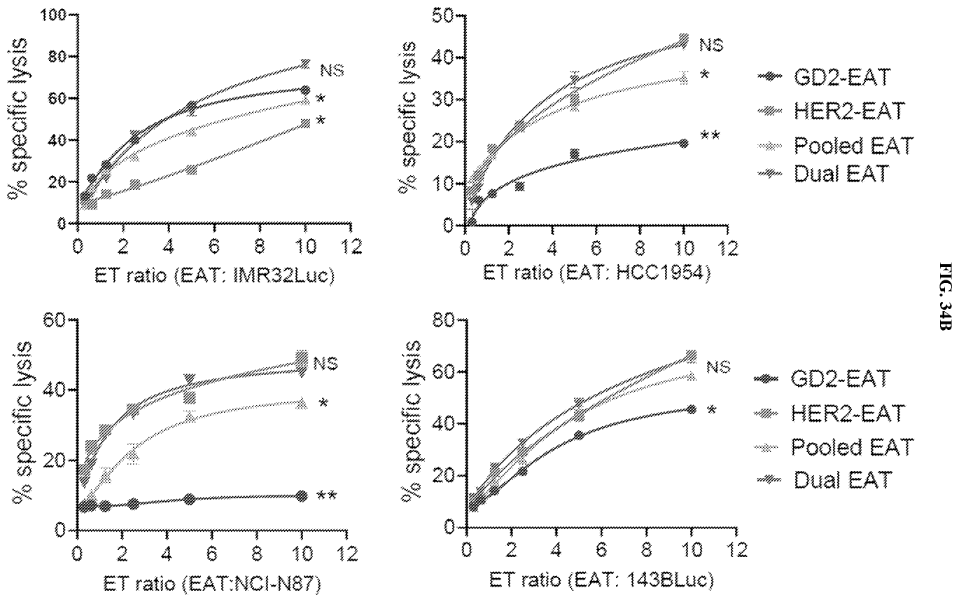

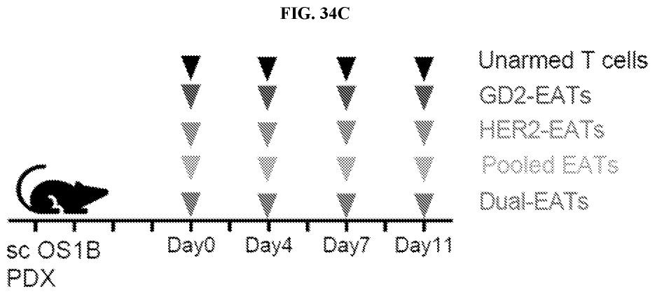

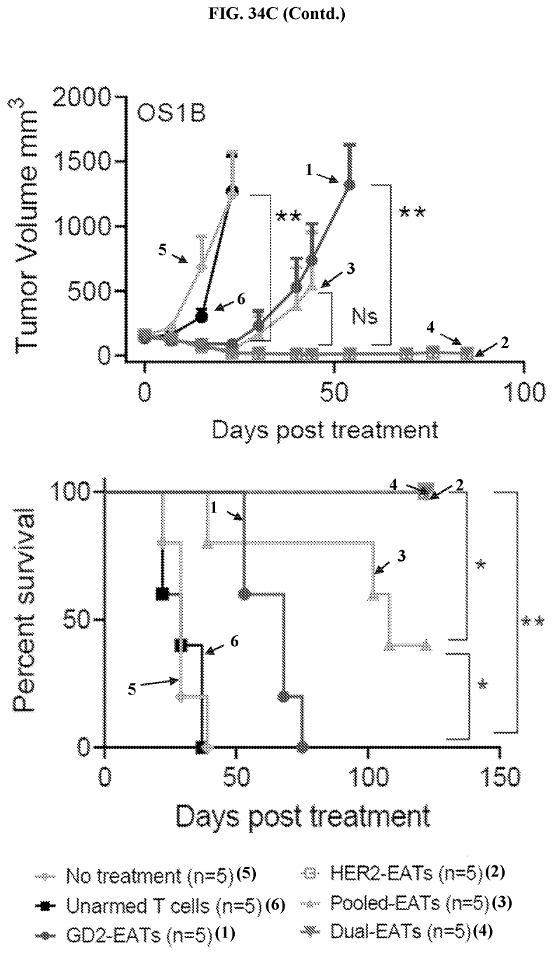

2 target antigens GD2 (disialogangliosides) and HER2 were chosen to test the efficacy of dual-antigens targeting strategies including pooled EATs (co-administering GD2-EATs and HER2-EATs), dual-EATs (T cells simultaneously armed with GD2-BsAb and HER2-BsAb), alternate EATs (GD2-EATs alternating with HER2-EATs), and TriAb-EATs (T cells armed with trispecific antibody (HER2×GD2×CD3 TriAb)] (FIG. 34A).

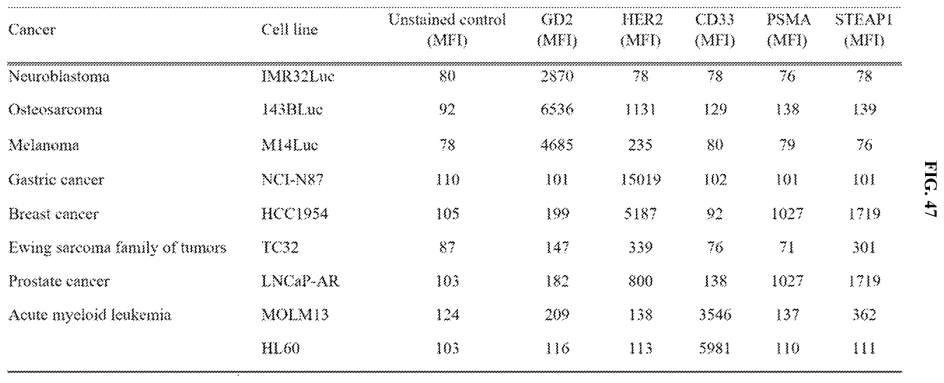

First, in vitro tumor cell killing by EATs was tested at fixed BsAb arming dose (0.5 μg of each BsAb/1×10⁶ T cells) with increasing ET ratios (FIG. 34B). Pooled-EATs and dual-EATs showed comparable tumor cell killing against GD2(+) and/or HER2(+) tumor cell lines (FIG. 47) when compared with mono-EATs (GD2-EATs or HER2-EATs). While pooled EATs presented an intermediate potency and efficacy between mono-EATs, dual-EATs showed similar potency when compared to individual mono-EATs. In vivo anti-tumor effect of multi-EATs was also evaluated using GD2(+) and HER2(+) osteosarcoma PDXs (FIG. 34C). While pooled-EATs showed an intermediate potency between mono-EATs, dual-EATs were equally effective as HER2-EATs; all 5 mice in the dual-EATs or HER2-EATs remained progression-free during follow-up period (up to 150 days post treatment), while none in the GD2-EATs group and only 2 of 5 in the pooled-EATs group showed a long-term remission. In vivo efficacy of dual-EATs compared to alternate-EATs was also tested using the osteosarcoma 143B CDX model (FIG. 41A). In alternate-EATs, GD2-EATs administration was alternated with HER2-EATs. These double antigen targeting approaches did not compromise the anti-tumor activities of mono-EATs or increase toxicities. However, there was no substantial improvement of dual-EATs over mono-EATs or over alternate EATs in this CDX model (FIG. 41B).

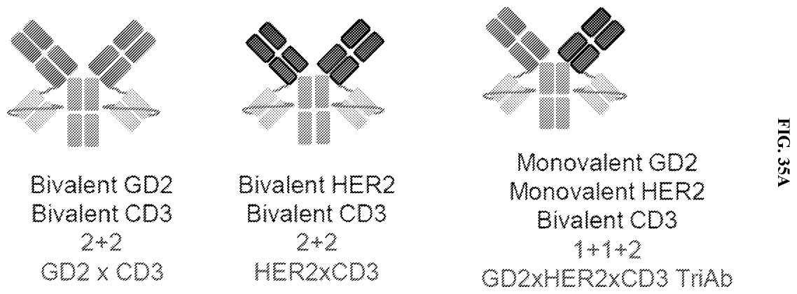

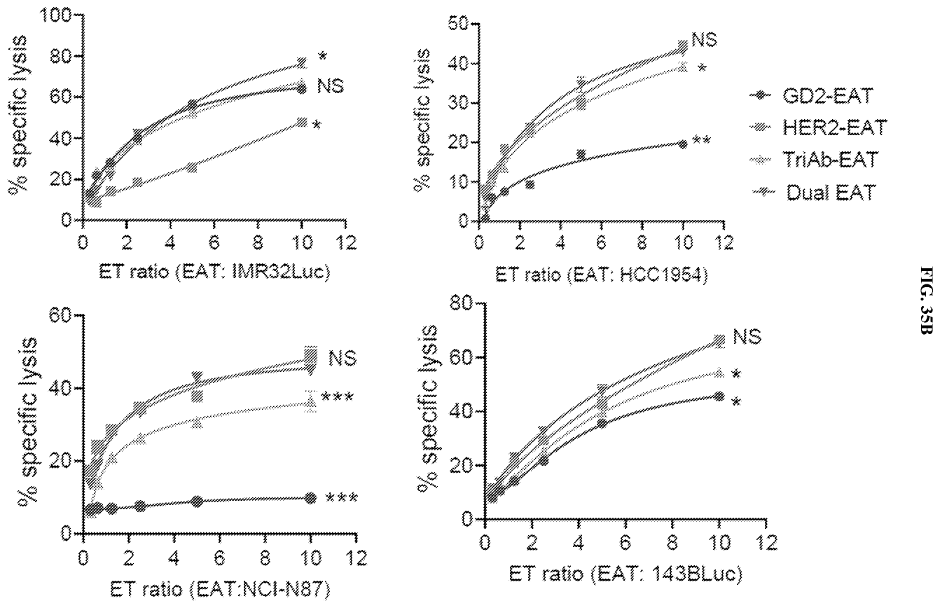

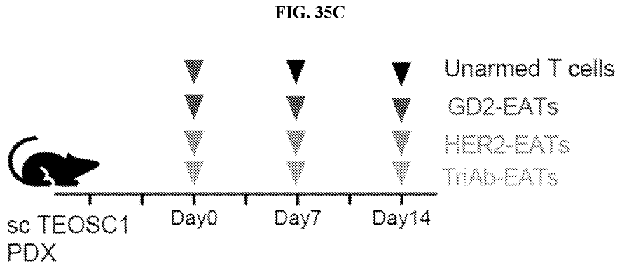

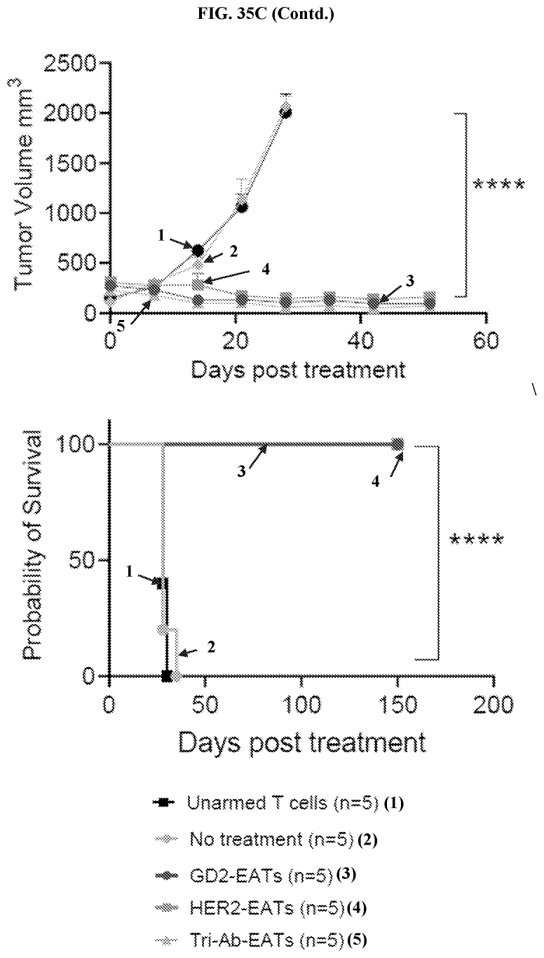

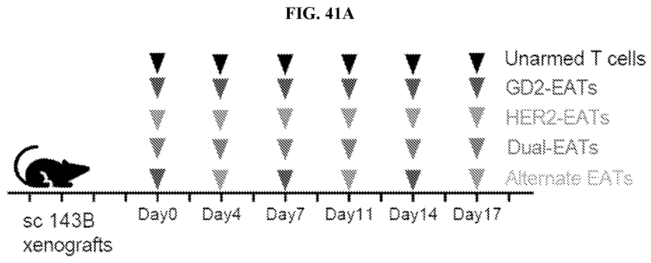

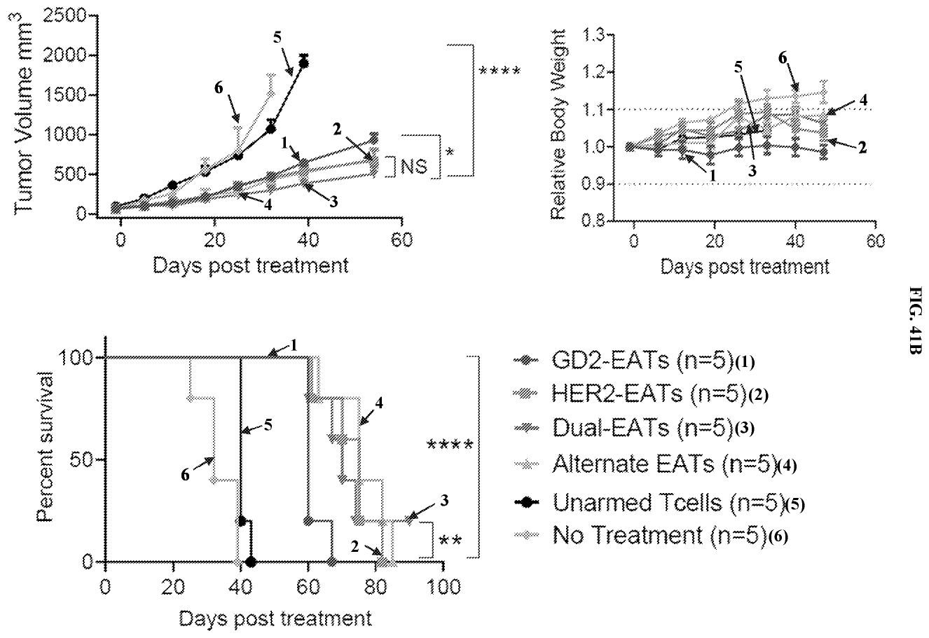

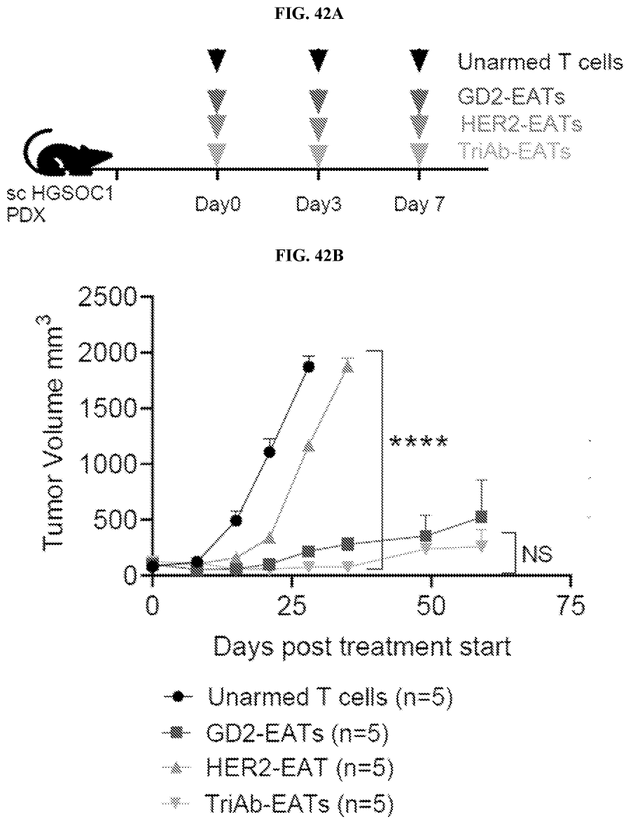

Next, the anti-tumor efficacy of dual-EATs was compared with TriAb-EATs. A novel GD2×HER2×CD3 trispecific antibody (TriAb) built on the IgG-[L]-scFv platform was developed using a heterodimeric approach (FIG. 35A) as previously described in Santich et al., Sci Transl Med 12, eaax1315 (2020), which is incorporated by reference herein. HER2×GD2×CD3 TriAb's cytotoxicity against multiple cancer cell lines was tested in vitro at fixed BsAb arming dose (0.5 μg of each BsAb/1×10⁶ T cells) with increasing ET ratios (FIG. 35B). While TriAb-EATs (0.5 μg of TriAb/1×10⁶ cells) were more effective than GD2-EATs (0.5 μg of GD2-BsAb/1×10⁶ cells) but less potent than HER2-EATs against HER2(+) cancer cell lines, dual-EATs exerted consistently potent cytotoxicity against a variety of cancer cell lines. In vivo anti-tumor efficacy of TriAb-EATs was also tested against two different osteosarcoma PDXs. Three doses of TriAb-EATs successfully ablated PDX tumors, prolonging survival without obvious toxicity in TEOSC1 PDX model (FIG. 35C). TriAb-EATs were also effective in HGSOC1 PDX model which was more sensitive to GD2-EATs than HER2-EATs, presenting a compelling anti-tumor effect to GD2-EATs (FIGS. 42A-42B).

### Example 18: Optimizing BsAb Densities on Multi EATs

T cells were simultaneously armed with multiple T-BsAbs specific for GD2, HER2, CD33, STEAP-1, or PSMA, all built on the IgG-[L]-scFv platform. Given the finite CD3 density on human T cells, the range and the optimal BsAb surface density as a function of arming dose was set out to be identified. Surface BsAb density on EAT was analyzed using anti-human IgG Fc-specific antibody. Precise quantification of BsAb density was measured as antibody-binding capacity (ABC) by flow cytometry referenced to anti-rat quantum beads (FIG. 36A). As the BsAb dose and number have increased, BsAb surface density also has increased. Arming with 5 BsAbs at high arming dose (25 μg of each BsAb/10⁶ cells), surface density of BsAbs plateaued at approximately 50,000 molecules per T cell.

To identify the range of optimal surface density of BsAb for multi-EATs, in vitro cytotoxicity against CD33(+) leukemia cell line (MOLM13) was studied over a range of E:T ratios and BsAb arming doses (FIG. 36B). Multi-EATs (armed with 5 BsAbs each targeting GD2, HER2, CD33, STEAP-1, and PSMA, respectively) showed the best cytotoxicity at the arming dose for each BsAb between 0.05 μg/1×10⁶ T cells and 1 μg/1×10⁶ T cells, corresponding to the BsAb densities between 5,000 and 20,000 molecules per T cell. When referenced to the BsAb density on CD33-EATs which showed the best efficacy between 0.5 μg and 5 μg of BsAb/1×10⁶ T cells, EATs appear to show the best tumoricidal activity between 5,000 and 20,000 BsAb molecules per T cell.

In vitro anti-tumor activity of multi-EATs targeting 5 antigens (GD2, HER2, CD33, PSMA, and STEAP1) was evaluated against varieties of tumor target (FIG. 47) over a range of BsAb arming doses and compared with the cytotoxicity of mono-EATs (FIG. 36C). Despite the presence of multiple BsAbs on the same EAT, multi-EATs exerted consistently potent anti-tumor activities against each tumor target, comparable to those of mono-EATs, although the maximal cytotoxicity (Emax) did vary depending on the specific targets studied.

### Example 19: Ex Vivo Arming of T Cells Attenuated Cytokine Surge from Multiple BsAbs

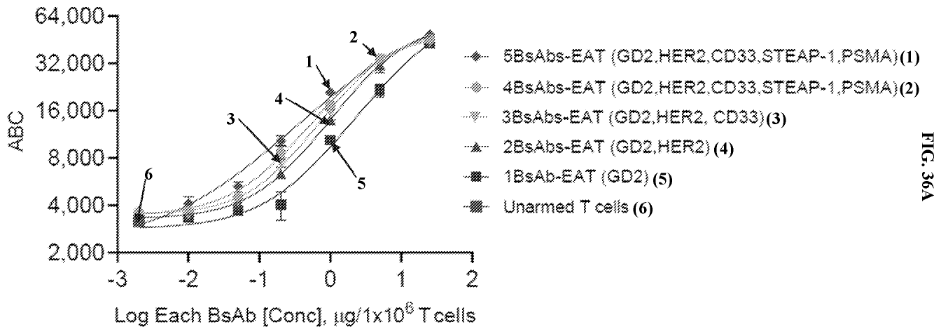

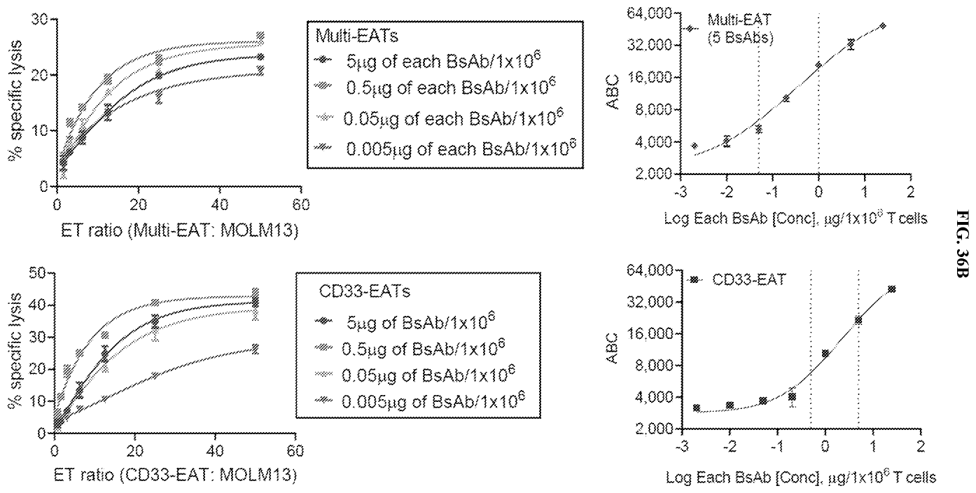

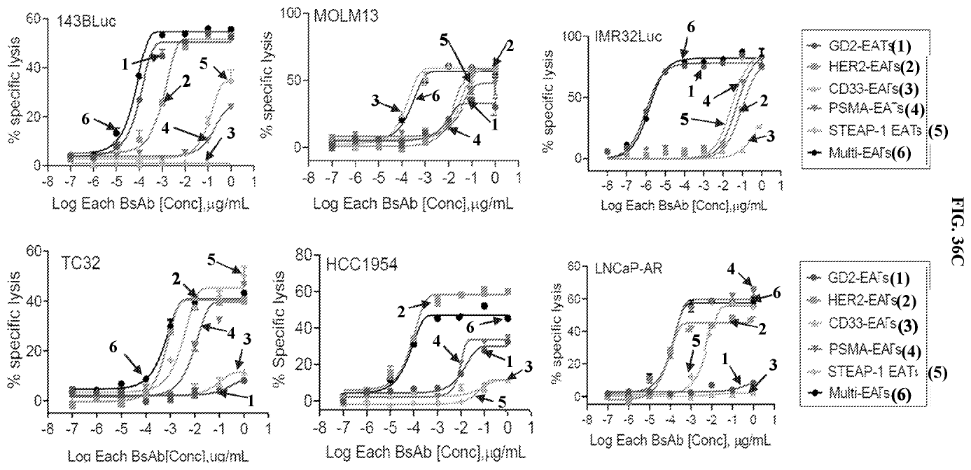

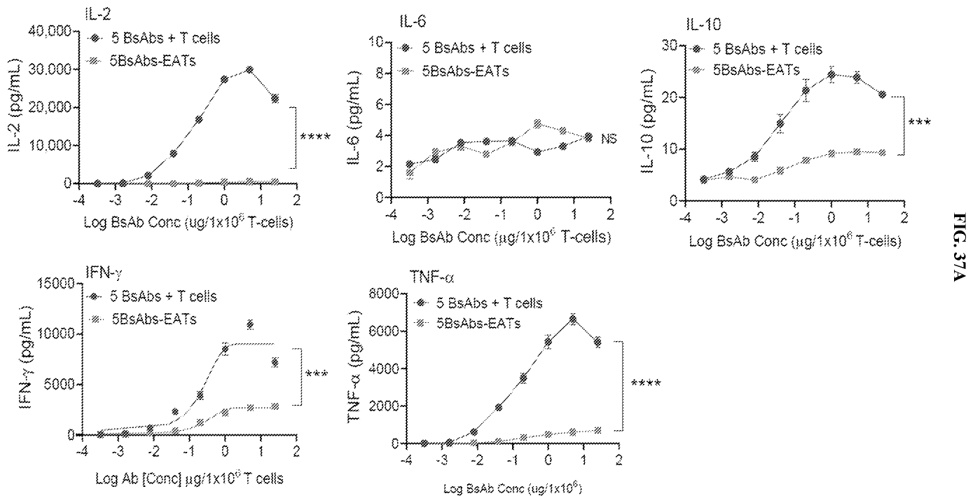

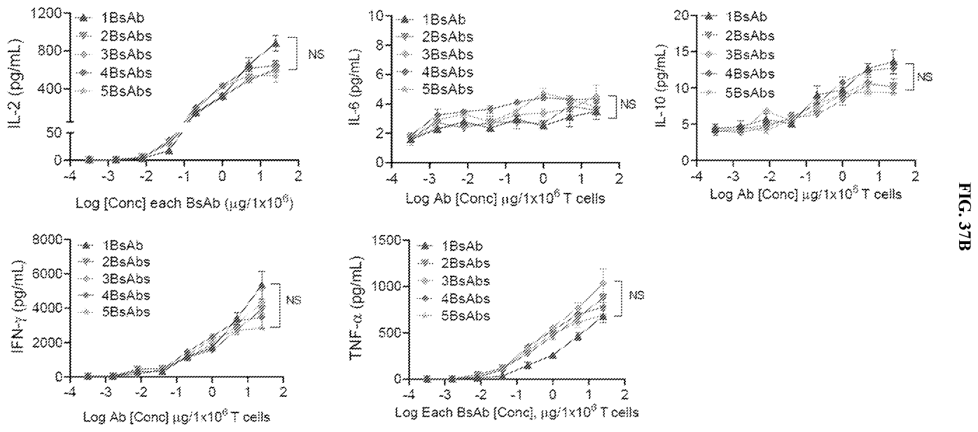

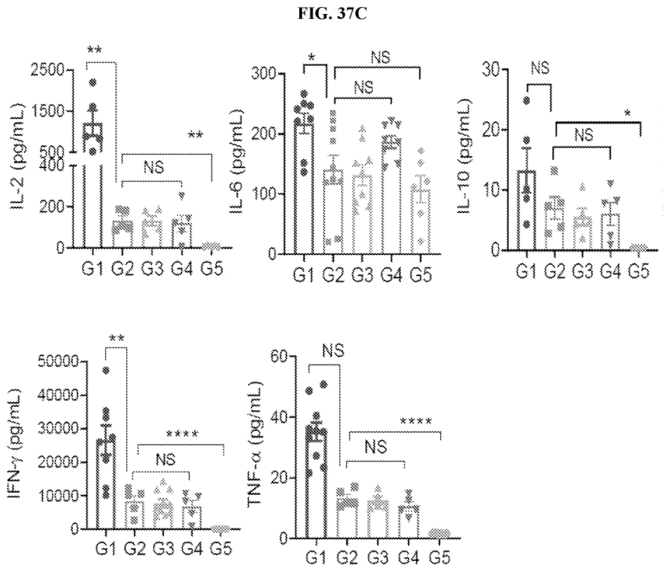

Because simultaneous administration of multiple BsAbs may precipitate a cytokine storm, cytokine release was compared between multi-EATs and multiple BsAbs plus T cells at increasing doses of BsAb. Multi-EATs or multiple BsAb plus T cells were incubated with target cells at 37° C. for 4 hours. Cytokine release by multiple BsAbs plus T cells and multi-EATs increased by BsAb dose, but reached plateaus at 1 μg of each BsAb/1×10⁶ cells. However, the cytokine levels of multi-EATs were significantly lower than those of multiple-BsAbs plus T cells over a range of BsAb doses (FIG. 37A). When the levels of cytokines released by mono-EATs (HER2-EATs), dual-EATs (HER2/GD2-EATs), triple-EATs (HER2/GD2/CD33-EATs), quadruple-EATs (HER2/GD2/CD33/PSMA-EATs), and quintuple-EATs (HER2/GD2/CD33/PSMA/STEAP1-EATs) were compared, the differences were not significant among groups (FIG. 37B). Although IL-2, IL-10, IFN-γ, and TNF-α levels increased with BsAb arming dose, there was no excessive cytokine release with additional BsAbs for multi-EATs. In vivo cytokine levels by multi-EATs were also analyzed post treatment and compared among groups (FIG. 37C). Multi-EATs (50 μg of total BsAb/2×10⁷ cells, G2) released significantly less IL-2, IL-6, IFN-γ, and TNF-α than administering GD2-BsAb (10 μg) plus unarmed T cells (2×10⁷ cells) (G1); there was no significant difference in cytokine release among mono-EATs (G3, GD2-EATs; G4, HER2-EATs) and multi-EATs (G2).

### Example 20: Multi-EATs were Efficient Multi-Specific Cytotoxic Lymphocytes

In Vivo Anti-Tumor Properties Against Diverse Cancer Types

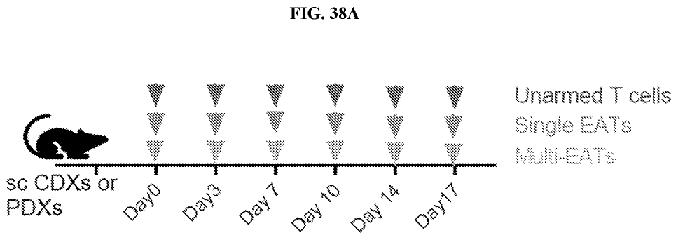

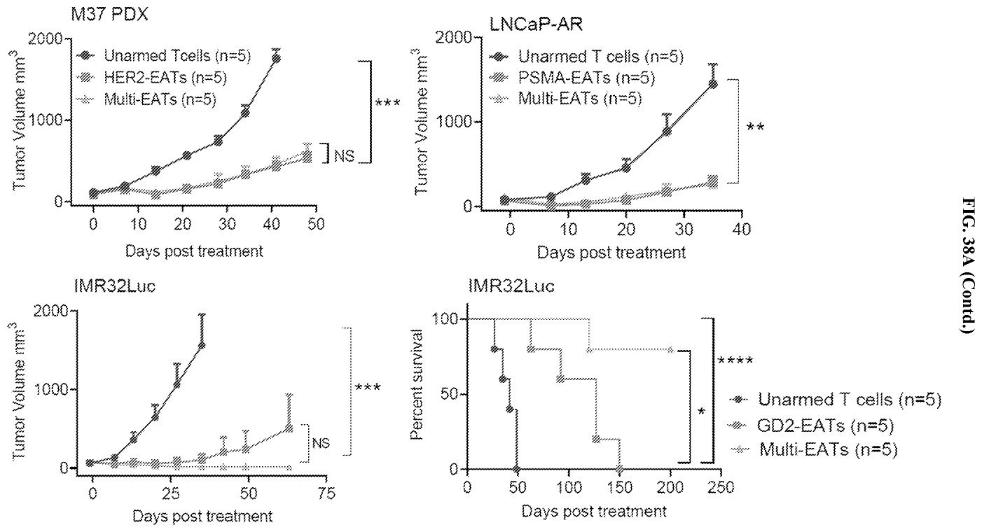

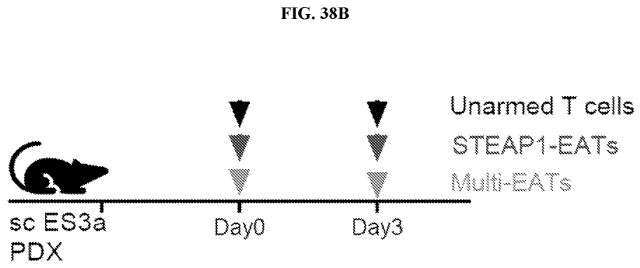

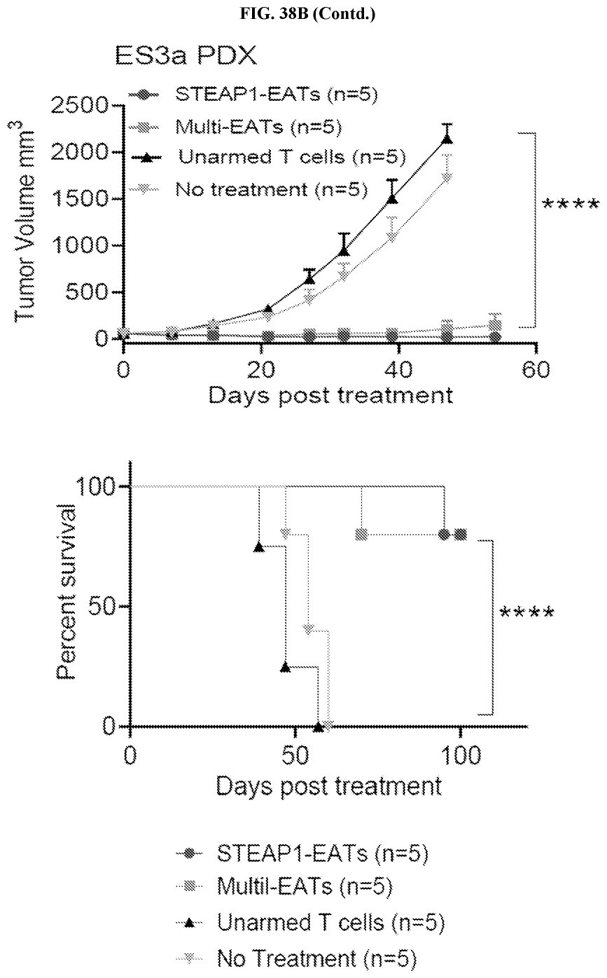

In vivo anti-tumor effect of multi-EATs was tested against xenografts representing diverse cancer diagnoses (FIG. 38A). Multi-EATs (2 μg of each BsAb×5 BsAbs/2×10⁷ T cells per injection) significantly suppressed tumor growth and consistently showed competitive anti-tumor effect to mono-EATs against a panel of target appropriate cancer xenografts, including HER2(+) M37 breast cancer PDX, PSMA(+) LNCaP-AR prostate cancer CDX, GD2(+) IMR32Luc neuroblastoma CDX, and STEAP1(+) ES3a Ewing sarcoma PDXs (FIG. 38B), without clinical toxicities. For IMR32Luc CDXs, multi-EATs exerted a robust anti-tumor effect surpassing the efficacy of GD2-EATs and significantly prolonging survival.

Multi EATs were Highly Effective Against Tumor Models with Antigen Heterogeneity

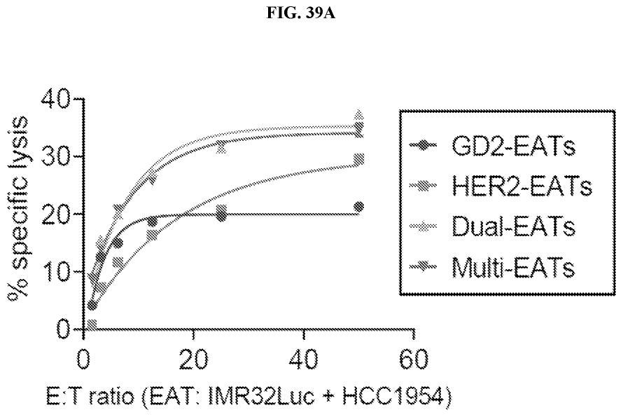

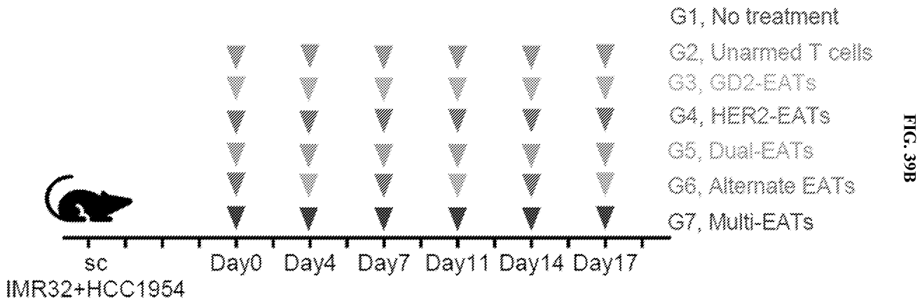

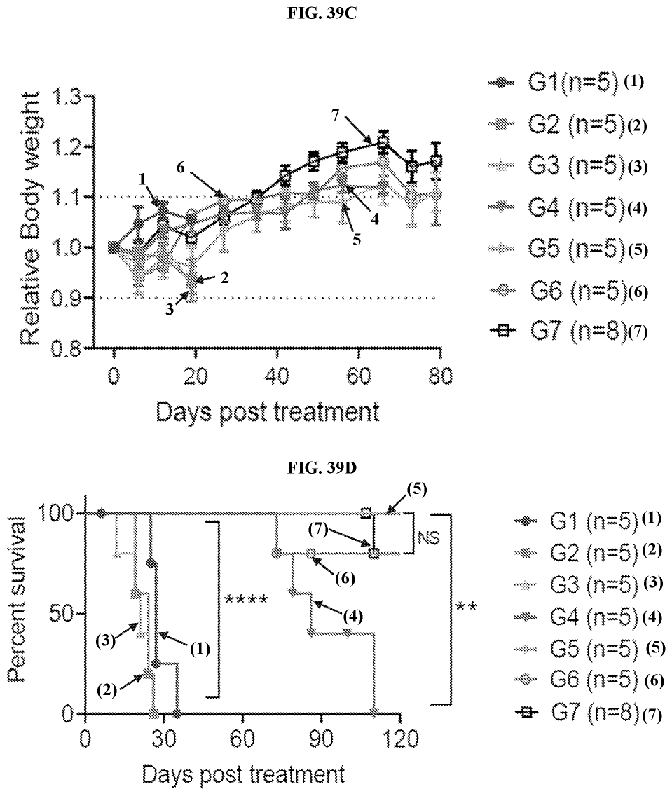

The ability of multi-EATs to overcome tumor heterogeneity was studied by creating a mixed lineage, i.e., GD2(+) HER^{low} IMR32Luc mixed with GD2^{low}HER2(+) HCC1954 (1:1 ratio) (FIG. 6C). Dual-EATs (T cells armed with GD2-BsAb and HER2-BsAb) and multi-EATs (EATs armed with 5 BsAbs targeting GD2, HER2, CD33, PSMA, and STEAP1, respectively) mediated stronger cytotoxicity against this mixed lineage than GD2-EATs or HER2-EATs (FIG. 39A). This enhanced in vitro cytotoxicity of dual- or multi-EATs was next tested for their in vivo anti-tumor response. A mixture of the two cell lines was xenografted subcutaneously and treated with dual- or multi-EATs when compared to mono-EATs (FIG. 39B). EATs were armed at 10 μg of each BsAb/2×10⁷ T cells for each injection. No clinical toxicities were observed and there was no weight loss throughout the follow-up period (FIG. 39C). While GD2-EATs or HER2-EATs failed to produce durable responses against this mixed lineage CDX, dual-, alternate-, or multi-EATs successfully achieved tumor regressions, producing long-term survival (FIGS. 39D-39E). Dual-EATs and multi-EATs both surpassed the efficacy of each mono-EATs significantly improved tumor-free survival (vs. HER2-EATs, P=0.0033; vs. GD2-EATs, P<0.0001).

The efficacy of TriAb-EATs against this mixed lineage was also tested. While TriAb-EATs showed enhanced in vitro cytotoxicity compared to GD2-EATs or HER2-EATs, it was not as effective when compared to dual- or multi-EATs (FIG. 43A). In vivo anti-tumor activity of TriAb-EATs was also tested against this mixed lineage CDXs (FIG. 43B). Tumors regressed following TriAb-EATs but the response was not durable: all 5 mice recurred in contrast to dual- or multi-EATs where long-term disease-free survival extended past 140 days in 3 out of 5 and 4 out of 5 mice, respectively.

### Example 21: Multi-EATs Overcame Tumoral Heterogeneity: Histologic Response of Mixed Lineage CDX to Multi-EATs

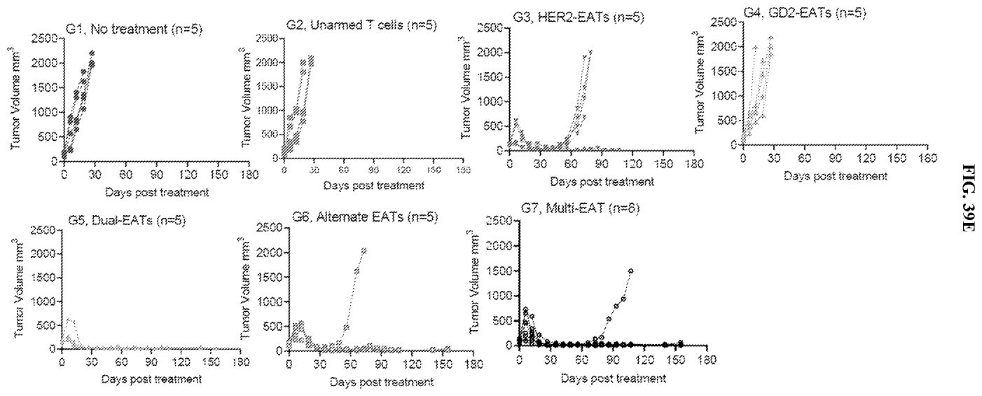

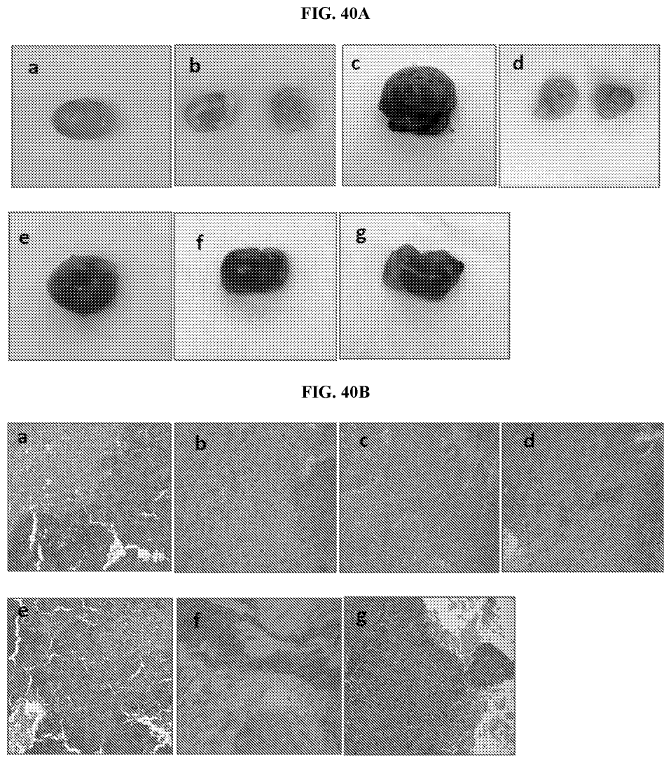

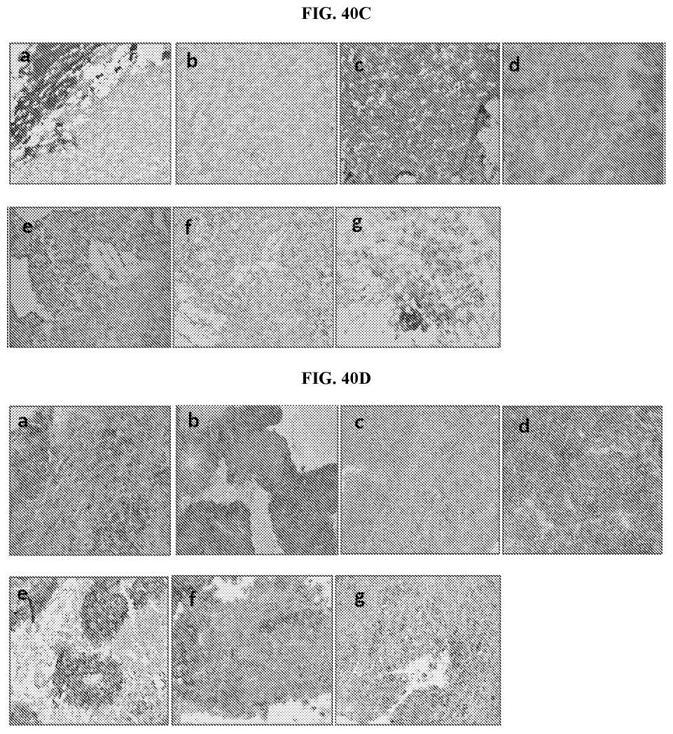

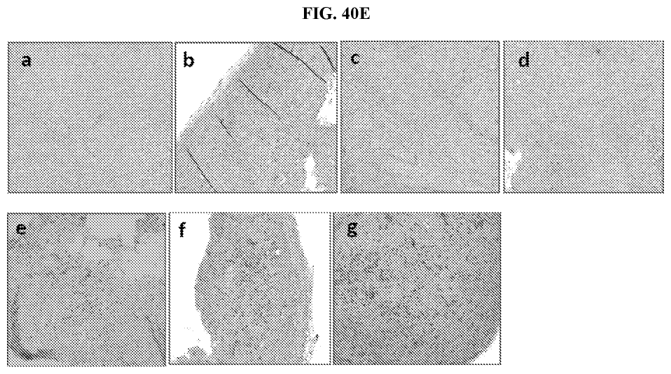

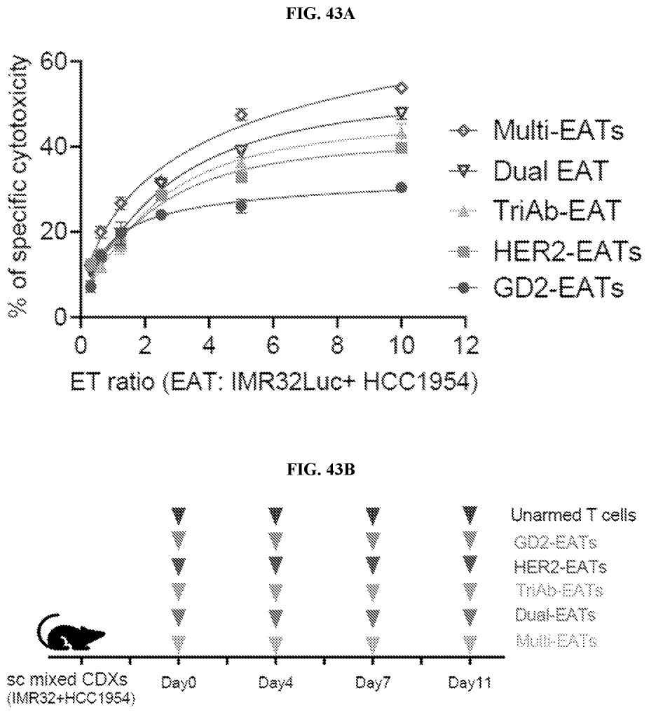

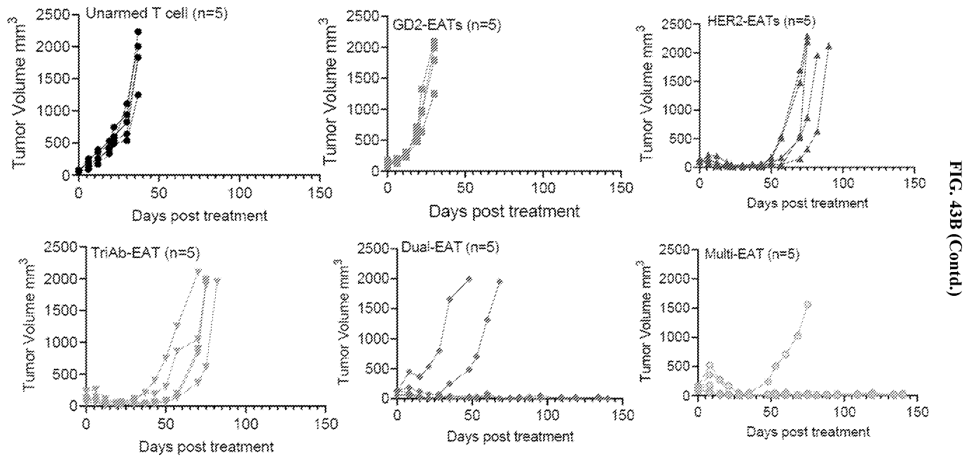

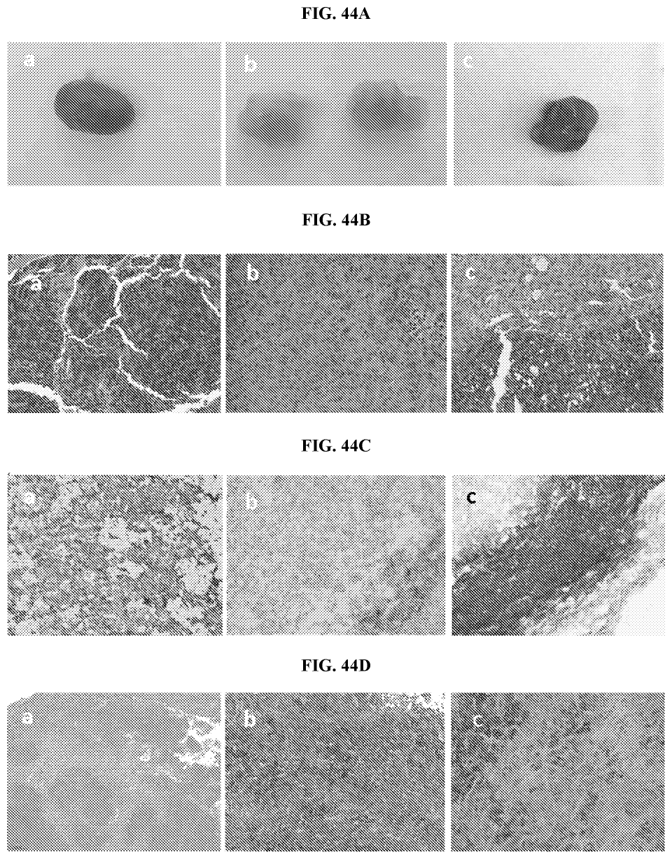

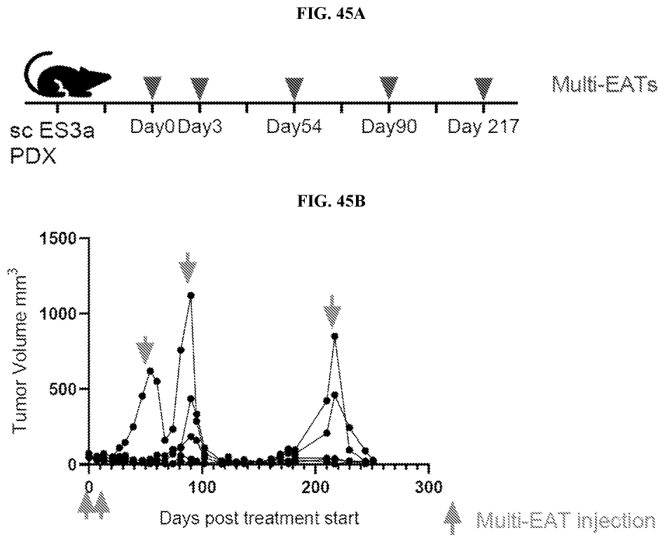

The mixed lineage CDXs were harvested after treatment and analyzed their antigen expression. Gross examination of these tumors presented distinct differences between GD2(+) IMR32Luc and HER2(+) HCC1954 lineages (FIG. 40A). Following treatment with GD2-EATs (b) or TriAb-EATs (d) tumors grossly resembled HCC1954 CDXs, while those following treatment with HER2-EATs (c) resembled IMR32Luc CDXs (FIGS. 44A-44D). Following treatment with alternate-EATs (e), dual-EATs (f), or multi-EATs (g) tumors acquired the appearance of a cross between IMR32Luc and HCC1954 xenografts, while untreated tumors or those treated with unarmed T cells (a) more resembled HCC1954 CDXs, consistent with rapid outgrowth of HCC1954 overtaking IMR32Luc. H&E staining results were consistent with their gross phenotypes (FIG. 40B). While following treatment with GD2-EATs or TriAb-EATs histology revealed poorly-differentiated invasive ductal breast carcinoma, following treatment with HER2-EATs, histology revealed immature, undifferentiated, small round neuroblasts accompanied by Homer-Wright pseudo-rosettes, typical characteristics of neuroblastoma. With no treatment or treatment with unarmed T cells, or with recurrence after initial response to alternate-, dual- or multi-EATs, tumor histology showed mixed lineage with a slight prominence of breast cancer features. Fresh frozen tumor staining with anti-GD2 antibody and FFPE tumor sections stained with anti-human HER2 also showed contrasting results following treatment (FIGS. 40C-40D). While the tumors without treatment or treated with unarmed T cells showed heterogenous GD2(+) and HER2(+) staining patterns, those following treatment with GD2-EATs or TriAb-EATs became GD2 negative while retaining strong HER2 positivity; vice versa, those tumors following HER2-EATs became strongly GD2 positive while losing HER2 staining. These results demonstrated that mono-EATs could ablate tumors in an exquisitely antigen specific manner, but unexpectedly were unable to control antigen negative clones in the mix. Escape tumors had antigen loss. On the other hand, treatment with dual-, alternate-, or multi-EATs could overcome tumor heterogeneity and the escape tumors were either GD2 or HER2 weakly positive, and total antigen loss was uncommon, permitting repeat response to multi-EATs (FIGS. 45A-45B).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein
(a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6,
wherein the at least one type of anti-CD3 multi-specific antibody is an immunoglobulin comprising two heavy chains and two light chains, wherein each of the light chains is fused to a single chain variable fragment (scFv), and
wherein the ex vivo armed T cell is or has been cryopreserved.

2. The ex vivo armed T cell of claim 1, wherein the ex vivo armed T cell is a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell; or
wherein the ex vivo armed T cell has been cryopreserved for a period of about 2 hours to about 6 months; or
wherein at least one scFv of the at least one type of anti-CD3 multi-specific antibody comprises the CD3 binding domain; or
wherein the at least one type of anti-CD3 multi-specific antibody binds one or more additional target antigens, optionally wherein the additional target antigens are selected from the group consisting of CD3, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, LMP2, p53, lung resistance protein (LRP), Bcl-2, prostate specific antigen (PSA), Ki-67, CEACAM6, colon-specific antigen-p (CSAp), HLA-DR, CD40, CD74, CD138, EGFR, EGP-1, EGP-2, VEGF, PlGF, insulin-like growth factor (ILGF), tenascin, platelet-derived growth factor, IL-6, CD20, CD19, PSMA, CD33, CD123, MET, DLL4, Ang-2, HER3, IGF-1R, CD30, TAG-72, SPEAP, CD45, L1-CAM, Lewis Y (Ley) antigen, E-cadherin, V-cadherin, GPC3, EpCAM, CD4, CD8, CD21, CD23, CD46, CD80, HLA-DR, CD74, CD22, CD14, CD15, CD16, CD123, TCR gamma/delta, NKp46, KIR, CD56, DLL3, PD-1, PD-L1, CD28, CD137, CD99, GloboH, CD24, STEAP1, B7H3, Polysialic Acid, OX40, OX40-ligand, peptide MHC complexes (with peptides derived from TP53, KRAS, MYC, EBNA1-6, PRAME, MART, tyronsinase, MAGEA1-A6, pmel17, LMP2, or WT1), and a DOTA-based hapten; or
wherein the V_{H} of the CD3 binding domain comprises the amino acid sequence of any one of SEQ ID NOs: 7-32, and/or wherein the V_{L} of the CD3 binding domain comprises the amino acid sequence of any one of SEQ ID NOs: 33-70; or
wherein the at least one type of anti-CD3 multi-specific antibody exhibits surface densities between about 500 to about 20,000 molecules per T cell; or
wherein the effective arming dose of the at least one type of anti-CD3 multi-specific antibody is between about 0.05 μg/10⁶ T cells to about 5 μg/10⁶ T cells.

3. (canceled)

4. An ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein
(a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6,
wherein the at least one type of anti-CD3 multi-specific antibody is an immunoglobulin comprising two heavy chains and two light chains, wherein each of the light chains is fused to a single chain variable fragment (scFv), and
wherein the ex vivo armed T cell is a γδ T cell, optionally wherein the ex vivo armed T cell is generated by contacting peripheral blood mononuclear cells with zoledronate and IL-15, wherein the IL-15 is administered as an IL15Rα-IL15 complex.

5. (canceled)

6. (canceled)

7. (canceled)

8. (canceled)

9. An ex vivo armed T cell that is coated or complexed with an effective arming dose of at least two types of anti-CD3 multi-specific antibodies, wherein each of the at least two types of anti-CD3 multi-specific antibodies includes a CD3 binding domain comprising a heavy chain immunoglobulin variable domain (V_{H}) and a light chain immunoglobulin variable domain (V_{L}), wherein
(a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6, and
wherein each of the at least two types of anti-CD3 multi-specific antibodies is an immunoglobulin comprising two heavy chains and two light chains, wherein each of the light chains is fused to a single chain variable fragment (scFv), optionally wherein
the at least two types of anti-CD3 multi-specific antibodies bind two or more additional target antigens; or
the ex vivo armed T cell is a helper T cell, a cytotoxic T cell, a memory T cell, a stem-cell-like memory T cell, an effector memory T cell, a regulatory T cell, a Natural killer T cell, a Mucosal associated invariant T cell, an EBV-specific cytotoxic T cell (EBV-CTL), an αβ T cell, or a γδ T cell.

10. The ex vivo armed T cell of claim 9, comprising 2, 3, 4, or 5 types of anti-CD3 multi-specific antibodies; or
wherein at least one scFv of each of the at least two types of anti-CD3 multi-specific antibodies comprises the CD3 binding domain, optionally wherein one or more of the at least two types of anti-CD3 multi-specific antibodies comprises a DOTA binding domain or comprise a scFv that includes the DOTA binding domain; or
wherein the at least two types of anti-CD3 multi-specific antibodies exhibit surface densities between about 1,500 to 10,000 molecules per T cell; or
wherein the effective arming dose of the at least two types of anti-CD3 multi-specific antibodies is between about 0.05 μg/10⁶ T cells to about 5 μg/10⁶ T cells.

11. (canceled)

12. (canceled)

13. (canceled)

14. An ex vivo armed T cell that is coated or complexed with an effective arming dose of at least one type of anti-CD3 multi-specific antibody, wherein the at least one type of anti-CD3 multi-specific antibody includes a CD3 binding domain comprising a heavy chain immunoglobulin variable domain (V_{H}) and a light chain immunoglobulin variable domain (V_{L}), wherein
(a) the V_{H} comprises a V_{H}-CDR1 sequence of SEQ ID NO: 1, a V_{H}-CDR2 sequence of SEQ ID NO: 2, and a V_{H}-CDR3 sequence of SEQ ID NO: 3, and
(b) the V_{L} comprises a V_{L}-CDR1 sequence of SEQ ID NO: 4, a V_{L}-CDR2 sequence of SEQ ID NO: 5, and a V_{L}-CDR3 sequence of SEQ ID NO: 6,
wherein the at least one type of anti-CD3 multi-specific antibody is an immunoglobulin comprising two heavy chains and two light chains, wherein each of the light chains is fused to a single chain variable fragment (scFv),
wherein at least one scFv of the at least one type of anti-CD3 multi-specific antibody comprises the CD3 binding domain, and
wherein at least one scFv of the at least one type of anti-CD3 multi-specific antibody comprises a DOTA binding domain.

15. (canceled)

16. (canceled)

17. (canceled)

18. (canceled)

19. (canceled)

20. (canceled)

21. The ex vivo armed T cell of claim 1, wherein the at least one type of anti-CD3 multi-specific antibody comprises a heavy chain (HC) amino acid sequence comprising SEQ ID NO: 82, SEQ ID NO: 84, SEQ ID NO: 86, SEQ ID NO: 88, SEQ ID NO: 90, SEQ ID NO: 94, SEQ ID NO: 96, SEQ ID NO: 98, SEQ ID NO: 100, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 163, SEQ ID NO: 165, SEQ ID NO: 167, SEQ ID NO: 169, or a variant thereof having one or more conservative amino acid substitutions, and/or a light chain (LC) amino acid sequence comprising SEQ ID NO: 81, SEQ ID NO: 83, SEQ ID NO: 85, SEQ ID NO: 87, SEQ ID NO: 89, SEQ ID NO: 93, SEQ ID NO: 95, SEQ ID NO: 97, SEQ ID NO: 99, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 162, SEQ ID NO: 164, SEQ ID NO: 166, SEQ ID NO: 168, or a variant thereof having one or more conservative amino acid substitutions,
optionally wherein
the at least one type of anti-CD3 multi-specific antibody comprises a HC amino acid sequence and a LC amino acid sequence selected from the group consisting of:
SEQ ID NO: 82 and SEQ ID NO: 81,
SEQ ID NO: 84 and SEQ ID NO: 83,
SEQ ID NO: 86 and SEQ ID NO: 85,
SEQ ID NO: 88 and SEQ ID NO: 87,
SEQ ID NO: 90 and SEQ ID NO: 89,
SEQ ID NO: 94 and SEQ ID NO: 93,
SEQ ID NO: 96 and SEQ ID NO: 95,
SEQ ID NO: 98 and SEQ ID NO: 97,
SEQ ID NO: 100 and SEQ ID NO: 99,
SEQ ID NO: 115 and SEQ ID NO: 114,
SEQ ID NO: 117 and SEQ ID NO: 116,
SEQ ID NO: 119 and SEQ ID NO: 118,
SEQ ID NO: 121 and SEQ ID NO: 120,
SEQ ID NO: 123 and SEQ ID NO: 122,
SEQ ID NO: 125 and SEQ ID NO: 124,
SEQ ID NO: 127 and SEQ ID NO: 126,
SEQ ID NO: 129 and SEQ ID NO: 128,
SEQ ID NO: 131 and SEQ ID NO: 130,
SEQ ID NO: 133 and SEQ ID NO: 132,
SEQ ID NO: 135 and SEQ ID NO: 134,
SEQ ID NO: 137 and SEQ ID NO: 136,
SEQ ID NO: 139 and SEQ ID NO: 138,
SEQ ID NO: 141 and SEQ ID NO: 140,
SEQ ID NO: 143 and SEQ ID NO: 142,
SEQ ID NO: 145 and SEQ ID NO: 144,
SEQ ID NO: 147 and SEQ ID NO: 146,
SEQ ID NO: 149 and SEQ ID NO: 148,
SEQ ID NO: 151 and SEQ ID NO: 150,
SEQ ID NO: 153 and SEQ ID NO: 152,
SEQ ID NO: 155 and SEQ ID NO: 154,
SEQ ID NO: 157 and SEQ ID NO: 156,
SEQ ID NO: 163 and SEQ ID NO: 162,
SEQ ID NO: 165 and SEQ ID NO: 164,
SEQ ID NO: 167 and SEQ ID NO: 166,
SEQ ID NO: 169 and SEQ ID NO: 168, respectively; or
the at least one type of anti-CD3 multi-specific antibody comprises a first LC amino acid sequence, a first HC amino acid sequence, a second LC amino acid sequence, and a second HC amino acid sequence selected from the group consisting of
SEQ ID NO: 114, SEQ ID NO: 115, SEQ ID NO: 116, and SEQ ID NO: 117; SEQ ID NO: 118, SEQ ID NO: 119, SEQ ID NO: 120, and SEQ ID NO: 121; SEQ ID NO: 122, SEQ ID NO: 123, SEQ ID NO: 124, and SEQ ID NO: 125; SEQ ID NO: 126, SEQ ID NO: 127, SEQ ID NO: 128, and SEQ ID NO: 129; SEQ ID NO: 130, SEQ ID NO: 131, SEQ ID NO: 132, and SEQ ID NO: 133; SEQ ID NO: 134, SEQ ID NO: 135, SEQ ID NO: 136, and SEQ ID NO: 137; SEQ ID NO: 138, SEQ ID NO: 139, SEQ ID NO: 140, and SEQ ID NO: 141; SEQ ID NO: 142, SEQ ID NO: 143, SEQ ID NO: 144, and SEQ ID NO: 145; SEQ ID NO: 146, SEQ ID NO: 147, SEQ ID NO: 148, and SEQ ID NO: 149; SEQ ID NO: 150, SEQ ID NO: 151, SEQ ID NO: 152, and SEQ ID NO: 153; SEQ ID NO: 154, SEQ ID NO: 155, SEQ ID NO: 156, and SEQ ID NO: 157; SEQ ID NO: 162, SEQ ID NO: 163, SEQ ID NO: 164, and SEQ ID NO: 165; and SEQ ID NO: 166, SEQ ID NO: 167, SEQ ID NO: 168, and SEQ ID NO: 169; respectively.

22. (canceled)

23. (canceled)

24. (canceled)

25. (canceled)

26. A method for tracking ex vivo armed T cells in a subject in vivo comprising
(A)
(a) administering to the subject an effective amount of the ex vivo armed T cell of claim 2, wherein at least one scFv of the at least one type of anti-CD3 multi-specific antibody comprises a DOTA binding domain, wherein the DOTA binding domain comprises the amino acid sequence of any one of SEQ ID NOs: 77-80, and wherein the ex vivo armed T cell is configured to localize to a tissue expressing one or more target antigens recognized by the at least one type of anti-CD3 multi-specific antibody of the ex vivo armed T cell;
(b) administering to the subject an effective amount of a radiolabeled DOTA-based hapten, wherein the radiolabeled DOTA-based hapten is configured to bind to the at least one type of anti-CD3 multi-specific antibody of the ex vivo armed T cell; and
(c) determining the biodistribution of the ex vivo armed T cell in the subject by detecting radioactive levels emitted by the radiolabeled DOTA-based hapten that are higher than a reference value; or
(B)
(a) administering to the subject an effective amount of a complex comprising the ex vivo armed T cell of claim 2 and a radiolabeled DOTA-based hapten, wherein at least one scFv of the at least one type of anti-CD3 multi-specific antibody comprises a DOTA binding domain, wherein the DOTA binding domain comprises the amino acid sequence of any one of SEQ ID NOs: 77-80, and wherein the complex is configured to localize to a tissue expressing one or more target antigens recognized by the at least one type of anti-CD3 multi-specific antibody of the ex vivo armed T cell; and
(b) determining the biodistribution of the ex vivo armed T cell in the subject by detecting radioactive levels emitted by the complex that are higher than a reference value.

27. (canceled)

28. A method for detecting tumors in a subject in need thereof comprising
(A)
(a) administering to the subject an effective amount of the ex vivo armed T cell of claim 2, wherein at least one scFv of the at least one type of anti-CD3 multi-specific antibody comprises a DOTA binding domain, wherein the DOTA binding domain comprises the amino acid sequence of any one of SEQ ID NOs: 77-80, and wherein the ex vivo armed T cell is configured to localize to a tissue expressing one or more target antigens recognized by the at least one type of anti-CD3 multi-specific antibody of the ex vivo armed T cell;
(b) administering to the subject an effective amount of a radiolabeled DOTA-based hapten, wherein the radiolabeled DOTA-based hapten is configured to bind to the at least one type of anti-CD3 multi-specific antibody of the ex vivo armed T cell; and
(c) detecting the presence of tumors in the subject by detecting radioactive levels emitted by the radiolabeled DOTA-based hapten that are higher than a reference value; or
(B)
(a) administering to the subject an effective amount of a complex comprising the ex vivo armed T cell of claim 2 and a radiolabeled DOTA-based hapten, wherein at least one scFv of the at least one type of anti-CD3 multi-specific antibody comprises a DOTA binding domain, wherein the DOTA binding domain comprises the amino acid sequence of any one of SEQ ID NOs: 77-80, and wherein the complex is configured to localize to a tissue expressing one or more target antigens recognized by the at least one type of anti-CD3 multi-specific antibody of the ex vivo armed T cell; and
(b) detecting the presence of tumors in the subject by detecting radioactive levels emitted by the complex that are higher than a reference value.

29. (canceled)

30. A method for assessing the in vivo durability or persistence of ex vivo armed T cells in a subject comprising
(A)
(a) administering to the subject an effective amount of the ex vivo armed T cell of claim 2, wherein at least one scFv of the at least one type of anti-CD3 multi-specific antibody comprises a DOTA binding domain, wherein the DOTA binding domain comprises the amino acid sequence of any one of SEQ ID NOs: 77-80, and wherein the ex vivo armed T cell is configured to localize to a tissue expressing one or more target antigens recognized by the at least one type of anti-CD3 multi-specific antibody of the ex vivo armed T cell;
(b) administering to the subject a first effective amount of a radiolabeled DOTA-based hapten, wherein the radiolabeled DOTA-based hapten is configured to bind to the at least one type of anti-CD3 multi-specific antibody of the ex vivo armed T cell; and
(c) detecting radioactive levels emitted by the radiolabeled DOTA-based hapten that are higher than a reference value at a first time point;
(d) detecting radioactive levels emitted by the radiolabeled DOTA-based hapten that are higher than a reference value at a second time point; and
(e) determining that the ex vivo armed T cells show in vivo durability or persistence when the radioactive levels emitted by the radiolabeled DOTA-based hapten at the second time point are comparable to that observed at the first time point, optionally wherein the method further comprises administering to the subject a second effective amount of the radiolabeled DOTA-based hapten after step (c); or
(B)
(a) administering to the subject an effective amount of a complex comprising the ex vivo armed T cell of claim 2 and a radiolabeled DOTA-based hapten, wherein at least one scFv of the at least one type of anti-CD3 multi-specific antibody comprises a DOTA binding domain, wherein the DOTA binding domain comprises the amino acid sequence of any one of SEQ ID NOs: 77-80, and wherein the complex is configured to localize to a tissue expressing one or more target antigens recognized by the at least one type of anti-CD3 multi-specific antibody of the ex vivo armed T cell;
(b) detecting radioactive levels emitted by the complex that are higher than a reference value at a first time point;
(c) detecting radioactive levels emitted by the complex that are higher than a reference value at a second time point; and
(d) determining that the ex vivo armed T cells show in vivo durability or persistence when the radioactive levels emitted by the complex at the second time point are comparable to that observed at the first time point.

31. (canceled)

32. (canceled)

33. The method of claim 28, wherein the radioactive levels emitted by the complex or the radiolabeled DOTA-based hapten are detected using positron emission tomography or single photon emission computed tomography; or
wherein the radioactive levels emitted by the complex or the radiolabeled DOTA-based hapten are detected between 4 to 24 hours after the complex or the radiolabeled DOTA-based hapten is administered; or
wherein the radioactive levels emitted by the complex or the radiolabeled DOTA-based hapten are expressed as the percentage injected dose per gram tissue (% ID/g); or
wherein the DOTA-based hapten is selected from the group consisting of benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), and DOTATyrLysDOTA.

34. (canceled)

35. (canceled)

36. A method for detecting the presence of a DOTA-based hapten in a subject comprising
(A)
(a) administering to the subject an effective amount of a DOTA-based hapten, wherein the DOTA-based hapten comprises a radionuclide, and is configured to localize to the ex vivo armed T cell; and
(b) detecting the presence of the DOTA-based hapten in the subject by detecting radioactive levels emitted by the DOTA-based hapten that are higher than a reference value, wherein the ex vivo armed T cell is configured to localize to a tumor expressing one or more target antigens recognized by the at least one type of anti-CD3 multi-specific antibody of the ex vivo armed T cell,
wherein the subject has been administered the ex vivo armed T cell of claim 2, wherein at least one scFv of the at least one type of anti-CD3 multi-specific antibody comprises a DOTA binding domain, wherein the DOTA binding domain comprises the amino acid sequence of any one of SEQ ID NOs: 77-80; or
(B)
detecting the presence of the DOTA-based hapten in the subject by detecting radioactive levels emitted by the complex that are higher than a reference value,
wherein the ex vivo armed T cell is configured to localize to a tumor expressing one or more target antigens recognized by the at least one type of anti-CD3 multi-specific antibody of the ex vivo armed T cell,
wherein the subject has been administered a complex comprising a DOTA-based hapten including a radionuclide and the ex vivo armed T cell of claim 2, wherein at least one scFv of the at least one type of anti-CD3 multi-specific antibody comprises a DOTA binding domain, wherein the DOTA binding domain comprises the amino acid sequence of any one of SEQ ID NOs: 77-80.

37. (canceled)

38. The method of claim 36, wherein the radioactive levels emitted by the DOTA-based hapten or complex are detected using positron emission tomography (PET) or single photon emission computed tomography (SPECT); or
wherein the method further comprises quantifying radioactive levels emitted by the DOTA-based hapten or complex that is localized to the tumor; or
wherein the method further comprises quantifying radioactive levels emitted by the DOTA-based hapten or the complex that is localized in one or more normal tissues or organs of the subject, wherein the one or more normal tissues or organs are selected from the group consisting of heart, muscle, gallbladder, esophagus, stomach, small intestine, large intestine, liver, pancreas, lungs, bone, bone marrow, kidneys, urinary bladder, brain, skin, spleen, thyroid, and soft tissue, and optionally determining biodistribution scores by computing a ratio of the radioactive levels emitted by the DOTA-based hapten or complex that is localized to the tumor relative to the radioactive levels emitted by the DOTA-based hapten or complex that is localized in the one or more normal tissues or organs of the subject, calculating estimated absorbed radiation doses for the tumor and the one or more normal tissues or organs of the subject based on the biodistribution scores, and computing a therapeutic index for the DOTA-based hapten or complex based on the estimated absorbed radiation doses for the tumor and the one or more normal tissues or organs of the subject.

39. (canceled)

40. (canceled)

41. (canceled)

42. (canceled)

43. (canceled)

44. (canceled)

45. A method for determining the antibody binding capacity of the ex vivo armed T cell of claim 1 in vitro comprising
contacting the ex vivo armed T cell with an agent that binds to the at least one type of anti-CD3 multi-specific antibody of the ex vivo armed T cell, wherein the agent is directly or indirectly linked to a detectable label, and
determining the antibody binding capacity of the ex vivo armed T cell by detecting the level or intensity of signal emitted by the detectable label.

46. A method for treating cancer or inhibiting tumor growth or metastasis in a subject in need thereof comprising administering to the subject an effective amount of the ex vivo armed T cell of claim 1, optionally wherein the ex vivo armed T cell is administered intravenously, intraperitoneally, subcutaneously, intramuscularly, or intratumorally.

47. (canceled)

48. A method for treating cancer or inhibiting tumor growth or metastasis in a subject in need thereof comprising
(a) administering to the subject a first effective amount of the ex vivo armed T cell of claim 1,
(b) administering to the subject a second effective amount of the ex vivo armed T cell about 72 hours after administration of the first effective amount of the ex vivo armed T cell,
(c) administering to the subject a third effective amount of the ex vivo armed T cell about 96 hours after administration of the second effective amount of the ex vivo armed T cell, and
(d) repeating steps (a)-(c) for at least three additional cycles, optionally wherein the subject exhibits sustained cancer remission after completion of step (d).

49. (canceled)

50. (canceled)

51. The method of claim 46, further comprising administering a cytokine to the subject or separately, simultaneously, or sequentially administering an additional cancer therapy to the subject,
optionally wherein
the additional cancer therapy is selected from among chemotherapy, radiation therapy, immunotherapy, monoclonal antibodies, anti-cancer nucleic acids or proteins, anti-cancer viruses or microorganisms, and any combinations thereof; or
the additional cancer therapy is an immune checkpoint inhibitor selected from among pembrolizumab, nivolumab, cemiplimab, atezolizumab, avelumab, durvalumab, and ipilimumab, or
the cytokine is selected from the group consisting of interferon α, interferon β, interferon γ, complement C5a, IL-2, TNFα, CD40L, IL12, IL-23, IL15, IL17, CCL1, CCL11, CCL12, CCL13, CCL14-1, CCL14-2, CCL14-3, CCL15-1, CCL15-2, CCL16, CCL17, CCL18, CCL19, CCL2, CCL20, CCL21, CCL22, CCL23-1, CCL23-2, CCL24, CCL25-1, CCL25-2, CCL26, CCL27, CCL28, CCL3, CCL3L1, CCL4, CCL4L1, CCL5, CCL6, CCL7, CCL8, CCL9, CCR10, CCR2, CCR5, CCR6, CCR7, CCR8, CCRL1, CCRL2, CX3CL1, CX3CR, CXCL1, CXCL10, CXCL11, CXCL12, CXCL13, CXCL14, CXCL15, CXCL16, CXCL2, CXCL3, CXCL4, CXCL5, CXCL6, CXCL7, CXCL8, CXCL9, CXCR1, CXCR2, CXCR4, CXCR5, CXCR6, CXCR7 and XCL2, or
wherein the cytokine is administered prior to, during, or subsequent to administration of the ex vivo armed T cell.

52. (canceled)

53. (canceled)

54. (canceled)

55. (canceled)

56. The method of claim 46, wherein the ex vivo armed T cell is autologous, non-autologous, or derived in vitro from lymphoid progenitor cells.

57. The method of claim 46, wherein the subject is diagnosed with, or is suspected of having cancer,
optionally wherein
the cancer or tumor is a carcinoma, sarcoma, a melanoma, or a hematopoietic cancer; or
the cancer is selected from the group consisting of osteosarcoma, Ewing's sarcoma, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, seminomas, skin cancers, stomach cancers, teratomas, testicular cancers, thyroid cancers, uterine cancers, vaginal cancers, vascular tumors, and metastases thereof.

58. (canceled)

59. (canceled)

60. The method of claim 46, wherein cytokine levels released by the ex vivo armed T cell are reduced compared to unarmed T cells mixed with an anti-CD3 multi-specific antibody.


SEQUENCE LISTING (169 sequences)

SEQ ID NO: 1 - protein, 8 aa, Artificial Sequence
  Description of Artificial Sequence Synthetic peptide
GYTFTRYT

SEQ ID NO: 2 - protein, 8 aa, Artificial Sequence
  Description of Artificial Sequence Synthetic peptide
INPSRGYT

SEQ ID NO: 3 - protein, 12 aa, Artificial Sequence
  Description of Artificial Sequence Synthetic peptide
ARYYDDHYSLDY

SEQ ID NO: 4 - protein, 5 aa, Artificial Sequence
  Description of Artificial Sequence Synthetic peptide
SSVSY

SEQ ID NO: 5 - protein, 2 aa, Artificial Sequence
  Description of Artificial Sequence Synthetic peptide
DT

SEQ ID NO: 6 - protein, 9 aa, Artificial Sequence
  Description of Artificial Sequence Synthetic peptide
QQWSSNPFT

SEQ ID NO: 7 - protein, 119 aa, Artificial Sequence
  Description of Artificial Sequence Synthetic polypeptide
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSS

SEQ ID NO: 8 - protein, 119 aa, Artificial Sequence
  Description of Artificial Sequence Synthetic polypeptide
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSS

SEQ ID NO: 9 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSLDYWGQGTTLTVSS

SEQ ID NO: 10 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGQGTTLTVSS

SEQ ID NO: 11 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSLDYWGQGTTLTVSS

SEQ ID NO: 12 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSLDYWGQGTTLTVSS

SEQ ID NO: 13 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSLDYWGCGTTLTVSS

SEQ ID NO: 14 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGCGTTLTVSS

SEQ ID NO: 15 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSLDYWGCGTTLTVSS

SEQ ID NO: 16 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSLDYWGCGTTLTVSS

SEQ ID NO: 17 - protein, 119 aa, Artificial Sequence
  Description of Artificial Sequence Synthetic polypeptide
QVQLQQSGAEVAKPGASVKMSCKASGYTFTRYTMHWVRQAPGQCLEWIGYINPSRGYTNYAQKFQGRATLTTDKSISTAYMELSRLRSDDTAVYYCARYYDDHYSLDYWGQGTTLTVSS

SEQ ID NO: 18 - protein, 119 aa, Artificial Sequence
  Description of Artificial Sequence Synthetic polypeptide
QVQLQQSGAEVAKPGASVKVSCKASGYTFTRYTMHWVRQAPGQCLEWMGYINPSRGYTNYNQKFKDRATLTRDKSISTAYMELSRLRSDDTAVYYCARYYDDHYSLDYWGQGTTLTVSS

SEQ ID NO: 19 - protein, 119 aa, Artificial Sequence
  Description of Artificial Sequence Synthetic polypeptide
QVQLVQSGAEVKKPGASVKMSCKASGYTFTRYTMHWVRQAPGQCLEWIGYINPSRGYTNYNQKFKDRATMTTDKSISTAYMELSRLRSDDTAVYYCARYYDDHYSLDYWGQGTTLTVSS

SEQ ID NO: 20 - protein, 119 aa, Artificial Sequence
  Description of Artificial Sequence Synthetic polypeptide
QVQLQQSGAEVKKPGASVKMSCKASGYTFTRYTMHWVRQAPGQCLEWIGYINPSRGYTNYNQKFKDRVTLTTDTSISTAYMELSRLRSDDTAVYYCARYYDDHYSLDYWGQGTTLTVSS

SEQ ID NO: 21 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSCDYWGQGTTLTVSS

SEQ ID NO: 22 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSCDYWGQGTTLTVSS

SEQ ID NO: 23 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSCDYWGQGTTLTVSS

SEQ ID NO: 24 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSCDYWGQGTTLTVSS

SEQ ID NO: 25 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHCSLDYWGQGTTLTVSS

SEQ ID NO: 26 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHCSLDYWGQGTTLTVSS

SEQ ID NO: 27 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHCSLDYWGQGTTLTVSS

SEQ ID NO: 28 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHCSLDYWGQGTTLTVSS

SEQ ID NO: 29 - protein, 119 aa, Artificial Sequence
  Description of Artificial Sequence Synthetic polypeptide
QVQLQQSGAEVAKPGASVKMSCKASGYTFTRYTMHWVRQAPGQGLEWIGYINPSRGYTNYAQKFQGRATLTTDKSISTAYMELSRLRSDDTAVYYCARYYDDHYSLCYWGQGTTLTVSS

SEQ ID NO: 30 - protein, 119 aa, Artificial Sequence
  Description of Artificial Sequence Synthetic polypeptide
QVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLCYWGQGTTLTVSS

SEQ ID NO: 31 - protein, 119 aa, Artificial Sequence
  Description of Artificial Sequence Synthetic polypeptide
QVQLVQSGAEVKKPGASVKMSCKASGYTFTRYTMHWVRQAPGQGLEWIGYINPSRGYTNYNQKFKDRATMTTDKSISTAYMELSRLRSDDTAVYYCARYYDDHYSLCYWGQGTTLTVSS

SEQ ID NO: 32 - protein, 119 aa, Artificial Sequence
  Description of Artificial Sequence Synthetic polypeptide
QVQLQQSGAEVKKPGASVKMSCKASGYTFTRYTMHWVRQAPGQGLEWIGYINPSRGYTNYNQKFKDRVTLTTDTSISTAYMELSRLRSDDTAVYYCARYYDDHYSLCYWGQGTTLTVSS

SEQ ID NO: 33 - protein, 107 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 34 - protein, 107 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 35 - protein, 107 aa, Artificial Sequence
  Description of Artificial Sequence Synthetic polypeptide
DIQMTQSPSSLSASVGDRVTMTCSASSSVSYMNWYQQKPGKAPKRLIYDTSKLASGVPSRFSGSGSGTDFTLTISSLQPEDFATYYCQQWSSNPFTFGSGTKLEINR

SEQ ID NO: 36 - protein, 107 aa, Artificial Sequence
  Description of Artificial Sequence Synthetic polypeptide
DIQMTQSPSSLSASVGDRVTMTCSASSSVSYMNWYQQKPGKAPKLLIYDTSKLASGVPSRFSGSGSGTDFTLTISSMQPEDFATYYCQQWSSNPFTFGSGTKLEINR

SEQ ID NO: 37 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDYTLTISSLQPEDFATYYCQQWSSNPFTFGSGTKLEINR

SEQ ID NO: 38 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDFTLTISSMQPEDFATYYCQQWSSNPFTFGSGTKLEINR

SEQ ID NO: 39 - protein, 107 aa, Artificial Sequence
  Description of Artificial Sequence Synthetic polypeptide
DIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGSGTKLEINR

SEQ ID NO: 40 - protein, 107 aa, Artificial Sequence
  Description of Artificial Sequence Synthetic polypeptide
DIQMTQSPSSLSASVGDRVTITCSASSSVSYMNWYQQKPGKAPKLWIYDTSKLASGVPSRFSGSGSGTDYTLTISSLQPEDFATYYCQQWSSNPFTFGSGTKLEINR

SEQ ID NO: 41 - protein, 107 aa, Artificial Sequence
  Description of Artificial Sequence Synthetic polypeptide
DIQMTQSPSSLSASVGDRVTMTCSASSSVSYMNWYQQKPGKAPKRLIYDTSKLASGVPSRFSGSGSGTDFTLTISSLQPEDFATYYCQQWSSNPFTFGCGTKLEINR

SEQ ID NO: 42 - protein, 107 aa, Artificial Sequence
  Description of Artificial Sequence Synthetic polypeptide
DIQMTQSPSSLSASVGDRVTMTCSASSSVSYMNWYQQKPGKAPKLLIYDTSKLASGVPSRFSGSGSGTDFTLTISSMQPEDFATYYCQQWSSNPFTFGCGTKLEINR

SEQ ID NO: 43 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDYTLTISSLQPEDFATYYCQQWSSNPFTFGCGTKLEINR

SEQ ID NO: 44 - protein, 107 aa, Artificial Sequence
  Description of Artificial Sequence Synthetic polypeptide
DIQLTQSPSSLSASVGDRVTITCSASSSVSYMNWYQQKPGKAPKLLIYDTSKLASGVPSRFSGSGSGTDFTLTISSMQPEDFATYYCQQWSSNPFTFGCGTKLEINR

SEQ ID NO: 45 - protein, 107 aa, Artificial Sequence
  Description of Artificial Sequence Synthetic polypeptide
DIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGCGTKLEINR

SEQ ID NO: 46 - protein, 107 aa, Artificial Sequence
  Description of Artificial Sequence Synthetic polypeptide
DIQMTQSPSSLSASVGDRVTITCSASSSVSYMNWYQQKPGKAPKLWIYDTSKLASGVPSRFSGSGSGTDYTLTISSLQPEDFATYYCQQWSSNPFTFGCGTKLEINR

SEQ ID NO: 47 - protein, 107 aa, Artificial Sequence
  Description of Artificial Sequence Synthetic polypeptide
DIQMTQSPSSLSASVGDRVTMTCSASSSVSYMNWYQQKPGKCPKRLIYDTSKLASGVPSRFSGSGSGTDFTLTISSLQPEDFATYYCQQWSSNPFTFGSGTKLEINR

SEQ ID NO: 48 - protein, 107 aa, Artificial Sequence
  Description of Artificial Sequence Synthetic polypeptide
DIQMTQSPSSLSASVGDRVTMTCSASSSVSYMNWYQQKPGKCPKLLIYDTSKLASGVPSRFSGSGSGTDFTLTISSMQPEDFATYYCQQWSSNPFTFGSGTKLEINR

SEQ ID NO: 49 - protein, 107 aa, Artificial Sequence
  Description of Artificial Sequence Synthetic polypeptide
DIQLTQSPSSLSASVGDRVTITCSASSSVSYMNWYQQKPGKCPKLLIYDTSKLASGVPSRFSGSGSGTDYTLTISSLQPEDFATYYCQQWSSNPFTFGSGTKLEINR

SEQ ID NO: 50 - protein, 107 aa, Artificial Sequence
  Description of Artificial Sequence Synthetic polypeptide
DIQLTQSPSSLSASVGDRVTITCSASSSVSYMNWYQQKPGKCPKLLIYDTSKLASGVPSRFSGSGSGTDFTLTISSMQPEDFATYYCQQWSSNPFTFGSGTKLEINR

SEQ ID NO: 51 - protein, 107 aa, Artificial Sequence
  Description of Artificial Sequence Synthetic polypeptide
DIQMTQSPSSLSASVGDRVTITCSASSSVSYMNWYQQKPGKCPKRWIYDTSKLASGVPSRFSGSGSGTDFTLTISSLQPEDFATYYCQQWSSNPFTFGSGTKLEINR

SEQ ID NO: 52 - protein, 107 aa, Artificial Sequence
  Description of Artificial Sequence Synthetic polypeptide
DIQMTQSPSSLSASVGDRVTITCSASSSVSYMNWYQQKPGKCPKLWIYDTSKLASGVPSRFSGSGSGTDYTLTISSLQPEDFATYYCQQWSSNPFTFGSGTKLEINR

SEQ ID NO: 53 - protein, 107 aa, Artificial Sequence
  Description of Artificial Sequence Synthetic polypeptide
DIQMTQSPSSLSASVGDRVTMTCSASSSVSYMNWYQQKPGKAPKRLICDTSKLASGVPSRFSGSGSGTDFTLTISSLQPEDFATYYCQQWSSNPFTFGSGTKLEINR

SEQ ID NO: 54 - protein, 107 aa, Artificial Sequence
  Description of Artificial Sequence Synthetic polypeptide
DIQMTQSPSSLSASVGDRVTMTCSASSSVSYMNWYQQKPGKAPKLLICDTSKLASGVPSRFSGSGSGTDFTLTISSMQPEDFATYYCQQWSSNPFTFGSGTKLEINR

SEQ ID NO: 55 - protein, 107 aa, Artificial Sequence
  Description of Artificial Sequence Synthetic polypeptide
DIQLTQSPSSLSASVGDRVTITCSASSSVSYMNWYQQKPGKAPKLLICDTSKLASGVPSRFSGSGSGTDYTLTISSLQPEDFATYYCQQWSSNPFTFGSGTKLEINR

SEQ ID NO: 56 - protein, 107 aa, Artificial Sequence
  Description of Artificial Sequence Synthetic polypeptide
DIQLTQSPSSLSASVGDRVTITCSASSSVSYMNWYQQKPGKAPKLLICDTSKLASGVPSRFSGSGSGTDFTLTISSMQPEDFATYYCQQWSSNPFTFGSGTKLEINR

SEQ ID NO: 57 - protein, 107 aa, Artificial Sequence
  Description of Artificial Sequence Synthetic polypeptide
DIQMTQSPSSLSASVGDRVTITCSASSSVSYMNWYQQKPGKAPKRWICDTSKLASGVPSRFSGSGSGTDFTLTISSLQPEDFATYYCQQWSSNPFTFGSGTKLEINR

SEQ ID NO: 58 - protein, 107 aa, Artificial Sequence
  Description of Artificial Sequence Synthetic polypeptide
DIQMTQSPSSLSASVGDRVTITCSASSSVSYMNWYQQKPGKAPKLWICDTSKLASGVPSRFSGSGSGTDYTLTISSLQPEDFATYYCQQWSSNPFTFGSGTKLEINR

SEQ ID NO: 59 - protein, 107 aa, Artificial Sequence
  Description of Artificial Sequence Synthetic polypeptide
DIQMTQSPSSLSASVGDRVTMTCSASSSVSYMNWYQQKPGKAPKRLIYCTSKLASGVPSRFSGSGSGTDFTLTISSLQPEDFATYYCQQWSSNPFTFGSGTKLEINR

SEQ ID NO: 60 - protein, 107 aa, Artificial Sequence
  Description of Artificial Sequence Synthetic polypeptide
DIQMTQSPSSLSASVGDRVTMTCSASSSVSYMNWYQQKPGKAPKLLIYCTSKLASGVPSRFSGSGSGTDFTLTISSMQPEDFATYYCQQWSSNPFTFGSGTKLEINR

SEQ ID NO: 61 - protein, 107 aa, Artificial Sequence
  Description of Artificial Sequence Synthetic polypeptide
DIQLTQSPSSLSASVGDRVTITCSASSSVSYMNWYQQKPGKAPKLLIYCTSKLASGVPSRFSGSGSGTDYTLTISSLQPEDFATYYCQQWSSNPFTFGSGTKLEINR

SEQ ID NO: 62 - protein, 107 aa, Artificial Sequence
  Description of Artificial Sequence Synthetic polypeptide
DIQLTQSPSSLSASVGDRVTITCSASSSVSYMNWYQQKPGKAPKLLIYCTSKLASGVPSRFSGSGSGTDFTLTISSMQPEDFATYYCQQWSSNPFTFGSGTKLEINR

SEQ ID NO: 63 - protein, 107 aa, Artificial Sequence
  Description of Artificial Sequence Synthetic polypeptide
DIQMTQSPSSLSASVGDRVTITCSASSSVSYMNWYQQKPGKAPKRWIYCTSKLASGVPSRFSGSGSGTDFTLTISSLQPEDFATYYCQQWSSNPFTFGSGTKLEINR

SEQ ID NO: 64 - protein, 107 aa, Artificial Sequence
  Description of Artificial Sequence Synthetic polypeptide
DIQMTQSPSSLSASVGDRVTITCSASSSVSYMNWYQQKPGKAPKLWIYCTSKLASGVPSRFSGSGSGTDYTLTISSLQPEDFATYYCQQWSSNPFTFGSGTKLEINR

SEQ ID NO: 65 - protein, 107 aa, Artificial Sequence
  Description of Artificial Sequence Synthetic polypeptide
DIQMTQSPSSLSASVGDRVTMTCSASSSVSYMNWYQQKPGKAPKCLIYDTSKLASGVPSRFSGSGSGTDFTLTISSLQPEDFATYYCQQWSSNPFTFGSGTKLEINR

SEQ ID NO: 66 - protein, 107 aa, Artificial Sequence
  Description of Artificial Sequence Synthetic polypeptide
DIQMTQSPSSLSASVGDRVTMTCSASSSVSYMNWYQQKPGKAPKCLIYDTSKLASGVPSRFSGSGSGTDFTLTISSMQPEDFATYYCQQWSSNPFTFGSGTKLEINR

SEQ ID NO: 67 - protein, 107 aa, Artificial Sequence
  Description of Artificial Sequence Synthetic polypeptide
DIQLTQSPSSLSASVGDRVTITCSASSSVSYMNWYQQKPGKAPKCLIYDTSKLASGVPSRFSGSGSGTDYTLTISSLQPEDFATYYCQQWSSNPFTFGSGTKLEINR

SEQ ID NO: 68 - protein, 107 aa, Artificial Sequence
  Description of Artificial Sequence Synthetic polypeptide
DIQLTQSPSSLSASVGDRVTITCSASSSVSYMNWYQQKPGKAPKCLIYDTSKLASGVPSRFSGSGSGTDFTLTISSMQPEDFATYYCQQWSSNPFTFGSGTKLEINR

SEQ ID NO: 69 - protein, 107 aa, Artificial Sequence
  Description of Artificial Sequence Synthetic polypeptide
DIQMTQSPSSLSASVGDRVTITCSASSSVSYMNWYQQKPGKAPKCWIYDTSKLASGVPSRFSGSGSGTDFTLTISSLQPEDFATYYCQQWSSNPFTFGSGTKLEINR

SEQ ID NO: 70 - protein, 107 aa, Artificial Sequence
  Description of Artificial Sequence Synthetic polypeptide
DIQMTQSPSSLSASVGDRVTITCSASSSVSYMNWYQQKPGKAPKCWIYDTSKLASGVPSRFSGSGSGTDYTLTISSLQPEDFATYYCQQWSSNPFTFGSGTKLEINR

SEQ ID NO: 71 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 72 - protein, 330 aa, Homo sapiens [sequence omitted]

SEQ ID NO: 73 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 74 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 75 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 119 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSS

SEQ ID NO: 78 - protein, 110 aa, Artificial Sequence
  Description of Artificial Sequence Synthetic polypeptide
QAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 79 - protein, 244 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 80 - protein, 259 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 81 - protein, 484 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 82 - protein, 449 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 487 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 84 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 487 aa, Artificial Sequence
  Description of Artificial Sequence Synthetic polypeptide
DIQMTQSQSSLSTSVGDRVTITCKASQNVRTVVAWYQQKPGKSPKTLIYLASNRHTGVPSRFSGSGSGTEFTLTISNVQPEDFADYFCLQHWSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 86 - protein, 447 aa, Artificial Sequence
  Description of Artificial Sequence Synthetic polypeptide
EVQLVESGGGLVKPGGSLRLSCAASGFAFSTYDMSWVRQAPGKRLEWVATISSGGSYTYYLDSVKGRFTISRDNAKNSLYLQMNSLRAEDTAVYYCAPTTVVPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 493 aa, Artificial Sequence
  Description of Artificial Sequence Synthetic polypeptide
DIVMTQSPDSLAVSVGERVTMNCKSSQSLLYRSNQKNYLAWYQQKPGQPPKLLIYWASTRESGVPDRFSGSGSGTDFTLTISSVQAEDVAVYYCQQYYNYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 88 - protein, 454 aa, Artificial Sequence
  Description of Artificial Sequence Synthetic polypeptide
DVQVQESGPGLVKPSQTLSLTCTVTGYSITSDYAWNWIRQPPGKGLEWMGYISNSGSTSYNPSLKSRITISRDTSKNQFSLKLSSVTAADTAVYYCARERNYDYDDYYYAMDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 491 aa, Artificial Sequence
  Description of Artificial Sequence Synthetic polypeptide
EIVLTQSPATLSVSLGERATISCRASESVDNYGISFMNWFQQKPGQPPRLLIYAASNQGSGVPARFSGSGPGTDFTLTISSMEPEDFAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 90 - protein, 446 aa, Artificial Sequence
  Description of Artificial Sequence Synthetic polypeptide
EVQLVQSGPEVVKPGASVKISCKASGYTFTDYNMHWVRQAHGQSLEWIGYIYPYNGGTGYNQKFKSRATLTVDNSASTAYMEVSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 487 aa, Artificial Sequence
  Description of Artificial Sequence Synthetic polypeptide
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 94 - protein, 448 aa, Artificial Sequence
  Description of Artificial Sequence Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 487 aa, Artificial Sequence
  Description of Artificial Sequence Synthetic polypeptide
EIVLTQSPATLSVSPGETVTLSCRASQTIYKNLHWYQQKSGLSPRLLIKYGSDSISGIPARFSGSGSGTDYTLTINSVEPEDEGIYYCLQGYSTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 96 - protein, 443 aa, Artificial Sequence
  Description of Artificial Sequence Synthetic polypeptide
QVQLVQSGPEVKKPGASVKISCKASGYTFTDYSMHWVKKAPGQGLKWLGWINTATGEPTYADDFKGRFTITLDTSARTVYLEISSLRSEDTATYFCFSYYDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 493 aa, Artificial Sequence
  Description of Artificial Sequence Synthetic polypeptide
DIVMSQSPSSLAVSVGEKVTMSCKSSQSLLYSSNQKNYLAWYQQKPGQSPKLLIYWASTRESGVPDRFTGSGSGTDFTLTISSVKAEDLALYYCQQYHSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 98 - protein, 448 aa, Artificial Sequence
  Description of Artificial Sequence Synthetic polypeptide
QVQLVQPGDELVKPGASVKLSCKASGYTFTSYWMQWVKQRPGQGLEWIGEINPSNGRTNYNEMFKSKAVLSVDKSVSTAYMQLSSLTAEDTAVYYCALYDGYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 492 aa, Artificial Sequence
  Description of Artificial Sequence Synthetic polypeptide
DIQMTQSPSSLSASVGDRVTITCRSSQRIVHSNGNTYLEWYQQTPGKAPKLLIYKVSNRFSGVPSRFSGSGSGTDFTFTISSLQPEDIATYYCFQGSHVPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 100 - protein, 449 aa, Artificial Sequence
  Description of Artificial Sequence Synthetic polypeptide
EVQLVESGGGVVQPGRSLRLSCSTSGFTFSDYYMYWVRQAPGKGLEWVAYMSNVGAITDYPDTVKGRFTISRDNSKNTLFLQMDSLRPEDTGVYFCARGTRDGSWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 501 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRGGGGSGGGGSGGGGSGGGGSQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRHHHHHH

SEQ ID NO: 102 - protein, 211 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 449 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 104 - protein, 213 aa, Artificial Sequence
  Description of Artificial Sequence Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 449 aa, Artificial Sequence
  Description of Artificial Sequence Synthetic polypeptide
QVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 106 - protein, 211 aa, Artificial Sequence; identical to SEQ ID NO: 102
  Description of Artificial Sequence Synthetic polypeptide

SEQ ID NO: 107 - protein, 449 aa, Artificial Sequence; identical to SEQ ID NO: 82
  Description of Artificial Sequence Synthetic polypeptide

SEQ ID NO: 108 - protein, 213 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 109 - protein, 450 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 211 aa, Artificial Sequence; identical to SEQ ID NO: 102
  Description of Artificial Sequence Synthetic polypeptide

SEQ ID NO: 111 - protein, 721 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 112 - protein, 550 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRGGGGSGGGGSGGGGSGGGGSQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTPLGDTTHTSGMVSKLSQLQTELLAALLESGLSKEALIQALGEGSGGAPHHHHHH

SEQ ID NO: 113 - protein, 742 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRGGGGSGGGGSGGGGSGGGGSQVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGGGGSQVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGGGGSGGGGSGGGGSGGGGPDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 484 aa, Artificial Sequence; identical to SEQ ID NO: 81
  Description of Artificial Sequence Synthetic polypeptide

SEQ ID NO: 115 - protein, 449 aa, Artificial Sequence
  Description of Artificial Sequence Synthetic polypeptide
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 116 - protein, 487 aa, Artificial Sequence
  Description of Artificial Sequence Synthetic polypeptide
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 117 - protein, 449 aa, Artificial Sequence; identical to SEQ ID NO: 103
  Description of Artificial Sequence Synthetic polypeptide

SEQ ID NO: 118 - protein, 487 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 119 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 120 - protein, 490 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 121 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 122 - protein, 493 aa, Artificial Sequence
  Description of Artificial Sequence Synthetic polypeptide
DIVMTQTPPSLPVNPGEPASISCRSSQSLVHRNGNTYLHWYLQKPGQSPQLLIHKVSNRFSGVPDRFSGSGSGSDFTLKISWVEAEDVGVYYCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 123 - protein, 443 aa, Artificial Sequence
  Description of Artificial Sequence Synthetic polypeptide
QVQLLQSGAEVKKPGASVKISCKASGSSFTGYNMNWVRQAPGQGLEWIGAIDPYYGGTSYNQKFKGRATLTVDKSISTAYMELSRLRSDDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 124 - protein, 496 aa, Artificial Sequence
  Description of Artificial Sequence Synthetic polypeptide
DIVMTQTPPSLPVNPGEPASISCRSSQSLVHRNGNTYLHWYLQKPGQSPQLLIHKVSNRFSGVPDRFSGSGSGSDFTLKISWVEAEDVGVYYCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 125 - protein, 443 aa, Artificial Sequence
  Description of Artificial Sequence Synthetic polypeptide
QVQLLQSGAEVKKPGASVKISCKASGSSFTGYNMNWVRQAPGQGLEWIGAIDPYYGGTSYNQKFKGRATLTVDKSISTAYMELSRLRSDDTAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 126 - protein, 487 aa, Artificial Sequence; identical to SEQ ID NO: 85
  Description of Artificial Sequence Synthetic polypeptide

SEQ ID NO: 127 - protein, 447 aa, Artificial Sequence
  Description of Artificial Sequence Synthetic polypeptide
EVQLVESGGGLVKPGGSLRLSCAASGFAFSTYDMSWVRQAPGKRLEWVATISSGGSYTYYLDSVKGRFTISRDNAKNSLYLQMNSLRAEDTAVYYCAPTTVVPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 128 - protein, 490 aa, Artificial Sequence
  Description of Artificial Sequence Synthetic polypeptide
DIQMTQSQSSLSTSVGDRVTITCKASQNVRTVVAWYQQKPGKSPKTLIYLASNRHTGVPSRFSGSGSGTEFTLTISNVQPEDFADYFCLQHWSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 129 - protein, 447 aa, Artificial Sequence
  Description of Artificial Sequence Synthetic polypeptide
EVQLVESGGGLVKPGGSLRLSCAASGFAFSTYDMSWVRQAPGKRLEWVATISSGGSYTYYLDSVKGRFTISRDNAKNSLYLQMNSLRAEDTAVYYCAPTTVVPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 130 - protein, 493 aa, Artificial Sequence
  Description of Artificial Sequence Synthetic polypeptide
DIVMTQSPSSLVVSIGERVTMNCKSSQSLLYSSNQKNYLAWYQQKPGQSPKLLIYWASSRESGVPDRFSGSGSGTDFTLTISSVKAEDVAVYYCQQYYNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 131 - protein, 447 aa, Artificial Sequence
  Description of Artificial Sequence Synthetic polypeptide
EVQLVESGGGLVQPEGSLKLSCAASGFTFNKNAMNWVRQAPGKGLEWVARIRNKTNNYATYYADSVKARFTISRDDSQSMLYLQMNSLKIEDTAMYYCVAGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 132 - protein, 496 aa, Artificial Sequence
  Description of Artificial Sequence Synthetic polypeptide
DIVMTQSPSSLVVSIGERVTMNCKSSQSLLYSSNQKNYLAWYQQKPGQSPKLLIYWASSRESGVPDRFSGSGSGTDFTLTISSVKAEDVAVYYCQQYYNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 133 - protein, 447 aa, Artificial Sequence
  Description of Artificial Sequence Synthetic polypeptide
EVQLVESGGGLVQPEGSLKLSCAASGFTFNKNAMNWVRQAPGKGLEWVARIRNKTNNYATYYADSVKARFTISRDDSQSMLYLQMNSLKIEDTAMYYCVAGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 134 - protein, 493 aa, Artificial Sequence; identical to SEQ ID NO: 87
  Description of Artificial Sequence Synthetic polypeptide

SEQ ID NO: 135 - protein, 454 aa, Artificial Sequence
  Description of Artificial Sequence Synthetic polypeptide
DVQVQESGPGLVKPSQTLSLTCTVTGYSITSDYAWNWIRQPPGKGLEWMGYISNSGSTSYNPSLKSRITISRDTSKNQFSLKLSSVTAADTAVYYCARERNYDYDDYYYAMDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 136 - protein, 496 aa, Artificial Sequence
  Description of Artificial Sequence Synthetic polypeptide
DIVMTQSPDSLAVSVGERVTMNCKSSQSLLYRSNQKNYLAWYQQKPGQPPKLLIYWASTRESGVPDRFSGSGSGTDFTLTISSVQAEDVAVYYCQQYYNYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 137 - protein, 454 aa, Artificial Sequence
  Description of Artificial Sequence Synthetic polypeptide
DVQVQESGPGLVKPSQTLSLTCTVTGYSITSDYAWNWIRQPPGKGLEWMGYISNSGSTSYNPSLKSRITISRDTSKNQFSLKLSSVTAADTAVYYCARERNYDYDDYYYAMDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 138 - protein, 487 aa, Artificial Sequence
  Description of Artificial Sequence Synthetic polypeptide
DIQMTQSPSSLSASVGDRVTITCQASQDISKYLNWYQQKPGKAVKLLIYHTSRLHSGVPSRFSGSGSGTDYTLTISSLQPEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGQGTTLTVSSGGGGSGGGGSGGGGSGGGGSGGGGSGGGGSDIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGSGTKLEINR

SEQ ID NO: 139 - protein, 450 aa, Artificial Sequence
  Description of Artificial Sequence Synthetic polypeptide
QVQLQESGPGLVKPSETLSVTCTVSGVSLPDYGVSWIRQPPGKGLEWIGVIWGSETTYYNPSLKSRVTISVDTSKNQVSLKLSSVTAADTAVYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 140 - protein, 490 aa, Artificial Sequence
  Description of Artificial Sequence Synthetic polypeptide
DIQMTQSPSSLSASVGDRVTITCQASQDISKYLNWYQQKPGKAVKLLIYHTSRLHSGVPSRFSGSGSGTDYTLTISSLQPEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 141 - protein, 450 aa, Artificial Sequence
  Description of Artificial Sequence Synthetic polypeptide
QVQLQESGPGLVKPSETLSVTCTVSGVSLPDYGVSWIRQPPGKGLEWIGVIWGSETTYYNPSLKSRVTISVDTSKNQVSLKLSSVTAADTAVYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 142 - protein, 491 aa, Artificial Sequence; identical to SEQ ID NO: 89
  Description of Artificial Sequence Synthetic polypeptide

SEQ ID NO: 143 - protein, 446 aa, Artificial Sequence
  Description of Artificial Sequence Synthetic polypeptide
EVQLVQSGPEVVKPGASVKISCKASGYTFTDYNMHWVRQAHGQSLEWIGYIYPYNGGTGYNQKFKSRATLTVDNSASTAYMEVSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 144 - protein, 494 aa, Artificial Sequence
  Description of Artificial Sequence Synthetic polypeptide
EIVLTQSPATLSVSLGERATISCRASESVDNYGISFMNWFQQKPGQPPRLLIYAASNQGSGVPARFSGSGPGTDFTLTISSMEPEDFAMYFCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 145 - protein, 446 aa, Artificial Sequence
  Description of Artificial Sequence Synthetic polypeptide
EVQLVQSGPEVVKPGASVKISCKASGYTFTDYNMHWVRQAHGQSLEWIGYIYPYNGGTGYNQKFKSRATLTVDNSASTAYMEVSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 146 - protein, 486 aa, Artificial Sequence
  Description of Artificial Sequence Synthetic polypeptide
DIQMTQSPSSLSASVGDRVTITCKASQDINKYIAWYQHKPGKAPKLLIYYASNLQPGVPSRFSGSGSGRDFTFTISSLQPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLQQSGAEVAKPGASVKVSCKASGYTFTRYTMHWVRQAPGQGLEWMGYINPSRGYTNYNQKFKDRATLTRDKSISTAYMELSRLRSDDTAVYYCARYYDDHYSLDYWGQGTTLTVSSGGGGSGGGGSGGGGSGGGGSGGGGSGGGGSDIQMTQSPSSLSASVGDRVTITCSASSSVSYMNWYQQKPGKAPKRWIYDTSKLASGVPSRFSGSGSGTDFTLTISSLQPEDFATYYCQQWSSNPFTFGSGTKLEINR

SEQ ID NO: 147 - protein, 449 aa, Artificial Sequence
  Description of Artificial Sequence Synthetic polypeptide
QVQLQQSGAEVVKPGASVKVSCKASGYSFTDYNMYWVRQAPGQGLEWMGYIDPYKGGTIYNQKFQGRATLTRDTSISTAYMELSRLRSDDTAVYYCAREMITAYYFDYWGQGSSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 148 - protein, 489 aa, Artificial Sequence
  Description of Artificial Sequence Synthetic polypeptide
DIQMTQSPSSLSASVGDRVTITCKASQDINKYIAWYQHKPGKAPKLLIYYASNLQPGVPSRFSGSGSGRDFTFTISSLQPEDIATYYCLQYDNLLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 149 - protein, 449 aa, Artificial Sequence
  Description of Artificial Sequence Synthetic polypeptide
QVQLQQSGAEVVKPGASVKVSCKASGYSFTDYNMYWVRQAPGQGLEWMGYIDPYKGGTIYNQKFQGRATLTRDTSISTAYMELSRLRSDDTAVYYCAREMITAYYFDYWGQGSSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 150 - protein, 492 aa, Artificial Sequence
  Description of Artificial Sequence Synthetic polypeptide
DIVMTQSPDSLAVSLGERATINCKSSQSVFFSSSQKNYLAWYQQKPGQPPKLLIYWASTRESGVPDRFSGSGSGTDFTLTISSLQAEDVAVYYCHQYLSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 151 - protein, 448 aa, Artificial Sequence
  Description of Artificial Sequence Synthetic polypeptide
QVQLVQSGAEVKKPGASVKVSCKASGYTFTSYYIHWVRQAPGQGLEWMGVIYPGNDDISYAQKFQGRVTITADKSATTAYMELSSLRSEDTAVYYCAREVRLRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 152 - protein, 495 aa, Artificial Sequence
  Description of Artificial Sequence Synthetic polypeptide
DIVMTQSPDSLAVSLGERATINCKSSQSVFFSSSQKNYLAWYQQKPGQPPKLLIYWASTRESGVPDRFSGSGSGTDFTLTISSLQAEDVAVYYCHQYLSSRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 153 - protein, 448 aa, Artificial Sequence
  Description of Artificial Sequence Synthetic polypeptide
QVQLVQSGAEVKKPGASVKVSCKASGYTFTSYYIHWVRQAPGQGLEWMGVIYPGNDDISYAQKFQGRVTITADKSATTAYMELSSLRSEDTAVYYCAREVRLRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 154 - protein, 487 aa, Artificial Sequence; identical to SEQ ID NO: 93
  Description of Artificial Sequence Synthetic polypeptide

SEQ ID NO: 155 - protein, 448 aa, Artificial Sequence
  Description of Artificial Sequence Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 156 - protein, 490 aa, Artificial Sequence
  Description of Artificial Sequence Synthetic polypeptide
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 157 - protein, 448 aa, Artificial Sequence
  Description of Artificial Sequence Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 158 - protein, 15 aa, Artificial Sequence
  Description of Artificial Sequence Synthetic peptide
GGGGSGGGGSGGGGS

SEQ ID NO: 159 - protein, 20 aa, Artificial Sequence
  Description of Artificial Sequence Synthetic peptide
GGGGSGGGGSGGGGSGGGGS

SEQ ID NO: 160 - protein, 25 aa, Artificial Sequence
  Description of Artificial Sequence Synthetic peptide
GGGGSGGGGSGGGGSGGGGSGGGGS

SEQ ID NO: 161 - protein, 30 aa, Artificial Sequence
  Description of Artificial Sequence Synthetic polypeptide
GGGGSGGGGSGGGGSGGGGSGGGGSGGGGS

SEQ ID NO: 162 - protein, 487 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQAYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 163 - protein, 450 aa, Artificial Sequence; identical to SEQ ID NO: 119
  Description of Artificial Sequence Synthetic polypeptide

SEQ ID NO: 164 - protein, 490 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQAYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 165 - protein, 450 aa, Artificial Sequence; identical to SEQ ID NO: 121
  Description of Artificial Sequence Synthetic polypeptide

SEQ ID NO: 166 - protein, 486 aa, Artificial Sequence
  Description of Artificial Sequence Synthetic polypeptide
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSQVQLVQSGGGVVQPGRSLRLSCKASGYTFTRYTMHWVRQAPGKCLEWIGYINPSRGYTNYNQKFKDRFTISRDNSKNTAFLQMDSLRPEDTGVYFCARYYDDHYSLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGCGTKLQITR

SEQ ID NO: 167 - protein, 449 aa, Artificial Sequence
  Description of Artificial Sequence Synthetic polypeptide
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 168 - protein, 489 aa, Artificial Sequence
  Description of Artificial Sequence Synthetic polypeptide
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 169 - protein, 449 aa, Artificial Sequence
  Description of Artificial Sequence Synthetic polypeptide
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSRLTVDKSRWQQGNVFSCSVMHEALHNHYTQKSLSLSPGK